(19) United States
(12) Patent Application Publication
(10) Pub. No.: US 2026/0337196 A1
(43) Pub. Date: Oct. 29, 2026

(54) TRIAZOLOPYRIDINYL ETHER LINKED COMPOUNDS AS KINASE INHIBITORS

(71) Applicant: Bristol-Myers Squibb Company, Princeton, NJ (US)

(72) Inventors: Guanglin Luo, Newtown, PA (US); David B. Frennesson, Naugatuck, CT (US); Jeffrey Michael Schkeryantz, Harvard, MA (US); Jianliang Shi, Furlong, PA (US); Brian Lee Venables, Durham, CT (US); Karin Irmgard Worm, Jackson, NJ (US); Yong-Jin Wu, Madison, CT (US); Murali T. G. Dhar, Newtown, PA (US)

(21) Appl. No.: 19/576,108

(22) Filed: Mar. 24, 2026

Related U.S. Application Data
(63) Continuation of application No. PCT/US2024/048494, filed on Sep. 26, 2024.
(60) Provisional application No. 63/585,642, filed on Sep. 27, 2023.

(51) Int. Cl. C07D 471/04; A61K 31/437; A61P 25/28

(52) U.S. Cl. CPC C07D 471/04; A61K 31/437; A61P 25/28

(57) ABSTRACT

Compounds having formula (I), and enantiomers, and diastereomers, stereoisomers, pharmaceutically-acceptable salts thereof,

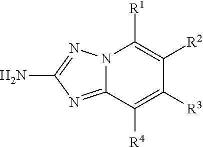

are useful as kinase modulators, including RIPK1 modulation. All the variables are as defined herein.

### CROSS-REFERENCE TO RELATED APPLICATIONS

This application claims the benefit of U.S. Provisional Application No. 63/585,642 filed Sep. 27, 2023, the disclosure of which are incorporated herein by reference in its entirety.

### FIELD OF THE INVENTION

The present invention relates to novel compounds that inhibit receptor interacting protein kinases and methods of making and using the same. Specifically, the present invention relates to triazolopyridinyl compounds as receptor interacting protein kinase 1 (RIPK1) inhibitors.

### BACKGROUND OF THE INVENTION

Apoptosis and necrosis represent two different mechanisms of cell death. Apoptosis is a highly regulated process involving the caspase family of cysteine proteases, and characterized by cellular shrinkage, chromatin condensation, and DNA degradation. In contrast, necrosis is associated with cellular and organelle swelling and plasma membrane rupture with ensuing release of intracellular contents and secondary inflammation (Kroemer et al., (2009) Cell Death Differ 16:3-11). Necrosis has been considered a passive, unregulated form of cell death; however, recent evidence indicates that some necrosis can be induced by regulated signal transduction pathways such as those mediated by receptor interacting protein kinases (RIPKs) especially in conditions where caspases are inhibited or cannot be activated efficiently (Golstein P & Kroemer G (2007) Trends Biochem. Sci. 32:37-43; Festjens et al. (2006) Biochim. Biophys. Acta 1757:1371-1387). Stimulation of the Fas and TNFR family of death domain receptors (DRs) is known to mediate apoptosis in most cell types through the activation of the extrinsic caspase pathway. In addition, in certain cells deficient for caspase-8 or treated with pan-caspase inhibitor Z-VAD, stimulation of death domain receptors (DR) causes a receptor interacting protein kinase 1 (RIPK1) dependent programmed necrotic cell death instead of apoptosis (Holler et al. (2000) Nat. Immunol. 1:489-495; Degterev et al. (2008) Nat. Chem. Biol. 4:313-321). This novel mechanism of cell death is termed “programmed necrosis” or “necroptosis” (Degterev et al., (2005) Nat Chem Biol 1:112-119).

Necroptosis can be triggered by a number of mechanisms including of TNF receptor activation, Toll-like receptor engagement, genotoxic stress and viral infection. Downstream of the various stimuli, the signaling pathway that results in necroptosis is dependent on RIPK1 and RIPK3 kinase activity. (He et al., (2009) Cell 137:1100-1111; Cho et. al., (2009) Cell 137:1112-1123; Zhang et al., (2009) Science 325:332-336).

Dysregulation of the necroptosis signaling pathway has been linked to neuroinflammation and neurodegeneration such as multiple sclerosis (MS), amyotrophic lateral sclerosis (ALS), Parkinson's and Alzheimer's disease, spinal cord injury, and traumatic brain injury (TBI), detachment of the retina, ischemia, Gaucher's disease, and AAV (ANCA-associated vasculitis) and other inflammatory diseases such as macrophage necrosis in atheroscelerosis development, virus-induced inflammation, systemic inflammatory response syndrome and ethanol-induced liver injury—(Yan et al., (2019) Nature Rev. Neuro. 20, 19-33; Trichonas et al., (2010) Proc. Natl. Acad. Sci. 107, 21695-21700; Lin et al., (2013) Cell Rep. 3, 200-210; Cho et al., (2009) Cell, 137, 1112-1123; Duprez et al., (2011) Immunity 35, 908-918; Roychowdhury et al., Hepatology 57, 1773-1783; Vandenabeele et al., (2010) Nature 10, 700-714; Vandenabeele et al., (2010) Sci. Signalling 3, 1-8; Zhang et al., (2010) Cellular & Mol. Immunology 7, 243-249; Moriwaki et al., (2013) Genes Dev. 27, 1640-1649; Ito et al., (2016) Science 353, 603-608; Vitner et al., (2014) Nature Med. 20, 204-208) (Schreiber et al., (2017) Proc. Natl. Acad. Sci. 114, E9618-E9625). To be useful in the treatment of neurodegenerative diseases, compounds should preferably be brain penetrant. Brain penetrance can be predicted by a brain transporter assay and measured through direct determination of brain concentration.

A potent, selective, small molecule inhibitor of RIPK1 activity would block RIPK1-dependent pro-inflammatory signaling and thereby provide a therapeutic benefit in inflammatory diseases characterized by increased and/or dysregulated RIPK1 kinase activity.

WO2022/086828 discloses triazolopyridinyl compounds having RIPK1 activity.

The present invention is directed to compounds having RIPK1 activity and which are brain penetrant, and therefore are useful in the treatment of diseases modulated by RIPK1 kinase activity, and useful in the treatment of neurodegenerative diseases.

### SUMMARY OF THE INVENTION

The present invention provides novel triazolopyridinyl compounds including stereoisomers, tautomers, isotopes, prodrugs, pharmaceutically acceptable salts, salts, or solvates thereof, which are useful as inhibitors of RIPK1.

The present invention also provides processes and intermediates for making the compounds of the present invention.

The present invention also provides pharmaceutical compositions comprising a pharmaceutically acceptable carrier and at least one of the compounds of the present invention or stereoisomers, tautomers, isotopes, prodrugs, pharmaceutically acceptable salts, salts, or solvates thereof.

The compounds of the invention may be used in the treatment and/or prophylaxis of conditions associated with aberrant RIPK1 activity.

The compounds of the present invention may be used in therapy.

The compounds of the present invention may be used for the manufacture of a medicament for the treatment and/or prophylaxis of a condition associated with aberrant RIPK1 activity.

In another aspect, the present invention is directed to a method of treating diseases mediated at least partially by RIPK1 including inflammatory diseases and neurological inflammatory diseases, which method comprises administering to a patient in need of such treatment a compound of the present invention as described above.

In another aspect, the present invention is directed to a method of treating diseases mediated at least partially by RIPK1 including inflammatory diseases and neurological inflammatory diseases, such as multiple sclerosis (MS), amyotrophic lateral sclerosis (ALS), Parkinson's and Alzheimer's disease, spinal cord injury, and traumatic brain injury (TBI), detachment of the retina, ischemia, Gaucher's disease, and AAV (ANCA-associated vasculitis) and other inflammatory diseases such as macrophage necrosis in atheroscelerosis development, virus-induced inflammation, systemic inflammatory response syndrome and ethanol-induced liver injury, which method comprises administering to a patient in need of such treatment a compound of the present invention as described above.

The compounds of the invention can be used alone, in combination with other compounds of the present invention, or in combination with one or more, or with one to two other agent(s).

These and other features of the invention will be set forth in expanded form as the disclosure continues.

### DETAILED DESCRIPTION OF EMBODIMENTS OF THE INVENTION

In one aspect, the present invention provides, inter alia, compounds of Formula (I) or stereoisomers, tautomers, isotopes, salts, pharmaceutically acceptable salts, solvates, or prodrugs thereof, wherein

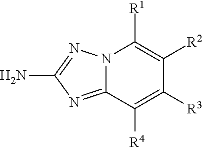

wherein, independently for each occurrence:
R¹ is H or F, C₁₋₄ alkyl, C₁₋₄ alkoxy, or C₃₋₆ cycloalkyl;
one of R² or R³ is H and the other is

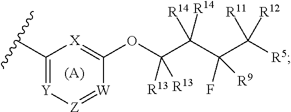

X is N or C(R⁸);
Y is N or C(R⁶)
Z is N or C(R⁷);
W is N or C(R⁹);
wherein ring A contains 0, 1, 2, or 3 nitrogen atoms;
R⁴ is H or F, C₁₋₄ alkyl, C₃₋₆ cycloalkyl, or C₁₋₃ alkoxy;
R⁵ is C₁₋₆ alkyl, phenyl or pyridyl, wherein the phenyl or pyridyl are substituted with 0-1 R¹⁰;
R⁶ is —H, —F, —Cl, —C₁₋₃ alkyl, —CF₃, or C₁₋₃ alkoxy;
R⁷ is —H, —F, —Cl, —C₁₋₃ alkyl, —CF₃, or CHF₂;
R⁸ is —H, or —F, or C₁₋₃ alkyl or C₁₋₃ alkoxy;
R⁹ is —H, C₁₋₃ alkyl, —F, —Cl, —OCH₃, —CN, —CF₃, —OCF3, or —CHF₂;
R¹⁰ is —H, —F, or —Cl or —C₁₋₃ alkyl, or C₁₋₃ alkoxy;
R¹¹ is —H, —NH₂, —OH, or —F;
R¹² is —H, —C₁₋₃ alkyl, or —F;
R¹³ is —H or -D, or —C₁₋₃ alkyl; and
R¹⁴ is —H, -D, or —C₁₋₃ alkyl.

Another embodiment provides a compound of Formula (I), or stereoisomers, tautomers, isotopes, salts, pharmaceutically acceptable salts, solvates, or prodrugs thereof, wherein
X and Z are N;
Y is C(R⁶); and
W is C(R⁹).

Another embodiment provides a compound of Formula (I), or any other aspect or embodiment, or stereoisomers, tautomers, isotopes, salts, pharmaceutically acceptable salts, solvates, or prodrugs thereof, wherein
X is N;
Y is C(R⁶);
Z is C(R⁷); and
W is C(R⁹).

Another embodiment provides a compound of Formula (I), or any other aspect or embodiment, or stereoisomers, tautomers, isotopes, salts, pharmaceutically acceptable salts, solvates, or prodrugs thereof, wherein
X is C(R⁸)
Y is C(R⁶);
Z is C(R⁷); and
W is C(R⁹).

Another embodiment provides a compound of Formula (I), or any other aspect or embodiment, or stereoisomers, tautomers, isotopes, salts, pharmaceutically acceptable salts, solvates, or prodrugs thereof, wherein
R² is H; and
R³ is

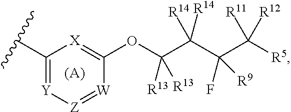

Another embodiment provides a compound of Formula (I), or any other aspect or embodiment, or stereoisomers, tautomers, isotopes, salts, pharmaceutically acceptable salts, solvates, or prodrugs thereof, wherein
R⁵ is phenyl or pyridyl, wherein the phenyl or pyridyl are substituted with 0-1 R¹⁰

Another embodiment provides a compound of Formula (I), or any other aspect or embodiment, or stereoisomers, tautomers, isotopes, salts, pharmaceutically acceptable salts, solvates, or prodrugs thereof, wherein
R¹⁴ is H or D.

Another embodiment provides a compound of Formula (I), or any other aspect or embodiment, or stereoisomers, tautomers, isotopes, salts, pharmaceutically acceptable salts, solvates, or prodrugs thereof, wherein
R¹ and R⁴ are H;
R⁵ is phenyl substituted with 0-1 R¹⁰;
R¹³ is H; and
R¹⁴ is H.

Another embodiment provides a compound of Formula (I), or any other aspect or embodiment, or stereoisomers, tautomers, isotopes, salts, pharmaceutically acceptable salts, solvates, or prodrugs thereof, wherein
R⁸ is F;
R⁹ is F;
R¹¹ is —OH; and
R¹² is H.

Another embodiment provides a compound of Formula (I), or any other aspect or embodiment, or stereoisomers, tautomers, isotopes, salts, pharmaceutically acceptable salts, solvates, or prodrugs thereof, wherein
R⁸ is F;
R⁹ is F;
R¹¹ is —OH; and
R¹² is C₁₋₃ alkyl.

Another embodiment provides a compound of Formula (I), or any other aspect or embodiment, or stereoisomers, tautomers, isotopes, salts, pharmaceutically acceptable salts, solvates, or prodrugs thereof, wherein
R⁸ is F;
R⁹ is F;
R¹¹ is —OH; and
R¹² is CH₃.

Another embodiment provides a compound of Formula (I), or any other aspect or embodiment, or stereoisomers, tautomers, isotopes, salts, pharmaceutically acceptable salts, solvates, or prodrugs thereof, wherein
X is C(R⁸);
Y is CH;
Z is CH;
W is C(R⁹);
R⁸ is F;
R⁹ is F;
R¹¹ is —OH; and
R¹² is CH₃.

Another embodiment provides a compound of Formula (I), or any other aspect or embodiment, or stereoisomers, tautomers, isotopes, salts, pharmaceutically acceptable salts, solvates, or prodrugs thereof, wherein the compound of Formula (I) is:

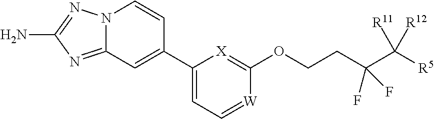

and
R¹¹ is —OH; and
R¹² is CH₃.

Another embodiment provides a compound of Formula (I), or any other aspect or embodiment, or stereoisomers, tautomers, isotopes, salts, pharmaceutically acceptable salts, solvates, or prodrugs thereof, wherein the compound of Formula (I) is:

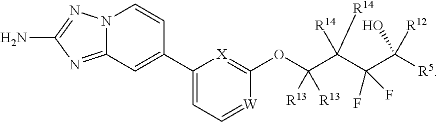

Another embodiment provides a compound of Formula (I), or any other aspect or embodiment, or stereoisomers, tautomers, isotopes, salts, pharmaceutically acceptable salts, solvates, or prodrugs thereof, wherein the compound of Formula (I) is:

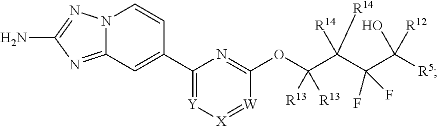

Y is C(R⁶);
Z is C(R⁷); and
W is C(R⁹).

Another embodiment provides a compound of Formula (I), or any other aspect or embodiment, or stereoisomers, tautomers, isotopes, salts, pharmaceutically acceptable salts, solvates, or prodrugs thereof, wherein the compound of Formula (I) is:

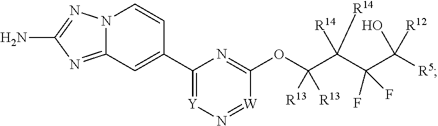

Y is C(R⁶); and
W is C(R⁹).

Another embodiment provides a compound of Formula (I), or any other aspect or embodiment, or stereoisomers, tautomers, isotopes, salts, pharmaceutically acceptable salts, solvates, or prodrugs thereof, wherein
W is CR^{9a};
R^{9a} is —H, C₁₋₃ alkyl, —F, —Cl, —OCH₃, —CN, —CF₃, —OCF3, or —CHF₂;
R⁹ is —H, or —F.

Another embodiment provides a compound of Formula (I), or any other aspect or embodiment, or stereoisomers, tautomers, isotopes, salts, pharmaceutically acceptable salts, solvates, or prodrugs thereof, wherein
W is CR^{9a};
R^{9a} is —H, C₁₋₃ alkyl, or —F; and
R⁹ is —H, or —F.

Another embodiment provides a compound of Formula (I), or any other aspect or embodiment, or stereoisomers, tautomers, isotopes, salts, pharmaceutically acceptable salts, solvates, or prodrugs thereof, wherein
W is CR^{9a};
R^{9a} is —H, or —F; and
R⁹ is —H, or —F.

Another embodiment provides a compound of Formula (I), or stereoisomers, tautomers, isotopes, salts, pharmaceutically acceptable salts, solvates, or prodrugs thereof, wherein the compound is selected from the examples.

The present invention is also directed to pharmaceutical compositions useful in treating diseases associated with kinase modulation, including the modulation of receptor interacting protein kinases such as RIPK1, comprising compounds of formula (I), or pharmaceutically-acceptable salts thereof, and pharmaceutically-acceptable carriers or diluents.

The invention further relates to methods of treating diseases associated with kinase modulation, including the modulation of receptor interacting protein kinases such as RIPK1, comprising administering to a patient in need of such treatment a therapeutically-effective amount of a compound according to formula (I) or pharmaceutically acceptable salt thereof.

The present invention also provides processes and intermediates for making the compounds of the present invention or stereoisomers, tautomers, pharmaceutically acceptable salts, solvates, or prodrugs thereof.

The present invention also provides a method for treating proliferative diseases, allergic diseases, autoimmune diseases and inflammatory diseases and fibrotic diseases, comprising administering to a host in need of such treatment a therapeutically effective amount of at least one of the compounds of the present invention or stereoisomers, tautomers, pharmaceutically acceptable salts, solvates, or prodrugs thereof.

The present invention also provides a method for treating a disease, comprising administering to a patient in need of such treatment a therapeutically-effective amount of a compound of formula (I), or pharmaceutically acceptable salt thereof, wherein the disease is inflammatory bowel disease, Crohn's disease or ulcerative colitis, poriasis, systemic lupus erythematosus (SLE), rheumatoid arthritis, multiple sclerosis (MS), alzheimer's disease (AD), amyotrophic lateral sclerosis (ALS), transplant rejection, nonalcoholic steatohepatitis (NASH), or ischemia reperfusion.

The present invention also provides a method of treating a condition comprising administering to a patient in need of such treatment a therapeutically-effective amount of a compound of formula (I), or pharmaceutically acceptable salt thereof, wherein the condition is selected from systemic lupus erythematosus (SLE), multiple sclerosis (MS), alzheimer's disease (AD), amyotrophic lateral sclerosis (ALS), transplant rejection, acute myelogenous leukemia, chronic myelogenous leukemia, metastatic melanoma, Kaposi's sarcoma, multiple myeloma, solid tumors, ocular neovasculization, and infantile haemangiomas, B cell lymphoma, systemic lupus erythematosus (SLE), psoriatic arthritis, multiple vasculitides, idiopathic thrombocytopenic purpura (ITP), myasthenia gravis, allergic rhinitis, multiple sclerosis (MS), transplant rejection, Type I diabetes, membranous nephritis, autoimmune hemolytic anemia, autoimmune thyroiditis, cold and warm agglutinin diseases, Evan's syndrome, hemolytic uremic syndrome/thrombotic thrombocytopenic purpura (HUS/TTP), sarcoidosis, Sjogren's syndrome, peripheral neuropathies, pemphigus vulgaris and asthma, nonalcoholic steatohepatitis (NASH), or ischemia reperfusion.

The present invention also provides a method of treating a condition comprising administering to a patient in need of such treatment a therapeutically-effective amount of a compound of formula (I), or pharmaceutically acceptable salt thereof, wherein the condition is selected from macrophage necrosis in atheroscelerosis development, virus-induced inflammation, systemic inflammatory response syndrome and ethanol-induced liver injury, neurodegeneration such as detachment of the retina, retinal degeneration, wet and dry age-related macular degeneration (AMD), ischemia, amyotrophic lateral sclerosis (ALS), and Gaucher's disease.

The present invention also provides a method for treating a disease, comprising administering to a patient in need of such treatment a therapeutically-effective amount of a compound of formula (I), or pharmaceutically acceptable salt thereof, wherein the disease is multiple sclerosis (MS), amyotrophic lateral sclerosis (ALS), Parkinson's and Alzheimer's disease, spinal cord injury, and traumatic brain injury (TBI), detachment of the retina, ischemia, Gaucher's disease, and AAV (ANCA-associated vasculitis) and other inflammatory diseases such as macrophage necrosis in atheroscelerosis development, virus-induced inflammation, systemic inflammatory response syndrome and ethanol-induced liver injury.

The present invention also provides a method of treating a condition comprising administering to a patient in need of such treatment a therapeutically-effective amount of a compound of formula (I), or pharmaceutically acceptable salt thereof, wherein the condition is selected from multiple sclerosis (MS), alzheimer's disease (AD), amyotrophic lateral sclerosis (ALS), Parkinson's and Alzheimer's disease.

The present invention also provides a method of treating a condition comprising administering to a patient in need of such treatment a therapeutically-effective amount of a compound of formula (I), or pharmaceutically acceptable salt thereof, wherein the condition is selected from inflammatory bowel disease, Crohn's disease, ulcerative colitis, and psoriasis. In another embodiment, the condition is selected from inflammatory bowel disease, Crohn's disease, and ulcerative colitis.

The present invention also provides a method of treating a condition comprising administering to a patient in need of such treatment a therapeutically-effective amount of a compound of formula (I), or pharmaceutically acceptable salt thereof, wherein the condition is selected from nonalcoholic steatohepatitis (NASH), and ischemia reperfusion.

The present invention also provides a method for treating multiple sclerosis (MS), comprising administering to a patient in need of such treatment a therapeutically-effective amount of a compound of formula (I) or pharmaceutically acceptable salt thereof.

The present invention also provides a method for treating alzheimer's disease (AD), comprising administering to a patient in need of such treatment a therapeutically-effective amount of a compound of formula (I) or pharmaceutically acceptable salt thereof.

The present invention also provides a method for treating amyotrophic lateral sclerosis (ALS), comprising administering to a patient in need of such treatment a therapeutically-effective amount of a compound of formula (I) or pharmaceutically acceptable salt thereof.

The present invention also provides a method of treating diseases, comprising administering to a patient in need of such treatment a therapeutically-effective amount of a compound of formula (I), or pharmaceutically acceptable salt thereof, in combination with other therapeutic agents.

The present invention also provides the compounds of the present invention or stereoisomers, tautomers, isotopes, salts, pharmaceutically acceptable salts, solvates, or prodrugs thereof, for use in therapy.

In another embodiment, compounds of formula (I), or pharmaceutically acceptable salt thereof, are selected from exemplified examples or combinations of exemplified examples or other embodiments herein.

The present invention also provides the use of the compounds of the present invention or stereoisomers, tautomers, isotopes, salts, pharmaceutically acceptable salts, solvates, or prodrugs thereof, for the manufacture of a medicament for the treatment of cancers, an allergic disease, an autoimmune disease or an inflammatory disease.

The present invention may be embodied in other specific forms without departing from the spirit or essential attributes thereof. This invention encompasses all combinations of preferred aspects and/or embodiments of the invention noted herein. It is understood that any and all embodiments of the present invention may be taken in conjunction with any other embodiment or embodiments to describe additional embodiments. It is also to be understood that each individual element of the embodiments is its own independent embodiment. Furthermore, any element of an embodiment is meant to be combined with any and all other elements from any embodiment to describe an additional embodiment.

The following are definitions of terms used in this specification and appended claims. The initial definition provided for a group or term herein applies to that group or term throughout the specification and claims, individually or as part of another group, unless otherwise indicated.

Unless specifically stated otherwise herein, references made in the singular may also include the plural. For example, “a” and “an” may refer to either one, or one or more.

When any variable (e.g., R³) occurs more than one time in any constituent or formula for a compound, its definition at each occurrence is independent of its definition at every other occurrence. Thus, for example, if a group is shown to be substituted with 0-2 R³, then said group may optionally be substituted with up to two R³ groups and R³ at each occurrence is selected independently from the definition of R³. Also, combinations of substituents and/or variables are permissible only if such combinations result in stable compounds.

Unless otherwise indicated, any carbon or heteroatom with unsatisfied valences is assumed to have hydrogen atoms sufficient to satisfy the valences.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

In cases wherein there are nitrogen atoms (e.g., amines) on compounds of the present invention, these can be converted to N-oxides by treatment with an oxidizing agent (e.g., MCPBA and/or hydrogen peroxides) to afford other compounds of this invention. Thus, all shown and claimed nitrogen atoms are considered to cover both the shown nitrogen and its N-oxide (N→O) derivative.

In accordance with a convention used in the art,

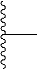

is used in structural formulas herein to depict the bond that is the point of attachment of the moiety or substituent to the core or backbone structure.

A dash “-” that is not between two letters or symbols is used to indicate a point of attachment for a substituent. For example, —CONH₂ is attached through the carbon atom.

The term “optionally substituted” in reference to a particular moiety of the compound of Formula (I), (e.g., an optionally substituted heteroaryl group) refers to a moiety having 0, 1, 2, or more substituents. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “alkyl” or “alkylene” is intended to include both branched and straight-chain saturated aliphatic hydrocarbon groups having the specified number of carbon atoms. For example, “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁-C₆ alkyl” denotes alkyl having 1 to 6 carbon atoms. Alkyl groups can be unsubstituted or substituted so that one or more of its hydrogens are replaced by another chemical group. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

When the term “alkyl” is used together with another group, such as in “arylalkyl”, this conjunction defines with more specificity at least one of the substituents that the substituted alkyl will contain. For example, “arylalkyl” refers to a substituted alkyl group as defined above where at least one of the substituents is an aryl, such as benzyl. Thus, the term aryl(C₀₋₄)alkyl includes a substituted lower alkyl having at least one aryl substituent and also includes an aryl directly bonded to another group, i.e., aryl(Co)alkyl.

The term “heteroarylalkyl” refers to a substituted alkyl group as defined above where at least one of the substituents is a heteroaryl. “Alkenyl” or “alkenylene” is intended to include hydrocarbon chains of either straight or branched configuration and having one or more double carbon-carbon bonds that may occur in any stable point along the chain. For example, “C₂₋₆ alkenyl” (or alkenylene), is intended to include C₂, C₃, C₄, C₅, and C₆ alkenyl groups. Examples of alkenyl include, but are not limited to, ethenyl, 1-propenyl, 2-propenyl, 2-butenyl, 3-butenyl, 2-pentenyl, 3, pentenyl, 4-pentenyl, 2-hexenyl, 3-hexenyl, 4-hexenyl, 5-hexenyl, 2-methyl-2-propenyl, 4-methyl-3-pentenyl, and the like.

“Alkynyl” or “alkynylene” is intended to include hydrocarbon chains of either straight or branched configuration and having one or more triple carbon-carbon bonds that may occur in any stable point along the chain. For example, “C₂₋₆ alkynyl” (or alkynylene), is intended to include C₂, C₃, C₄, C₅, and C₆ alkynyl groups; such as ethynyl, propynyl, butynyl, pentynyl, hexynyl and the like.

When reference is made to a substituted alkenyl, alkynyl, alkylene, alkenylene, or alkynylene group, these groups are substituted with one to three substituents as defined above for substituted alkyl groups.

The term “alkoxy” refers to an oxygen atom substituted by alkyl or substituted alkyl, as defined herein. For example, the term “alkoxy” includes the group —O—C₁₋₆alkyl such as methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. “Lower alkoxy” refers to alkoxy groups having one to four carbons.

It should be understood that the selections for all groups, including for example, alkoxy, thioalkyl, and aminoalkyl, will be made by one skilled in the field to provide stable compounds.

The term “substituted”, as used herein, means that any one or more hydrogens on the designated atom or group is replaced with a selection from the indicated group, provided that the designated atom's normal valence is not exceeded. When a substituent is oxo, or keto, (i.e., ═O) then 2 hydrogens on the atom are replaced. Unless otherwise specified, substituents are named into the core structure. For example, it is to be understood that when (cycloalkyl)alkyl is listed as a possible substituent, the point of attachment of this substituent to the core structure is in the alkyl portion. Ring double bonds, as used herein, are double bonds that are formed between two adjacent ring atoms (e.g., C═C, C═N, or N═N).

Combinations of substituents and/or variables are permissible only if such combinations result in stable compounds or useful synthetic intermediates. A stable compound or stable structure is meant to imply a compound that is sufficiently robust to survive isolation from a reaction mixture to a useful degree of purity, and subsequent formulation into an efficacious therapeutic agent. It is preferred that the presently recited compounds do not contain a N-halo, S(O)₂H, or S(O)H group.

The term “carbocyclyl” or “carbocyclic” refers to a saturated or unsaturated, or partially unsaturated, monocyclic or bicyclic ring in which all atoms of all rings are carbon. Thus, the term includes cycloalkyl and aryl rings, or bicyclic rings containing both saturated or unsaturated rings, or rings being partially unsaturated. Monocyclic carbocycles have 3 to 6 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocycle (e.g., [2.2.2]bicyclooctane). Carbocycles, can include cyclopropyl, cyclobutyl, cyclopentyl, cyclohexyl, and phenyl. When the term “carbocycle” is used, it is intended to include “aryl”. A bridged ring occurs when one or more carbon atoms link two non-adjacent carbon atoms. Preferred bridges are one or two carbon atoms. It is noted that a bridge always converts a monocyclic ring into a bicyclic ring. When a ring is bridged, the substituents recited for the ring may also be present on the bridge.

The term “aryl” refers to monocyclic or bicyclic aromatic hydrocarbon groups having 6 to 12 carbon atoms in the ring portion, such as phenyl, and naphthyl groups, each of which may be substituted. A preferred aryl group is optionally-substituted phenyl.

The term “cycloalkyl” refers to cyclized alkyl groups, including mono-, bi- or poly-cyclic ring systems. C₃₋₇ cycloalkyl is intended to include C₃, C₄, C₅, C₆, and C₇ cycloalkyl groups. Example cycloalkyl groups include, but are not limited to, cyclopropyl, cyclobutyl, cyclopentyl, cyclohexyl, norbornyl, and the like, which optionally may be substituted at any available atoms of the ring(s).

The terms “heterocycloalkyl”, “heterocyclo”, “heterocycle”, “heterocyclic”, or “heterocyclyl” may be used interchangeably and refer to substituted and unsubstituted aromatic or non-aromatic, or partially unsaturated, 3- to 7-membered monocyclic groups, 7- to 11-membered bicyclic groups, and 10- to 15-membered tricyclic groups, in which at least one of the rings has at least one heteroatom (0, S or N), said heteroatom containing ring preferably having 1, 2, or 3 heteroatoms selected from O, S, and N. Each ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The fused rings completing the bicyclic and tricyclic groups may contain only carbon atoms and may be saturated, partially saturated, or unsaturated. The heterocyclo group may be attached at any available nitrogen or carbon atom. The term “heterocycle” includes “heteroaryl” groups. As valence allows, if said further ring is cycloalkyl or heterocyclo it is additionally optionally substituted with ═O (oxo).

Exemplary monocyclic heterocyclyl groups include azetidinyl, pyrrolidinyl, oxetanyl, imidazolinyl, oxazolidinyl, isoxazolinyl, thiazolidinyl, isothiazolidinyl, tetrahydrofuranyl, piperidyl, piperazinyl, 2-oxopiperazinyl, 2-oxopiperidyl, 2-oxopyrrolodinyl, 2-oxoazepinyl, azepinyl, 1-pyridonyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like, including the exemplary groups listed under “heteroaryl”. Exemplary bicyclic heterocyclo groups include quinuclidinyl.

The term “heteroaryl” refers to substituted and unsubstituted aromatic 5- or 6-membered monocyclic groups, 9- or 10-membered bicyclic groups, and 11- to 14-membered tricyclic groups which have at least one heteroatom (0, S or N) in at least one of the rings, said heteroatom-containing ring preferably having 1, 2, or 3 heteroatoms selected from O, S, and N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated.

The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. As valence allows, if said further ring is cycloalkyl or heterocyclo it is additionally optionally substituted with ═O (oxo).

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and the like.

Exemplary bicyclic heteroaryl groups include indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridyl, dihydroisoindolyl, tetrahydroquinolinyl, and the like.

Exemplary tricyclic heteroaryl groups include carbazolyl, benzindolyl, phenanthrollinyl, acridinyl, phenanthridinyl, xanthenyl and the like.

Unless otherwise indicated, when reference is made to a specifically-named aryl (e.g., phenyl), cycloalkyl (e.g., cyclohexyl), heterocyclo (e.g., pyrrolidinyl, piperidinyl, and morpholinyl) or heteroaryl (e.g., tetrazolyl, imidazolyl, pyrazolyl, triazolyl, thiazolyl, and furyl) the reference is intended to include rings having 0 to 3, preferably 0-2, substituents, as appropriate.

The term “halo” or “halogen” refers to chloro, bromo, fluoro and iodo.

The term “haloalkyl” means a substituted alkyl having one or more halo substituents. For example, “haloalkyl” includes mono, bi, and trifluoromethyl.

The term “haloalkoxy” means an alkoxy group having one or more halo substituents. For example, “haloalkoxy” includes OCF3.

The term “deuteroalkyl” means a substituted alkyl having one or more deuterium atom. For example, the term “deuteroalkyl” includes mono, bi, and trideuteromethyl.

The term “heteroatoms” shall include oxygen, sulfur and nitrogen.

When the term “unsaturated” is used herein to refer to a ring or group, the ring or group may be fully unsaturated or partially unsaturated.

One skilled in the field will understand that, when the designation “CO₂” is used herein, this is intended to refer to the group

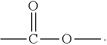

Throughout the specification, groups and substituents thereof may be chosen by one skilled in the field to provide stable moieties and compounds and compounds useful as pharmaceutically-acceptable compounds and/or intermediate compounds useful in making pharmaceutically-acceptable compounds.

The compounds of formula (I) may exist in a free form (with no ionization) or can form salts which are also within the scope of this invention. Unless otherwise indicated, reference to an inventive compound is understood to include reference to the free form and to salts thereof. The term “salt(s)” denotes acidic and/or basic salts formed with inorganic and/or organic acids and bases. In addition, the term “salt(s) may include zwitterions (inner salts), e.g., when a compound of formula (I), contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of the formula (I) may be formed, for example, by reacting a compound of the formula (I) with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-p-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. In one embodiment, salts include monohydrochloride, hydrogensulfate, methanesulfonate, phosphate or nitrate salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, PA, 1990, the disclosure of which is hereby incorporated by reference.

The present invention is intended to include all isotopes of atoms occurring in the present compounds. Isotopes include those atoms having the same atomic number but different mass numbers. By way of general example and without limitation, isotopes of hydrogen include deuterium and tritium. As an example, an alkyl substituent is intended to cover alkyl groups have either hydrogen, deuterium, and/or some combination thereof. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

Prodrugs and solvates of the inventive compounds are also contemplated. The term “prodrug” denotes a compound which, upon administration to a subject, undergoes chemical conversion by metabolic or chemical processes to yield a compound of the formula (I), and/or a salt and/or solvate thereof. Any compound that will be converted in vivo to provide the bioactive agent (i.e., the compound for formula (I)) is a prodrug within the scope and spirit of the invention. For example, compounds containing a carboxy group can form physiologically hydrolyzable esters which serve as prodrugs by being hydrolyzed in the body to yield formula (I) compounds per se. Such prodrugs are preferably administered orally since hydrolysis in many instances occurs principally under the influence of the digestive enzymes. Parenteral administration may be used where the ester per se is active, or in those instances where hydrolysis occurs in the blood. Examples of physiologically hydrolyzable esters of compounds of formula (I) include C₁₋₆alkylbenzyl, 4-methoxybenzyl, indanyl, phthalyl, methoxymethyl, C₁₋₆alkanoyloxy-C₁₋₆alkyl, e.g. acetoxymethyl, pivaloyloxymethyl or propionyloxymethyl, C₁₋₆alkoxycarbonyloxy-C₁₋₆alkyl, e.g. methoxycarbonyl-oxymethyl or ethoxycarbonyloxymethyl, glycyloxymethyl, phenylglycyloxymethyl, (5-methyl-2-oxo-1,3-dioxolen-4-yl)-methyl and other well known physiologically hydrolyzable esters used, for example, in the penicillin and cephalosporin arts. Such esters may be prepared by conventional techniques known in the art.

Various forms of prodrugs are well known in the art. For examples of such prodrug derivatives, see:
a) Design of Prodrugs, edited by H. Bundgaard, (Elsevier, 1985) and Methods in Enzymology, Vol. 112, pp. 309-396, edited by K. Widder, et al. (Academic Press, 1985);
b) A Textbook of Drug Design and Development, edited by Krosgaard-Larsen and H. Bundgaard, Chapter 5, “Design and Application of Prodrugs,” by H. Bundgaard, pp. 113-191 (1991); and
c) H. Bundgaard, Advanced Drug Delivery Reviews, Vol. 8, pp. 1-38 (1992), each of which is incorporated herein by reference.

Compounds of the formula (I) and salts thereof may exist in their tautomeric form, in which hydrogen atoms are transposed to other parts of the molecules and the chemical bonds between the atoms of the molecules are consequently rearranged. It should be understood that the all tautomeric forms, insofar as they may exist, are included within the invention.

Compounds of this invention may have one or more asymmetric centers. Unless otherwise indicated, all chiral (enantiomeric and diastereomeric) and racemic forms of compounds of the present invention are included in the present invention. Many geometric isomers of olefins, C═N double bonds, and the like can also be present in the compounds, and all such stable isomers are contemplated in the present invention. Cis and trans geometric isomers of the compounds of the present invention are described and may be isolated as a mixture of isomers or as separated isomeric forms. The present compounds can be isolated in optically active or racemic forms. The racemic forms can be resolved by physical methods, such as, for example, fractional crystallization, separation, or crystallization of diastereomeric derivatives or separation by chiral column chromatography. All chiral, (enantiomeric and diastereomeric) and racemic forms and all geometric isomeric forms of a structure are intended, unless the specific stereochemistry or isomer form is specifically indicated. All geometric isomers, tautomers, atropisomers, hydrates, solvates, polymorphs, and isotopically labeled forms of the compounds referred to herein, and mixtures thereof, are considered within the scope of the present invention. Methods of solvation are generally known in the art.

For some examples of the present invention, the absolute stereochemistry of the enantiomers and/or diasteriomers has not been specifically identified. However, the racemic mixtures and all enantiomers and diasteriomers are included in the present invention. Even where the specific enantiomers and/or diastereomers are isolated, but the absolute stereochemistry was not specifically determined and drawn, one of skill in the art can easily identify and draw the structures of the individual stereoisomers or diasteriomers. For example, Examples 110, 111, 112 and 113 are represented by the structure:

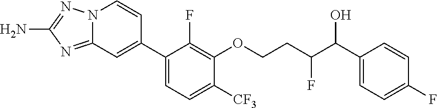 [SMILES: Nc1nc2cc(-c3ccc(C(F)(F)F)c(OCCC(F)C(O)c4ccc(F)cc4)c3F)ccn2n1]

The 4 diasteriomers are:

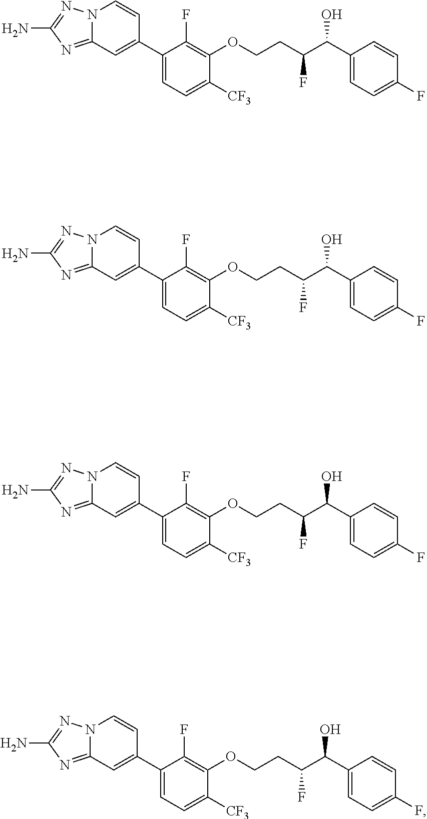 [SMILES: Nc1nc2cc(-c3ccc(C(F)(F)F)c(OCC[C@H](F)[C@H](O)c4ccc(F)cc4)c3F)ccn2n1 ; Nc1nc2cc(-c3ccc(C(F)(F)F)c(OCC[C@@H](F)[C@H](O)c4ccc(F)cc4)c3F)ccn2n1 ; Nc1nc2cc(-c3ccc(C(F)(F)F)c(OCC[C@H](F)[C@@H](O)c4ccc(F)cc4)c3F)ccn2n1 ; Nc1nc2cc(-c3ccc(C(F)(F)F)c(OCC[C@@H](F)[C@@H](O)c4ccc(F)cc4)c3F)ccn2n1]

which one of skill in the art is able to identify, even if the structure of each of the examples is not specifically described.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

### Utility

The compounds of the invention modulate kinase activity, including the modulation of RIPK1. Accordingly, compounds of formula (I) have utility in treating conditions associated with the modulation of kinase activity, and particularly the selective inhibition of RIPK1 activity. In another embodiment, compounds of formula (I) have advantageous selectivity for RIPK1 activity preferably from at least 10 fold, or alternatively, 20 fold, to over 1,000 fold more selective over other kinases.

As used herein, the terms “treating” or “treatment” encompass the treatment of a disease state in a mammal, particularly in a human, and include: (a) preventing or delaying the occurrence of the disease state in a mammal, in particular, when such mammal is predisposed to the disease state but has not yet been diagnosed as having it; (b) inhibiting the disease state, i.e., arresting its development; and/or (c) achieving a full or partial reduction of the symptoms or disease state, and/or alleviating, ameliorating, lessening, or curing the disease or disorder and/or its symptoms.

In view of their activity as inhibitors of RIPK1, compounds of Formula (I) are useful in treating RIPK1-associated conditions including, but not limited to, inflammatory diseases such as Crohn's disease and ulcerative colitis, inflammatory bowel disease, asthma, graft versus host disease, chronic obstructive pulmonary disease; autoimmune diseases such as Graves' disease, rheumatoid arthritis, systemic lupus erythematosis, psoriasis; destructive bone disorders such as bone resorption disease, osteoarthritis, osteoporosis, multiple myeloma-related bone disorder; proliferative disorders such as acute myelogenous leukemia, chronic myelogenous leukemia; angiogenic disorders such as angiogenic disorders including solid tumors, ocular neovasculization, and infantile haemangiomas; infectious diseases such as sepsis, septic shock, and Shigellosis; neurodegenerative diseases such as Alzheimer's disease, Parkinson's disease, cerebral ischemias or neurodegenerative disease caused by traumatic injury, multiple sclerosis (MS), amyotrophic lateral sclerosis (ALS), spinal cord injury, and traumatic brain injury (TBI), detachment of the retina, ischemia, Gaucher's disease, and AAV (ANCA-associated vasculitis) and other inflammatory diseases such as macrophage necrosis in atheroscelerosis development, virus-induced inflammation, systemic inflammatory response syndrome and ethanol-induced liver injury, oncologic and viral diseases such as metastatic melanoma, Kaposi's sarcoma, multiple myeloma, and HIV infection and CMV retinitis, AIDS; fibrotic conditions such as, nonalcoholic steatohepatitis (NASH); and cardiac conditions such as, ischemia reperfusion; respectively.

More particularly, the specific conditions or diseases that may be treated with the inventive compounds include, without limitation, pancreatitis (acute or chronic), asthma, allergies, adult respiratory distress syndrome, chronic obstructive pulmonary disease, glomerulonephritis, rheumatoid arthritis, systemic lupus erythematosis, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, ALS, multiple sclerosis, inflammatory bowel disease, ulcerative colitis, Crohn's disease, psoriasis, graft vs. host disease, inflammatory reaction induced by endotoxin, tuberculosis, atherosclerosis, muscle degeneration, cachexia, psoriatic arthritis, Reiter's syndrome, gout, traumatic arthritis, rubella arthritis, acute synovitis, pancreatic p-cell disease; diseases characterized by massive neutrophil infiltration; rheumatoid spondylitis, gouty arthritis and other arthritic conditions, cerebral malaria, chronic pulmonary inflammatory disease, silicosis, pulmonary sarcoisosis, bone resorption disease, allograft rejections, fever and myalgias due to infection, cachexia secondary to infection, meloid formation, scar tissue formation, ulcerative colitis, pyresis, influenza, osteoporosis, osteoarthritis, acute myelogenous leukemia, chronic myelogenous leukemia, metastatic melanoma, Kaposi's sarcoma, multiple myeloma, sepsis, septic shock, and Shigellosis; Alzheimer's disease, Parkinson's disease, cerebral ischemias or neurodegenerative disease caused by traumatic injury; angiogenic disorders including solid tumors, ocular neovasculization, and infantile haemangiomas; viral diseases including acute hepatitis infection (including hepatitis A, hepatitis B and hepatitis C), HIV infection and CMV retinitis, AIDS, ARC or malignancy, and herpes; stroke, myocardial ischemia, ischemia in stroke heart attacks, organ hyposia, vascular hyperplasia, cardiac and renal reperfusion injury, thrombosis, cardiac hypertrophy, thrombin-induced platelet aggregation, endotoxemia and/or toxic shock syndrome, conditions associated with prostaglandin endoperoxidase syndase-2, and pemphigus vulgaris. In another aspect, methods of treatment are those wherein the condition is selected from inflammatory bowel disease, Crohn's disease and ulcerative colitis, allograft rejection, rheumatoid arthritis, psoriasis, ankylosing spondylitis, psoriatic arthritis, and pemphigus vulgaris, and nonalcoholic steatohepatitis (NASH), and ischemia reperfusion. In a further aspect, methods of treatment are those wherein the condition is selected from multiple sclerosis, amyotrophic latereal sclerosis, and Alzheimers.

Alternatively preferred methods of treatment are those wherein the condition is selected from ischemia reperfusion injury, including cerebral ischemia reperfusions injury arising from stroke and cardiac ischemia reperfusion injury arising from myocardial infarction.

When the terms “RIPK1-associated condition” or “RIPK1-associated disease or disorder” are used herein, each is intended to encompass all of the conditions identified above as if repeated at length, as well as any other condition that is affected by RIPK1 kinase activity.

The present invention thus provides methods for treating such conditions, comprising administering to a subject in need thereof a therapeutically-effective amount of at least one compound of Formula (I) or a salt thereof. “Therapeutically effective amount” is intended to include an amount of a compound of the present invention that is effective when administered alone or in combination to inhibit RIPK1.

The methods of treating RIPK1 kinase-associated conditions may comprise administering compounds of Formula (I) alone or in combination with each other and/or other suitable therapeutic agents useful in treating such conditions. Accordingly, “therapeutically effective amount” is also intended to include an amount of the combination of compounds claimed that is effective to inhibit RIPK1 and/or treat diseases associated with RIPK1.

Exemplary of such other therapeutic agents include corticosteroids, rolipram, calphostin, cytokine-suppressive anti-inflammatory drugs (CSAIDs), Interleukin-10, glucocorticoids, salicylates, nitric oxide, and other immunosuppressants; nuclear translocation inhibitors, such as deoxyspergualin (DSG); non-steroidal antiinflammatory drugs (NSAIDs) such as ibuprofen, celecoxib and rofecoxib; steroids such as prednisone or dexamethasone; anti-inflammatory anti-bodies such as vedolizumab and ustekinumab, anti-infammatory kinase inhibitors such as TYK2 inhibitors, antiviral agents such as abacavir; antiproliferative agents such as methotrexate, leflunomide, FK506 (tacrolimus, Prograf); cytotoxic drugs such as azathiprine and cyclophosphamide; TNF-α inhibitors such as tenidap, anti-TNF antibodies or soluble TNF receptor, rapamycin (sirolimus or Rapamune) or derivatives thereof, and agonists of FGF21.

Combination startegies include therapies intended to target the accumulation and/or downstream effects of amyloid beta and/or tau, including but not limited to antibodies that bind to these toxic species, ligand-directed degradation or other degraders such as CELMoD agents. In addition to small molecule and/or antibody-based therapeutic combinations, other options include siRNA and/or ASO approaches as well as the use of delivery technologies designed to augment central exposure to peripherally administered therapeutic agents in addition to ICV or intrathecal administration.

The above other therapeutic agents, when employed in combination with the compounds of the present invention, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds. The present invention also provides pharmaceutical compositions capable of treating RIPK1 kinase-associated conditions, including IL-1, IL-6, IL-8, IFNγ and TNF-α-mediated conditions, as described above.

The inventive compositions may contain other therapeutic agents as described above and may be formulated, for example, by employing conventional solid or liquid vehicles or diluents, as well as pharmaceutical additives of a type appropriate to the mode of desired administration (e.g., excipients, binders, preservatives, stabilizers, flavors, etc.) according to techniques such as those well known in the art of pharmaceutical formulation.

Accordingly, the present invention further includes compositions comprising one or more compounds of Formula (I) and a pharmaceutically acceptable carrier.

A “pharmaceutically acceptable carrier” refers to media generally accepted in the art for the delivery of biologically active agents to animals, in particular, mammals. Pharmaceutically acceptable carriers are formulated according to a number of factors well within the purview of those of ordinary skill in the art. These include without limitation the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 17th ed., 1985, which is incorporated herein by reference in its entirety.

The compounds of Formula (I) may be administered by any means suitable for the condition to be treated, which may depend on the need for site-specific treatment or quantity of drug to be delivered. Topical administration is generally preferred for skin-related diseases, and systematic treatment preferred for cancerous or pre-cancerous conditions, although other modes of delivery are contemplated. For example, the compounds may be delivered orally, such as in the form of tablets, capsules, granules, powders, or liquid formulations including syrups; topically, such as in the form of solutions, suspensions, gels or ointments; sublingually; bucally; parenterally, such as by subcutaneous, intravenous, intramuscular or intrasternal injection or infusion techniques (e.g., as sterile injectable aq. or non-aq. solutions or suspensions); nasally such as by inhalation spray; topically, such as in the form of a cream or ointment; rectally such as in the form of suppositories; or liposomally. Dosage unit formulations containing non-toxic, pharmaceutically acceptable vehicles or diluents may be administered. The compounds may be administered in a form suitable for immediate release or extended release. Immediate release or extended release may be achieved with suitable pharmaceutical compositions or, particularly in the case of extended release, with devices such as subcutaneous implants or osmotic pumps.

Exemplary compositions for topical administration include a topical carrier such as PLASTIBASE® (mineral oil gelled with polyethylene).

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The inventive compounds may also be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

The therapeutically-effective amount of a compound of the present invention may be determined by one of ordinary skill in the art, and includes exemplary dosage amounts for a mammal of from about 0.05 to 1000 mg/kg; 1-1000 mg/kg; 1-50 mg/kg; 5-250 mg/kg; 250-1000 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like. Thus, when the term “patient” is used herein, this term is intended to include all subjects, most preferably mammalian species, that are affected by mediation of RIPK1 enzyme levels.

### Biological Assays

Necroptosis is a regulated pathway of cell death that is induced by both inflammatory factors (TNFalpha) as well as viral triggers such as TLR agonists. The process of necroptosis induction occurs following activation and phosphorlylation of RIPK1 to form a complex with RIPK3 (referred to as necrosome). Mixed Lineage Kinase domain-like protein (MLKL) is recruited to RIPK3 and is a downstream target of RIPK3 kinase, leading to MLKL phosphorylation at Thr357 and Ser358. Phosphorylated MLKL (pMLKL) leads to MLKL oligomerization, translocation to the plasma membrane, and subsequent pore formation leading to membrane integrity defects (Moriwaki, K., and F. K. Chan. 2013. RIP3: a molecular switch for necrosis and inflammation. Genes Dev. 27: 1640-1649). Thus, understanding the potency of RIPK1 compounds based on RIPK1 direct binding as well as a functional readout of necroptosis activity (pMLKL) is important for evaluating RIPK1 inhibitor activity and potency.

MLKL Phosphorylation High-Content Assay HT29-L23 human colorectal adenocarcinoma cells were maintained in RPMI 1640 medium containing 10% heat-inactivated FBS, 1% Penicillin-Streptomycin and 10 mM HEPES. Cells were seeded at 2,000 cells/well in 384 well tissue culture-treated microplates (Greiner #781090-3B) and incubated at 37° C. (5% CO₂/95% O₂) for 2 d. On the day of the assay, the cells were treated with test compounds at final concentrations of 6.25 to 0.106 μM for 30 min at 37° C. (5% CO₂/95% 02). Necroptopsis was induced using a mixture of human TNFα (35 ng/mL) (Peprotech #300-01A), SMAC mimetic (from US 2015/0322111 A1) (700 nM) and Z-VAD (140 nM) (BD pharmingen #51-6936). Following 6 h incubation at 37° C. (5% CO₂/95% O₂), the cells were fixed with 4% formaldehyde (ACROS 11969-0010) for 15 min at rt, then permeabilized with phosphate buffered saline (PBS) containing 0.2% Triton-X-100 for 10 min. MLKL phosphorylation was detected using anti-MLKL (phospho S358) antibody (Abcam #ab187091) (1:1000 dilution in Blocking Buffer [PBS supplemented with 0.1% BSA]) with ON incubation at 4° C. After washing three times in PBS, goat anti-rabbit Alexa-488 (1:1000 dilution) (Life Technologies, A11008) and Hoechst 33342 (Life Technologies, H3570) (1:2000 dilution) in Blocking Buffer were added for 1 h at rt.

Following another three cycles of washes in PBS, the microplates were sealed, and cellular images were acquired in the Cellomics ArrayScan VTI high-content imager equipped with an X1 camera. Fluorescent images were taken using a 10× objective and the 386-23 BGRFRN_BGRFRN and 485-20 BGRFRN_BGRFRN filter sets, for nuclei and MLKL phosphorylation, respectively. The image sets were analyzed using the Compartmental Analysis Bioapplication software (Cellomics). The level of MLKL phosphorylation was quantified as MEAN_CircRingAvgIntenRatio. The maximal inhibitory response was defined by the activity induced by Nec1s (CAS #: 852391-15-2, 6.25 μM). The IC50 value was defined as the concentration of compound that produces 50% of the maximal inhibition. The data were fitted using the 4-parameter logistic equation to calculate the IC₅₀ and Yₘₐₓ values.

The following table shows the activity obtained in the above identified assay for the indicated compounds
“- indicates same as previous

| | | LE pMLKL | |
|---|---|---|---|
| Example | name | HC IC50 (uM) | |
| 1 | 7-(3-(4-((4-chloropyridin-2-yl)oxy)-3,3- | 0.14 | achiral |
| | difluorobutoxy)-2-fluorophenyl)- | | |
| | [1,2,4]triazolo[1,5-a]pyridin-2-amine | | |
| 2 | 5-(3-(2-amino-[1,2,4]triazolo[1,5- | 0.004 | Enantiomer 1 |
| | a]pyridin-7-yl)-2-fluorophenoxy)-3,3- | | |
| | difluoro-2-(4-fluorophenyl)pentan-2-ol | | |
| 3 | ″ | 0.0001 | Enantiomer 2 |
| 4 | 5-(3-(2-amino-[1,2,4]triazolo[1,5- | 0.0005 | Enantiomer 1 |
| | a]pyridin-7-yl)-6-chloro-2- | | |
| | fluorophenoxy)-3,3-difluoro-2-(4- | | |
| | fluorophenyl)pentan-2-ol | | |
| 5 | ″ | 0.15 | Enantiomer 2 |
| 6 | 1-(3-(2-amino-[1,2,4]triazolo[1,5- | 0.08 | Enantiomer 1 |
| | a]pyridin-7-yl)-2-fluorophenoxy)-3,3- | | |
| | difluoro-6-methylheptan-4-ol | | |
| 7 | ″ | 0.16 | Enantiomer 2 |
| 8 | 5-(3-(2-amino-[1,2,4]triazolo[1,5- | 0.009 | Enantiomer 1 |
| | a]pyridin-7-yl)-2,4-difluorophenoxy)- | | |
| | 3,3-difluoro-2-(4-fluorophenyl)pentan- | | |
| | 2-ol | | |
| 9 | ″ | 0.0001 | Enantiomer 2 |
| 10 | 5-(3-(2-amino-[1,2,4]triazolo[1,5- | 0.001 | Enantiomer 1 |
| | a]pyridin-7-yl)-2,5-difluorophenoxy)- | | |
| | 3,3-difluoro-2-(4-fluorophenyl)pentan- | | |
| | 2-ol | | |
| 11 | ″ | 0.0002 | Enantiomer 2 |
| 12 | 5-(3-(2-amino-[1,2,4]triazolo[1,5- | 0.001 | Enantiomer 1 |
| | a]pyridin-7-yl)-2-fluorophenoxy)-3,3- | | |
| | difluoro-2-(4-fluorophenyl)pentan-5,5- | | |
| | d2-2-ol | | |
| 13 | ″ | 0.0001 | Enantiomer 2 |
| 14 | 5-(3-(2-amino-[1,2,4]triazolo[1,5- | 0.03 | Enantiomer 1 |
| | a]pyridin-7-yl)-2,6-difluorophenoxy)- | | |
| | 3,3-difluoro-2-(4-fluorophenyl)pentan- | | |
| | 5,5-d2-2-ol | | |
| 15 | ″ | 0.0003 | Enantiomer 2 |
| 16 | 7-(3-((4-amino-3,3-difluoro-4-(4- | 0.02 | Enantiomer 1 |
| | fluorophenyl)pentyl)oxy)-2,4- | | |
| | difluorophenyl)-[1,2,4]triazolo[1,5- | | |
| | a]pyridin-2-amine | | |
| 17 | | 0.06 | Enantiomer 2 |
| 18 | 5-(3-(2-amino-[1,2,4]triazolo[1,5- | 0.08 | Enantiomer 1 |
| | a]pyridin-7-yl)-2-fluoro-6- | | |
| | methoxyphenoxy)-3,3-difluoro-2-(4- | | |
| | fluorophenyl)pentan-2-ol | | |
| 19 | ″ | 0.0004 | Enantiomer 2 |
| 20 | 5-(3-(2-amino-[1,2,4]triazolo[1,5- | 0.11 | Enantiomer 1 |
| | a]pyridin-7-yl)-2,6-difluoro-4- | | |
| | methoxyphenoxy)-3,3-difluoro-2-(4- | | |
| | fluorophenyl)pentan-2-ol | | |
| 21 | ″ | 0.0011 | Enantiomer 2 |
| 22 | 5-(3-(2-amino-[1,2,4]triazolo[1,5- | 0.0009 | racemic |
| | a]pyridin-7-yl)-2-fluoro-4- | | |
| | methoxyphenoxy)-3,3-difluoro-2-(4- | | |
| | fluorophenyl)pentan-2-ol | | |
| 23 | 5-(3-(2-amino-[1,2,4]triazolo[1,5- | 6.0 | Enantiomer 1 |
| | a]pyridin-7-yl)-2- | | |
| | (difluoromethyl)phenoxy)-3,3-difluoro- | | |
| | 2-(4-fluorophenyl)pentan-2-ol | | |
| 24 | ″ | 6.0 | Enantiomer 2 |
| 25 | 5-(3-(2-amino-[1,2,4]triazolo[1,5- | 0.0015 | Enantiomer 1 |
| | a]pyridin-7-yl)-6-(difluoromethyl)-2- | | |
| | fluorophenoxy)-3,3-difluoro-2-(4- | | |
| | fluorophenyl)pentan-2-ol | | |
| 26 | ″ | 0.0003 | Enantiomer 2 |
| 27 | 5-(3-(2-amino-[1,2,4]triazolo[1,5- | hWB 64nM | Enantiomer 1 |
| | a]pyridin-7-yl)-2,6-difluorophenoxy)- | | |
| | 3,3-difluoro-2-(5-fluoropyridin-2- | | |
| | yl)pentan-2-ol | | |
| 28 | ″ | hWB 82nM | Enantiomer 2 |
| 29 | 5-(3-(2-amino-[1,2,4]triazolo[1,5- | hWB 4nM | Single enantiomer |
| | a]pyridin-7-yl)-2,4,6-trifluorophenoxy)- | | |
| | 3,3-difluoro-2-(4-fluorophenyl)pentan- | | |
| | 2-ol | | |
| 30 | 5-(3-(2-amino-[1,2,4]triazolo[1,5- | hWB | Single enantiomer |
| | a]pyridin-7-yl)-2,5,6-trifluorophenoxy)- | 8 nM | |
| | 3,3-difluoro-2-(4-fluorophenyl)pentan- | | |
| | 2-ol | | |
| 31 | 5-(3-(2-amino-[1,2,4]triazolo[1,5- | 0.002 | Enantiomer 1 |
| | a]pyridin-7-yl)-6-chloro-2- | | |
| | fluorophenoxy)-3,3-difluoro-2-(5- | | |
| | fluoropyridin-2-yl)pentan-2-ol | | |
| 32 | ″ | 0.03 | Enantiomer 2 |
| 33 | 4-(3-(2-amino-[1,2,4]triazolo[1,5- | 0.085 | Enantiomer 1 |
| | a]pyridin-7-yl)-2,6-difluorophenoxy)- | | |
| | 2,2-difluoro-1-(5-fluoropyridin-2- | | |
| | yl)butan-1-ol | | |
| 34 | ″ | 0.008 | Enantiomer 2 |
| 35 | 5-(3-(2-amino-[1,2,4]triazolo[1,5- | 0.35 | Enantiomer 1 |
| | a]pyridin-7-yl)-2,6-difluorophenoxy)- | | |
| | 3,3-difluoro-2-(pyridin-3-yl)pentan-2-ol | | |
| 36 | ″ | 0.005 | Enantiomer 2 |
| 37 | 5-(3-(2-amino-8-fluoro- | 0.11 | Enantiomer 1 |
| | [1,2,4]triazolo[1,5-a]pyridin-7-yl)-2,6- | | |
| | difluorophenoxy)-3,3-difluoro-2- | | |
| | phenylpentan-2-ol | | |
| 38 | ″ | 0.0003 | Enantiomer 2 |
| 39 | 5-(3-(2-amino-[1,2,4]triazolo[1,5- | 0.13 | Enantiomer 1 |
| | a]pyridin-7-yl)-2,6-difluorophenoxy)- | | |
| | 3,3-difluoro-2-phenylpentan-2-ol | | |
| 40 | ″ | 0.0006 | Enantiomer 2 |
| 41 | 5-(3-(2-amino-[1,2,4]triazolo[1,5- | 0.0004 | Enantiomer 1 |
| | a]pyridin-7-yl)-6-chloro-2- | | |
| | fluorophenoxy)-3,3-difluoro-2- | | |
| | phenylpentan-2-ol | | |
| 42 | ″ | 0.017 | Enantiomer 2 |
| 43 | 5-(3-(2-amino-[1,2,4]triazolo[1,5- | 0.33 | Enantiomer 1 |
| | a]pyridin-7-yl)-6-chloro-2- | | |
| | fluorophenoxy)-2-(4-chlorophenyl)-3,3- | | |
| | difluoropentan-2-ol | | |
| 44 | ″ | 0.0006 | Enantiomer 2 |
| 45 | 5-(3-(2-amino-[1,2,4]triazolo[1,5- | 0.08 | Enantiomer 1 |
| | a]pyridin-7-yl)-2,6-difluorophenoxy)-2- | | |
| | (4-chlorophenyl)-3,3-difluoropentan-2- | | |
| | ol | | |
| 46 | ″ | 0.0004 | Enantiomer 2 |
| 47 | 5-(3-(2-amino-[1,2,4]triazolo[1,5- | 0.023 | Enantiomer 1 |
| | a]pyridin-7-yl)-2-fluorophenoxy)-2-(4- | | |
| | chlorophenyl)-3,3-difluoropentan-2-ol | | |
| 48 | ″ | 0.0001 | Enantiomer 2 |
| 49 | 5-(3-(2-amino-[1,2,4]triazolo[1,5- | 0.034 | Enantiomer 1 |
| | a]pyridin-7-yl)-2,6-difluorophenoxy)- | | |
| | 3,3-difluoro-2-(4-fluorophenyl)pentan- | | |
| | 2-ol | | |
| 50 | ″ | 0.0004 | Enantiomer 2 |
| 51 | 5-(3-(2-amino-[1,2,4]triazolo[1,5- | 0.15 | Enantiomer 1 |
| | a]pyridin-7-yl)-6-chloro-2- | | |
| | fluorophenoxy)-3,3-difluoro-2-(4- | | |
| | fluorophenyl)pentan-2-ol | | |
| 52 | ″ | 0.0005 | Enantiomer 2 |
| 53 | 4-(5-(2-amino-[1,2,4]triazolo[1,5- | 0.0015 | Enantiomer 1 |
| | a]pyridin-7-yl)-2-fluorophenoxy)-2,2- | | |
| | difluoro-1-(4-fluorophenyl)butan-1-ol | | |
| 54 | ″ | 0.032 | Enantiomer 2 |
| 55 | 4-(3-(2-amino-[1,2,4]triazolo[1,5- | 0.0003 | Enantiomer 1 |
| | a]pyridin-7-yl)-2,6-difluorophenoxy)- | | |
| | 2,2-difluoro-1-(4-fluorophenyl)butan-1- | | |
| | ol | | |
| 56 | ″ | 0.004 | Enantiomer 2 |
| 57 | 5-(5-(2-amino-[1,2,4]triazolo[1,5- | 0.13 | Enantiomer 1 |
| | a]pyridin-7-yl)-2-fluorophenoxy)-3,3- | | |
| | difluoro-2-(4-fluorophenyl)pentan-2-ol | | |
| 58 | ″ | 0.0050 | Enantiomer 2 |
| 59 | 4-(3-(2-amino-[1,2,4]triazolo[1,5- | 0.002 | Enantiomer 1 |
| | a]pyridin-7-yl)-6-chloro-2- | | |
| | fluorophenoxy)-2,2-difluoro-1-(4- | | |
| | fluorophenyl)butan-1-ol | | |
| 60 | ″ | 0.005 | Enantiomer 2 |
| 61 | 7-(3-(4-amino-3,3-difluoro-4-(4- | 0.0006 | Enantiomer 1 |
| | fluorophenyl)butoxy)-2-fluorophenyl)- | | |
| | [1,2,4]triazolo[1,5-a]pyridin-2-amine | | |
| 62 | ″ | 0.006 | Enantiomer 2 |
| 63 | 5-(3-(2-amino-[1,2,4]triazolo[1,5- | 0.008 | Enantiomer 1 |
| | a]pyridin-7-yl)-2-fluoro-6- | | |
| | methylphenoxy)-3,3-difluoro-2-(4- | | |
| | fluorophenyl)pentan-2-ol | | |
| 64 | ″ | 0.0001 | Enantiomer 2 |
| 65 | 7-(3-(3,3-difluoro-3-(4- | 0.14 | achiral |
| | fluorophenoxy)propoxy)-2- | | |
| | fluorophenyl)-[1,2,4]triazolo[1,5- | | |
| | a]pyridin-2-amine | | |
| 66 | 4-(3-(2-amino-[1,2,4]triazolo[1,5- | 0.0003 | racemic |
| | a]pyridin-7-yl)-2-fluoro-6- | | |
| | methylphenoxy)-2,2-difluoro-1-(4- | | |
| | fluorophenyl)butan-1-ol | | |
| 67 | 5-(3-(2-amino-[1,2,4]triazolo[1,5- | 0.004 | Enantiomer 1 |
| | a]pyridin-7-yl)-2-fluorophenoxy)-3,3- | | |
| | difluoro-2-phenylpentan-2-ol | | |
| 68 | ″ | 0.0001 | Enantiomer 2 |
| 69 | 4-(3-(2-amino-[1,2,4]triazolo[1,5- | 0.003 | racemic |
| | a]pyridin-7-yl)-2-fluorophenoxy)-2,2- | | |
| | difluoro-1-phenylbutan-1-ol | | |
| 70 | 4-(3-(2-amino-[1,2,4]triazolo[1,5- | 0.001 | racemic |
| | a]pyridin-7-yl)-2-fluorophenoxy)-1-(4- | | |
| | chlorophenyl)-2,2-difluorobutan-1-ol | | |
| 71 | 4-(3-(2-amino-[1,2,4]triazolo[1,5- | 0.0006 | Enantiomer 1 |
| | a]pyridin-7-yl)-2-fluorophenoxy)-2,2- | | |
| | difluoro-1-(4-fluorophenyl)butan-1-ol | | |
| 72 | ″ | 0.001 | Enantiomer 2 |
| 73 | 5-(3-(2-amino-[1,2,4]triazolo[1,5- | 0.0005 | racemic |
| | a]pyridin-7-yl)-2-fluorophenoxy)-3,3- | | |
| | difluoro-2-(4-fluorophenyl)pentan-2-ol | | |
| 74 | 7-(3-(3,3-difluoro-4-phenylbutoxy)-2- | 0.005 | achiral |
| | fluorophenyl)-[1,2,4]triazolo[1,5- | | |
| | a]pyridin-2-amine | | |
| 75 | 5-(3-(2-amino-[1,2,4]triazolo[1,5- | 0.20 | Enantiomer 1 |
| | a]pyridin-6-yl)-2,6-difluorophenoxy)- | | |
| | 3,3-difluoro-2-(4-fluorophenyl)pentan- | | |
| | 2-ol | | |
| 76 | ″ | 0.002 | Enantiomer 2 |
| 77 | 5-((6-(2-amino-[1,2,4]triazolo[1,5- | 0.43 | Enantiomer 1 |
| | a]pyridin-7-yl)-5-fluoropyridin-2- | | |
| | yl)oxy)-3,3-difluoro-2-(4- | | |
| | fluorophenyl)pentan-2-ol | | |
| 78 | ″ | 0.013 | Enantiomer 2 |
| 79 | 5-((6-(2-amino-[1,2,4]triazolo[1,5- | 0.16 | Enantiomer 1 |
| | a]pyridin-7-yl)-5-chloropyridin-2- | | |
| | yl)oxy)-3,3-difluoro-2-(4- | | |
| | fluorophenyl)pentan-2-ol | | |
| 80 | ″ | 0.0068 | Enantiomer 2 |
| 81 | 5-((6-(2-amino-[1,2,4]triazolo[1,5- | 0.0002 | racemic |
| | a]pyridin-7-yl)-5-chloropyrazin-2- | | |
| | yl)oxy)-3,3-difluoro-2-(4- | | |
| | fluorophenyl)pentan-2-ol | | |
| 82 | 4-(3-(2-amino-[1,2,4]triazolo[1,5- | 0.026 | Isomer 1 |
| | a]pyridin-7-yl)-6-chloro-2- | | |
| | fluorophenoxy)-2-fluoro-1-(4- | | |
| | fluorophenyl)butan-1-ol | | |
| 83 | ″ | 0.026 | Isomer 2 |
| 84 | 4-(3-(2-amino-[1,2,4]triazolo[1,5- | 0.0032 | Isomer 1 |
| | a]pyridin-7-yl)-2-fluorophenoxy)-2- | | |
| | fluoro-1-(4-fluorophenyl)butan-1-ol | | |
| 85 | ″ | 0.0009 | Isomer 2 |
| 86 | ″ | 0.017 | Isomer 3 |
| 87 | ″ | 0.0004 | Isomer 4 |
| 88 | 5-(3-(2-amino-[1,2,4]triazolo[1,5- | 0.013 | Isomer 1 |
| | a]pyridin-7-yl)-2-fluorophenoxy)-3- | | |
| | fluoro-2-(4-fluorophenyl)pentan-2-ol | | |
| 89 | ″ | 0.015 | Isomer 2 |
| 90 | ″ | 0.003 | Isomer 3 |
| 91 | ″ | 0.0003 | Isomer 4 |
| 92 | 5-(5-(2-amino-[1,2,4]triazolo[1,5- | 1.39 | Isomer 1 |
| | a]pyridin-7-yl)-2,3-difluorophenoxy)-3- | | |
| | fluoro-2-(4-fluorophenyl)pentan-2-ol | | |
| 93 | ″ | 0.14 | Isomer 2 |
| 94 | 5-(3-(2-amino-[1,2,4]triazolo-[1,5- | 0.097 | Isomer 1 |
| | a]pyridin-7-yl)-2,6-difluorophenoxy)-3- | | |
| | fluoro-2-(4-fluorophenyl)pentan-2-ol | | |
| 95 | ″ | 0.004 | Isomer 2 |
| 96 | ″ | 0.002 | Isomer 3 |
| 97 | ″ | 0.029 | Isomer 4 |
| 98 | 4-(3-(2-amino-[1,2,4]triazolo[1,5- | 0.002 | Isomer 1 |
| | a]pyridin-7-yl)-6-chloro-2- | | |
| | fluorophenoxy)-2-fluoro-1-(4- | | |
| | fluorophenyl)butan-1-ol | | |
| 99 | ″ | 0.015 | Isomer 2 |
| 100 | ″ | 0.006 | Isomer 3 |
| 101 | ″ | 0.03 | Isomer 4 |
| 102 | 4-(3-(2-amino-[1,2,4]triazolo[1,5- | 0.003 | Isomer 1 |
| | a]pyridin-7-yl)-2,6-difluorophenoxy)-2- | | |
| | fluoro-1-(4-fluorophenyl)butan-1-ol | | |
| 103 | ″ | 0.0015 | Isomer 2 |
| 104 | ″ | 0.0028 | Isomer 3 |
| 105 | ″ | 0.012 | Isomer 4 |
| 106 | 4-(3-(2-amino-[1,2,4]triazolo[1,5- | 0.0030 | Isomer 1 |
| | a]pyridin-7-yl)-2-fluoro-6- | | |
| | methylphenoxy)-2-fluoro-1-(4- | | |
| | fluorophenyl)butan-1-ol | | |
| 107 | ″ | 0.0012 | Isomer 2 |
| 108 | ″ | 0.0024 | Isomer 3 |
| 109 | ″ | 0.0001 | Isomer 4 |
| 110 | 4-(3-(2-amino-[1,2,4]triazolo[1,5- | 0.36 | Isomer 1 |
| | a]pyridin-7-yl)-2-fluoro-6- | | |
| | (trifluoromethyl)phenoxy)-2-fluoro-1- | | |
| | (4-fluorophenyl)butan-1-ol | | |
| 111 | ″ | 0.029 | Isomer 2 |
| 112 | ″ | 0.73 | Isomer 3 |
| 113 | ″ | 0.22 | Isomer 4 |
| 114 | 5-(3-(2-amino-[1,2,4]triazolo[1,5- | 0.0001 | Isomer 1 |
| | a]pyridin-7-yl)-2-fluoro-6- | | |
| | methylphenoxy)-3-fluoro-2-(4- | | |
| | fluorophenyl)pentan-2-ol | | |
| 115 | ″ | 0.0026 | Isomer 2 |
| 116 | 4-(5-(2-amino-[1,2,4]triazolo[1,5- | 0.055 | Isomer 1 |
| | a]pyridin-7-yl)-2,3-difluorophenoxy)-2- | | |
| | fluoro-1-(4-fluorophenyl)butan-1-ol | | |
| 117 | ″ | 0.58 | Isomer 2 |
| 118 | ″ | 1.04 | Isomer 3 |
| 119 | ″ | 0.58 | Isomer 4 |
| 120 | 7-(2-fluoro-4-methyl-3-(3,4,4-trifluoro- | 0.0096 | Enantiomer 1 |
| | 4-(4-fluorophenyl)butoxy)phenyl)- | | |
| | [1,2,4]triazolo[1,5-a]pyridin-2-amine | | |
| 121 | ″ | 0.089 | Enantiomer 2 |
| 122 | 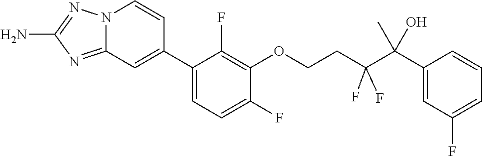 [SMILES: CC(O)(c1cccc(F)c1)C(F)(F)CCOc1c(F)ccc(-c2ccn3nc(N)nc3c2)c1F] | 0.13 | Enantiomer 1 |
| | 5-(3-(2-amino-[1,2,4]triazolo[1,5- | | |
| | a]pyridin-7-yl)-2,6-difluorophenoxy)- | | |
| | 3,3-difluoro-2-(3-fluorophenyl)pentan- | | |
| | 2-ol | | |
| 123 | | 0.0009 | Enantiomer 2 |
| 124 | 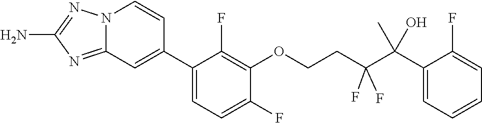 [SMILES: CC(O)(c1ccccc1F)C(F)(F)CCOc1c(F)ccc(-c2ccn3nc(N)nc3c2)c1F] | 0.16 | Enantiomer 1 |
| | 5-(3-(2-amino-[1,2,4]triazolo[1,5- | | |
| | a]pyridin-7-yl)-2,6-difluorophenoxy)- | | |
| | 3,3-difluoro-2-(2-fluorophenyl)pentan- | | |
| | 2-ol | | |
| 125 | | 0.0006 | Enantiomer 2 |
| 126 | 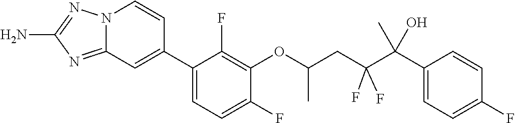 [SMILES: CC(CC(F)(F)C(C)(O)c1ccc(F)cc1)Oc1c(F)ccc(-c2ccn3nc(N)nc3c2)c1F] | 0.37 | Isomer 1 |
| | 5-(3-(2-amino-[1,2,4]triazolo[1,5- | | |
| | a]pyridin-7-yl)-2,6-difluorophenoxy)- | | |
| | 3,3-difluoro-2-(4-fluorophenyl)hexan- | | |
| | 2-ol | | |
| 127 | | 0.0011 | Isomer 2 |
| 128 | | 0.17 | Isomer 3 |
| 129 | | 0.070 | Isomer 4 |
| 130 | 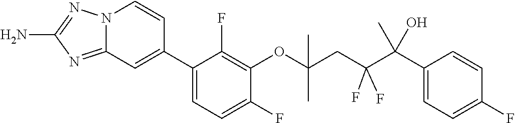 [SMILES: CC(C)(CC(F)(F)C(C)(O)c1ccc(F)cc1)Oc1c(F)ccc(-c2ccn3nc(N)nc3c2)c1F] | 4.2 | Enantiomer 1 |
| | 5-(3-(2-amino-[1,2,4]triazolo[1,5- | | |
| | a]pyridin-7-yl)-2,6-difluorophenoxy)- | | |
| | 3,3-difluoro-2-(4-fluorophenyl)-5- | | |
| | methylhexan-2-ol | | |
| 131 | | 0.0034 | Enantiomer 2 |

### Transporter Assays

Compounds were assessed as substrates of MDR1 P-glycoprotein (P-gp) and mouse Breast Cancer Resistance Protein (mBcrp) at 0.1 uM, for 2 hours, in bi-directional transport assays using recombinant MDCK cell lines as described previously (Feng et al. Drug Metabolism and Disposition 2008, Vol. 36:268-275). 1MDCK-MDR1 and MDCK-mBcrp cells were acquired from the National Institutes of Health (Bethesda, MID) and the Netherlands Cancer Institute respectively, and used under license agreement. Data are expressed as efflux ratios, where transport across the cell monolayer in the basolateral to apical direction is divided by transport in the apical to basolateral direction. Efflux ratios >2 indicate the test compound is a substrate of the transporter tested.

The following compounds were tested in the above assay and had the following results:

| Example | PGP | BCRP |
|---|---|---|
| 2 | 1.8 | 6.2 |
| 3 | 1.2 | 3.4 |
| 4 | 1.5 | 1.7 |
| 13 | 1.8 | 3.9 |
| 15 | 1.4 | 2.9 |
| 27 | 1.1 | 1.6 |
| 29 | 1.6 | 2.5 |
| 30 | 2.5 | 3.0 |
| 31 | 5.3 | 1.4 |
| 34 | 2.4 | 7.0 |
| 38 | 2.1 | 5.1 |
| 40 | 1.7 | 4.8 |
| 49 | 1.5 | 4.0 |
| 50 | 1.6 | 2.4 |
| 52 | 1.5 | 1.7 |
| 80 | 5.9 | 5.6 |

### Determining Actual Brain Concentrations

Aliquots of brain homogenate can be frozen at −80° C. in polypropylene tubes. The total concentration of Compound A in brain tissue can be measured using liquid chromatography tandem mass spectrometry (LC/MS) analysis. The samples for the LC/MS analysis can be prepared using a protein precipitation procedure described below.

Acetonitrile (100 μL), containing an internal standard [1 mM], along with the sample (30 μL), can be added to a 96-well Filter Plate (Multiscreen Solvinert 0.45 mm Low Binding Hydrophilic PTFE, Millipore) fitted on top of the final autosampler plate (96-well 1 ml Collection Plate, Waters) including an insert (Ultra Amp 96-well 0.2 ml Ultraplate, Sorenson Bioscience). The assembly can be vortex mixed for 5 min at room temperature, centrifuged at 3,700 rpm at 4° C. for 10 min, then capped. The supernatant (3 μL) can be injected to an Ultra Performance LC System (Waters Acquity iClass) interfaced with a Quadrapole MS/MS (Thermo Scientific TSQ Quantiva) tandem mass spectrometer. The analyte can be separated on a C₁₈ column (Waters Acquity HSS T3, 2.1×50 mm, 1.8 μm) at 40° C., with a gradient flow rate of 0.7 ml/min, consisting of two buffer solutions (A: water, 5 mM Ammonium Formate, 0.1% formic acid; B: acetonitrile, 0.1% formic acid). The detection is possible by using multiple reaction monitoring (MRM) in the positive electrospray ionization mode, representing the precursor (M+H)+ species.

The assays described above may be used to illustrate that compounds of the present invention are brain penetrant, and therefore are useful for the treatment of neurodegenerative diseases as described above.

By comparison, Example 30 from WO2022/086828 was tested in the above assays with the following results.

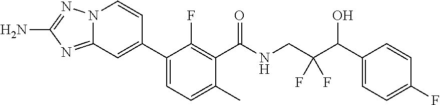 [SMILES: Cc1ccc(-c2ccn3nc(N)nc3c2)c(F)c1C(=O)NCC(F)(F)C(O)c1ccc(F)cc1]

### Example 30

| | PGP | BCRP |
|---|---|---|
| Example 30 | 35 | 43 |

### List of Abbreviations

AcOH or HOAc acetic acid
BISPIN bis(pinacolato)diboron
Bn benzyl
Boc tert-butyloxycarbonyl
BOP benzotriazol-1-yloxytris(dimethylamino)phosphonium hexafluorophosphate
Bu butyl
CBz carbobenzyloxy
DCE 1,2 dichloroethane
DCM dichloromethane
DIAD diisopropyl azodicarboxylate
DIEA/DIPEA/Hünig's Base diisopropyl ethylamine
DMAP 4-dimethylaminopyridine
DME 1,2-dimethoxyethane
DMF dimethyl formamide
DMSO dimethyl sulfoxide
EDC/EDCI N-(3-dimthylaminopropyl)-N′-ethylcarbodiimide
Et ethyl
Et₂O diethyl ether
Et₃N or TEA triethylamine
EtOAc ethyl acetate
EtOH ethanol
HATU O-(7-azabenzotriazol-1-yl)-N,N,N′,N′-tetramethyluronium hexafluorophosphate
HCl hydrochloric acid
Hex hexane
HOBt or HOBT 1-hydroxybenzotriazole
i-Bu isobutyl
i-Pr isopropyl
i-PrOH or IPA isopropanol
KOAc potassium acetate
LAH lithium aluminum hydride
LDA lithium diisopropylamide
LG leaving group
Me methyl
MeCN or ACN acetonitrile
Mel iodomethane
MeOH methanol
MgSO₄ magnesium sulfate
NBS N-bromosuccinimide
n-BuLi n-butyllithium
NCS N-chlorosuccinimide
NH₄OAc ammonium acetate
OTf triflate or trifluoromethanesulfonate
Pd(dppf)Cl₂ [1,1′-bis(diphenylphosphino)-ferrocene]dichloropalladium(II)
Pd(OAc)₂ palladium(II) acetate
Pd/C palladium on carbon
Pd₂(dba)₃ tris(dibenzylideneacetone)dipalladium(O) PdCl₂(dtbpf) [1,1′-Bis(di-tert-butylphosphino)ferrocene]dichloropalladium(II)
PG protecting group
Ph phenyl
Ph₃P Triphenyl phosphine
Pr propyl
PTFE polytetrafluoroethylene or Teflon
SiO₂ silica oxide or silica gel
TBAI tetra-n-butylammonium iodide
t-Bu tert-butyl
TES-OTf Triethylsilyl trifluoromethanesulfonate
TFA trifluoroacetic acid
THF tetrahydrofuran

### Methods of Preparation

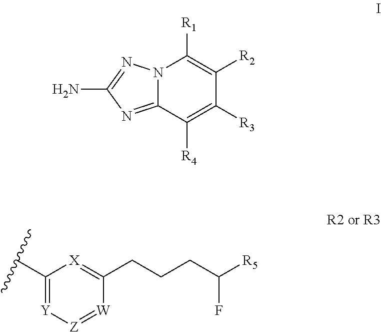

Compounds of Formula (I), and intermediates used in the preparation of compounds of Formula (I), can be prepared using procedures shown in the following examples and related procedures. The methods and conditions used in these examples, and the actual compounds prepared in these examples, are not meant to be limiting, but are meant to demonstrate how the compounds of Formula (I) can be prepared.

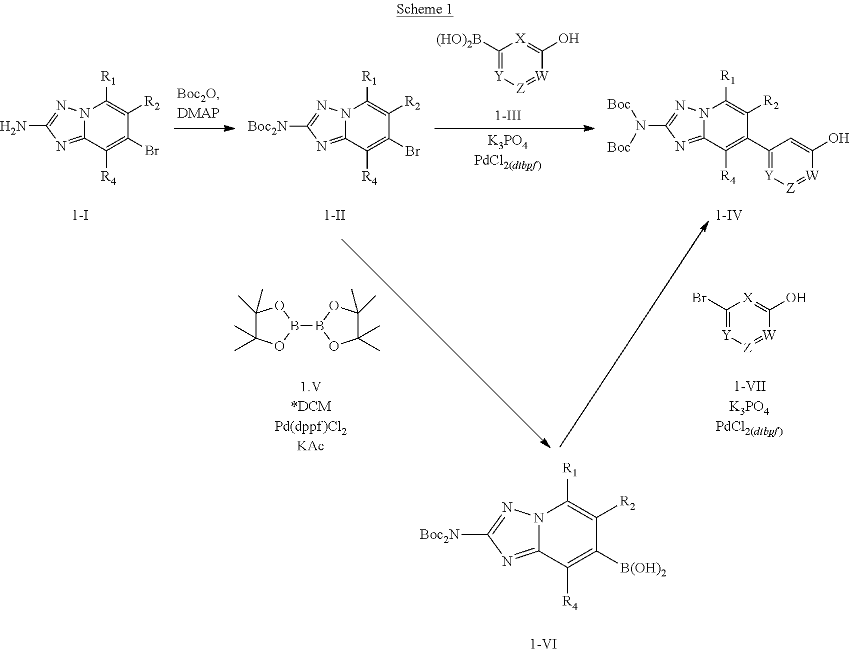

Scheme 1 illustrates 2 different ways to get to intermediates 1-IV. After Boc-protection of the bromo-substituted triazolo-pyridin-2-amine 1-I compound 1-II can be reacted with an appropriately substituted phenol boronate 1-ITT or converted to the boronic acid 1-VI and then coupled with a bromo substituted phenol 1-VII under standard Suzuki conditions to obtain intermediates 1-IV.

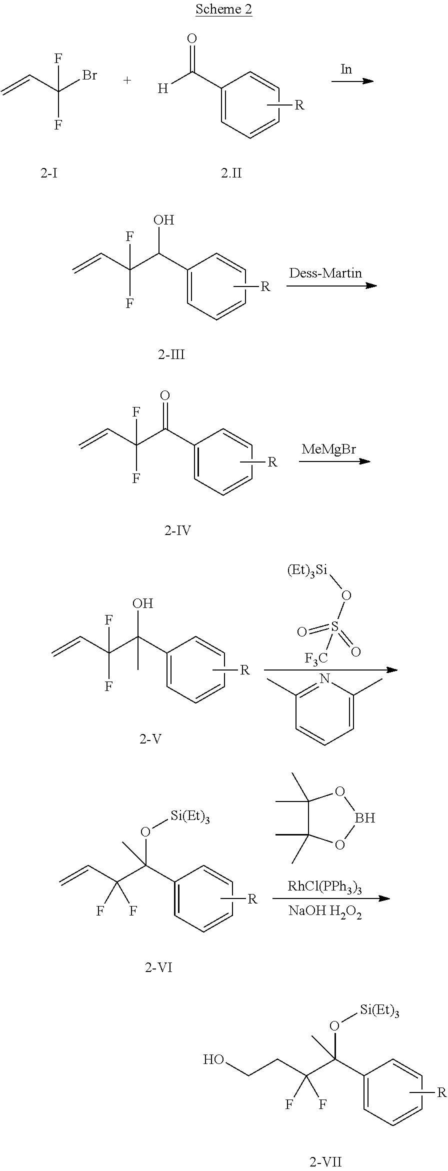

Scheme 2 shows the route to the right hand side alcohol precursor 2-VII. Starting from 3-bromo-3,3-difluoroprop-1-ene 2-I and the appropriately substituted aldehyde 2-II yielded secondary alcohol 2.III which was oxidized to the ketone 2-IV and then reacted with methyl Grignard to form tertiary alcohol 2-V. After silyl protection of the alcohol hydroboration yielded intermediates 2-VII.

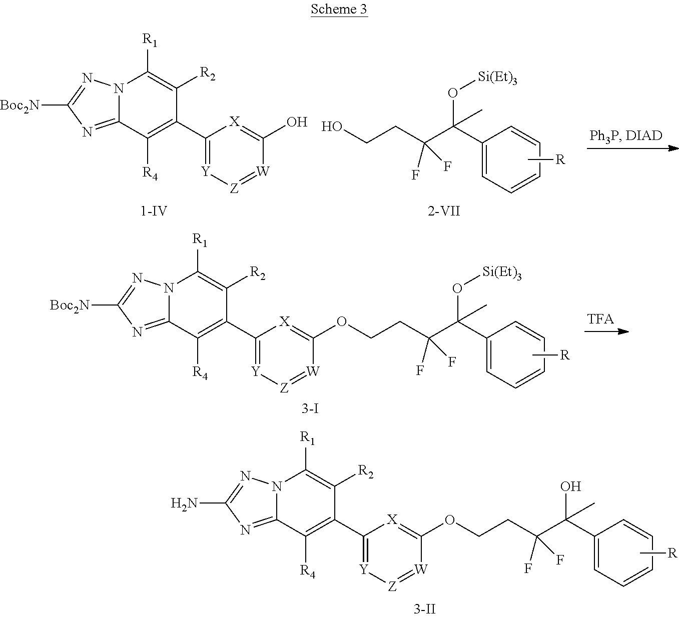

Intermediates 1-IV and 2-VII were coupled under Mitsunobu conditions. A deprotection with TFA yielded the final products 3-II.

### Intermediate A: tert-butyl (tert-butoxycarbonyl)(7-(2-fluoro-3-hydroxyphenyl)-[1,2,4]-triazolo[1,5-a]pyridin-2-yl)carbamate

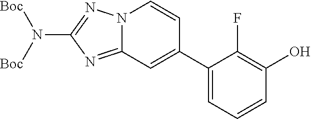 [SMILES: CC(C)(C)OC(=O)N(C(=O)OC(C)(C)C)c1nc2cc(-c3cccc(O)c3F)ccn2n1]

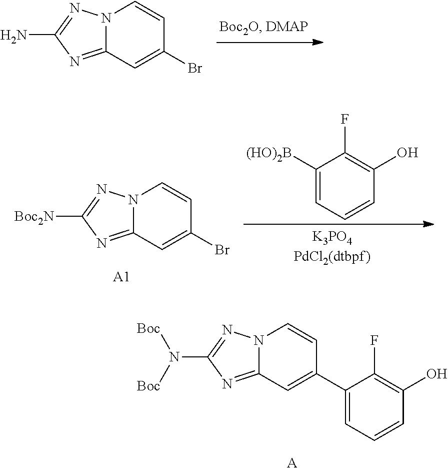

A1: tert-butyl (7-bromo-[1,2,4]triazolo[1,5-a]pyridin-2-yl)(tert-butoxycarbonyl)carbamate:

A mixture of 7-bromo-[1,2,4]triazolo[1,5-a]pyridin-2-amine (4.26 g, 20 mmol), BOC-anhydride (11.61 mL, 50.0 mmol) and DMAP (0.733 g, 6.00 mmol) in ACN (50 mL) was stirred at rt for 16 h. The mixture was concentrated. The residue was purified via silica gel chromatography (120 g, hexanes-100% EtOAc) to give the product tert-butyl (7-bromo-[1,2,4]triazolo[1,5-a]pyridin-2-yl)(tert-butoxycarbonyl)carbamate A1 (7.8 g, 18.87 mmol, 94% yield).

¹H NMR (499 MHz, chloroform-d) δ 8.42 (dd, J=7.3, 0.7 Hz, 1H), 7.92 (dd, J=2.0, 0.7 Hz, 1H), 7.18 (dd, J=7.2, 2.1 Hz, 1H), 1.49 (s, 18H).

MS ESI m/z 415.1 (M+H)⁺.

### A: tert-butyl (tert-butoxycarbonyl)(7-(2-fluoro-3-hydroxyphenyl)-[1,2,4]-triazolo[1,5-a]pyridin-2-yl)carbamate

To a solution of tert-butyl (7-bromo-[1,2,4]triazolo[1,5-a]pyridin-2-yl)(tert-butoxycarbonyl)-carbamate A1 (5.0 g, 12.10 mmol), (2-fluoro-3-hydroxyphenyl)boronic acid (2.264 g, 14.52 mmol) and 1,1′-bis(di-tert-butylphosphino)ferrocene palladium dichloride (0.394 g, 0.605 mmol) in dioxane (50 mL) was added 2.0 M tripotassium phosphate/water (18.15 mL, 36.3 mmol). The mixture was stirred at 85° C. under N₂ for 20 min. Water was added, and the mixture was extracted with 2× EtOAc. The organic layer was concentrated. The residue was purified via silica gel chromatography (120 g, hexanes-100% EtOAc) to give tert-butyl (tert-butoxycarbonyl)(7-(2-fluoro-3-hydroxyphenyl)-[1,2,4]-triazolo[1,5-a]pyridin-2-yl)carbamate A (4.6 g, 10.35 mmol, 86% yield).

¹H NMR (499 MHz, chloroform-d) δ 8.60 (dd, J=7.2, 0.8 Hz, 1H), 7.99 (s, 1H), 7.30-7.27 (m, 1H), 7.20-7.16 (m, 1H), 7.15-7.11 (m, 1H), 7.04 (td, J=7.2, 1.7 Hz, 1H), 6.57 (d, J=4.2 Hz, 1H), 1.50 (s, 18H).

MS ESI m/z 445.3 (M+H)⁺.

### Intermediate B: tert-butyl (tert-butoxycarbonyl)(7-(2-fluoro-3-hydroxy-4-methylphenyl)-[1,2,4]triazolo[1,5-a]pyridin-2-yl)carbamate

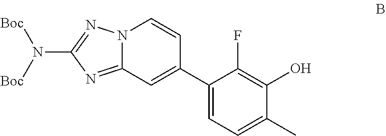 [SMILES: Cc1ccc(-c2ccn3nc(N(C(=O)OC(C)(C)C)C(=O)OC(C)(C)C)nc3c2)c(F)c1O]

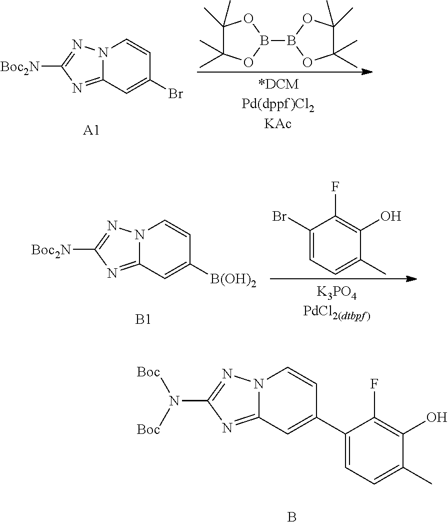

### B1: (2-(bis(tert-butoxycarbonyl)amino)-[1,2,4]triazolo[1,5-a]pyridin-7-yl)boronic acid

A mixture of A1 (5.0 g, 12.10 mmol), 4,4,4′,4′,5,5,5′,5′-octamethyl-2,2′-bi(1,3,2-dioxaborolane) (4.61 g, 18.15 mmol), PdCl₂(dppf)-CH₂Cl₂ adduct (0.593 g, 0.726 mmol) and potassium acetate (3.56 g, 36.3 mmol) in dioxane (60 mL) was stirred at 105° C. for 1 h. LCMS indicated the starting material was consumed, and the product was formed as a single major peak. The reaction mixture was used crude in the next step.

MS ESI m/z 379.2 (M+H)⁺.

### B: tert-butyl (tert-butoxycarbonyl)(7-(2-fluoro-3-hydroxy-4-methylphenyl)-[1,2,4]triazolo[1,5-a]pyridin-2-yl)carbamate

To a crude reaction mixture of B1 (2.214 g, 5.85 mmol) was added 3-bromo-2-fluoro-6-methylphenol (1.2 g, 5.85 mmol), 1,1′-bis(di-tert-butylphosphino)ferrocene palladium dichloride 98% (0.191 g, 0.293 mmol) and tripotassium phosphate (8.78 mL, 17.56 mmol). The mixture was stirred at 105° C. for 1 h. Water was added, and the mixture was extracted with 2× EtOAC. The organic layer was concentrated. The residue was purified via silica gel chromatography (40 g, hexanes-100% EtOAc) to give tert-butyl (tert-butoxycarbonyl)(7-(2-fluoro-3-hydroxy-4-methylphenyl)-[1,2,4]triazolo[1,5-a]pyridin-2-yl)carbamate B (2.5 g, 5.45 mmol, 93% yield).

¹H NMR (499 MHz, chloroform-d) δ 8.60-8.57 (m, 1H), 8.00 (s, 1H), 7.31-7.25 (m, 1H), 7.07-7.04 (m, 1H), 6.98-6.94 (m, 1H), 6.79-6.76 (m, 1H), 2.37 (s, 3H), 1.50 (s, 18H).

MS ESI m/z 459.3 (M+H)⁺.

### Intermediate C: tert-butyl (tert-butoxycarbonyl)(7-(2,4-difluoro-3-hydroxyphenyl)-[1,2,4]triazolo[1,5-a]pyridin-2-yl)carbamate

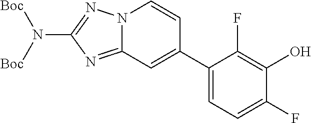 [SMILES: CC(C)(C)OC(=O)N(C(=O)OC(C)(C)C)c1nc2cc(-c3ccc(F)c(O)c3F)ccn2n1]

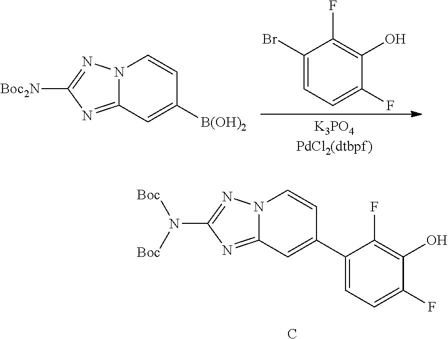

C: tert-butyl (tert-butoxycarbonyl)(7-(2,4-difluoro-3-hydroxyphenyl)-[1,2,4]triazolo[1,5-a]pyridin-2-yl)carbamate was prepared in a similar fashion to B using (2-(bis(tert-butoxycarbonyl)amino)-[1,2,4]triazolo[1,5-a]pyridin-7-yl)boronic acid B1 and 3-bromo-2,6-difluorophenol.

¹H NMR (499 MHz, chloroform-d) δ 8.62-8.58 (m, 1H), 8.02-8.01 (m, 1H), 7.26-7.22 (m, 1H), 7.08-7.02 (m, 1H), 7.01-6.95 (m, 1H), 1.50 (d, J=0.8 Hz, 18H).

MS ESI m/z 463.4 (M+H)⁺.

### Intermediate D: tert-butyl (tert-butoxycarbonyl)(7-(4-chloro-2-fluoro-3-hydroxyphenyl)-[1,2,4]triazolo[1,5-a]pyridin-2-yl)carbamate

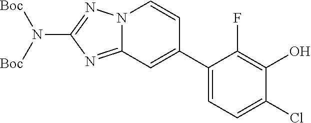 [SMILES: CC(C)(C)OC(=O)N(C(=O)OC(C)(C)C)c1nc2cc(-c3ccc(Cl)c(O)c3F)ccn2n1]

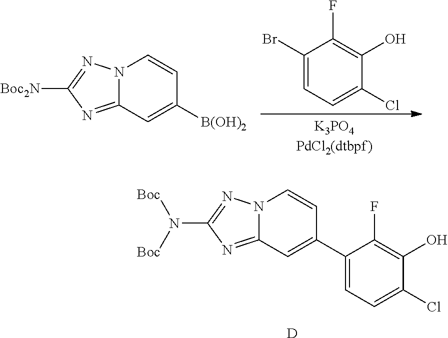

D: tert-butyl (tert-butoxycarbonyl)(7-(4-chloro-2-fluoro-3-hydroxyphenyl)-[1,2,4]triazolo[1,5-a]pyridin-2-yl)carbamate was prepared in a similar fashion to B using (2-(bis(tert-butoxycarbonyl)amino)-[1,2,4]triazolo[1,5-a]pyridin-7-yl)boronic acid B1 and 3-bromo-6-chloro-2-fluorophenol.

¹H NMR (499 MHz, chloroform-d) δ 8.61 (dd, J=7.2, 0.8 Hz, 1H), 8.00 (s, 1H), 7.82 (s, 1H), 7.30-7.24 (m, 2H), 7.01 (dd, J=8.5, 7.5 Hz, 1H), 1.50 (s, 18H).

MS ESI m/z 479.2 (M+H)⁺.

### Intermediate E: tert-butyl (7-(4-fluoro-3-hydroxyphenyl)-[1,2,4]triazolo[1,5-a]pyridin-2-yl)carbamate

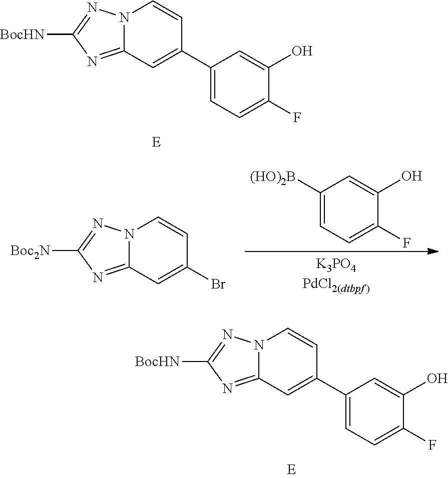

E: tert-butyl (7-(4-fluoro-3-hydroxyphenyl)-[1,2,4]triazolo[1,5-a]pyridin-2-yl)carbamate was prepared in a similar fashion to A using tert-butyl (7-bromo-[1,2,4]triazolo[1,5-a]pyridin-2-yl)(tert-butoxycarbonyl)carbamate A1 and (4-fluoro-3-hydroxyphenyl)boronic acid.

¹H NMR (499 MHz, chloroform-d) δ 8.57 (dd, J=7.2, 0.8 Hz, 1H), 7.82 (dd, J=1.9, 0.7 Hz, 1H), 7.31 (d, J=8.1 Hz, 1H), 7.27-7.14 (m, 3H), 5.84 (s, 1H), 1.51 (s, 18H).

MS ESI m/z 445.4 (M+H)⁺.

### Intermediate F: 3,3-difluoro-4-(4-fluorophenyl)-4-((triethylsilyl)oxy)pentan-1-ol

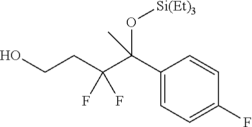

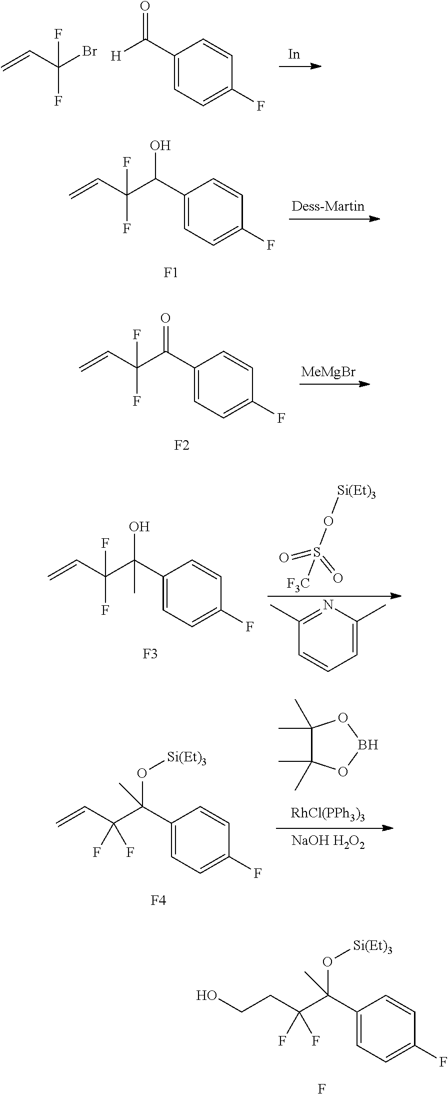

### F1: 2,2-difluoro-1-(4-fluorophenyl)but-3-en-1-ol

To a mixture of 4-fluorobenzaldehyde (3.29 g, 26.5 mmol) and indium (3.65 g, 31.8 mmol) in DMF (50 mL) was added 3-bromo-3,3-difluoroprop-1-ene (4.99 g, 31.8 mmol) dropwise in an ice bath. The mixture was stirred at rt for 20 h. Et₂O was added, the organic phase washed with 2× 1N HCl and 2× water, then dried over Na₂SO₄ and concentrated to give fairly clean 2,2-difluoro-1-(4-fluorophenyl)but-3-en-1-ol (5.3 g, 26.2 mmol, 99% yield).

¹H NMR (499 MHz, chloroform-d) δ 7.45-7.40 (m, 2H), 7.10-7.05 (m, 2H), 5.86 (ddt, J=17.4, 12.6, 11.1 Hz, 1H), 5.63-5.58 (m, 1H), 5.50 (d, J=11.1 Hz, 1H), 4.93 (t, J=9.4 Hz, 1H), 2.53 (br s, 1H).

### F2: 2,2-difluoro-1-(4-fluorophenyl)but-3-en-1-one

To a mixture of 2,2-difluoro-1-(4-fluorophenyl)but-3-en-1-ol (5.3 g, 26.2 mmol) in DCM (80 mL) was slowly added dess-martin periodinane (14.45 g, 34.1 mmol) in a water bath.

The mixture was stirred at rt for 2.5 h. TLC indicated the SM was consumed, and a new less polar spot was formed. The mixture was concentrated in vacuo at rt. EtOAc/Hexanes was added and the mixture was filtered through glass fiber paper. The filtrate was washed with 2× NaHCO₃/water and 2× water, dried over Na₂SO₄, then concentrated to dryness to obtain 2,2-difluoro-1-(4-fluorophenyl)but-3-en-1-one (4.8 g, 23.98 mmol, 91% yield).

¹H NMR (499 MHz, chloroform-d) δ 8.18-8.13 (m, 2H), 7.23-7.17 (m, 2H), 6.23 (dq, J=17.5, 11.2 Hz, 1H), 5.88 (dt, J=17.4, 2.7 Hz, 1H), 5.72 (d, J=11.2 Hz, 1H).

### F3: 3,3-difluoro-2-(4-fluorophenyl)pent-4-en-2-ol

To a mixture of 2,2-difluoro-1-(4-fluorophenyl)but-3-en-1-one (4.8 g, 23.98 mmol) in THF (80 mL) was added methylmagnesium bromide/Et₂O (11.19 mL, 33.6 mmol) slowly at −20° C. under N₂. The mixture was allowed to warm up to rt over 2 h. TLC monitored the reaction until the SM was consumed. The reaction was carefully quenched with water. Et₂O was added, washed with 1N HCl and water, then concentrated. The residue was purified via silica gel chromatography (120 g, hexanes-25% EtOAc) to give 3,3-difluoro-2-(4-fluorophenyl)pent-4-en-2-ol (4.0 g, 18.50 mmol, 77% yield).

¹H NMR (499 MHz, chloroform-d) δ 7.52 (dd, J=8.2, 5.9 Hz, 2H), 7.08-7.02 (m, 2H), 5.84 (ddt, J=17.4, 12.5, 11.4 Hz, 1H), 5.58-5.52 (m, 1H), 5.44-5.40 (m, 1H), 2.27 (s, 1H), 1.73 (t, J=1.3 Hz, 3H).

### F4: ((3,3-difluoro-2-(4-fluorophenyl)pent-4-en-2-yl)oxy)triethylsilane

To a solution of 3,3-difluoro-2-(4-fluorophenyl)pent-4-en-2-ol (4.0 g, 18.50 mmol) and 2,6-lutidine (3.02 mL, 25.9 mmol) in DCM (80 mL) was added triethylsilyl trifluoromethanesulfonate (5.87 g, 22.20 mmol) slowly at −60° C. under N₂. The mixture was allowed to warm up to rt and stirred at rt for 18 h. TLC indicated there was still some SM left. Another 0.6 eq. of 2,6-lutidine and 0.5 eq of TESOTf were added. The mixture was stirred at 45° C. for 3 h. TLC monitored the reaction until all the SM was consumed. The mixture was concentrated in vacuo. Hexane was added and the solid was filtered out and washed with hexanes. The filtrate was concentrated, then purified via silica gel chromatography (220 g, hexanes) to give ((3,3-difluoro-2-(4-fluorophenyl)pent-4-en-2-yl)oxy)triethylsilane (5.3 g, 16.04 mmol, 87% yield).

¹H NMR (499 MHz, chloroform-d δ 7.49-7.45 (m, 2H), 7.04-6.98 (m, 2H), 5.88 (ddt, J=17.5, 12.5, 11.3 Hz, 1H), 5.45-5.39 (m, 1H), 5.36 (d, J=11.1 Hz, 1H), 1.78 (t, J=1.3 Hz, 3H), 0.98-0.93 (m, 9H), 0.70-0.58 (m, 6H).

### F: 3,3-difluoro-4-(4-fluorophenyl)-4-((triethylsilyl)oxy)pentan-1-ol

To a solution of ((3,3-difluoro-2-(4-fluorophenyl)pent-4-en-2-yl)oxy)triethylsilane (5.3 g, 16.04 mmol) and 4,4,5,5-tetramethyl-1,3,2-dioxaborolane (2.463 g, 19.25 mmol) in THF (45 mL) was added rhodium(I) tris(triphenylphosphine) chloride (0.297 g, 0.321 mmol) at rt. The mixture was stirred at rt for 20 h. NaOH/water (21.38 mL, 64.2 mmol) was added in an ice bath. Then hydrogen peroxide (6.55 mL, 64.2 mmol) was added dropwise over 20 min. The mixture was stirred at rt for 2 h. Water was added, and extracted with 2×Et₂O. The organic layer was concentrated. The residue was purified via silica gel chromatography (160 g, hexanes-30% EtOAc), to give 3,3-difluoro-4-(4-fluorophenyl)-4-((triethylsilyl)oxy)pentan-1-ol (2.26 g, 6.49 mmol, 40.4% yield)

¹H NMR (499 MHz, chloroform-d) δ 7.52-7.47 (m, 2H), 7.07-7.01 (m, 2H), 3.86-3.75 (m, 2H), 2.35-2.20 (m, 1H), 1.84-1.70 (m, 4H), 1.59 (br t, J=6.1 Hz, 1H), 0.97 (t, J=7.9 Hz, 9H), 0.72-0.64 (m, 6H).

### Intermediate G: 3,3-difluoro-4-(4-fluorophenyl)-4-((triethylsilyl)oxy)pentan-1-ol (First eluting isomer)

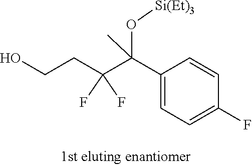

G and H: Racemic 3,3-difluoro-4-(4-fluorophenyl)-4-((triethylsilyl)oxy)pentan-1-ol F (2 g, 5.74 mmol) was chirally separated via preparative SFC chromatography with the following conditions: Column: CHIRALCEL OZ, 30 mm×250 mm, 5 m particles; Flow Rate: 0.200 mL/min; Column Temperature: 45° C. Mobile Phase A (96% C₀₂) Mobile Phase B (4% IPA/Hexanes with 0.1% DEA). Fraction collection was triggered by UV (220 nm). Fractions containing the desired product were combined and dried via centrifugal evaporation. The first eluting enantiomer G 3,3-difluoro-4-(4-fluorophenyl)-4-((triethylsilyl)oxy)pentan-1-ol (700 mg, 2.009 mmol, 35.0% yield), and the second eluting enantiomer H 3,3-difluoro-4-(4-fluorophenyl)-4-((triethylsilyl)oxy)pentan-1-ol (750 mg, 2.152 mmol, 37.5% yield) were obtained.

### Intermediate H: 3,3-difluoro-4-(4-fluorophenyl)-4-((triethylsilyl)oxy)pentan-1-ol (Second eluting isomer)

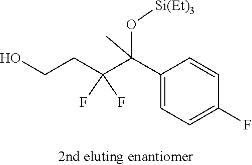

### Example 1: 7-(3-(4-((4-chloropyridin-2-yl)oxy)-3,3-difluorobutoxy)-2-fluorophenyl)-[1,2,4]triazolo[1,5-a]pyridin-2-amine

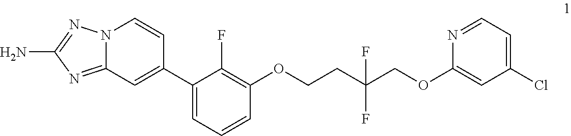 [SMILES: Nc1nc2cc(-c3cccc(OCCC(F)(F)COc4cc(Cl)ccn4)c3F)ccn2n1]

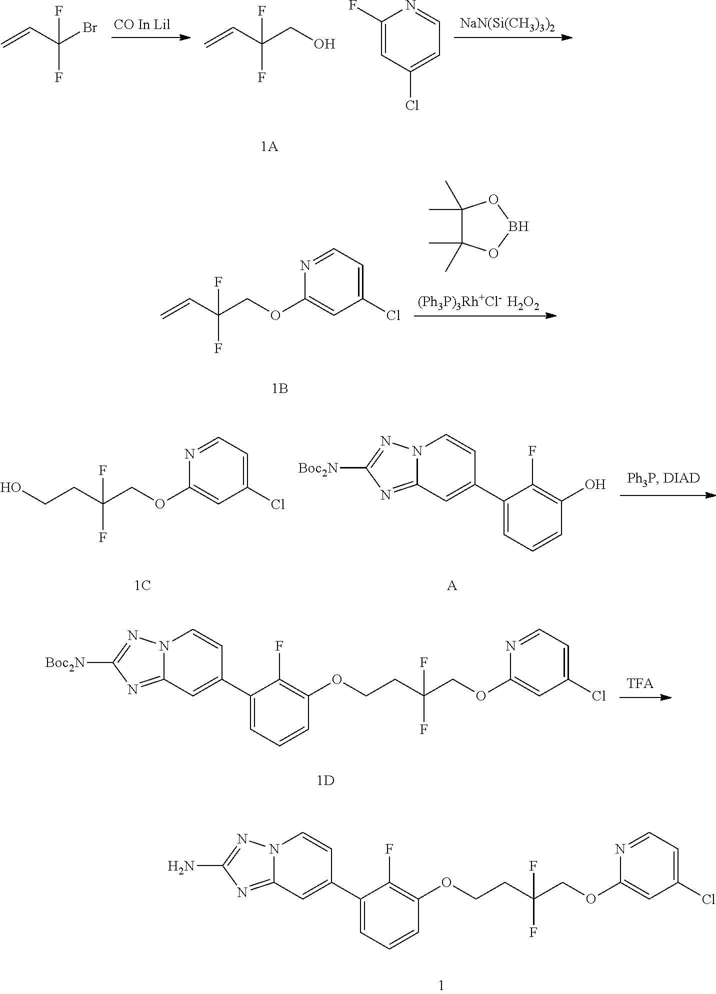

### 1A: 2,2-difluorobut-3-en-1-ol

To a solution of 3-bromo-3,3-difluoroprop-1-ene (1.24 g, 7.90 mmol) and paraformaldehyde (2.372 g, 79 mmol) in DMF (20 mL) was added indium (1.361 g, 11.85 mmol) and lithium iodide (0.085 g, 0.632 mmol) at rt. The mixture was stirred at 45° C. for 20 h. The reaction mixture was filtered through celite, and rinsed with EtOAc. The filtrate was washed with brine and water, dried over Na₂SO₄ then concentrated to give 850 mg of the crude product, which was used for the next step.

### 1B: 4-chloro-2-((2,2-difluorobut-3-en-1-yl)oxy)pyridine

To a solution of crude 2,2-difluorobut-3-en-1-ol (850 mg, 7.86 mmol) in DMF (20 mL) was added NaHMDS/THF (9.44 mL, 9.44 mmol) at rt. After stirring for 10 min, 4-chloro-2-fluoropyridine (1552 mg, 11.80 mmol) was added. The mixture was stirred at rt for 2 h, then at 75° C. o for 30 min for 1 h. EtOAc was added, washed with water, then concentrated. The residue was purified via silica gel chromatography (40 g, hexanes-20% EtOAc) to give 4-chloro-2-((2,2-difluorobut-3-en-1-yl)oxy)pyridine (710 mg, 3.23 mmol, 41.1% yield).

¹H NMR (499 MHz, chloroform-d δ 8.07-8.05 (m, 1H), 6.98-6.94 (m, 1H), 6.88 (d, J=1.8 Hz, 1H), 6.05 (dq, J=17.4, 11.1 Hz, 1H), 5.80 (dt, J=17.5, 2.5 Hz, 1H), 5.56 (d, J=11.1 Hz, 1H), 4.62 (t, J=12.3 Hz, 2H).

MS ESI m/z 220.0 (M+H)⁺.

### 1C: 4-((4-chloropyridin-2-yl)oxy)-3,3-difluorobutan-1-ol

To a soln of 4,4,5,5-tetramethyl-1,3,2-dioxaborolane (466 mg, 3.64 mmol) in THF (3 mL) was added rhodium(I) tris(triphenylphosphine) chloride (84 mg, 0.091 mmol) under N₂ at rt. After 5 min of stirring, 4-chloro-2-((2,2-difluorobut-3-en-1-yl)oxy)pyridine (200 mg, 0.911 mmol) was added. The mixture was stirred at rt for 16 h. Then 2 mL of 1 N NaOH was added dropwise followed by 30% wt hydrogen peroxide (1.0 mL, 9.79 mmol) dropwise. After stirring at rt for 30 min, EtOAc was added, washed with brine, then concentrated. The residue was purified via silica gel chromatography (12 g, hexanes-50% EtOAc) to give 4-((4-chloropyridin-2-yl)oxy)-3,3-difluorobutan-1-ol (150 mg, 0.631 mmol, 69.3% yield),

¹H NMR (499 MHz, chloroform-d) δ 8.06 (dd, J=5.7, 3.0 Hz, 1H), 6.96 (ddd, J=10.0, 5.5, 1.7 Hz, 1H), 6.88 (dd, J=8.6, 1.7 Hz, 1H), 4.60 (dt, J=15.0, 12.8 Hz, 2H), 4.12 (t, J=6.6 Hz, 1H), 3.99-3.91 (m, 2H), 2.39-2.28 (m, 2H).

MS ESI m/z 238.0 (M+H)⁺.

### 1D: tert-butyl (tert-butoxycarbonyl)(7-(3-(4-((4-chloropyridin-2-yl)oxy)-3,3-difluorobutoxy)-2-fluorophenyl)-[1,2,4]triazolo[1,5-a]pyridin-2-yl)carbamate

To a solution of Ph₃P (88 mg, 0.337 mmol) in THF (0.5 mL) was added DIAD (0.065 mL, 0.337 mmol) at rt. After stirring at rt for 5 min, 4-((4-chloropyridin-2-yl)oxy)-3,3-difluorobutan-1-ol (32 mg, 0.135 mmol) was added followed by A (150 mg, 0.337 mmol). The mixture was stirred at rt for 18 h. The mixture was concentrated. The residue was purified via silica gel chromatography (12 g, hexanes-40% EtOAc) to give tert-butyl (tert-butoxycarbonyl)(7-(3-(4-((4-chloropyridin-2-yl)oxy)-3,3-difluorobutoxy)-2-fluorophenyl)-[1,2,4]triazolo[1,5-a]pyridin-2-yl)carbamate 1D (30 mg, 0.045 mmol, 33.5% yield).

¹H NMR (499 MHz, chloroform-d) δ 8.58-8.56 (m, 1H), 8.05-8.03 (m, 1H), 7.83 (s, 1H), 7.24-7.18 (m, 2H), 7.12-7.05 (m, 2H), 6.94-6.92 (m, 1H), 6.83-6.81 (m, 1H), 4.69-4.62 (m, 2H), 4.39-4.34 (m, 2H), 2.69-2.58 (m, 2H), 1.49 (s, 18H).

MS ESI m/z 664.3 (M+H)⁺.

### 1: 7-(3-(4-((4-chloropyridin-2-yl)oxy)-3,3-difluorobutoxy)-2-fluorophenyl)-[1,2,4]triazolo[1,5-a]pyridin-2-amine

To a solution of 1D (30 mg, 0.045 mmol) in DCM (0.5 mL) was added TFA (0.4 mL, 5.19 mmol) at rt. The mixture was stirred at rt for 3 h. The mixture was concentrated. The residue was
was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 30% B, 30-70% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation to give 7-(3-(4-((4-chloropyridin-2-yl)oxy)-3,3-difluorobutoxy)-2-fluorophenyl)-[1,2,4]triazolo[1,5-a]pyridin-2-amine (8 mg, 0.017 mmol, 38.2% yield).

¹H NMR (500 MHz, DMSO-d₆) δ 8.60 (d, J=7.0 Hz, 1H), 8.16 (d, J=5.5 Hz, 1H), 7.45 (s, 1H), 7.33-7.14 (m, 4H), 7.07 (s, 1H), 6.99 (br d, J=6.7 Hz, 1H), 6.07 (s, 2H), 4.71 (br t, J=13.6 Hz, 2H), 4.35 (br t, J=6.1 Hz, 2H), 2.69-2.53 (m, 2H).

MS ESI m/z 464.1 (M+H)⁺.

### Example 2: 5-(3-(2-amino-[1,2,4]triazolo[1,5-a]pyridin-7-yl)-2-fluorophenoxy)-3,3-difluoro-2-(4-fluorophenyl)pentan-2-ol—(enantiomer 1)

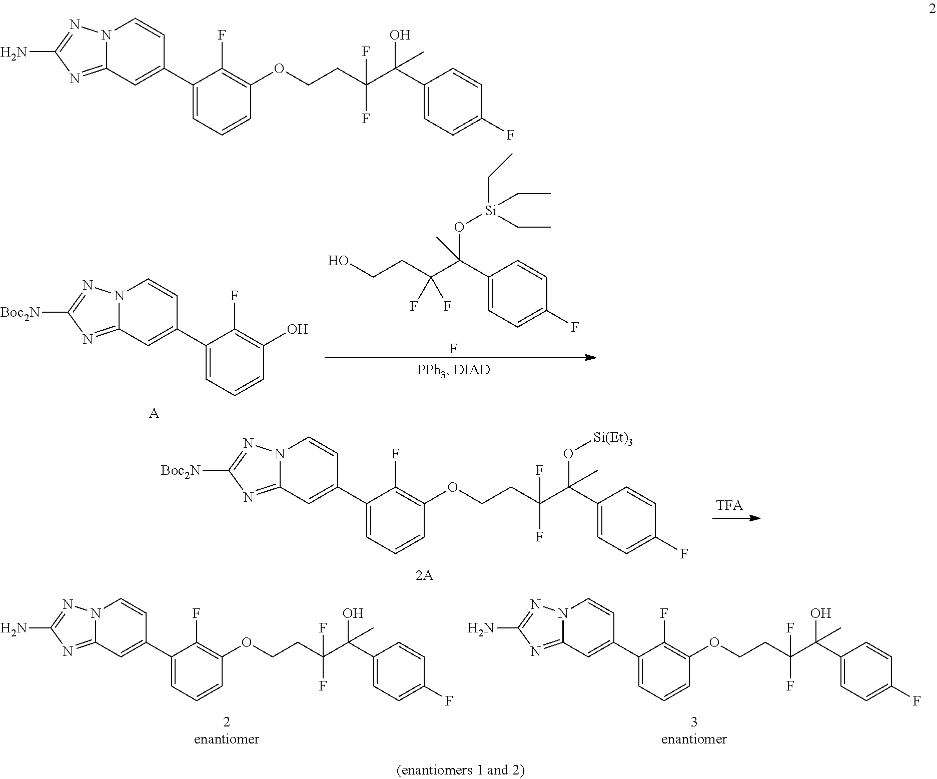

(The 2 unassigned enantiomers are:

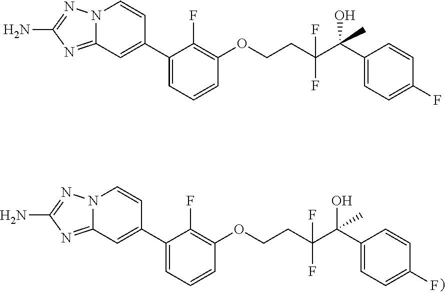 [SMILES: C[C@@](O)(c1ccc(F)cc1)C(F)(F)CCOc1cccc(-c2ccn3nc(N)nc3c2)c1F ; C[C@](O)(c1ccc(F)cc1)C(F)(F)CCOc1cccc(-c2ccn3nc(N)nc3c2)c1F]

### 2A: tert-butyl (tert-butoxycarbonyl)(7-(3-((3,3-difluoro-4-(4-fluorophenyl)-4-((triethylsilyl)oxy)-pentyl)oxy)-2-fluorophenyl)-[1,2,4]triazolo[1,5-a]pyridin-2-yl)carbamate

To a solution of A (140 mg, 0.316 mmol), 3,3-difluoro-4-(4-fluorophenyl)-4-((triethylsilyl)-oxy)pentan-1-ol (110 mg, 0.316 mmol) and Ph₃P (116 mg, 0.442 mmol) in THF (1.5 mL) was slowly added DIAD (0.086 mL, 0.442 mmol). The mixture was stirred at rt for 4 h. The mixture was concentrated, then purified via silica gel chromatography (12 g, hexanes-50% EtOAc) to give tert-butyl (tert-butoxycarbonyl)(7-(3-((3,3-difluoro-4-(4-fluorophenyl)-4-((triethylsilyl)oxy)pentyl)oxy)-2-fluorophenyl)-[1,2,4]triazolo[1,5-a]pyridin-2-yl)carbamate 2A (185 mg, 0.239 mmol, 76% yield).

¹H NMR (499 MHz, chloroform-d) δ 8.57 (dd, J=7.2, 0.7 Hz, 1H), 7.87-7.84 (m, 1H), 7.53 (dd, J=7.6, 5.3 Hz, 2H), 7.26 (dt, J=7.1, 1.8 Hz, 1H), 7.19-7.15 (m, 1H), 7.09-7.03 (m, 3H), 7.01 (td, J=8.0, 1.5 Hz, 1H), 4.28 (td, J=9.3, 5.9 Hz, 1H), 4.22-4.17 (m, 1H), 2.67-2.53 (m, 1H), 2.15-2.00 (m, 1H), 1.80 (s, 3H), 1.50 (s, 18H), 1.00-0.96 (m, 9H), 0.72-0.65 (m, 6H).

MS ESI m/z 775.5 (M+H)⁺.

### 2 and 3: 5-(3-(2-amino-[1,2,4]triazolo[1,5-a]pyridin-7-yl)-2-fluorophenoxy)-3,3-difluoro-2-(4-fluorophenyl)pentan-2-ol

To a solution of tert-butyl (tert-butoxycarbonyl)(7-(3-((3,3-difluoro-4-(4-fluorophenyl)-4-((triethylsilyl)oxy)pentyl)oxy)-2-fluorophenyl)-[1,2,4]triazolo[1,5-a]pyridin-2-yl)carbamate 2A (185 mg, 0.239 mmol) in DCM (1 mL) was slowly added TFA (1 mL, 12.98 mmol). The mixture was stirred at rt for 5 h, then stored in a fridge overnight. LCMS indicated both Boc and TES groups were deprotected. The mixture was concentrated, and purified via preparative Reverse Phase chromatography with the following conditions: Column: XBridge C18, 19 mm×200 mm, 5 m particles; Flow Rate: 20 mL/min; Column Temperature: 25° C. Fraction collection was triggered by UV (220 nm) and MS (ESI+). Fractions containing the desired product were combined and dried via centrifugal evaporation. The material was further purified via preparative chiral SFC with the following conditions: Column: Chiralcel OJ-H, 30 mm×250 mm, 5 m particles; Flow Rate: 100 mL/min; Column Temperature: 50° C. Fraction collection was triggered by UV (220 nm). Fractions containing the desired product were combined and dried via centrifugal evaporation. The first eluting isomer 2, 5-(3-(2-amino-[1,2,4]triazolo[1,5-a]pyridin-7-yl)-2-fluorophenoxy)-3,3-difluoro-2-(4-fluorophenyl)pentan-2-ol (21 mg, 0.045 mmol, 18.80% yield), and the second eluting isomer 3, 5-(3-(2-amino-[1,2,4]triazolo[1,5-a]pyridin-7-yl)-2-fluorophenoxy)-3,3-difluoro-2-(4-fluorophenyl)pentan-2-ol (22 mg, 0.047 mmol, 19.71% yield) were obtained.

2: ¹H NMR (500 MHz, DMSO-d₆) δ 8.60 (d, J=6.9 Hz, 1H), 7.59 (dd, J=8.3, 5.7 Hz, 2H), 7.47 (s, 1H), 7.25-7.15 (m, 5H), 7.02 (br d, J=6.9 Hz, 1H), 4.29-4.19 (m, 2H), 2.49-2.39 (m, 1H), 2.18-2.03 (m, 1H), 1.62 (s, 3H).

MS ESI m/z 461.2 (M+H)⁺.

### Example 3: 5-(3-(2-amino-[1,2,4]triazolo[1,5-a]pyridin-7-yl)-2-fluorophenoxy)-3,3-difluoro-2-(4-fluorophenyl)pentan-2-ol—(enantiomer 2)

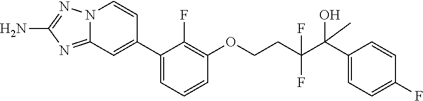 [SMILES: CC(O)(c1ccc(F)cc1)C(F)(F)CCOc1cccc(-c2ccn3nc(N)nc3c2)c1F]

3: ¹H NMR (500 MHz, DMSO-d₆) δ 8.59 (d, J=6.9 Hz, 1H), 7.59 (dd, J=8.3, 5.6 Hz, 2H), 7.47 (s, 1H), 7.24-7.15 (m, 5H), 7.02 (br d, J=7.0 Hz, 1H), 4.28-4.18 (m, 2H), 2.49-2.39 (m, 1H), 2.17-2.04 (m, 1H), 1.61 (s, 3H).

MS ESI m/z 461.2 (M+H)⁺.

### Example 4: 5-(3-(2-amino-[1,2,4]triazolo[1,5-a]pyridin-7-yl)-6-chloro-2-fluorophenoxy)-3,3-difluoro-2-(4-fluorophenyl)pentan-2-ol (enantiomer 1)

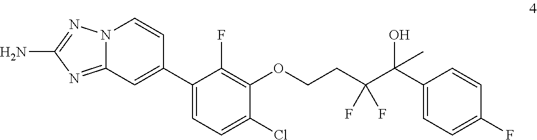 [SMILES: CC(O)(c1ccc(F)cc1)C(F)(F)CCOc1c(Cl)ccc(-c2ccn3nc(N)nc3c2)c1F]

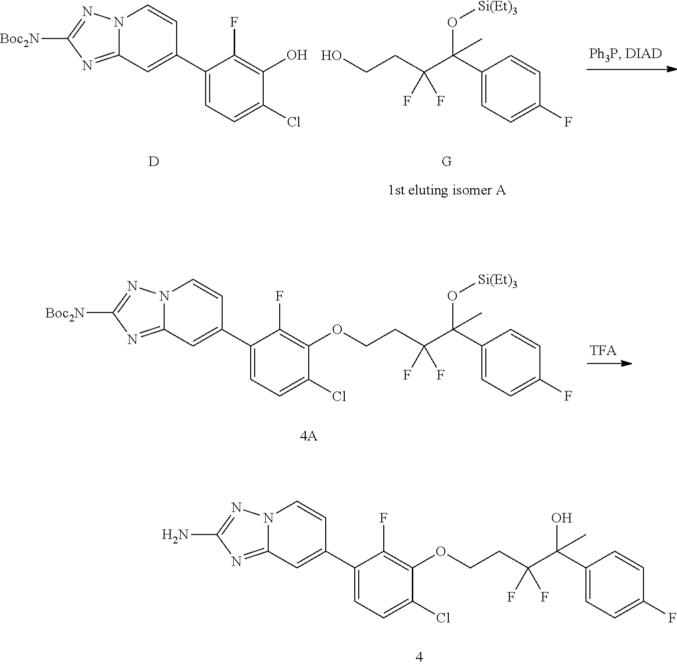

(The 2 unassigned enantiomers are:

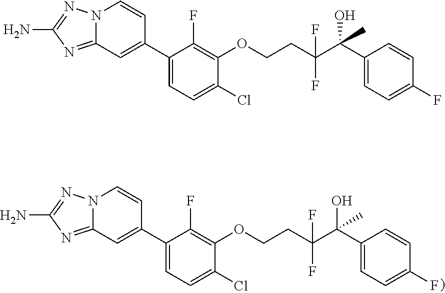 [SMILES: C[C@@](O)(c1ccc(F)cc1)C(F)(F)CCOc1c(Cl)ccc(-c2ccn3nc(N)nc3c2)c1F ; C[C@](O)(c1ccc(F)cc1)C(F)(F)CCOc1c(Cl)ccc(-c2ccn3nc(N)nc3c2)c1F]

4A: tert-butyl (tert-butoxycarbonyl)(7-(4-chloro-3-((3,3-difluoro-4-(4-fluorophenyl)-4-((triethylsilyl)oxy)pentyl)oxy)-2-fluorophenyl)-[1,2,4]triazolo[1,5-a]pyridin-2-yl)carbamate was prepared in a similar fashion to 2A using D and G as starting materials.

MS ESI m/z 809.6 (M+H)⁺.

### 4: 5-(3-(2-amino-[1,2,4]triazolo[1,5-a]pyridin-7-yl)-6-chloro-2-fluorophenoxy)-3,3-difluoro-2-(4-fluorophenyl)pentan-2-ol

To a solution of 4A (70 mg, 0.086 mmol) in DCM (0.5 mL) was added TFA dropwise (0.5 mL, 6.49 mmol) at rt. The mixture was stirred at rt for 20 h. LCMS indicated the deprotection was complete. The mixture was concentrated. The crude material was purified via preparative Reverse Phase chromatography with the following conditions: Column: XBridge C18, 19 mm×200 mm, 5 m particles; Flow Rate: 20 mL/min; Column Temperature: 25° C. Fraction collection was triggered by MS (ESI+). Fractions containing the desired product were combined and dried via centrifugal evaporation to obtain 5-(3-(2-amino-[1,2,4]triazolo[1,5-a]pyridin-7-yl)-6-chloro-2-fluorophenoxy)-3,3-difluoro-2-(4-fluorophenyl)pentan-2-ol (27 mg, 0.055 mmol, 63.1% yield).

¹H NMR (500 MHz, DMSO-d₆) δ 8.63 (d, J=6.9 Hz, 1H), 7.58-7.52 (m, 3H), 7.44-7.36 (m, 2H), 7.19-7.13 (m, 2H), 7.10 (br d, J=6.9 Hz, 1H), 4.27-4.15 (m, 2H), 2.49-2.39 (m, 1H), 2.18-2.04 (m, 1H), 1.59 (s, 3H) MS ESI m/z 495.1 (M+H)⁺.

### Example 5: 5-(3-(2-amino-[1,2,4]triazolo[1,5-a]pyridin-7-yl)-6-chloro-2-fluorophenoxy)-3,3-difluoro-2-(4-fluorophenyl)pentan-2-ol

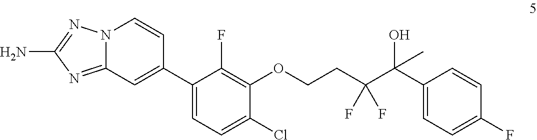 [SMILES: CC(O)(c1ccc(F)cc1)C(F)(F)CCOc1c(Cl)ccc(-c2ccn3nc(N)nc3c2)c1F]

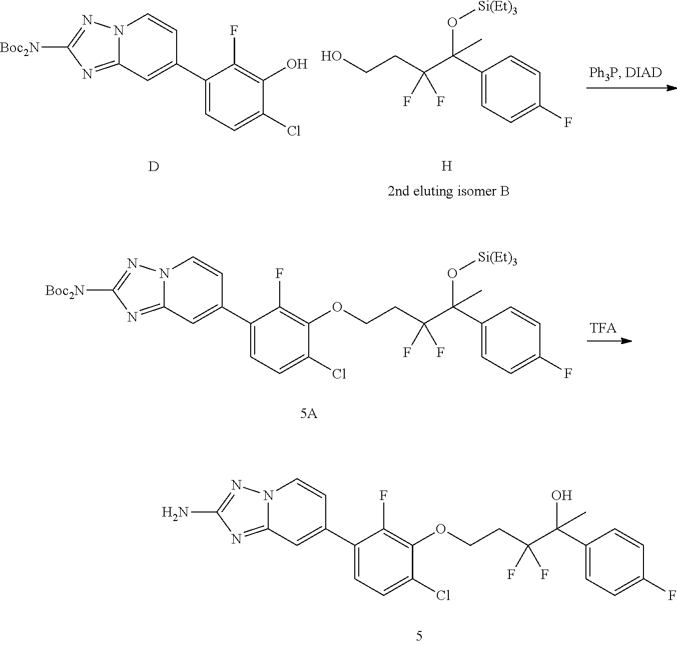

5A: tert-butyl (tert-butoxycarbonyl)(7-(4-chloro-3-((3,3-difluoro-4-(4-fluorophenyl)-4-((triethylsilyl)oxy)pentyl)oxy)-2-fluorophenyl)-[1,2,4]triazolo[1,5-a]pyridin-2-yl)carbamate was prepared in a similar fashion to 4A using D and H

MS ESI m/z 809.6 (M+H)⁺.

5: 5-(3-(2-amino-[1,2,4]triazolo[1,5-a]pyridin-7-yl)-6-chloro-2-fluorophenoxy)-3,3-difluoro-2-(4-fluorophenyl)pentan-2-ol was prepared in a similar fashion to 4 from 5A.

¹H NMR (500 MHz, DMSO-d₆) δ 8.59 (d, J=7.0 Hz, 1H), 7.58-7.52 (m, 2H), 7.50 (s, 1H), 7.43-7.35 (m, 2H), 7.18-7.13 (m, 2H), 7.04 (br d, J=7.1 Hz, 1H), 6.23 (s, 1H), 6.06 (s, 2H), 4.28-4.14 (m, 2H), 2.48-2.36 (m, 1H), 2.19-2.02 (m, 1H), 1.59 (s, 3H).

MS ESI m/z 495.1 (M+H)⁺.

### Example 6: 1-(3-(2-amino-[1,2,4]triazolo[1,5-a]pyridin-7-yl)-2-fluorophenoxy)-3,3-difluoro-6-methylheptan-4-ol (enantiomer 1)

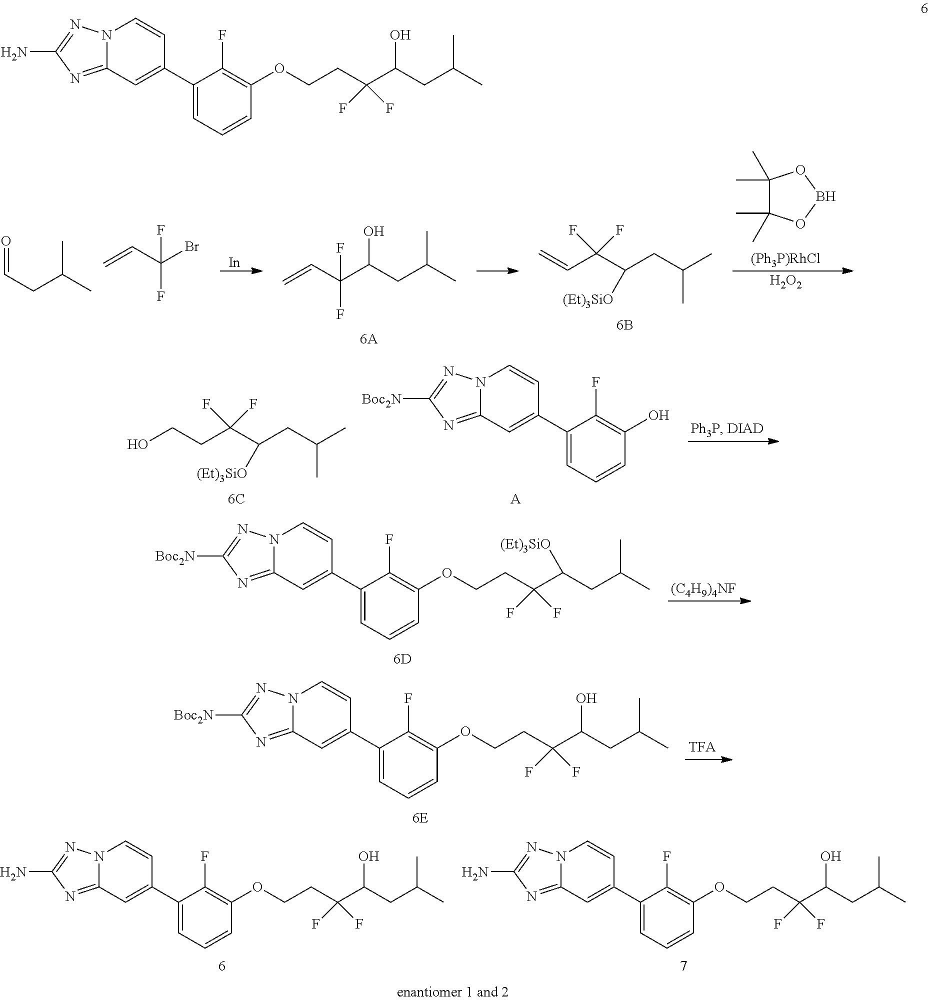

(The 2 unassigned enantiomers are:

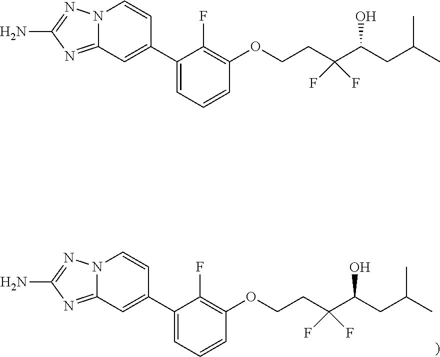 [SMILES: CC(C)C[C@@H](O)C(F)(F)CCOc1cccc(-c2ccn3nc(N)nc3c2)c1F ; CC(C)C[C@H](O)C(F)(F)CCOc1cccc(-c2ccn3nc(N)nc3c2)c1F]

6A: 3,3-difluoro-6-methylhept-1-en-4-ol was prepared in a similar fashion to F1.

¹H NMR (499 MHz, chloroform-d) δ 6.00 (ddt, J=17.4, 12.8, 11.1 Hz, 1H), 5.77-5.71 (m, 1H), 5.57 (d, J=11.1 Hz, 1H), 3.92-3.84 (m, 1H), 1.96-1.80 (m, 1H), 1.49-1.42 (m, 1H), 1.40-1.33 (m, 1H), 0.99 (d, J=6.8 Hz, 3H), 0.95 (d, J=6.7 Hz, 3H).

6B: ((3,3-difluoro-6-methylhept-1-en-4-yl)oxy)triethylsilane was prepared in a similar fashion to F4 from 6A.

¹H NMR (499 MHz, chloroform-d) δ 5.98 (dddd, J=17.5, 14.7, 11.1, 9.3 Hz, 1H), 5.68 (ddt, J=17.5, 3.0, 1.5 Hz, 1H), 5.51 (dt, J=11.1, 1.2 Hz, 1H), 3.92 (dddd, J=11.3, 9.4, 5.2, 3.1 Hz, 1H), 1.79 (dtd, J=10.0, 6.6, 3.6 Hz, 1H), 1.42-1.35 (m, 1H), 1.31-1.26 (m, 1H), 1.01-0.97 (t, 9H), 0.93 (dd, J=19.5, 6.6 Hz, 6H), 0.74-0.60 (m, 6H).

6C: 3,3-difluoro-6-methyl-4-((triethylsilyl)oxy)heptan-1-ol was prepared in a similar fashion to F from 6B.

¹H NMR (499 MHz, chloroform-d) δ 3.91-3.72 (m, 3H), 2.86 (br s, 1H), 2.32-2.16 (m, 1H), 2.08-1.95 (m, 1H), 1.80-1.70 (m, 1H), 1.52-1.45 (m, 1H), 1.36-1.29 (m, 1H), 0.96 (d, J=8.0 Hz, 15H), 0.73-0.60 (m, 6H).

6D: tert-butyl (tert-butoxycarbonyl)(7-(3-((3,3-difluoro-6-methyl-4-((triethylsilyl)oxy)-heptyl)oxy)-2-fluorophenyl)-[1,2,4]triazolo[1,5-a]pyridin-2-yl)carbamate was prepared in a similar fashion to 2A from 6C.

¹H NMR (499 MHz, chloroform-d) δ 8.58 (dd, J=7.1, 0.8 Hz, 1H), 7.88-7.87 (m, 1H), 7.30-7.27 (m, 1H), 7.23-7.19 (m, 1H), 7.12-7.07 (m, 2H), 4.41-4.32 (m, 2H), 3.96-3.88 (m, 1H), 2.68-2.52 (m, 1H), 2.46-2.31 (m, 1H), 1.85-1.76 (m, 1H), 1.50 (s, 18H), 1.48-1.43 (m, 1H), 1.40-1.35 (m, 1H), 1.00-0.92 (m, 15H), 0.72-0.61 (m, 6H).

MS ESI m/z 723.4 (M+H)⁺.

### 6E: tert-butyl (tert-butoxycarbonyl)(7-(3-((3,3-difluoro-4-hydroxy-6-methylheptyl)oxy)-2-fluorophenyl)-[1,2,4]triazolo[1,5-a]pyridin-2-yl)carbamate

To a solution of 6D (100 mg, 0.138 mmol) in THF (1 mL) was added TBAF/THF (36.2 mg, 0.138 mmol) at rt. The reaction was stirred at rt for 4 h. The mixture was concentrated. The residue was purified via silica gel chromatography (4 g, hexanes-100% EtOAc) to give 6E (75 mg, 0.123 mmol, 89% yield).

¹H NMR (499 MHz, chloroform-d) δ 8.59 (dd, J=7.2, 0.8 Hz, 1H), 7.88-7.86 (m, 1H), 7.30-7.26 (m, 1H), 7.25-7.20 (m, 1H), 7.12 (qd, J=7.4, 1.4 Hz, 2H), 4.40-4.31 (m, 2H), 3.97-3.87 (m, 1H), 2.75-2.60 (m, 1H), 2.54-2.42 (m, 1H), 1.97-1.86 (m, 1H), 1.64-1.54 (m, 1H), 1.51 (s, 18H), 1.48-1.43 (m, 1H), 1.01 (d, J=6.7 Hz, 3H), 0.95 (d, J=6.7 Hz, 3H)

MS ESI m/z 609.4 (M+H)⁺.

### 6 and 7: 1-(3-(2-amino-[1,2,4]triazolo[1,5-a]pyridin-7-yl)-2-fluorophenoxy)-3,3-difluoro-6-methylheptan-4-ol

To a solution of 6E (75 mg, 0.123 mmol) in DCM (1 mL) was added TFA (0.5 mL, 6.49 mmol) at rt. The reaction was stirred at rt for 4 h. The mixture was concentrated. The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 22% B, 22-62% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation. The material was further purified by SCP using SFC-chiral chromatography. Instrument: Waters 100 Prep SFC; Column: Chiral IC, 21×250 mm. 5 micron; Mobile Phase: 65% C_{02/35}% MeOH w/0.1% DEA; Flow Conditions: 60 mL/min; Detector Wavelength: 220 nm; Injection Details: 600 μL 40.7 mg dissolved in 3 mL MeOH. The first eluting enantiomer 6 1-(3-(2-amino-[1,2,4]triazolo[1,5-a]pyridin-7-yl)-2-fluorophenoxy)-3,3-difluoro-6-methylheptan-4-ol (14 mg, 0.034 mmol, 27.8% yield), and the second eluting enantiomer 7 1-(3-(2-amino-[1,2,4]triazolo[1,5-a]pyridin-7-yl)-2-fluorophenoxy)-3,3-difluoro-6-methylheptan-4-ol (13 mg, 0.032 mmol, 25.8% yield) were obtained. 6, ¹H NMR (500 MHz, DMSO-d₆) δ 8.61 (d, J=7.0 Hz, 1H), 7.49 (s, 1H), 7.33-7.24 (m, 2H), 7.22-7.18 (m, 1H), 7.04 (br d, J=6.7 Hz, 1H), 6.07 (s, 2H), 5.50 (d, J=7.0 Hz, 1H), 4.33 (br t, J=6.4 Hz, 2H), 3.77-3.67 (m, 1H), 2.50-2.40 (m, 2H), 1.86-1.77 (m, 1H), 1.48-1.40 (m, 1H), 1.32-1.25 (m, 1H), 0.94 (d, J=6.7 Hz, 3H), 0.88 (d, J=6.4 Hz, 3H).

MS ESI m/z 409.2 (M+H)⁺.

### Example 7: 1-(3-(2-amino-[1,2,4]triazolo[1,5-a]pyridin-7-yl)-2-fluorophenoxy)-3,3-difluoro-6-methylheptan-4-ol (enantiomer 2)

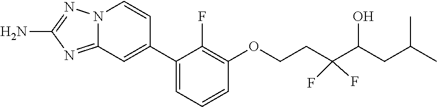 [SMILES: CC(C)CC(O)C(F)(F)CCOc1cccc(-c2ccn3nc(N)nc3c2)c1F]

7, ¹H NMR (500 MHz, DMSO-d₆) δ 8.60 (d, J=7.0 Hz, 1H), 7.49 (s, 1H), 7.27 (quin, J=7.9 Hz, 2H), 7.21-7.16 (m, 1H), 7.04 (br d, J=7.0 Hz, 1H), 4.32 (br t, J=6.3 Hz, 2H), 3.76-3.66 (m, 1H), 2.50-2.36 (m, 2H), 1.86-1.75 (m, 1H), 1.46-1.39 (m, 1H), 1.32-1.24 (m, 1H), 0.93 (d, J=6.7 Hz, 3H), 0.87 (d, J=6.7 Hz, 3H).

MS ESI m/z 409.2 (M+H)⁺.

### Example 8: 5-(3-(2-amino-[1,2,4]triazolo[1,5-a]pyridin-7-yl)-2,4-difluorophenoxy)-3,3-difluoro-2-(4-fluorophenyl)pentan-2-ol (enantiomer 1)

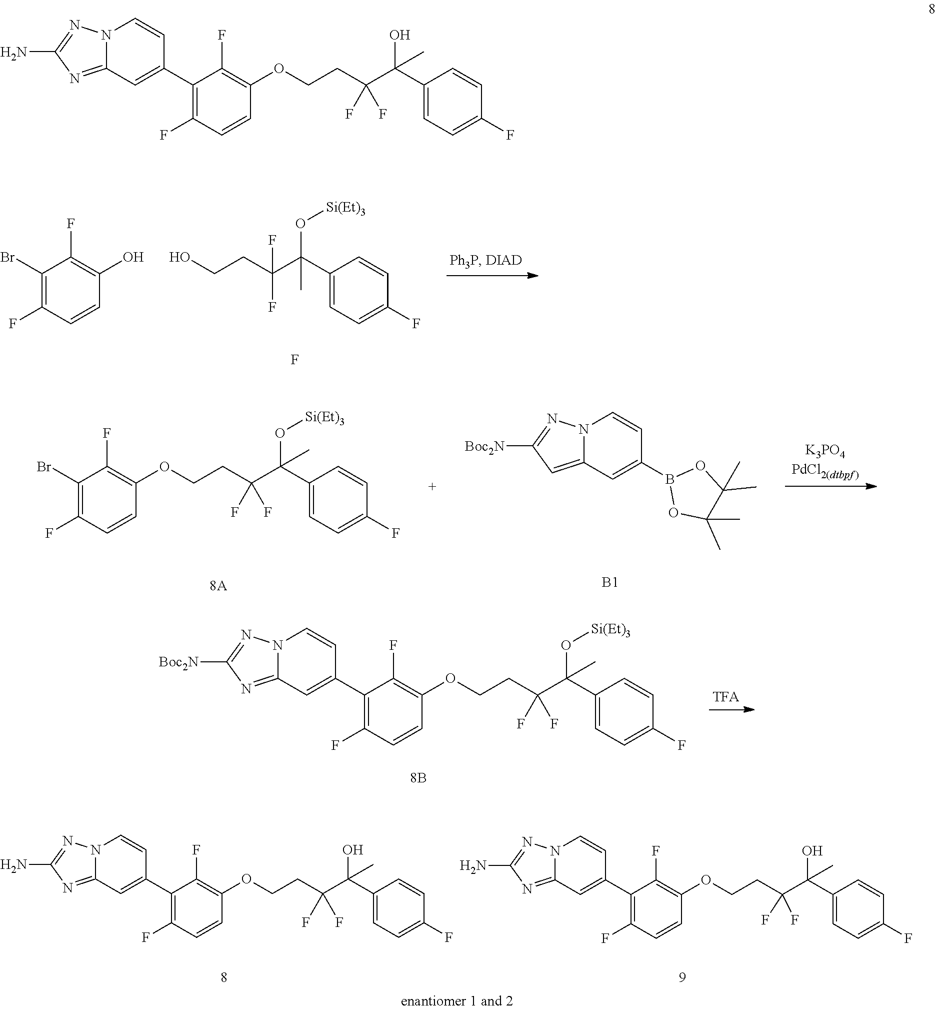

(The 2 unassigned enantiomers are:

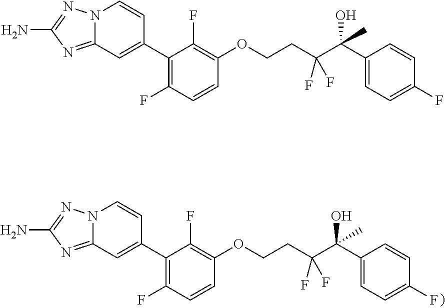 [SMILES: C[C@@](O)(c1ccc(F)cc1)C(F)(F)CCOc1ccc(F)c(-c2ccn3nc(N)nc3c2)c1F ; C[C@](O)(c1ccc(F)cc1)C(F)(F)CCOc1ccc(F)c(-c2ccn3nc(N)nc3c2)c1F]

8A: ((5-(3-bromo-2,4-difluorophenoxy)-3,3-difluoro-2-(4-fluorophenyl)pentan-2-yl)oxy)triethylsilane was prepared in a similar fashion to 2A from F and 3-bromo-2,4-difluorophenol.

¹H NMR (499 MHz, chloroform-d) δ 7.54-7.48 (m, 2H), 7.08-7.03 (m, 2H), 6.89-6.82 (m, 2H), 4.19 (td, J=9.2, 5.8 Hz, 1H), 4.11 (td, J=9.3, 5.7 Hz, 1H), 2.61-2.46 (m, 1H), 2.08-1.94 (m, 1H), 1.79 (s, 3H), 0.98 (t, J=7.9 Hz, 9H), 0.74-0.62 (m, 6H)

### 8B: tert-butyl (tert-butoxycarbonyl)(7-(3-((3,3-difluoro-4-(4-fluorophenyl)-4-((triethylsilyl)oxy)-pentyl)oxy)-2,6-difluorophenyl)-[1,2,4]triazolo[1,5-a]pyridin-2-yl)carbamate

To 8A ((5-(3-bromo-2,4-difluorophenoxy)-3,3-difluoro-2-(4-fluorophenyl)pentan-2-yl)oxy)-triethylsilane (75 mg, 0.139 mmol) in dioxane (0.8 mL) was added bis(diphenylphosphino)-ferrocene]dichloropalladium(II) 98% (4.53 mg, 6.95 μmol), B1 (77 mg, 0.167 mmol) and tripotassium phosphate (0.209 mL, 0.417 mmol). The mixture was purged with N₂, then stirred at 100° C. for about 40 min. Water was added, and extracted with 2× EtOAc. The organic layer was concentrated. The residue was purified via silica gel chromatography (4 g, hexanes-100% EtOAc) to give product tert-butyl (tert-butoxycarbonyl)(7-(3-((3,3-difluoro-4-(4-fluorophenyl)-4-((triethylsilyl)oxy)pentyl)oxy)-2,6-difluorophenyl)-[1,2,4]triazolo[1,5-a]pyridin-2-yl)carbamate 8B (100 mg, 0.126 mmol, 91% yield).

¹H NMR (499 MHz, chloroform-d) δ 8.60 (dd, J=7.2, 0.8 Hz, 1H), 7.84 (s, 1H), 7.54-7.50 (m, 2H), 7.15 (dd, J=7.2, 1.4 Hz, 1H), 7.08-7.03 (m, 2H), 6.99-6.93 (m, 2H), 4.24 (td, J=9.2, 5.9 Hz, 1H), 4.15 (td, J=9.2, 5.7 Hz, 1H), 2.65-2.50 (m, 1H), 2.11-1.97 (m, 1H), 1.79 (s, 3H), 1.51 (s, 18H), 1.00-0.95 (m, 9H), 0.75-0.62 (m, 6H).

MS ESI m/z 793.7 (M+H)⁺.

8 and 9: The enantiomers of 5-(3-(2-amino-[1,2,4]triazolo[1,5-a]pyridin-7-yl)-2,4-difluorophenoxy)-3,3-difluoro-2-(4-fluorophenyl)pentan-2-ol were prepared in a similar fashion to 6 and 7.

The first eluting enantiomer 8 5-(3-(2-amino-[1,2,4]triazolo[1,5-a]pyridin-7-yl)-2,4-difluorophenoxy)-3,3-difluoro-2-(4-fluorophenyl)pentan-2-ol (8 mg, 0.017 mmol, 13.26% yield), and the second eluting enantiomer 9 5-(3-(2-amino-[1,2,4]triazolo[1,5-a]pyridin-7-yl)-2,4-difluorophenoxy)-3,3-difluoro-2-(4-fluorophenyl)pentan-2-ol (13 mg, 0.027 mmol, 21.39% yield) were obtained.

8, ¹H NMR (500 MHz, DMSO-d₆) δ 8.62 (d, J=6.9 Hz, 1H), 7.59-7.54 (m, 2H), 7.43 (s, 1H), 7.26-7.20 (m, 1H), 7.20-7.13 (m, 3H), 6.92 (br d, J=6.4 Hz, 1H), 6.09 (s, 2H), 4.25-4.16 (m, 2H), 2.49-2.38 (m, 1H), 2.13-2.01 (m, 1H), 1.60 (s, 3H).

MS ESI m/z 479.2 (M+H)⁺.

### Example 9: 5-(3-(2-amino-[1,2,4]triazolo[1,5-a]pyridin-7-yl)-2,4-difluorophenoxy)-3,3-difluoro-2-(4-fluorophenyl)pentan-2-ol (enantiomer 2)

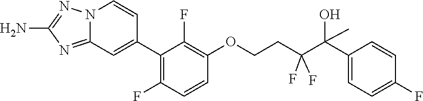 [SMILES: CC(O)(c1ccc(F)cc1)C(F)(F)CCOc1ccc(F)c(-c2ccn3nc(N)nc3c2)c1F]

9, ¹H NMR (500 MHz, DMSO-d₆) δ 8.62 (d, J=6.9 Hz, 1H), 7.57 (br dd, J=8.1, 6.0 Hz, 2H), 7.44 (s, 1H), 7.24 (td, J=9.0, 5.2 Hz, 1H), 7.21-7.13 (m, 3H), 6.92 (br d, J=6.8 Hz, 1H), 6.09 (s, 2H), 4.26-4.16 (m, 2H), 2.49-2.39 (m, 1H), 2.14-2.01 (m, 1H), 1.60 (s, 3H).

MS ESI m/z 479.2 (M+H)⁺.

### Example 10: 5-(3-(2-amino-[1,2,4]triazolo[1,5-a]pyridin-7-yl)-2,5-difluorophenoxy)-3,3-difluoro-2-(4-fluorophenyl)pentan-2-ol (enantiomer 1)

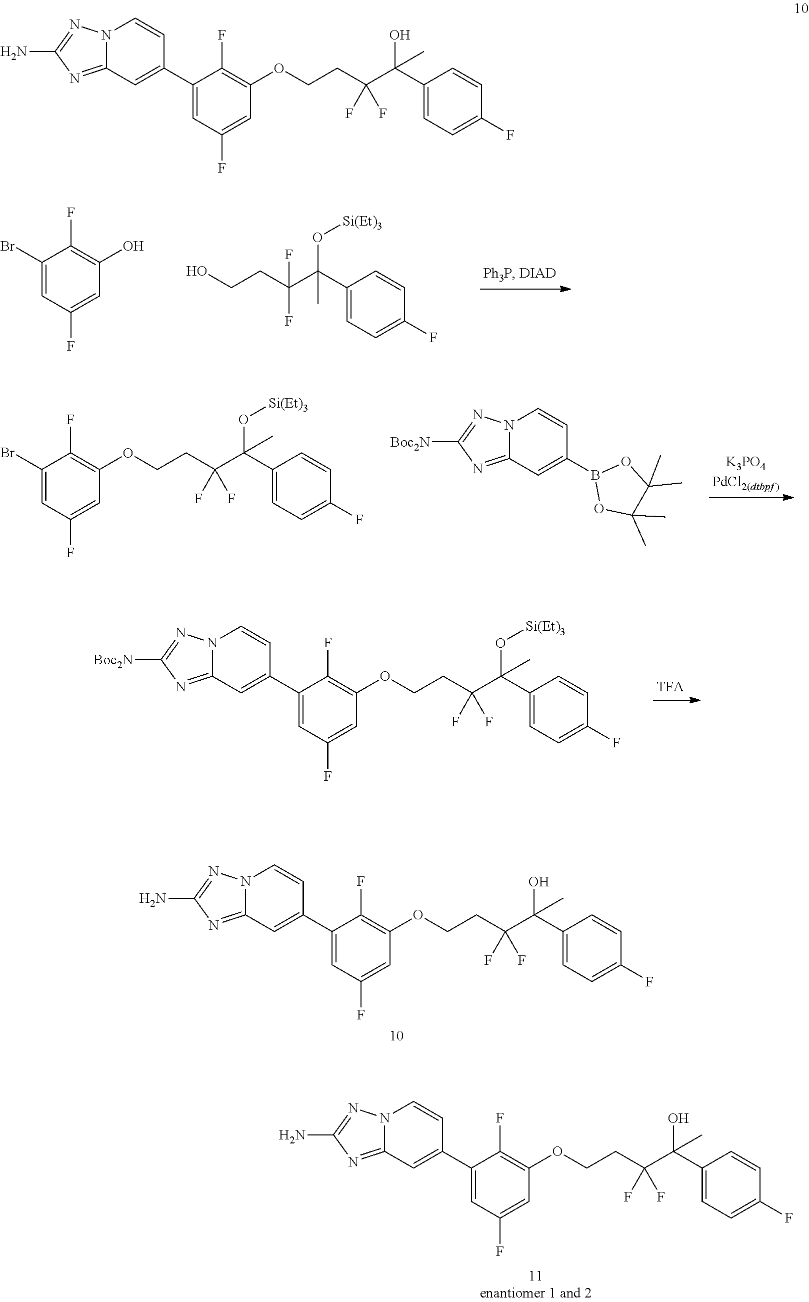

(The 2 unassigned enantiomers are:

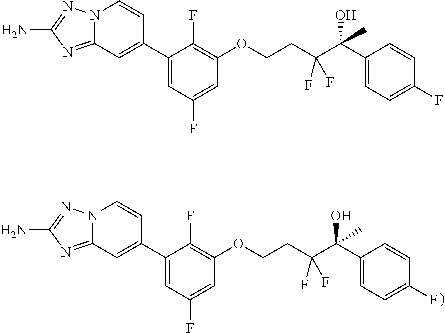 [SMILES: C[C@@](O)(c1ccc(F)cc1)C(F)(F)CCOc1cc(F)cc(-c2ccn3nc(N)nc3c2)c1F ; C[C@](O)(c1ccc(F)cc1)C(F)(F)CCOc1cc(F)cc(-c2ccn3nc(N)nc3c2)c1F]

10 and 11: Both enantiomers of 5-(3-(2-amino-[1,2,4]triazolo[1,5-a]pyridin-7-yl)-2,5-difluorophenoxy)-3,3-difluoro-2-(4-fluorophenyl)pentan-2-ol were prepared in a similar fashion to 8 and 9.

The first eluting enantiomer 10 5-(3-(2-amino-[1,2,4]triazolo[1,5-a]pyridin-7-yl)-2,5-difluorophenoxy)-3,3-difluoro-2-(4-fluorophenyl)pentan-2-ol (2 mg, 4.18 μmol, 4.14% yield), and the second eluting enantiomer 11 5-(3-(2-amino-[1,2,4]triazolo[1,5-a]pyridin-7-yl)-2,5-difluorophenoxy)-3,3-difluoro-2-(4-fluorophenyl)pentan-2-ol (2 mg, 4.18 μmol, 4.14% yield) were obtained.

10, ¹H NMR (500 MHz, DMSO-d₆) δ 8.37 (d, J=6.9 Hz, 1H), 7.38-7.33 (m, 2H), 7.28 (s, 1H), 6.96 (br t, J=8.7 Hz, 3H), 6.86-6.79 (m, 2H), 5.96 (s, 1H), 5.85 (s, 2H), 4.05-3.99 (m, 2H), 2.26-2.17 (m, 1H), 1.93-1.80 (m, 1H), 1.38 (s, 3H).

MS ESI m/z 479.2 (M+H)⁺.

### Example 11: 5-(3-(2-amino-[1,2,4]triazolo[1,5-a]pyridin-7-yl)-2,5-difluorophenoxy)-3,3-difluoro-2-(4-fluorophenyl)pentan-2-ol (enantiomer 2)

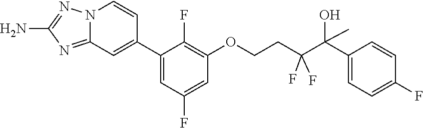 [SMILES: CC(O)(c1ccc(F)cc1)C(F)(F)CCOc1cc(F)cc(-c2ccn3nc(N)nc3c2)c1F]

11, ¹H NMR (500 MHz, DMSO-d₆) δ 8.61 (d, J=7.2 Hz, 1H), 7.61-7.57 (m, 2H), 7.52 (s, 1H), 7.19 (br t, J=8.9 Hz, 3H), 7.09-7.02 (m, 2H), 6.19 (s, 1H), 6.09 (s, 2H), 4.30-4.21 (m, 2H), 2.49-2.41 (m, 1H), 2.15-2.03 (m, 1H), 1.61 (s, 3H) MS ESI m/z 479.2 (M+H)⁺.

### Example 12: 5-(3-(2-amino-[1,2,4]triazolo[1,5-a]pyridin-7-yl)-2-fluorophenoxy)-3,3-difluoro-2-(4-fluorophenyl)pentan-5,5-d2-2-ol

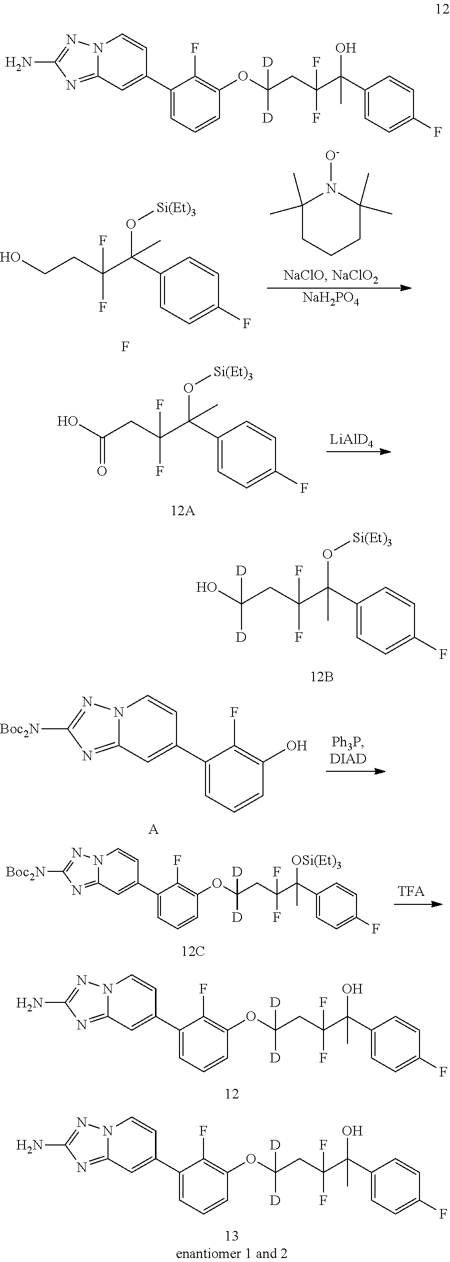

(The 2 unassigned enantiomers are:

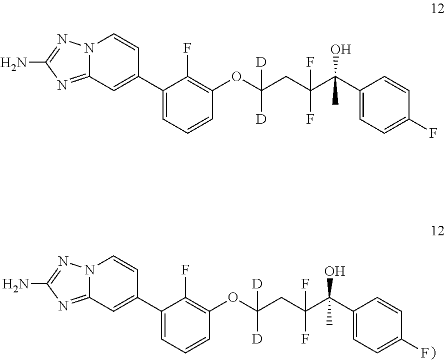

### 12A: 3,3-difluoro-4-(4-fluorophenyl)-4-((triethylsilyl)oxy)pentanoic acid

To a solution of F 3,3-difluoro-4-(4-fluorophenyl)-4-((triethylsilyl)oxy)pentan-1-ol (500 mg, 1.435 mmol) in THF (4 mL) and water (4 mL) was added sodium dihydrogen phosphate (430 mg, 3.59 mmol) followed by TEMPO (56.0 mg, 0.359 mmol), sodium chlorite (260 mg, 2.87 mmol) and sodium hypochlorite (0.221 mL, 0.359 mmol). The mixture was stirred at rt for 3 h. TLC indicated the SM was consumed. EtOAc was added, washed with 0.1 N HCl and water, then concentrated. The residue was purified via silica gel chromatography (12 g, hexanes-100% EtOAc) to give 12A 3,3-difluoro-4-(4-fluorophenyl)-4-((triethylsilyl)oxy)pentanoic acid (344 mg, 0.949 mmol, 66.1% yield).

¹H NMR (499 MHz, chloroform-d) δ 11.00-9.44 (m, 1H), 7.51 (ddd, J=8.7, 5.3, 1.2 Hz, 2H), 7.08-7.02 (m, 2H), 3.11-2.98 (m, 1H), 2.59-2.47 (m, 1H), 1.80 (s, 3H), 0.97 (t, J=8.0 Hz, 9H), 0.74-0.63 (m, 6H). 12B: 3,3-difluoro-4-(4-fluorophenyl)-4-((triethylsilyl)oxy)pentan-1,1-d2-1-ol:

To a solution of 12A 3,3-difluoro-4-(4-fluorophenyl)-4-((triethylsilyl)oxy)pentanoic acid (50 mg, 0.138 mmol) in THF (1 mL) was added lithium aluminum deuteride, 98% isotopic purity (11.58 mg, 0.276 mmol) at rt under N₂. The mixture was stirred at rt for 2 h. TLC indicated the SM was consumed. Et₂O was added followed by a couple of drops of water to quench the reaction. Na₂SO₄ was added. After stirring for 2 h, the solid was filtered out. The filtrate was concentrated to give crude 3,3-difluoro-4-(4-fluorophenyl)-4-((triethylsilyl)oxy)pentan-1,1-d2-1-ol 12B (45 mg, 0.128 mmol, 93% yield).

¹H NMR (499 MHz, chloroform-d) δ 7.49 (ddd, J=8.8, 5.3, 1.5 Hz, 2H), 7.07-7.01 (m, 2H), 2.32-2.20 (m, 1H), 1.81-1.71 (m, 4H), 0.97 (t, J=7.9 Hz, 9H), 0.68 (qd, J=8.0, 2.9 Hz, 6H).

### 12C: tert-butyl (tert-butoxycarbonyl)(7-(3-((3,3-difluoro-4-(4-fluorophenyl)-4-((triethylsilyl)oxy)pentyl-1,1-d2)oxy)-2-fluorophenyl)-[1,2,4]triazolo[1,5-a]pyridin-2-yl)carbamate

To a solution of A (57.1 mg, 0.128 mmol), Ph₃P (50.5 mg, 0.193 mmol) and 12B 3,3-difluoro-4-(4-fluorophenyl)-4-((triethylsilyl)oxy)pentan-1,1-d2-1-ol (45 mg, 0.128 mmol) in THF (0.6 mL) was added DIAD (0.037 mL, 0.193 mmol) dropwise. The mixture was stirred at rt for 20 h. LCMS/TLC indicated SM was consumed, and the product was formed. The mixture was concentrated. The residue was purified via silica gel chromatography (4 g, hexanes-40% EtOAc) to give product tert-butyl (tert-butoxycarbonyl)(7-(3-((3,3-difluoro-4-(4-fluorophenyl)-4-((triethylsilyl)oxy)pentyl-1,1-d2)oxy)-2-fluorophenyl)-[1,2,4]triazolo[1,5-a]pyridin-2-yl)carbamate 12C (80 mg, 0.103 mmol, 80% yield), (M+H)⁺=777.6.

¹H NMR (400 MHz, chloroform-d) δ 8.57 (dd, J=7.1, 0.9 Hz, 1H), 7.87-7.84 (m, 1H), 7.53 (ddd, J=8.7, 5.4, 1.3 Hz, 2H), 7.26 (dt, J=7.1, 1.8 Hz, 1H), 7.20-7.15 (m, 1H), 7.09-6.98 (m, 4H), 2.66-2.52 (m, 1H), 2.13-1.99 (m, 1H), 1.80 (s, 3H), 1.51 (s, 18H), 1.01-0.96 (m, 9H), 0.73-0.65 (m, 6H).

MS ESI m/z 777.6 (M+H)⁺.

12 and 13: 5-(3-(2-amino-[1,2,4]triazolo[1,5-a]pyridin-7-yl)-2-fluorophenoxy)-3,3-difluoro-2-(4-fluorophenyl)pentan-5,5-d2-2-ol was prepared in a similar fashion to 2 and 3.

The first eluting isomer 12 5-(3-(2-amino-[1,2,4]triazolo[1,5-a]pyridin-7-yl)-2-fluorophenoxy)-3,3-difluoro-2-(4-fluorophenyl)pentan-5,5-d2-2-ol (7 mg, 0.015 mmol, 14.70% yield) and the second eluting isomer 13 5-(3-(2-amino-[1,2,4]triazolo[1,5-a]pyridin-7-yl)-2-fluorophenoxy)-3,3-difluoro-2-(4-fluorophenyl)pentan-5,5-d2-2-ol (8 mg, 0.017 mmol, 16.80% yield) were obtained.

12, ¹H NMR (500 MHz, DMSO-d₆) δ 8.59 (d, J=7.0 Hz, 1H), 7.59 (br dd, J=7.9, 5.9 Hz, 2H), 7.47 (s, 1H), 7.25-7.14 (m, 5H), 7.02 (br d, J=6.9 Hz, 1H), 2.50-2.40 (m, 1H), 2.15-2.02 (m, 1H), 1.62 (s, 3H).

MS ESI m/z 463.2 (M+H)+

### Example 13: 5-(3-(2-amino-[1,2,4]triazolo[1,5-a]pyridin-7-yl)-2-fluorophenoxy)-3,3-difluoro-2-(4-fluorophenyl)pentan-5,5-d2-2-ol (enantiomer 2)

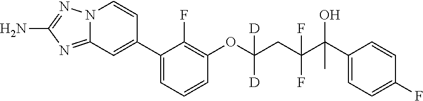 [SMILES: [2H]C([2H])(CC(F)(F)C(C)(O)c1ccc(F)cc1)Oc1cccc(-c2ccn3nc(N)nc3c2)c1F]

13, ¹H NMR (500 MHz, DMSO-d₆) δ 8.59 (d, J=7.0 Hz, 1H), 7.59 (dd, J=8.2, 5.8 Hz, 2H), 7.47 (s, 1H), 7.24-7.14 (m, 5H), 7.02 (br d, J=6.9 Hz, 1H), 2.49-2.40 (m, 1H), 2.14-2.02 (m, 1H), 1.61 (s, 3H).

MS ESI m/z 463.2 (M+H)⁺.

### Example 14: 5-(3-(2-amino-[1,2,4]triazolo[1,5-a]pyridin-7-yl)-2,6-difluorophenoxy)-3,3-difluoro-2-(4-fluorophenyl)pentan-5,5-d2-2-ol (enantiomer 1)

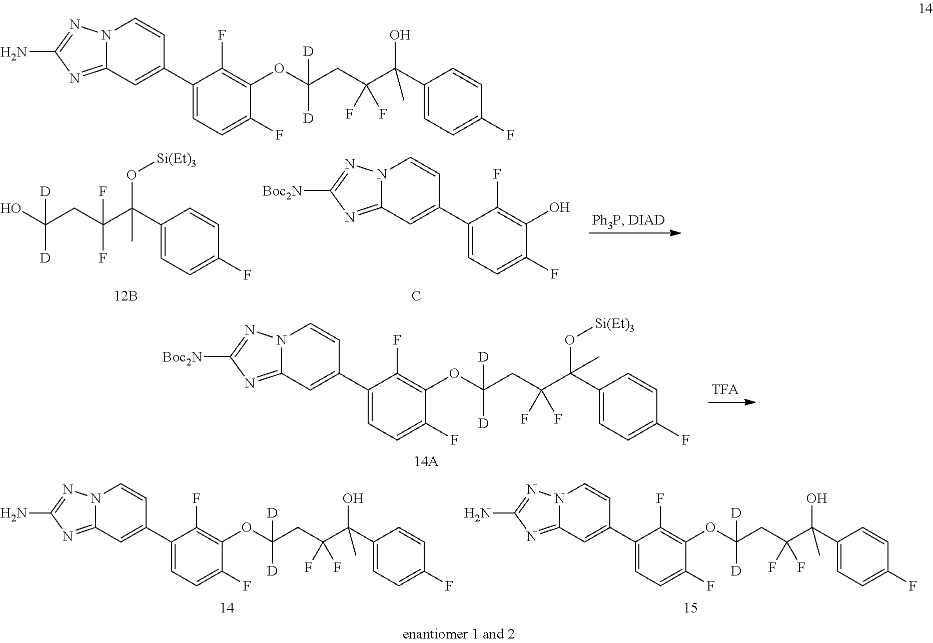

(The 2 unassigned enantiomers are:

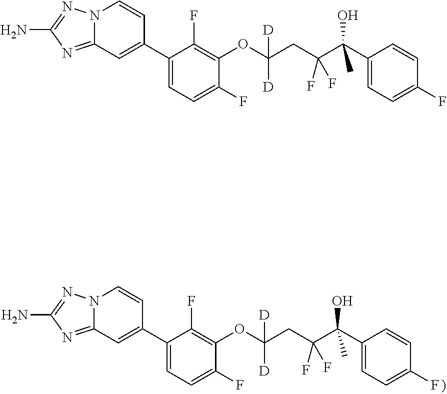 [SMILES: [2H]C([2H])(CC(F)(F)[C@@](C)(O)c1ccc(F)cc1)Oc1c(F)ccc(-c2ccn3nc(N)nc3c2)c1F ; [2H]C([2H])(CC(F)(F)[C@](C)(O)c1ccc(F)cc1)Oc1c(F)ccc(-c2ccn3nc(N)nc3c2)c1F]

14A: tert-butyl (tert-butoxycarbonyl)(7-(3-((3,3-difluoro-4-(4-fluorophenyl)-4-((triethylsilyl)-oxy)pentyl-1,1-d2)oxy)-2,4-difluorophenyl)-[1,2,4]triazolo[1,5-a]pyridin-2-yl)carbamate was prepared in similar fashion to 2A from 3,3-difluoro-4-(4-fluorophenyl)-4-((triethylsilyl)oxy)-pentan-1,1-d2-1-ol 12B and tert-butyl (tert-butoxycarbonyl)(7-(2,4-difluoro-3-hydroxyphenyl)-[1,2,4]triazolo[1,5-a]pyridin-2-yl)carbamate C.

¹H NMR (499 MHz, chloroform-d) δ 8.58 (dd, J=7.1, 0.8 Hz, 1H), 7.82-7.79 (m, 1H), 7.55-7.46 (m, 2H), 7.22-7.18 (m, 1H), 7.18-7.12 (m, 1H), 7.07-6.98 (m, 3H), 2.60-2.48 (m, 1H), 2.10-1.99 (m, 1H), 1.77 (s, 3H), 1.51 (s, 18H), 0.99-0.93 (m, 9H), 0.71-0.62 (m, 6H).

MS ESI m/z 795.7 (M+H)+

14 and 15: The two enantiomers of 5-(3-(2-amino-[1,2,4]triazolo[1,5-a]pyridin-7-yl)-2,6-difluorophenoxy)-3,3-difluoro-2-(4-fluorophenyl)pentan-5,5-d2-2-ol were prepared in a similar fashion to 2 and 3.

The first eluting isomer 14 5-(3-(2-amino-[1,2,4]triazolo[1,5-a]pyridin-7-yl)-2,6-difluorophenoxy)-3,3-difluoro-2-(4-fluorophenyl)pentan-5,5-d2-2-ol (7.5 mg, 0.015 mmol, 14.23% yield), and the second eluting isomer 15 5-(3-(2-amino-[1,2,4]triazolo[1,5-a]pyridin-7-yl)-2,6-difluorophenoxy)-3,3-difluoro-2-(4-fluorophenyl)pentan-5,5-d2-2-ol (7.4 mg, 0.015 mmol, 14.40% yield) were obtained.

14, ¹H NMR (500 MHz, DMSO-d₆) δ 8.60 (d, J=6.9 Hz, 1H), 7.56 (br dd, J=8.3, 5.7 Hz, 2H), 7.48 (s, 1H), 7.38 (td, J=8.4, 6.1 Hz, 1H), 7.25 (br t, J=9.3 Hz, 1H), 7.17 (t, J=8.9 Hz, 2H), 7.01 (br d, J=6.9 Hz, 1H), 6.19 (s, 1H), 6.07 (s, 2H), 2.48-2.36 (m, 1H), 2.13-2.02 (m, 1H), 1.60 (s, 3H).

MS ESI m/z 481.2 (M+H)⁺.

### Example 15: 5-(3-(2-amino-[1,2,4]triazolo[1,5-a]pyridin-7-yl)-2,6-difluorophenoxy)-3,3-difluoro-2-(4-fluorophenyl)pentan-5,5-d2-2-ol (enantiomer 2)

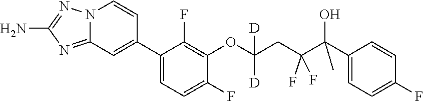 [SMILES: [2H]C([2H])(CC(F)(F)C(C)(O)c1ccc(F)cc1)Oc1c(F)ccc(-c2ccn3nc(N)nc3c2)c1F]

15, ¹H NMR (500 MHz, DMSO-d₆) δ 8.60 (d, J=6.9 Hz, 1H), 7.55 (br dd, J=8.0, 5.8 Hz, 2H), 7.48 (s, 1H), 7.40-7.34 (m, 1H), 7.25 (br t, J=9.7 Hz, 1H), 7.17 (t, J=8.9 Hz, 2H), 7.01 (br d, J=6.9 Hz, 1H), 6.19 (s, 1H), 6.06 (s, 2H), 2.48-2.35 (m, 1H), 2.13-2.01 (m, 1H), 1.59 (s, 3H) MS ESI m/z 481.2 (M+H)⁺.

### Example 16: 7-(3-((4-amino-3,3-difluoro-4-(4-fluorophenyl)pentyl)oxy)-2,4-difluorophenyl)-[1,2,4]triazolo[1,5-a]pyridin-2-amine (enantiomer 1)

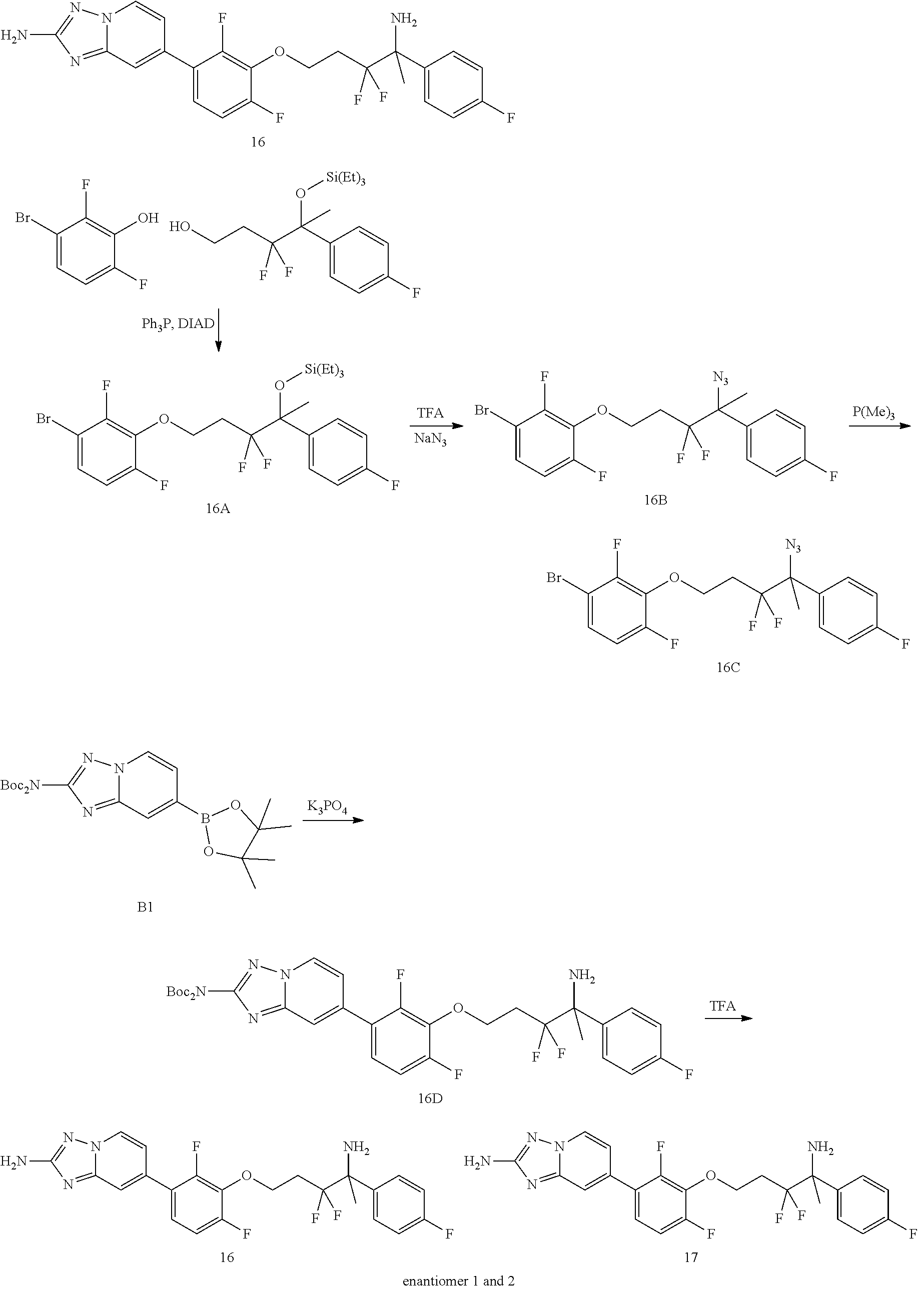

(The 2 unassigned enantiomers are:

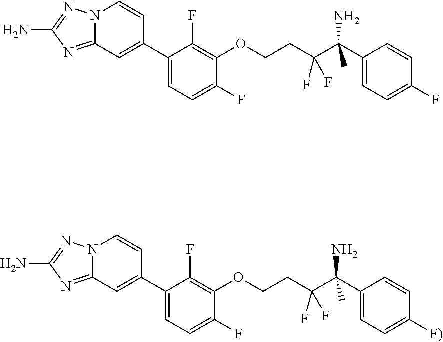

16A: ((5-(3-bromo-2,6-difluorophenoxy)-3,3-difluoro-2-(4-fluorophenyl)pentan-2-yl)oxy)triethylsilane was prepared in a similar fashion to 8A from 3-bromo-2,6-difluorophenol and 3,3-difluoro-4-(4-fluorophenyl)-4-((triethylsilyl)oxy)pentan-1-ol.

¹H NMR (499 MHz, chloroform-d) δ 7.55-7.49 (m, 2H), 7.20 (ddd, J=9.1, 7.2, 5.2 Hz, 1H), 7.09-7.03 (m, 2H), 6.82 (ddd, J=10.0, 9.1, 2.1 Hz, 1H), 4.34 (td, J=9.3, 6.0 Hz, 1H), 4.25 (td, J=9.3, 5.9 Hz, 1H), 2.61-2.46 (m, 1H), 2.11-1.97 (m, 1H), 1.79 (s, 3H), 0.98 (t, J=7.9 Hz, 9H), 0.74-0.64 (m, 6H).

### 16B: 2-((4-azido-3,3-difluoro-4-(4-fluorophenyl)pentyl)oxy)-4-bromo-1,3-difluorobenzene

To a solution of 16A ((5-(3-bromo-2,6-difluorophenoxy)-3,3-difluoro-2-(4-fluorophenyl)pentan-2-yl)oxy)triethylsilane (100 mg, 0.185 mmol) in DCM (0.5 mL) was added TFA (0.5 mL, 6.49 mmol). The mixture was stirred at rt for 20 h. LCMS indicated deprotection was complete. Sodium azide (60.3 mg, 0.927 mmol) was added. After stirring at 55° C. for 6 h. LCMS indicated most of SM was consumed. The mixture was concentrated. EtOAc was added, washed with NaHCO₃, then concentrated. The residue was purified via silica gel chromatography (4 g, hexanes-20% EtOAc) to give 2-((4-azido-3,3-difluoro-4-(4-fluorophenyl)pentyl)oxy)-4-bromo-1,3-difluorobenzene (15 mg, 0.033 mmol, 17.97% yield).

¹H NMR (499 MHz, chloroform-d) δ 7.56-7.51 (m, 2H), 7.21 (ddd, J=9.1, 7.2, 5.3 Hz, 1H), 7.13-7.07 (m, 2H), 6.83 (ddd, J=10.0, 9.2, 2.1 Hz, 1H), 4.33-4.23 (m, 2H), 2.54-2.42 (m, 1H), 2.10-1.97 (m, 1H), 1.90 (s, 3H).

### 16C: 5-(3-bromo-2,6-difluorophenoxy)-3,3-difluoro-2-(4-fluorophenyl)pentan-2-amine

To a solution of 16B 2-((4-azido-3,3-difluoro-4-(4-fluorophenyl)pentyl)oxy)-4-bromo-1,3-difluorobenzene (40 mg, 0.089 mmol) in THF (0.5 mL) was added 1.0 M trimethylphosphine/THF (0.178 mL, 0.178 mmol) followed by a drop of water. The mixture was stirred at rt for 4 h. LCMS indicated the SM was consumed, and the desired product was formed. EtOAc was added, washed with water, then concentrated. The residue was purified via silica gel chromatography (4 g, hexanes-60% EtOAc) to give 5-(3-bromo-2,6-difluorophenoxy)-3,3-difluoro-2-(4-fluorophenyl)pentan-2-amine 16C (40 mg, 0.094 mmol, 106% yield). (M+H)⁺=424.8.

¹H NMR (499 MHz, chloroform-d) δ 7.58 (dd, J=8.5, 5.5 Hz, 2H), 7.20 (ddd, J=9.1, 7.1, 5.4 Hz, 1H), 7.10-7.04 (m, 2H), 6.82 (ddd, J=10.0, 9.2, 2.1 Hz, 1H), 4.34-4.24 (m, 2H), 2.57-2.43 (m, 1H), 2.19-2.06 (m, 1H), 1.67 (s, 3H).

MS ESI m/z 424.8 (M+H)⁺.

16D: tert-butyl (7-(3-((4-amino-3,3-difluoro-4-(4-fluorophenyl)pentyl)oxy)-2,4-difluorophenyl)-[1,2,4]triazolo[1,5-a]pyridin-2-yl)(tert-butoxycarbonyl)carbamate was prepared in a similar fashion to 8B from 16C and B1.

MS ESI m/z 678.5 (M+H)⁺.

16 and 17: The two enantiomers of 7-(3-((4-amino-3,3-difluoro-4-(4-fluorophenyl)pentyl)oxy)-2,4-difluorophenyl)-[1,2,4]triazolo[1,5-a]pyridin-2-amine were prepared in a similar fashion to 2 and 3.

The first eluting enantiomer 16 7-(3-((4-amino-3,3-difluoro-4-(4-fluorophenyl)pentyl)oxy)-2,4-difluorophenyl)-[1,2,4]triazolo[1,5-a]pyridin-2-amine (6.6 mg, 0.014 mmol, 15.61% yield) and the second eluting enantiomer 17 7-(3-((4-amino-3,3-difluoro-4-(4-fluorophenyl)pentyl)oxy)-2,4-difluorophenyl)-[1,2,4]triazolo[1,5-a]pyridin-2-amine (6.6 mg, 0.014 mmol, 15.61% yield) were obtained.

16, ¹H NMR (500 MHz, DMSO-d₆) δ 8.59 (d, J=7.0 Hz, 1H), 7.63-7.57 (m, 2H), 7.48 (s, 1H), 7.40-7.34 (m, 1H), 7.25 (br t, J=9.6 Hz, 1H), 7.15 (t, J=8.9 Hz, 2H), 7.01 (br d, J=7.0 Hz, 1H), 6.06 (s, 2H), 4.30-4.20 (m, 2H), 2.49-2.42 (m, 1H), 2.22-2.09 (m, 1H), 1.51 (s, 3H).

MS ESI m/z 478.2 (M+H)⁺.

### Example 17: 7-(3-((4-amino-3,3-difluoro-4-(4-fluorophenyl)pentyl)oxy)-2,4-difluorophenyl)-[1,2,4]triazolo[1,5-a]pyridin-2-amine (enantiomer 2)

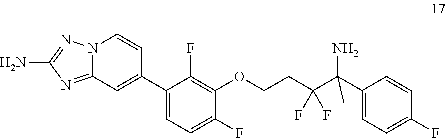 [SMILES: CC(N)(c1ccc(F)cc1)C(F)(F)CCOc1c(F)ccc(-c2ccn3nc(N)nc3c2)c1F]

17, ¹H NMR (500 MHz, DMSO-d₆) δ 8.59 (d, J=7.1 Hz, 1H), 7.60 (br dd, J=8.3, 5.6 Hz, 2H), 7.48 (s, 1H), 7.40-7.34 (m, 1H), 7.25 (br t, J=9.7 Hz, 1H), 7.15 (t, J=8.9 Hz, 2H), 7.01 (br d, J=6.9 Hz, 1H), 6.06 (s, 2H), 4.30-4.20 (m, 2H), 2.49-2.42 (m, 1H), 2.22-2.10 (m, 1H), 1.51 (s, 3H).

MS ESI m/z 478.2 (M+H)⁺.

### Example 18: 5-(3-(2-amino-[1,2,4]triazolo[1,5-a]pyridin-7-yl)-2-fluoro-6-methoxyphenoxy)-3,3-difluoro-2-(4-fluorophenyl)pentan-2-ol (enantiomer 2)

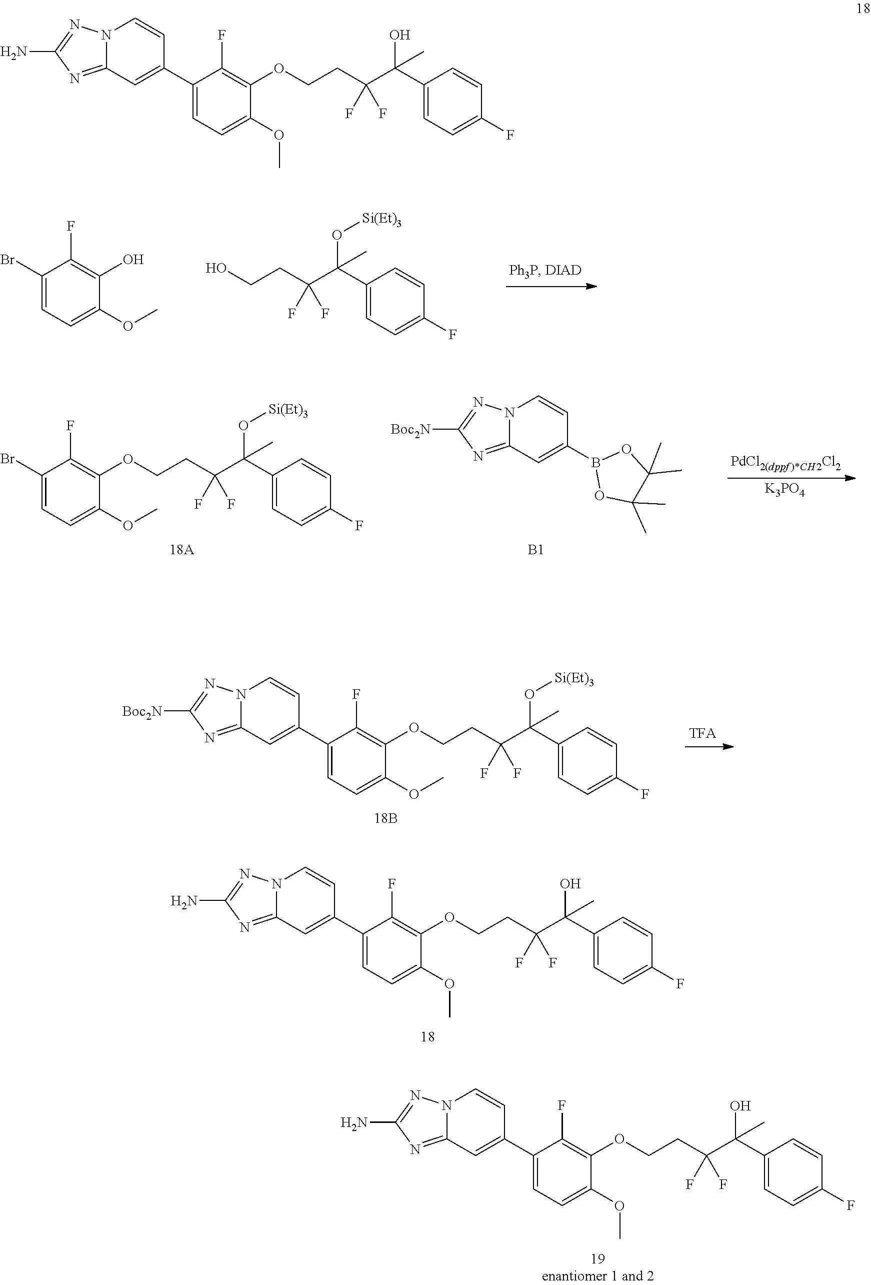

(The 2 unassigned enantiomers are:

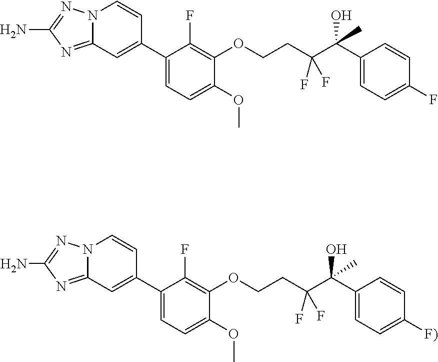

18A: ((5-(3-bromo-2-fluoro-6-methoxyphenoxy)-3,3-difluoro-2-(4-fluorophenyl)pentan-2-yl)-oxy)triethylsilane was prepared in a similar fashion to 8A from 3-bromo-2-fluoro-6-methoxyphenol and 3,3-difluoro-4-(4-fluorophenyl)-4-((triethylsilyl)oxy)pentan-1-ol.

¹H NMR (499 MHz, chloroform-d) δ 7.50 (ddd, J=8.7, 5.3, 1.3 Hz, 2H), 7.19 (dd, J=9.0, 7.4 Hz, 1H), 7.07-7.01 (m, 2H), 6.59 (dd, J=9.0, 1.8 Hz, 1H), 4.26-4.12 (m, 2H), 3.81 (s, 3H), 2.59-2.44 (m, 1H), 2.06-1.97 (m, 1H), 1.76 (s, 3H), 0.97 (t, J=8.0 Hz, 9H), 0.73-0.63 (m, 6H).

### 18B: tert-butyl (tert-butoxycarbonyl)(7-(3-((3,3-difluoro-4-(4-fluorophenyl)-4-((triethylsilyl)oxy)pentyl)oxy)-2-fluoro-4-methoxyphenyl)-[1,2,4]triazolo[1,5-a]pyridin-2-yl)carbamate

To a solution of 18A ((5-(3-bromo-2-fluoro-6-methoxyphenoxy)-3,3-difluoro-2-(4-fluorophenyl)pentan-2-yl)oxy)triethylsilane (170 mg, 0.308 mmol), B1 (199 mg, 0.432 mmol), tripotassium phosphate (0.462 mL, 0.925 mmol) was added PdCl₂(dppf)-CH₂Cl₂ adduct (12.59 mg, 0.015 mmol). The mixture was stirred 100° C. for 1 h. LCMS indicated the SM was consumed, and the product was formed. Water was added, and extracted with 2× EtOAc. The organic layer was concentrated to give a crude product tert-butyl (tert-butoxycarbonyl)(7-(3-((3,3-difluoro-4-(4-fluorophenyl)-4-((triethylsilyl)oxy)pentyl)oxy)-2-fluoro-4-methoxyphenyl)-[1,2,4]triazolo[1,5-a]pyridin-2-yl)carbamate 18B (185 mg, 0.230 mmol, 74.6% yield), which was used as was for the next step.

MS ESI m/z 805.8 (M+H)⁺.

18 and 19: The two enantiomers of 5-(3-(2-amino-[1,2,4]triazolo[1,5-a]pyridin-7-yl)-2-fluoro-6-methoxyphenoxy)-3,3-difluoro-2-(4-fluorophenyl)pentan-2-ol were prepared in a similar fashion to 2 and 3.

The first eluting isomer 18 5-(3-(2-amino-[1,2,4]triazolo[1,5-a]pyridin-7-yl)-2-fluoro-6-methoxyphenoxy)-3,3-difluoro-2-(4-fluorophenyl)pentan-2-ol (21 mg, 0.042 mmol, 18.98% yield) and the second eluting isomer 19 5-(3-(2-amino-[1,2,4]triazolo[1,5-a]pyridin-7-yl)-2-fluoro-6-methoxyphenoxy)-3,3-difluoro-2-(4-fluorophenyl)pentan-2-ol (20.3 mg, 0.041 mmol, 18.36% yield) were obtained.

18, ¹H NMR (500 MHz, DMSO-d₆) δ 8.55 (d, J=6.9 Hz, 1H), 7.54 (br dd, J=8.2, 5.7 Hz, 2H), 7.42 (s, 1H), 7.33 (t, J=8.6 Hz, 1H), 7.16 (t, J=8.9 Hz, 2H), 7.00 (br d, J=8.5 Hz, 2H), 6.02 (s, 2H), 4.15 (td, J=9.1, 6.1 Hz, 1H), 4.07 (td, J=9.2, 6.2 Hz, 1H), 3.83 (s, 3H), 2.44-2.32 (m, 1H), 2.11-1.96 (m, 1H), 1.58 (s, 3H).

MS ESI m/z 491.2 (M+H)⁺.

### Example 19: 5-(3-(2-amino-[1,2,4]triazolo[1,5-a]pyridin-7-yl)-2-fluoro-6-methoxyphenoxy)-3,3-difluoro-2-(4-fluorophenyl)pentan-2-ol (enantiomer 2)

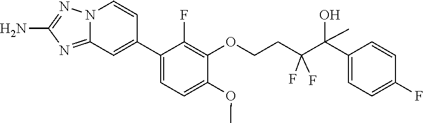 [SMILES: COc1ccc(-c2ccn3nc(N)nc3c2)c(F)c1OCCC(F)(F)C(C)(O)c1ccc(F)cc1]

19, ¹H NMR (500 MHz, DMSO-d₆) δ 8.55 (d, J=6.9 Hz, 1H), 7.55 (br dd, J=8.2, 5.7 Hz, 2H), 7.42 (s, 1H), 7.33 (t, J=8.7 Hz, 1H), 7.17 (t, J=8.9 Hz, 2H), 7.00 (br d, J=8.3 Hz, 2H), 6.02 (s, 2H), 4.18-4.11 (m, 1H), 4.07 (td, J=9.1, 6.0 Hz, 1H), 3.83 (s, 3H), 2.45-2.31 (m, 1H), 2.11-1.97 (m, 1H), 1.58 (s, 3H).

MS ESI m/z 491.2 (M+H)⁺.

### Example 20: 5-(3-(2-amino-[1,2,4]triazolo[1,5-a]pyridin-7-yl)-2,6-difluoro-4-methoxyphenoxy)-3,3-difluoro-2-(4-fluorophenyl)pentan-2-ol (enantiomer 1)

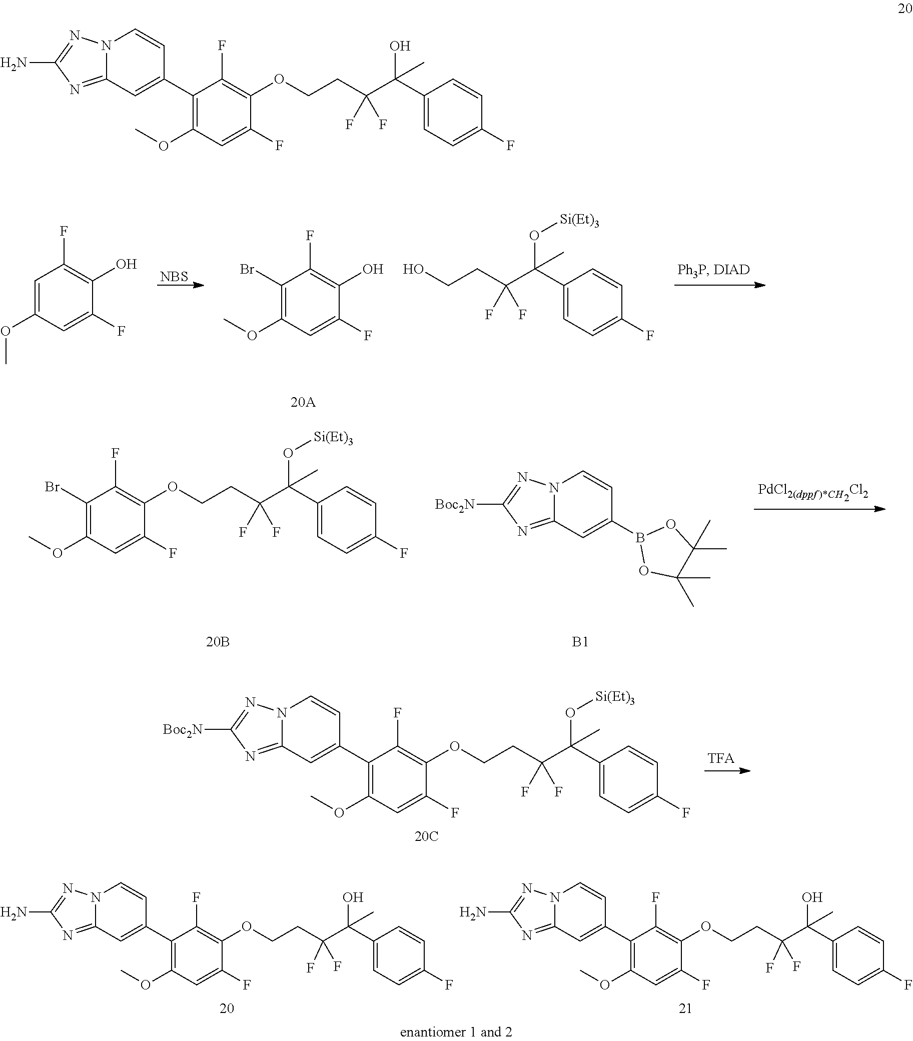

(The 2 unassigned enantiomers are:

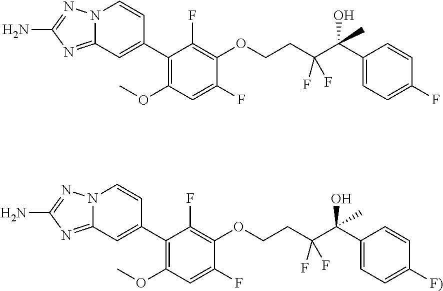

### 20A:3-bromo-2,6-difluoro-4-methoxyphenol

To a solution of 2,6-difluoro-4-methoxyphenol (160 mg, 0.999 mmol) in MeCN (5 mL) was slowly added NBS (196 mg, 1.099 mmol) at rt. The mixture was stirred rt for 2 h. LCMS indicated the SM was consumed, and a new peak was formed. The rxn was concentrated. EtOAc was added, washed with water, dried over Na₂SO₄, then concentrated. The residue was purified via silica gel chromatography (12 g, hexanes-50% EtOAc) to give 3-bromo-2,6-difluoro-4-methoxyphenol (125 mg, 0.523 mmol, 52.3% yield).

¹H NMR (499 MHz, chloroform-d) δ 6.58 (dd, J=11.9, 2.4 Hz, 1H), 4.98 (s, 1H), 3.86 (s, 3H).

20B: ((5-(3-bromo-2,6-difluoro-4-methoxyphenoxy)-3,3-difluoro-2-(4-fluorophenyl)pentan-2-yl)oxy)triethylsilane was prepared in a similar fashion to 8A from 20A and 3,3-difluoro-4-(4-fluorophenyl)-4-((triethylsilyl)oxy)pentan-1-ol.

20C: tert-butyl (tert-butoxycarbonyl)(7-(3-((3,3-difluoro-4-(4-fluorophenyl)-4-((triethylsilyl)-oxy)pentyl)oxy)-2,4-difluoro-6-methoxyphenyl)-[1,2,4]triazolo[1,5-a]pyridin-2-yl)carbamate prepared in a similar fashion to 18B from 20B and B1.

20 and 21: The two enantiomers of were 5-(3-(2-amino-[1,2,4]triazolo[1,5-a]pyridin-7-yl)-2,6-difluoro-4-methoxyphenoxy)-3,3-difluoro-2-(4-fluorophenyl)pentan-2-ol were prepared in a similar fashion to 2 and 3.

The first eluting isomer 20 5-(3-(2-amino-[1,2,4]triazolo[1,5-a]pyridin-7-yl)-2,6-difluoro-4-methoxyphenoxy)-3,3-difluoro-2-(4-fluorophenyl)pentan-2-ol (23.6 mg, 0.046 mmol, 20.65% yield) and the second eluting isomer 21 5-(3-(2-amino-[1,2,4]triazolo[1,5-a]pyridin-7-yl)-2,6-difluoro-4-methoxyphenoxy)-3,3-difluoro-2-(4-fluorophenyl)pentan-2-ol (23.6 mg, 0.046 mmol, 20.65% yield) were obtained.

20, ¹H NMR (500 MHz, DMSO-d₆) δ 8.56 (d, J=6.9 Hz, 1H), 7.55 (br dd, J=8.5, 5.8 Hz, 2H), 7.31 (s, 1H), 7.17 (t, J=8.9 Hz, 2H), 7.01 (br d, J=13.0 Hz, 1H), 6.81 (br d, J=6.9 Hz, 1H), 6.16 (s, 1H), 6.02 (s, 2H), 4.20-4.08 (m, 2H), 3.74 (s, 3H), 2.46-2.34 (m, 1H), 2.11-1.99 (m, 1H), 1.59 (s, 3H).

MS ESI m/z 509.2 (M+H)⁺.

### Example 21: 5-(3-(2-amino-[1,2,4]triazolo[1,5-a]pyridin-7-yl)-2,6-difluoro-4-methoxyphenoxy)-3,3-difluoro-2-(4-fluorophenyl)pentan-2-ol (enantiomer 2)

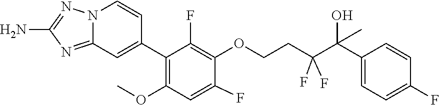 [SMILES: COc1cc(F)c(OCCC(F)(F)C(C)(O)c2ccc(F)cc2)c(F)c1-c1ccn2nc(N)nc2c1]

21, ¹H NMR (500 MHz, DMSO-d₆) δ 8.55 (d, J=6.9 Hz, 1H), 7.55 (br dd, J=8.3, 5.5 Hz, 2H), 7.31 (s, 1H), 7.17 (t, J=8.9 Hz, 2H), 7.01 (br d, J=12.4 Hz, 1H), 6.83-6.79 (m, 1H), 6.17 (s, 1H), 6.02 (s, 2H), 4.20-4.08 (m, 2H), 3.74 (s, 3H), 2.47-2.32 (m, 1H), 2.12-1.98 (m, 1H), 1.59 (s, 3H).

MS ESI m/z 509.2 (M+H)⁺.

### Example 22: 5-(3-(2-amino-[1,2,4]triazolo[1,5-a]pyridin-7-yl)-2-fluoro-4-methoxyphenoxy)-3,3-difluoro-2-(4-fluorophenyl)pentan-2-ol

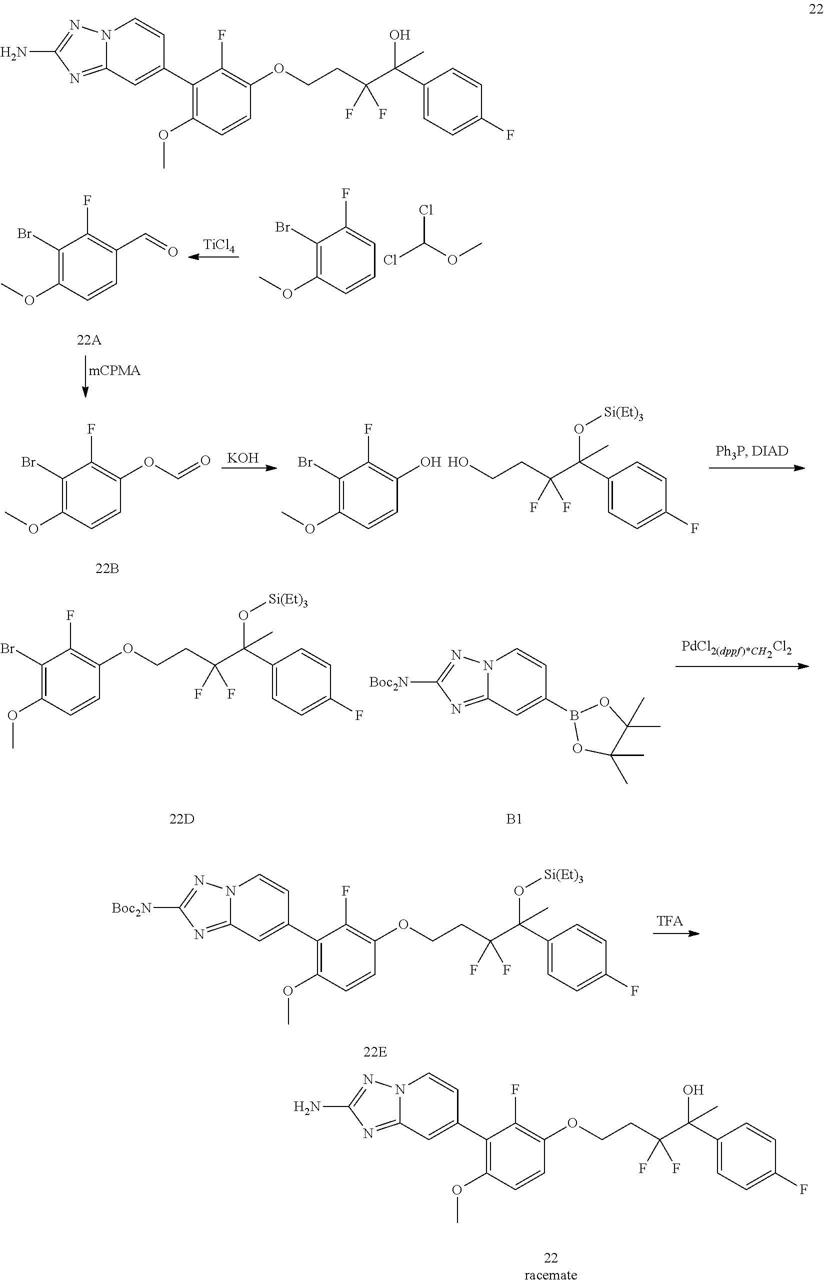

### 22A: 3-bromo-2-fluoro-4-methoxybenzaldehyde

To a solution of 2-bromo-1-fluoro-3-methoxybenzene (2.0 g, 9.75 mmol) in DCM (30 mL) was added dichloro(methoxy)methane (1.682 g, 14.63 mmol) followed by TiCl4/DCM (48.8 mL, 48.8 mmol) at 0° C. under N₂. The mixture was allowed to warm up to rt and stirred at rt for 2 days. The red solution was poured into ice water, and extracted with 2× DCM. The organic layer was washed with NaHCO₃/water, brine and water. After evaporation, crude 3-bromo-2-fluoro-4-methoxybenzaldehyde (2.2 g, 9.44 mmol, 97% yield) was obtained.

MS ESI m/z 234.7 (M+H)⁺.

### 22B: 3-bromo-2-fluoro-4-methoxyphenyl formate

To a solution of crude 3-bromo-2-fluoro-4-methoxybenzaldehyde (1.1 g, 4.72 mmol) in DCM (15 mL) was added mCPBA (3.67 g, 21.24 mmol) slowly. The mixture was stirred at rt for 20 h. DCM was added and washed with NaHSO₃/water and NaHCO₃/water, then concentrated to give crude 3-bromo-2-fluoro-4-methoxyphenyl formate (1.0 g, 4.02 mmol, 85% yield).

¹H NMR (499 MHz, chloroform-d) δ 8.29 (d, J=1.1 Hz, 1H), 7.14 (t, J=8.6 Hz, 1H), 6.72 (dd, J=9.1, 2.0 Hz, 1H), 3.94 (s, 3H).

### 22C: 3-bromo-2-fluoro-4-methoxyphenol

To a solution of 3-bromo-2-fluoro-4-methoxyphenyl formate 22B (1.0 g, 4.02 mmol) in EtOH (15 mL) was added KOH (0.676 g, 12.05 mmol). The mixture was stirred at rt for 3 h. 13 mL of 1N HCl was added, then extracted with EtOAc. The organic layer was concentrated. The residue was purified via silica gel chromatography (24 g, hexanes-40% EtOAc) to give 3-bromo-2-fluoro-4-methoxyphenol (470 mg, 2.126 mmol, 53.0% yield).

¹H NMR (499 MHz, chloroform-d) δ 6.95 (t, J=9.3 Hz, 1H), 6.64 (dd, J=9.2, 2.1 Hz, 1H), 4.88 (br d, J=1.1 Hz, 1H), 3.88 (s, 3H).

22D: ((5-(3-bromo-2-fluoro-4-methoxyphenoxy)-3,3-difluoro-2-(4-fluorophenyl)pentan-2-yl)oxy)triethylsilane was prepared in a similar fashion to 8A from 22C and 3,3-difluoro-4-(4-fluorophenyl)-4-((triethylsilyl)oxy)pentan-1-ol.

22E: tert-butyl (tert-butoxycarbonyl)(7-(3-((3,3-difluoro-4-(4-fluorophenyl)-4-((triethylsilyl)-oxy)pentyl)oxy)-2-fluoro-6-methoxyphenyl)-[1,2,4]triazolo[1,5-a]pyridin-2-yl)carbamate was prepared in a similar fashion to 18B from 22D and B1.

22: 5-(3-(2-amino-[1,2,4]triazolo[1,5-a]pyridin-7-yl)-2-fluoro-4-methoxyphenoxy)-3,3-difluoro-2-(4-fluorophenyl)pentan-2-ol was prepared in a similar fashion to 1 from 22E. Racemic 5-(3-(2-amino-[1,2,4]triazolo[1,5-a]pyridin-7-yl)-2-fluoro-4-methoxyphenoxy)-3,3-difluoro-2-(4-fluorophenyl)pentan-2-ol (10 mg, 0.020 mmol, 40.3% yield) was obtained.

¹H NMR (500 MHz, DMSO-d₆) δ 8.56 (br d, J=4.9 Hz, 1H), 7.56 (br t, J=5.9 Hz, 2H), 7.33 (br s, 1H), 7.16-7.10 (m, 3H), 6.90-6.82 (m, 2H), 4.19-4.07 (m, 2H), 3.71 (d, J=2.8 Hz, 3H), 2.48-2.36 (m, 1H), 2.11-1.96 (m, 1H), 1.59 (s, 3H).

MS ESI m/z 491.2 (M+H)⁺.

### Example 23: 5-(3-(2-amino-[1,2,4]triazolo[1,5-a]pyridin-7-yl)-2-(difluoromethyl)phenoxy)-3,3-difluoro-2-(4-fluorophenyl)pentan-2-ol (enantiomer 1)

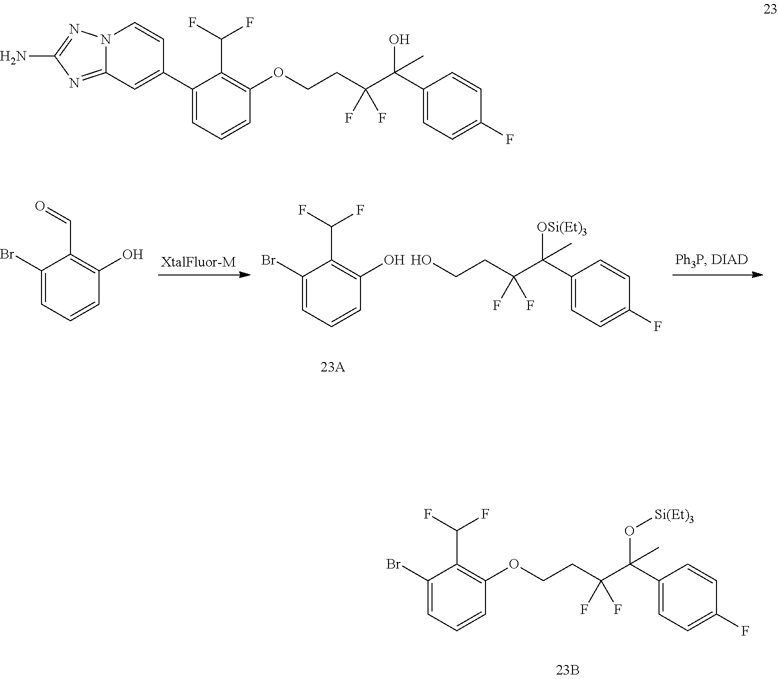

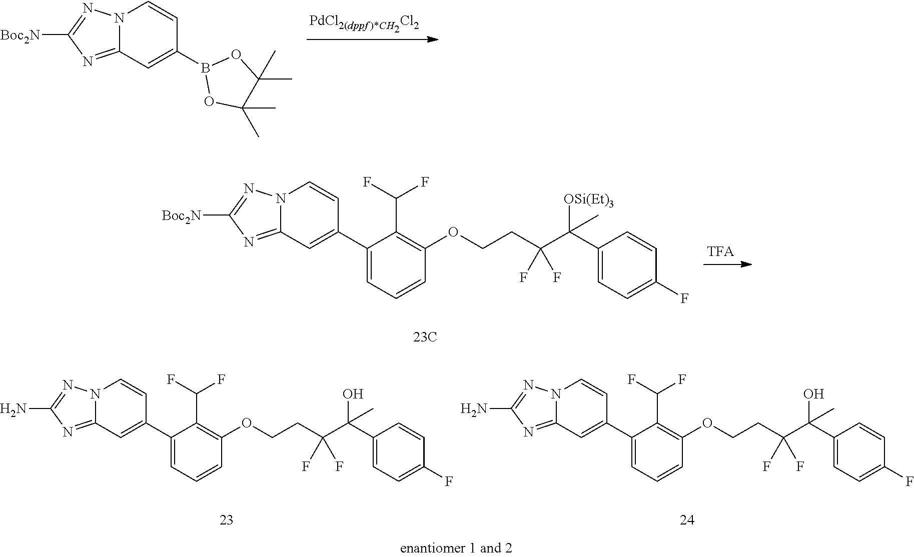

(The 2 unassigned enantiomers are:

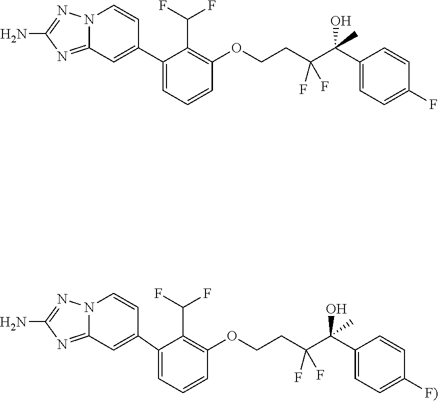

### 23A: 3-bromo-2-(difluoromethyl)phenol

To a solution of 2-bromo-6-hydroxybenzaldehyde (300 mg, 1.492 mmol) in DCM (5 mL) was added triethylamine trihydrofluoride (481 mg, 2.98 mmol) followed by XtalFluor-M® (544 mg, 2.239 mmol). The mixture was stirred rt for 3 h. Another 1 eq. of XtalFluor-M® was added. The mixture was stirred at rt for 18 h. EtOAc was added, washed with brine, then concentrated in vacuo at rt. The residue was purified via silica gel chromatography (24 g, hexanes-40% EtOAc) to give 3-bromo-2-(difluoromethyl)phenol (270 mg, 1.211 mmol, 81% yield).

¹H NMR (499 MHz, chloroform-d) δ 7.26-7.10 (m, 3H), 6.96 (d, J=7.9 Hz, 1H), 6.23 (t, J=8.2 Hz, 1H).

23B: ((5-(3-bromo-2-(difluoromethyl)phenoxy)-3,3-difluoro-2-(4-fluorophenyl)pentan-2-yl)oxy)triethylsilane was prepared in a similar fashion to 8A from 24A and F.

23C: tert-butyl (tert-butoxycarbonyl)(7-(3-((3,3-difluoro-4-(4-fluorophenyl)-4-((triethylsilyl)oxy)pentyl)oxy)-2-(difluoromethyl)phenyl)-[1,2,4]triazolo[1,5-a]pyridin-2-yl)carbamate was prepared in a similar fashion to 18B from 23B and tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate.

23 and 24: The two enantiomers of 5-(3-(2-amino-[1,2,4]triazolo[1,5-a]pyridin-7-yl)-2-(difluoromethyl)phenoxy)-3,3-difluoro-2-(4-fluorophenyl)pentan-2-ol were prepared in a similar fashion to 2 and 3.

The first eluting isomer 23 5-(3-(2-amino-[1,2,4]triazolo[1,5-a]pyridin-7-yl)-2-(difluoromethyl)phenoxy)-3,3-difluoro-2-(4-fluorophenyl)pentan-2-ol (22.4 mg, 0.045 mmol, 18.62% yield) and the second eluting isomer 24 5-(3-(2-amino-[1,2,4]triazolo[1,5-a]pyridin-7-yl)-2-(difluoromethyl)phenoxy)-3,3-difluoro-2-(4-fluorophenyl)pentan-2-ol (22.1 mg, 0.044 mmol, 18.39% yield) were obtained.

23, ¹H NMR (500 MHz, DMSO-d₆) δ 8.55 (d, J=6.7 Hz, 1H), 7.63-7.53 (m, 3H), 7.23-7.16 (m, 4H), 7.09-6.85 (m, 2H), 6.78 (dd, J=6.8, 1.7 Hz, 1H), 6.19 (s, 1H), 6.03 (s, 2H), 4.29-4.21 (m, 2H), 2.49-2.41 (m, 1H), 2.19-2.05 (m, 1H), 1.61 (s, 3H).

MS ESI m/z 493.2 (M+H)⁺.

### Example 24: 5-(3-(2-amino-[1,2,4]triazolo[1,5-a]pyridin-7-yl)-2-(difluoromethyl)phenoxy)-3,3-difluoro-2-(4-fluorophenyl)pentan-2-ol (enantiomer 2)

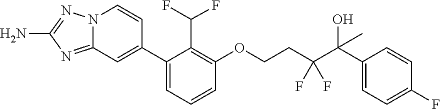 [SMILES: CC(O)(c1ccc(F)cc1)C(F)(F)CCOc1cccc(-c2ccn3nc(N)nc3c2)c1C(F)F]

24, ¹H NMR (500 MHz, DMSO-d₆) δ 8.55 (d, J=6.9 Hz, 1H), 7.62-7.52 (m, 3H), 7.23-7.16 (m, 4H), 7.09-6.85 (m, 2H), 6.78 (dd, J=6.9, 1.6 Hz, 1H), 6.19 (s, 1H), 6.03 (s, 2H), 4.29-4.21 (m, 2H), 2.50-2.43 (m, 1H), 2.19-2.04 (m, 1H), 1.61 (s, 3H).

MS ESI m/z 493.2 (M+H)⁺.

### Example 25: 5-(3-(2-amino-[1,2,4]triazolo[1,5-a]pyridin-7-yl)-6-(difluoromethyl)-2-fluorophenoxy)-3,3-difluoro-2-(4-fluorophenyl)pentan-2-ol (enantiomer 1)

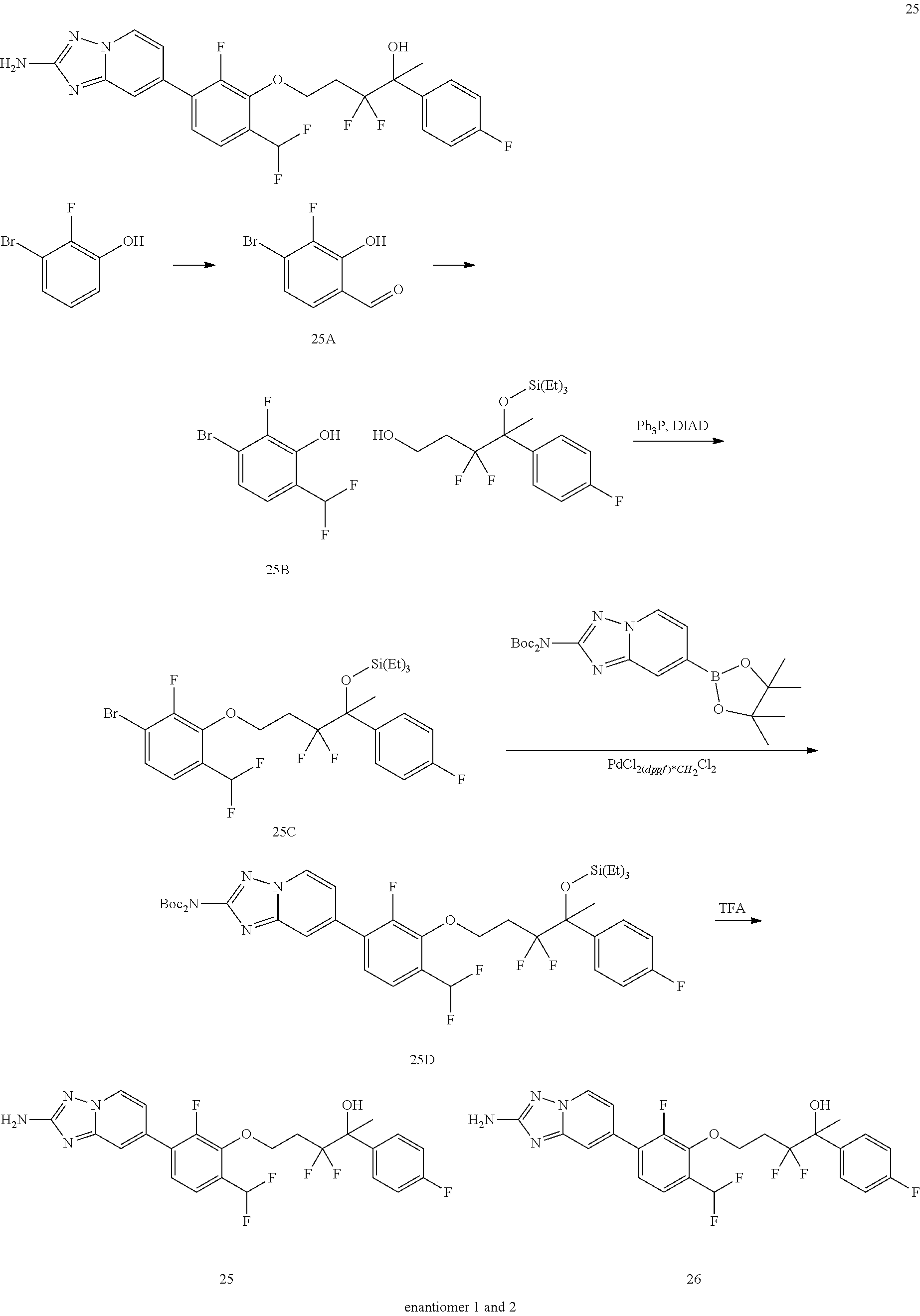

(The 2 unassigned enantiomers are:

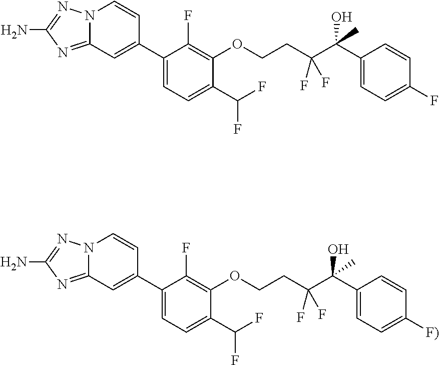

### 25A: 4-bromo-3-fluoro-2-hydroxybenzaldehyde

To a solution of 3-bromo-2-fluorophenol (0.955 g, 5 mmol) in THF (15 mL) was added anhydrous magnesium chloride (0.714 g, 7.50 mmol), paraformaldehyde (1.1 g, 36.6 mmol) followed by DIPEA (3.49 mL, 20.00 mmol). The mixture was stirred 58° C. for 18 h. Et₂O was added, washed with 1N HCl, then concentrated. The residue was purified via silica gel chromatography (24 g, hexanes-50% EtOAc) to give 4-bromo-3-fluoro-2-hydroxybenzaldehyde (275 mg, 1.256 mmol, 25.1% yield).

¹H NMR (499 MHz, chloroform-d) δ 11.16 (s, 1H), 9.92 (d, J=1.8 Hz, 1H), 7.29-7.21 (m, 2H).

25B: 3-bromo-6-(difluoromethyl)-2-fluorophenol was prepared in a similar fashion to 23A from 25A.

25C: ((5-(3-bromo-6-(difluoromethyl)-2-fluorophenoxy)-3,3-difluoro-2-(4-fluorophenyl)pentan-2-yl)oxy)triethylsilane was prepared in a similar fashion to 8A from 25B and F.

25D: tert-butyl (tert-butoxycarbonyl)(7-(3-((3,3-difluoro-4-(4-fluorophenyl)-4-((triethylsilyl)oxy)pentyl)oxy)-4-(difluoromethyl)-2-fluorophenyl)-[1,2,4]triazolo[1,5-a]pyridin-2-yl)carbamate was prepared in a similar fashion to 18B from 25C.

25 and 26: The two enantiomers of 5-(3-(2-amino-[1,2,4]triazolo[1,5-a]pyridin-7-yl)-6-(difluoromethyl)-2-fluorophenoxy)-3,3-difluoro-2-(4-fluorophenyl)pentan-2-ol were prepared in a similar fashion to 2 and 3.

The first eluting isomer 25 5-(3-(2-amino-[1,2,4]triazolo[1,5-a]pyridin-7-yl)-6-(difluoromethyl)-2-fluorophenoxy)-3,3-difluoro-2-(4-fluorophenyl)pentan-2-ol (14.9 mg, 0.029 mmol, 12.80% yield) and the second eluting isomer 26 5-(3-(2-amino-[1,2,4]triazolo[1,5-a]pyridin-7-yl)-6-(difluoromethyl)-2-fluorophenoxy)-3,3-difluoro-2-(4-fluorophenyl)pentan-2-ol (13.6 mg, 0.026 mmol, 11.68% yield) were obtained. 25, ¹H NMR (500 MHz, DMSO-d₆) δ 8.63 (d, J=7.1 Hz, 1H), 7.60-7.54 (m, 3H), 7.50-7.44 (m, 2H), 7.21-6.97 (m, 4H), 6.21 (s, 1H), 6.10 (s, 2H), 4.34-4.24 (m, 2H), 2.49-2.41 (m, 1H), 2.19-2.06 (m, 1H), 1.60 (s, 3H).

MS ESI m/z 511.2 (M+H)⁺.

### Example 26: 5-(3-(2-amino-[1,2,4]triazolo[1,5-a]pyridin-7-yl)-6-(difluoromethyl)-2-fluorophenoxy)-3,3-difluoro-2-(4-fluorophenyl)pentan-2-ol (enantiomer 2)

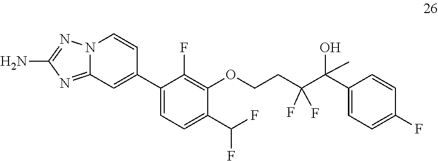 [SMILES: CC(O)(c1ccc(F)cc1)C(F)(F)CCOc1c(C(F)F)ccc(-c2ccn3nc(N)nc3c2)c1F]

26, ¹H NMR (500 MHz, DMSO-d₆) δ 8.64 (d, J=6.9 Hz, 1H), 7.60-7.55 (m, 3H), 7.50-7.44 (m, 2H), 7.22-6.99 (m, 4H), 6.19 (s, 1H), 6.11 (s, 1H), 4.35-4.24 (m, 2H), 2.50-2.43 (m, 1H), 2.20-2.06 (m, 1H), 1.61 (s, 3H).

MS ESI m/z 511.2 (M+H)⁺.

### Example 27: 5-(3-(2-amino-[1,2,4]triazolo[1,5-a]pyridin-7-yl)-2,6-difluorophenoxy)-3,3-difluoro-2-(5-fluoropyridin-2-yl)pentan-2-ol (enantiomer 1)

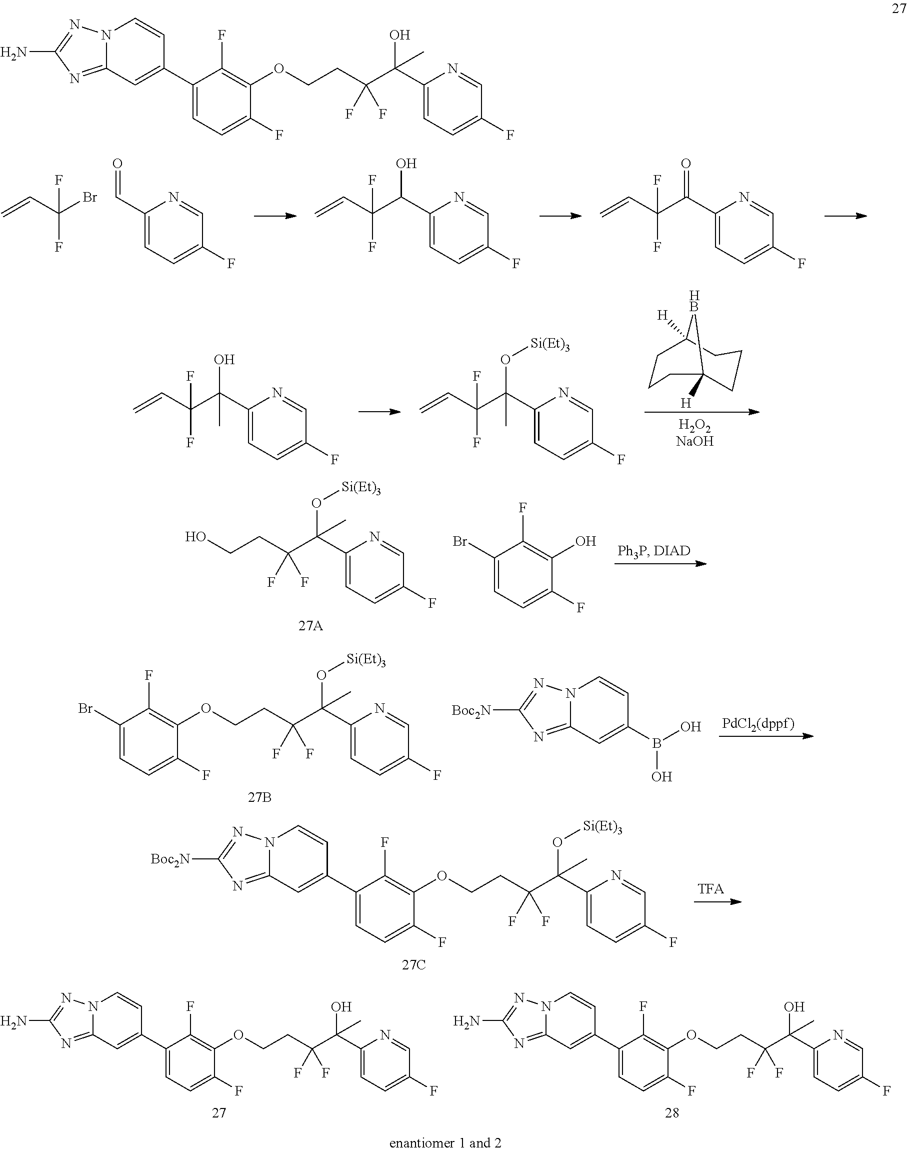

(The 2 unassigned enantiomers are:

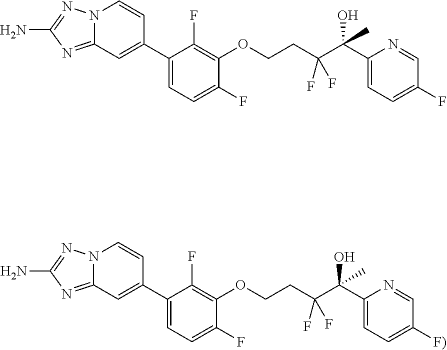

### 27A: 3,3-difluoro-4-(5-fluoropyridin-2-yl)-4-((triethylsilyl)oxy)pentan-1-ol

To a solution of 2-(3,3-difluoro-2-((triethylsilyl)oxy)pent-4-en-2-yl)-5-fluoropyridine (480 mg, 1.448 mmol) in THF (6 mL) was added 9-BBN (14.48 mL, 7.24 mmol) slowly at rt. The mixture was stirred at rt for 20 h. Another 2 eq. of 9-BBN were added. The mixture was stirred at rt over weekend. At 0° C., NaOH (3.86 mL, 11.59 mmol) was added followed by dropwise addition of H₂O₂ (1.183 mL, 11.59 mmol). The mixture was stirred at rt for 1 h. Water was added and extracted with 2× Et₂O. The organic layer was concentrated. The residue was purified via silica gel chromatography (24 g, hexanes-15% EtOAc) to give crude 3,3-difluoro-4-(5-fluoropyridin-2-yl)-4-((triethylsilyl)oxy)pentan-1-ol (400 mg, 1.145 mmol, 79% yield.

¹H NMR (499 MHz, chloroform-d) δ 8.44 (d, J=2.9 Hz, 1H), 7.74 (dd, J=8.8, 4.5 Hz, 1H), 7.45-7.40 (m, 1H), 3.90-3.84 (m, 2H), 2.31-2.17 (m, 1H), 2.05-1.92 (m, 1H), 1.85-1.84 (m, 3H), 0.97-0.94 (m, 9H), 0.70-0.61 (m, 6H).

MS ESI m/z 350.2 (M+H)⁺.

27B: 2-(5-(3-bromo-2,6-difluorophenoxy)-3,3-difluoro-2-((triethylsilyl)oxy)pentan-2-yl)-5-fluoropyridine was prepared in a similar fashion to 8A from 27A and 3-bromo-2,6-difluorophenol.

27C: tert-butyl (tert-butoxycarbonyl)(7-(3-((3,3-difluoro-4-(5-fluoropyridin-2-yl)-4-((triethylsilyl)oxy)pentyl)oxy)-2,4-difluorophenyl)-[1,2,4]triazolo[1,5-a]pyridin-2-yl)carbamate was prepared in a similar fashion to 18B from 27B and B1.

27 and 28: The two enantiomers of 5-(3-(2-amino-[1,2,4]triazolo[1,5-a]pyridin-7-yl)-2,6-difluorophenoxy)-3,3-difluoro-2-(5-fluoropyridin-2-yl)pentan-2-ol were prepared in a similar fashion to 2 and 3

The first eluting isomer 27 5-(3-(2-amino-[1,2,4]triazolo[1,5-a]pyridin-7-yl)-2,6-difluorophenoxy)-3,3-difluoro-2-(5-fluoropyridin-2-yl)pentan-2-ol (12.5 mg, 0.025 mmol, 16.42% yield) and the second eluting isomer 28 5-(3-(2-amino-[1,2,4]triazolo[1,5-a]pyridin-7-yl)-2,6-difluorophenoxy)-3,3-difluoro-2-(5-fluoropyridin-2-yl)pentan-2-ol (12.4 mg, 0.025 mmol, 16.26% yield) were obtained.

27, ¹H NMR (500 MHz, DMSO-d₆) δ 8.59 (d, J=6.9 Hz, 1H), 8.53 (d, J=2.7 Hz, 1H), 7.80-7.71 (m, 2H), 7.47 (s, 1H), 7.37 (td, J=8.4, 6.0 Hz, 1H), 7.25 (t, J=9.0 Hz, 1H), 7.02 (br d, J=7.0 Hz, 1H), 6.06 (s, 2H), 4.33-4.24 (m, 2H), 2.49-2.40 (m, 1H), 2.29-2.17 (m, 1H), 1.62 (s, 3H) MS ESI m/z 480.1 (M+H)⁺.

### Example 28: 5-(3-(2-amino-[1,2,4]triazolo[1,5-a]pyridin-7-yl)-2,6-difluorophenoxy)-3,3-difluoro-2-(5-fluoropyridin-2-yl)pentan-2-ol (enantiomer 2)

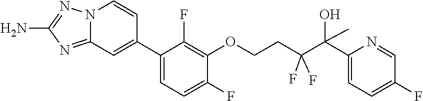 [SMILES: CC(O)(c1ccc(F)cn1)C(F)(F)CCOc1c(F)ccc(-c2ccn3nc(N)nc3c2)c1F]

28, ¹H NMR (500 MHz, DMSO-d₆) δ 8.58 (d, J=6.9 Hz, 1H), 8.52 (d, J=2.7 Hz, 1H), 7.79-7.71 (m, 2H), 7.47 (s, 1H), 7.36 (td, J=8.5, 5.8 Hz, 1H), 7.27-7.22 (m, 1H), 7.02 (br d, J=7.0 Hz, 1H), 6.05 (s, 2H), 4.31-4.24 (m, 2H), 2.49-2.40 (m, 1H), 2.29-2.15 (m, 1H), 1.62 (s, 3H).

MS ESI m/z 480.1 (M+H)⁺.

### Example 29: 5-(3-(2-amino-[1,2,4]triazolo[1,5-a]pyridin-7-yl)-2,4,6-trifluorophenoxy)-3,3-difluoro-2-(4-fluorophenyl)pentan-2-ol (single enantiomer)

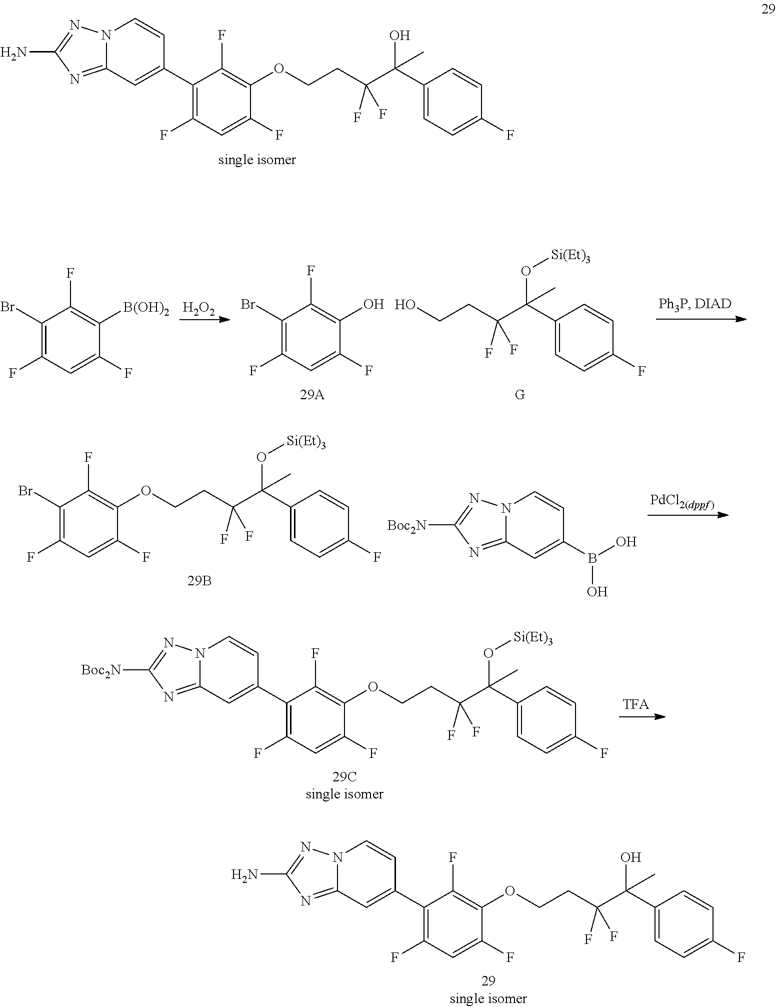

### 29A: 3-bromo-2,4,6-trifluorophenol

To a solution of (3-bromo-2,4,6-trifluorophenyl)boronic acid (200 mg, 0.785 mmol) in dioxane (1 mL) was added H₂O₂ (0.241 mL, 2.355 mmol). The mixture was stirred at rt for 5 days. The mixture was concentrated. The residue was purified via silica gel chromatography (12 g, hexanes-20% EtOAc) to give 3-bromo-2,4,6-trifluorophenol (150 mg, 0.661 mmol, 84% yield).

¹H NMR (499 MHz, chloroform-d) δ 6.86 (ddd, J=10.3, 8.1, 2.4 Hz, 1H), 5.10 (br s, 1H)

29B: ((5-(3-bromo-2,4,6-trifluorophenoxy)-3,3-difluoro-2-(4-fluorophenyl)pentan-2-yl)oxy)triethylsilane was prepared in a similar fashion to 8A using 29A and the single enantiomer G.

29C: tert-butyl (tert-butoxycarbonyl)(7-(3-((3,3-difluoro-4-(4-fluorophenyl)-4-((triethylsilyl)oxy)pentyl)oxy)-2,4,6-trifluorophenyl)-[1,2,4]triazolo[1,5-a]pyridin-2-yl)carbamate was prepared in a similar fashion to 18B from 29B and B1.

29: 5-(3-(2-amino-[1,2,4]triazolo[1,5-a]pyridin-7-yl)-2,4,6-trifluorophenoxy)-3,3-difluoro-2-(4-fluorophenyl)pentan-2-ol was prepared in a similar fashion to 1. 5-(3-(2-amino-[1,2,4]triazolo[1,5-a]pyridin-7-yl)-2,4,6-trifluorophenoxy)-3,3-difluoro-2-(4-fluorophenyl)pentan-2-ol (47 mg, 0.095 mmol, 59.1% yield) was obtained.

¹H NMR (500 MHz, DMSO-d₆) δ 8.66 (d, J=6.9 Hz, 1H), 7.60-7.52 (m, 2H), 7.49 (s, 1H), 7.43 (br t, J=10.2 Hz, 1H), 7.17 (s, 2H), 6.96 (br d, J=6.9 Hz, 1H), 4.28-4.13 (m, 2H), 2.49-2.35 (m, 1H), 2.15-2.01 (m, 1H), 1.59 (s, 3H).

MS ESI m/z 497.1 (M+H)+

### Example 30: 5-(3-(2-amino-[1,2,4]triazolo[1,5-a]pyridin-7-yl)-2,5,6-trifluorophenoxy)-3,3-difluoro-2-(4-fluorophenyl)pentan-2-ol (single enantiomer)

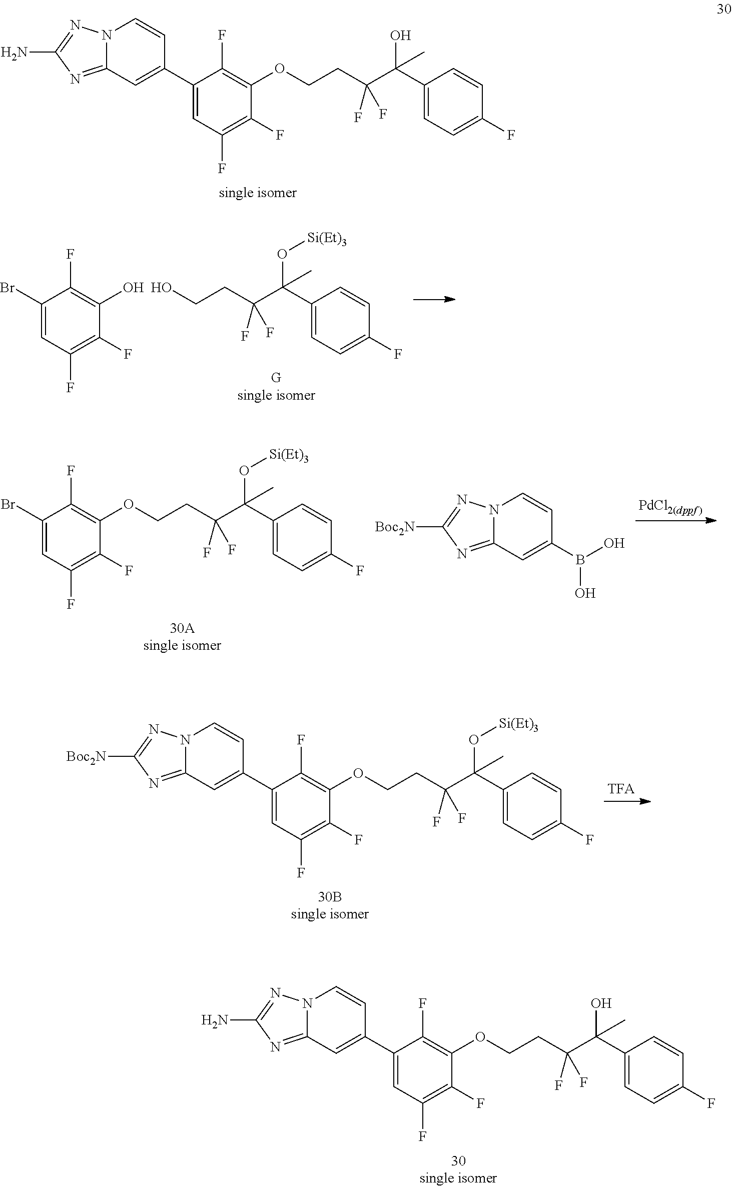

30A: ((5-(3-bromo-2,5,6-trifluorophenoxy)-3,3-difluoro-2-(4-fluorophenyl)pentan-2-yl)oxy)triethylsilane was prepared in a similar fashion to 8A using the single enantiomer G and 3-bromo-2,5,6-trifluorophenol.

30B: tert-butyl (tert-butoxycarbonyl)(7-(3-((3,3-difluoro-4-(4-fluorophenyl)-4-((triethylsilyl)-oxy)pentyl)oxy)-2,4,5-trifluorophenyl)-[1,2,4]triazolo[1,5-a]pyridin-2-yl)carbamate was prepared in a similar fashion to 18B from 30A and B1.

30: 5-(3-(2-amino-[1,2,4]triazolo[1,5-a]pyridin-7-yl)-2,5,6-trifluorophenoxy)-3,3-difluoro-2-(4-fluorophenyl)pentan-2-ol prepared in a similar fashion to 1. 5-(3-(2-amino-[1,2,4]triazolo[1,5-a]pyridin-7-yl)-2,5,6-trifluorophenoxy)-3,3-difluoro-2-(4-fluorophenyl)pentan-2-ol (49.7 mg, 0.100 mmol, 79% yield) was obtained.

¹H NMR (500 MHz, DMSO-d₆) δ 8.62 (d, J=7.0 Hz, 1H), 7.59-7.51 (m, 4H), 7.20-7.15 (m, 2H), 7.05-7.01 (m, 1H), 6.18 (s, 1H), 6.10 (s, 2H), 4.39-4.29 (m, 2H), 2.49-2.40 (m, 1H), 2.17-2.03 (m, 1H), 1.60 (s, 3H).

MS ESI m/z 497.1 (M+H)⁺.

### Example 31: 5-(3-(2-amino-[1,2,4]triazolo[1,5-a]pyridin-7-yl)-6-chloro-2-fluorophenoxy)-3,3-difluoro-2-(5-fluoropyridin-2-yl)pentan-2-ol (enantiomer 1)

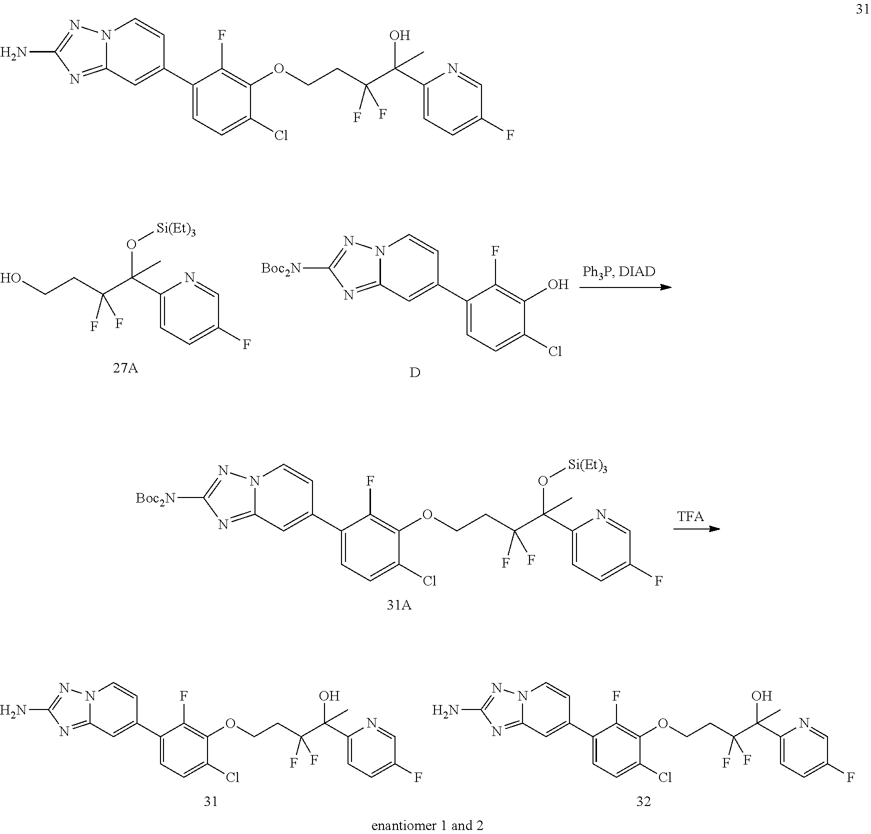

(The 2 unassigned enantiomers are:

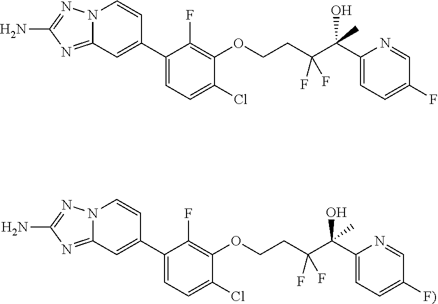

31A: tert-butyl (tert-butoxycarbonyl)(7-(4-chloro-3-((3,3-difluoro-4-(5-fluoropyridin-2-yl)-4-((triethylsilyl)oxy)pentyl)oxy)-2-fluorophenyl)-[1,2,4]triazolo[1,5-a]pyridin-2-yl)carbamate was prepared in a similar fashion to 2A from 27A and D.

31 and 32: The two enantiomers of 5-(3-(2-amino-[1,2,4]triazolo[1,5-a]pyridin-7-yl)-6-chloro-2-fluorophenoxy)-3,3-difluoro-2-(5-fluoropyridin-2-yl)pentan-2-ol were prepared in a similar fashion to 2 and 3.

31, H NMR (500 MHz, DMSO-d₆) δ 8.62 (d, J=6.9 Hz, 1H), 8.56 (d, J=2.7 Hz, 1H), 7.83-7.73 (m, 2H), 7.53 (s, 1H), 7.45-7.38 (m, 2H), 7.04 (br d, J=6.9 Hz, 1H), 6.09 (s, 2H), 4.30-4.21 (m, 2H), 2.59-2.47 (m, 1H), 2.34-2.22 (m, 1H), 1.64 (s, 3H).

MS ESI m/z 496.1 (M+H)⁺.

### Example 32: 5-(3-(2-amino-[1,2,4]triazolo[1,5-a]pyridin-7-yl)-6-chloro-2-fluorophenoxy)-3,3-difluoro-2-(5-fluoropyridin-2-yl)pentan-2-ol (enantiomer 2)

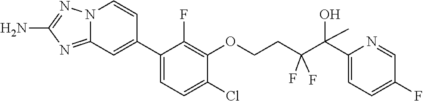 [SMILES: CC(O)(c1ccc(F)cn1)C(F)(F)CCOc1c(Cl)ccc(-c2ccn3nc(N)nc3c2)c1F]

32, ¹H NMR (500 MHz, DMSO-d₆) δ 8.62 (d, J=7.0 Hz, 1H), 8.56 (d, J=2.6 Hz, 1H), 7.82-7.73 (m, 2H), 7.53 (s, 1H), 7.45-7.38 (m, 2H), 7.04 (br d, J=6.9 Hz, 1H), 6.09 (s, 2H), 4.31-4.20 (m, 2H), 2.60-2.46 (m, 1H), 2.36-2.21 (m, 1H), 1.64 (s, 3H).

MS ESI m/z 496.1 (M+H)⁺.

### Example 33: 4-(3-(2-amino-[1,2,4]triazolo[1,5-a]pyridin-7-yl)-2,6-difluorophenoxy)-2,2-difluoro-1-(5-fluoropyridin-2-yl)butan-1-ol (enantiomer 1)

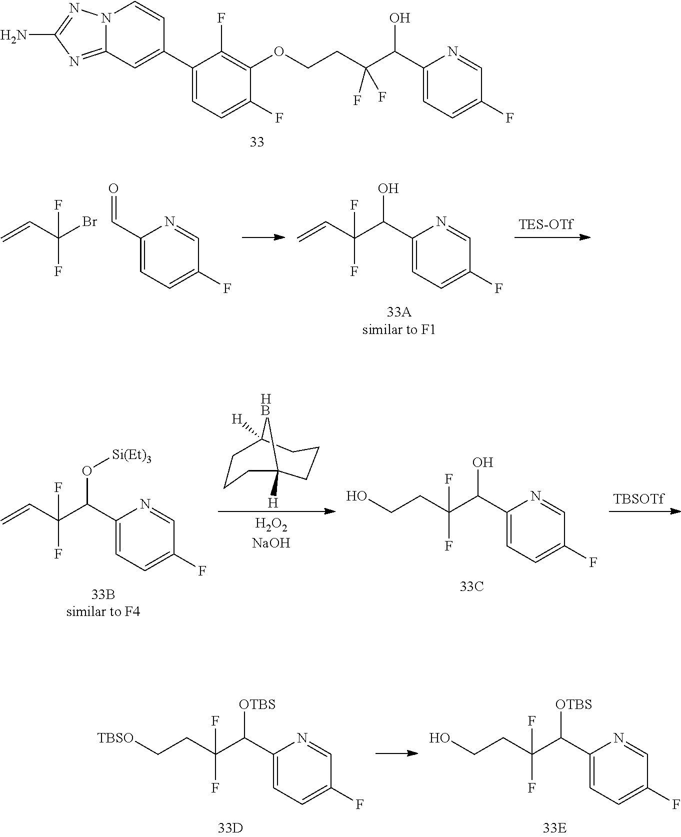

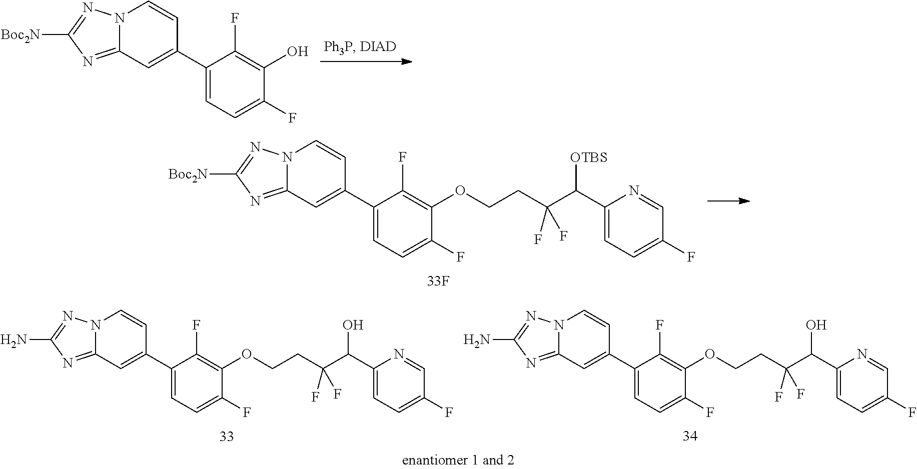

(The 2 unassigned enantiomers are:

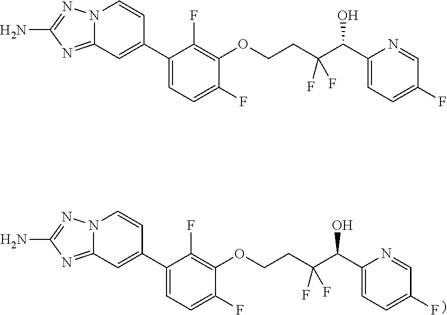 [SMILES: Nc1nc2cc(-c3ccc(F)c(OCCC(F)(F)[C@H](O)c4ccc(F)cn4)c3F)ccn2n1 ; Nc1nc2cc(-c3ccc(F)c(OCCC(F)(F)[C@@H](O)c4ccc(F)cn4)c3F)ccn2n1]

33A: 2,2-difluoro-1-(5-fluoropyridin-2-yl)but-3-en-1-ol was prepared in a similar fashion to F1 from 5-fluoropicolinaldehyde and 3-bromo-3,3-difluoroprop-1-ene.

33B: 2-(2,2-difluoro-1-((triethylsilyl)oxy)but-3-en-1-yl)-5-fluoropyridine was prepared in a similar fashion to F4

33C: 2,2-difluoro-1-(5-fluoropyridin-2-yl)butane-1,4-diol was prepared in a similar fashion to 27A, but the TES was deprotected during the reaction.

¹H NMR (499 MHz, chloroform-d) δ 8.49-8.46 (m, 1H), 7.53-7.49 (m, 2H), 5.14 (br d, J=4.7 Hz, 1H), 4.96 (br d, J=16.2 Hz, 1H), 3.97-3.88 (m, 2H), 2.67-2.45 (m, 1H), 2.44-2.43 (m, 1H), 2.36 (ddddd, J=18.3, 16.8, 15.2, 6.9, 5.0 Hz, 1H), 2.25-2.12 (m, 1H)

MS ESI m/z 221.8 (M+H)⁺.

### 33D: 2-(6,6-difluoro-2,2,3,3,10,10,11,11-octamethyl-4,9-dioxa-3,10-disiladodecan-5-yl)-5-fluoropyridine

To a solution of 2,2-difluoro-1-(5-fluoropyridin-2-yl)butane-1,4-diol (290 mg, 1.311 mmol) in DCM (6 mL) was added tert-butyldimethylsilyl trifluoromethanesulfonate (1040 mg, 3.93 mmol) followed by 2,6-lutidine (4.72 mmol, 506 mg) slowly at rt. The mixture was stirred at rt for 2 days. The reaction was concentrated. The residue was purified via silica gel chromatography (24 g, hexanes-20% EtOAc) to give 2-(6,6-difluoro-2,2,3,3,10,10,11,11-octamethyl-4,9-dioxa-3,10-disiladodecan-5-yl)-5-fluoropyridine (520 mg, 1.156 mmol, 88% yield).

MS ESI m/z 450.7 (M+H)⁺.

### 33E: 4-((tert-butyldimethylsilyl)oxy)-3,3-difluoro-4-(5-fluoropyridin-2-yl)butan-1-ol

A solution of 2-(6,6-difluoro-2,2,3,3,10,10,11,11-octamethyl-4,9-dioxa-3,10-disiladodecan-5-yl)-5-fluoropyridine (520 mg, 1.156 mmol) in AcOH (4 mL)/H₂O (2 mL)/THF (1 mL) was stirred at 30° C. for 6 h. TLC showed SM was consumed, and no diol was detected. The mixture was concentrated. EtOAc was added, extracted with 3× NaHCO₃/water and water, dried over Na₂SO₄/NaHCO₃, then concentrated to give clean 4-((tert-butyldimethylsilyl)oxy)-3,3-difluoro-4-(5-fluoropyridin-2-yl)butan-1-ol (380 mg, 1.133 mmol, 98% yield).

¹H NMR (499 MHz, CHLOROFORM-d) δ 8.43 (d, J=2.9 Hz, 1H), 7.66 (dd, J=8.7, 4.4 Hz, 1H), 7.49 (td, J=8.4, 2.9 Hz, 1H), 5.12 (dd, J=13.3, 7.6 Hz, 1H), 3.96-3.84 (m, 2H), 2.99 (t, J=5.8 Hz, 1H), 2.24-2.02 (m, 2H), 0.92 (s, 9H), 0.15 (s, 3H), −0.02 (s, 3H) MS ESI m/z 336.1 (M+H)⁺.

33F: tert-butyl (tert-butoxycarbonyl)(7-(3-(4-((tert-butyldimethylsilyl)oxy)-3,3-difluoro-4-(5-fluoropyridin-2-yl)butoxy)-2,4-difluorophenyl)-[1,2,4]triazolo[1,5-a]pyridin-2-yl)carbamate was prepared in a similar fashion to 2A from 33E and tert-butyl (tert-butoxycarbonyl)(7-(2,4-difluoro-3-hydroxyphenyl)-[1,2,4]triazolo[1,5-a]pyridin-2-yl)carbamate.

33 and 34: The two enantiomers of 4-(3-(2-amino-[1,2,4]triazolo[1,5-a]pyridin-7-yl)-2,6-difluorophenoxy)-2,2-difluoro-1-(5-fluoropyridin-2-yl)butan-1-ol were prepared in a similar fashion to 2 and 3.

The first eluting isomer as 33 4-(3-(2-amino-[1,2,4]triazolo[1,5-a]pyridin-7-yl)-2,6-difluorophenoxy)-2,2-difluoro-1-(5-fluoropyridin-2-yl)butan-1-ol (6.7 mg, 0.014 mmol, 6.24% yield) and the second eluting isomer as 34 4-(3-(2-amino-[1,2,4]triazolo[1,5-a]pyridin-7-yl)-2,6-difluorophenoxy)-2,2-difluoro-1-(5-fluoropyridin-2-yl)butan-1-ol (7.2 mg, 0.015 mmol, 6.41% yield) were obtained. 33: ¹H NMR (500 MHz, DMSO-d₆) δ 8.62 (d, J=6.9 Hz, 1H), 8.54 (d, J=2.7 Hz, 1H), 7.78 (td, J=8.8, 2.8 Hz, 1H), 7.63 (dd, J=8.7, 4.5 Hz, 1H), 7.51 (s, 1H), 7.44-7.37 (m, 1H), 7.29 (br t, J=9.5 Hz, 1H), 7.03 (br d, J=7.0 Hz, 1H), 6.08 (s, 2H), 4.98-4.90 (m, 1H), 4.38 (br t, J=6.7 Hz, 2H), 2.61-2.41 (m, 2H).

MS ESI m/z 466.1 (M+H)⁺.

### Example 34: 4-(3-(2-amino-[1,2,4]triazolo[1,5-a]pyridin-7-yl)-2,6-difluorophenoxy)-2,2-difluoro-1-(5-fluoropyridin-2-yl)butan-1-ol (enantiomer 1)

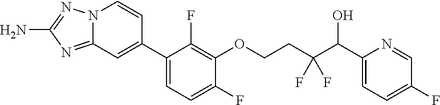 [SMILES: Nc1nc2cc(-c3ccc(F)c(OCCC(F)(F)C(O)c4ccc(F)cn4)c3F)ccn2n1]

34, ¹H NMR (500 MHz, DMSO-d₆) δ 8.61 (d, J=6.9 Hz, 1H), 8.53 (d, J=2.8 Hz, 1H), 7.78 (td, J=8.8, 3.0 Hz, 1H), 7.63 (dd, J=8.8, 4.6 Hz, 1H), 7.51 (s, 1H), 7.41 (td, J=8.5, 5.8 Hz, 1H), 7.28 (br t, J=9.6 Hz, 1H), 7.04 (br d, J=7.0 Hz, 1H), 6.08 (s, 2H), 4.98-4.90 (m, 1H), 4.38 (br t, J=6.6 Hz, 2H), 2.58-2.42 (m, 2H).

MS ESI m/z 466.1 (M+H)⁺.

### Example 35: 5-(3-(2-amino-[1,2,4]triazolo[1,5-a]pyridin-7-yl)-2,6-difluorophenoxy)-3,3-difluoro-2-(pyridin-3-yl)pentan-2-ol (enantiomer 1)

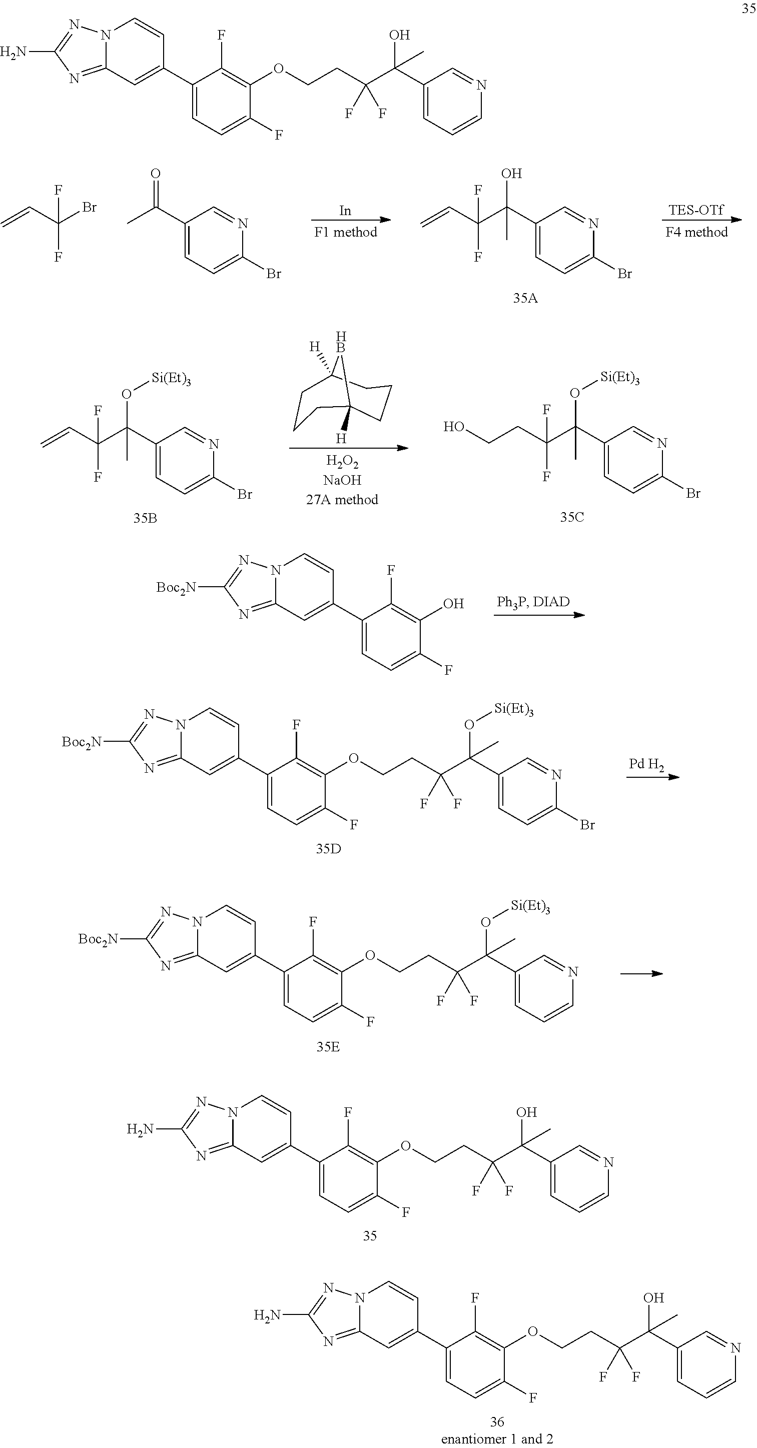

(The 2 unassigned enantiomers are:

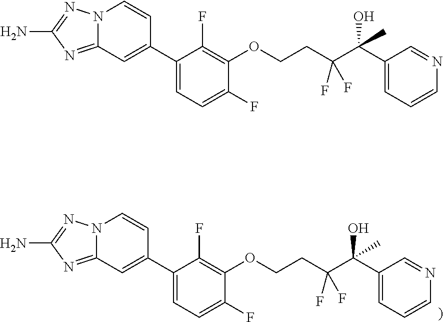 [SMILES: C[C@@](O)(c1cccnc1)C(F)(F)CCOc1c(F)ccc(-c2ccn3nc(N)nc3c2)c1F ; C[C@](O)(c1cccnc1)C(F)(F)CCOc1c(F)ccc(-c2ccn3nc(N)nc3c2)c1F]

35A: 2-(6-bromopyridin-3-yl)-3,3-difluoropent-4-en-2-ol was prepared in a similar fashion to F1 using 3-bromo-3,3-difluoroprop-1-ene and 1-(6-bromopyridin-3-yl)ethan-1-one.

35B: 2-bromo-5-(3,3-difluoro-2-((triethylsilyl)oxy)pent-4-en-2-yl)pyridine was prepared in a similar fashion to F4 from 35A.

35C: 4-(6-bromopyridin-3-yl)-3,3-difluoro-4-((triethylsilyl)oxy)pentan-1-ol was prepared in a similar fashion to 27A.

MS ESI m/z 410.1 (M+H)⁺.

35D: tert-butyl (7-(3-((4-(6-bromopyridin-3-yl)-3,3-difluoro-4-((triethylsilyl)oxy)pentyl)oxy)-2,4-difluorophenyl)-[1,2,4]triazolo[1,5-a]pyridin-2-yl)(tert-butoxycarbonyl)carbamate was prepared in a similar fashion to 2A.

MS ESI m/z 854.3 (M+H)⁺.

35E: tert-butyl (tert-butoxycarbonyl)(7-(3-((3,3-difluoro-4-(pyridin-3-yl)-4-((triethylsilyl)oxy)pentyl)oxy)-2,4-difluorophenyl)-[1,2,4]triazolo[1,5-a]pyridin-2-yl)carbamate: To a solution of 35D (75 mg, 0.088 mmol) in MeOH (2 mL) was added Pd—C (46.7 mg, 0.044 mmol) under N₂. A few drops of TFA was added. The mixture was stirred under H2 balloon at rt for 2 h. The Pd/C was filtered out. The filtrate was concentrated to give a crude 35E (68 mg, 0.088 mmol, 100% yield), which was used for the next step.

35 and 36: The two enantiomers of 5-(3-(2-amino-[1,2,4]triazolo[1,5-a]pyridin-7-yl)-2,6-difluorophenoxy)-3,3-difluoro-2-(pyridin-3-yl)pentan-2-ol were prepared in a similar fashion to 2 and 3.

The first eluting peak 35 5-(3-(2-amino-[1,2,4]triazolo[1,5-a]pyridin-7-yl)-2,6-difluorophenoxy)-3,3-difluoro-2-(pyridin-3-yl)pentan-2-ol (3 mg, 6.45 μmol, 7.36% yield) and the second eluting peak 36 5-(3-(2-amino-[1,2,4]triazolo[1,5-a]pyridin-7-yl)-2,6-difluorophenoxy)-3,3-difluoro-2-(pyridin-3-yl)pentan-2-ol (1.9 mg, 4.07 μmol, 4.65% yield) were obtained.

35, ¹H NMR (500 MHz, DMSO-d₆) δ 8.71 (s, 1H), 8.60 (d, J=7.1 Hz, 1H), 8.51 (d, J=3.4 Hz, 1H), 7.91 (br d, J=8.4 Hz, 1H), 7.48 (s, 1H), 7.42-7.35 (m, 2H), 7.28-7.22 (m, 1H), 7.02 (d, J=6.9 Hz, 1H), 6.36 (s, 1H), 6.07 (s, 2H), 4.35-4.25 (m, 2H), 2.50-2.41 (m, 1H), 2.23-2.10 (m, 1H), 1.64 (s, 3H).

MS ESI m/z 462.2 (M+H)⁺.

### Example 36: 5-(3-(2-amino-[1,2,4]triazolo[1,5-a]pyridin-7-yl)-2,6-difluorophenoxy)-3,3-difluoro-2-(pyridin-3-yl)pentan-2-ol (enantiomer 2)

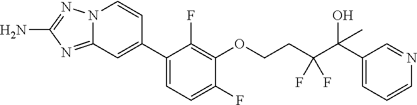 [SMILES: CC(O)(c1cccnc1)C(F)(F)CCOc1c(F)ccc(-c2ccn3nc(N)nc3c2)c1F]

36, ¹H NMR (500 MHz, DMSO-d₆) δ 8.71 (s, 1H), 8.60 (d, J=6.9 Hz, 1H), 8.52-8.50 (m, 1H), 7.91 (br d, J=7.9 Hz, 1H), 7.48 (s, 1H), 7.42-7.35 (m, 2H), 7.28-7.23 (m, 1H), 7.02 (br d, J=7.2 Hz, 1H), 6.36 (s, 1H), 6.07 (s, 2H), 4.34-4.25 (m, 2H), 2.49-2.41 (m, 1H), 2.23-2.12 (m, 1H), 1.64 (s, 3H).

MS ESI m/z 462.2 (M+H)⁺.

### Example 37: 5-(3-(2-amino-8-fluoro-[1,2,4]triazolo[1,5-a]pyridin-7-yl)-2,6-difluorophenoxy)-3,3-difluoro-2-phenylpentan-2-ol (enantiomer 1)

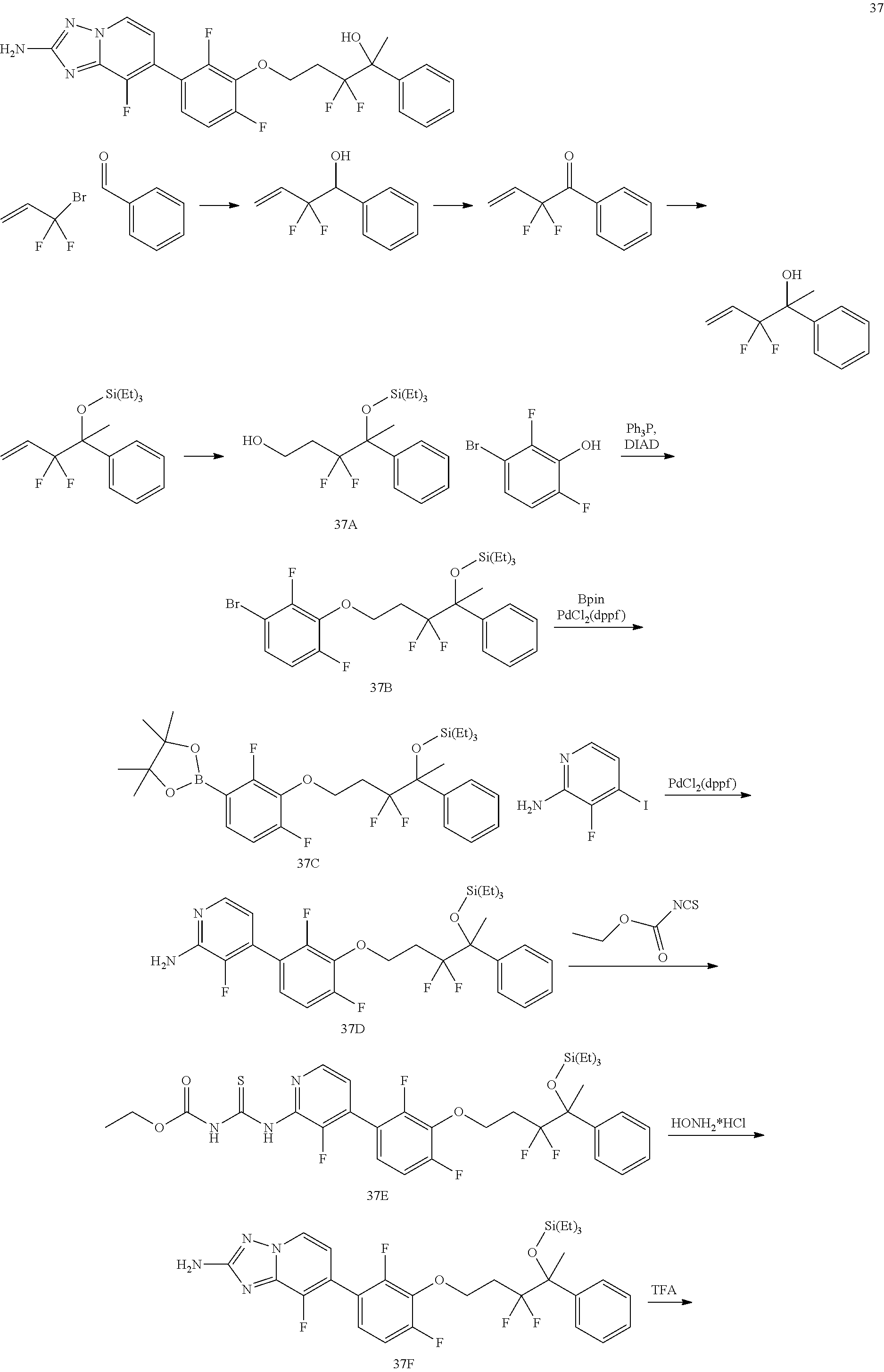

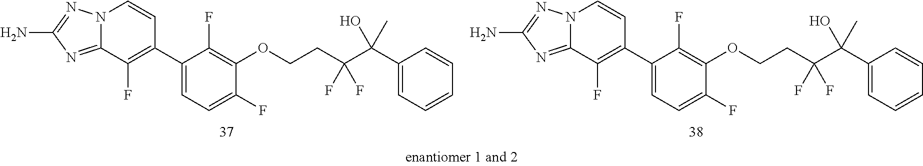 [SMILES: CC(O)(c1ccccc1)C(F)(F)CCOc1c(F)ccc(-c2ccn3nc(N)nc3c2F)c1F ; CC(O)(c1ccccc1)C(F)(F)CCOc1c(F)ccc(-c2ccn3nc(N)nc3c2F)c1F]

(The 2 unassigned enantiomers are:

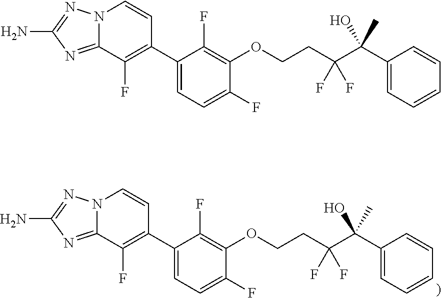 [SMILES: C[C@@](O)(c1ccccc1)C(F)(F)CCOc1c(F)ccc(-c2ccn3nc(N)nc3c2F)c1F ; C[C@](O)(c1ccccc1)C(F)(F)CCOc1c(F)ccc(-c2ccn3nc(N)nc3c2F)c1F]

37A: 3,3-difluoro-4-phenyl-4-((tri ethyl silyl)oxy)pentan-1-ol was prepared in a similar fashion to intermediate F.

37B: ((5-(3-bromo-2,6-difluorophenoxy)-3,3-difluoro-2-phenylpentan-2-yl)oxy)triethylsilane was prepared in a similar fashion to 8A.

37C: ((5-(2,6-difluoro-3-(4,4,5,5-tetramethyl-1,3,2-dioxaborolan-2-yl)phenoxy)-3,3-difluoro-2-phenylpentan-2-yl)oxy)triethylsilane:

A mixture of ((5-(3-bromo-2,6-difluorophenoxy)-3,3-difluoro-2-phenylpentan-2-yl)oxy)triethylsilane (171 mg, 0.328 mmol), bis(pinacolato)diboron (92 mg, 0.361 mmol), potassium acetate (97 mg, 0.984 mmol) and PdCl₂(dppf)-DCM adduct (26.8 mg, 0.033 mmol) in dioxane (1 mL) was purged with nitrogen, sealed, and stirred at 100° C. for 3 h.

The material was directly used in the next step.

### 37D: 4-(3-((3,3-difluoro-4-phenyl-4-((triethylsilyl)oxy)pentyl)oxy)-2,4-difluorophenyl)-3-fluoropyridin-2-amine

A mixture of ((5-(2,6-difluoro-3-(4,4,5,5-tetramethyl-1,3,2-dioxaborolan-2-yl)phenoxy)-3,3-difluoro-2-phenylpentan-2-yl)oxy)triethylsilane (186 mg, 0.328 mmol) (37C), 3-fluoro-4-iodopyridin-2-amine (86 mg, 0.361 mmol), potassium phosphate tribasic anhydrous, 99% (0.492 mL, 0.984 mmol) (2.0 M) and PdCl₂(dppf)-DCM adduct (26.8 mg, 0.033 mmol) in dioxane (1 mL) was stirred at 80° C. for 1.5 h. The mixture was diluted with EtOAc, dried with Na₂SO₄, and filtered. The organic solution was concentrated. The residue was purified via silica gel chromatography (24 g column, up to 80% EtOAc in hexane) to give the desired product 4-(3-((3,3-difluoro-4-phenyl-4-((triethylsilyl)oxy)pentyl)oxy)-2,4-difluorophenyl)-3-fluoropyridin-2-amine (123 mg, 0.223 mmol, 67.9% yield) as a colorless oil.

¹H NMR (499 MHz, chloroform-d) δ 7.90 (d, J=5.2 Hz, 1H), 7.58-7.51 (m, 2H), 7.39-7.26 (m, 3H), 7.08-6.93 (m, 2H), 6.66-6.58 (m, 1H), 4.82 (s, 2H), 4.35 (td, J=9.4, 6.0 Hz, 1H), 4.25 (td, J=9.4, 5.8 Hz, 1H), 2.69-2.47 (m, 1H), 2.15-1.97 (m, 1H), 1.79 (s, 3H), 0.97 (t, J=7.9 Hz, 9H), 0.75-0.62 (m, 6H).

¹⁹F NMR (470 MHz, chloroform-d) δ −108.20-−110.79 (m, 1F), −113.08 (br d, J=241.6 Hz, 1F), −126.06 (d, J=10.6 Hz, 1F), −127.14 (dd, J=19.1, 10.6 Hz, 1F), −142.35 (d, J=19.1 Hz, 1F).

MS ESI m/z 553.4 (M+H)⁺.

37E: A 20 mL reaction vial was charged with a stir bar, and 4-(3-((3,3-difluoro-4-phenyl-4-((triethylsilyl)oxy)pentyl)oxy)-2,4-difluorophenyl)-3-fluoropyridin-2-amine (123 mg, 0.223 mmol). The flask was evacuated and backfilled with nitrogen, followed by the addition of dioxane (0.5 mL), DCM (0.500 mL) and ethoxycarbonyl isothiocyanate (0.039 mL, 0.334 mmol) dropwise at rt. The resulting mixture was stirred at rt over the weekend. Another 0.010 mL of reagent was added and the reaction continued for another 20 h. LCMS showed complete conversion. The mixture was concentrated and the residue was purified by FCC up to 80% EtOAc/hexane to afford the desired product (130 mg, 85%) as a light yellow oil.

MS ESI m/z 684.6 (M+H)⁺.

### 37F: 7-(3-((3,3-difluoro-4-phenyl-4-((triethylsilyl)oxy)pentyl)oxy)-2,4-difluorophenyl)-8-fluoro-[1,2,4]triazolo[1,5-a]pyridin-2-amine

A 250 mL flask was charged with a stir bar, crude 37E (130 mg, 0.190 mmol) and hydroxylamine hydrochlorid (66.1 mg, 0.951 mmol) were added. The flask was evacuated and backfilled with nitrogen, followed by the addition of ethanol (1267 μl) and DIPEA (100 μl, 0.570 mmol). The resulting mixture was stirred at rt for 15 min and then at 80° C. for 3 h. The volatiles were stripped off and the residue was directly used in the deprotection step.

MS ESI m/z 593.4 (M+H)⁺.

37 and 38: The two enantiomers of 5-(3-(2-amino-8-fluoro-[1,2,4]triazolo[1,5-a]pyridin-7-yl)-2,6-difluorophenoxy)-3,3-difluoro-2-phenylpentan-2-ol were prepared in a similar fashion to 2 and 3.

37 (26.6 mg, 29%): ¹H NMR (500 MHz, DMSO-d₆) δ 8.54 (d, J=6.8 Hz, 1H), 7.53 (br d, J=7.6 Hz, 2H), 7.39-7.33 (m, 2H), 7.32-7.26 (m, 3H), 6.90 (t, J=6.6 Hz, 1H), 6.29 (s, 2H), 6.08 (s, 1H), 4.35-4.18 (m, 2H), 2.48-2.36 (m, 1H), 2.16-1.99 (m, 1H), 1.60 (s, 3H);

¹⁹F NMR (471 MHz, DMSO-d₆) δ −106.69-−109.03 (m, 1F), −110.51-−112.21 (m, 1F), −125.36-−127.27 (m, 1F), −127.48-−128.97 (m, 1F), −134.06-−136.18 (m, 1F).

MS ESI m/z 479.2 (M+H)⁺.

### Example 38: 5-(3-(2-amino-8-fluoro-[1,2,4]triazolo[1,5-a]pyridin-7-yl)-2,6-difluorophenoxy)-3,3-difluoro-2-phenylpentan-2-ol (enantiomer 2)

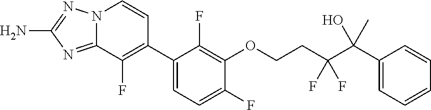 [SMILES: CC(O)(c1ccccc1)C(F)(F)CCOc1c(F)ccc(-c2ccn3nc(N)nc3c2F)c1F]

38 (30.5 mg, 34%): ¹H NMR (500 MHz, DMSO-d₆) δ 8.53 (d, J=6.9 Hz, 1H), 7.53 (br d, J=7.9 Hz, 2H), 7.39-7.33 (m, 2H), 7.32-7.26 (m, 3H), 6.90 (t, J=6.6 Hz, 1H), 6.29 (s, 2H), 6.09 (s, 1H), 4.33-4.18 (m, 2H), 2.49-2.36 (m, 1H), 2.12-1.99 (m, 1H), 1.60 (s, 3H);

¹⁹F NMR (471 MHz, DMSO-d₆) δ −107.12-−109.24 (m, 1F), −110.72-−112.42 (m, 1F), −126.42 (br d, J=8.7 Hz, 1F), −128.03 (br s, 1F), −135.07 (br s, 1F).

MS ESI m/z 479.2 (M+H)⁺.

### Example 39: 5-(3-(2-amino-[1,2,4]triazolo[1,5-a]pyridin-7-yl)-2,6-difluorophenoxy)-3,3-difluoro-2-phenylpentan-2-ol (enantiomer 1)

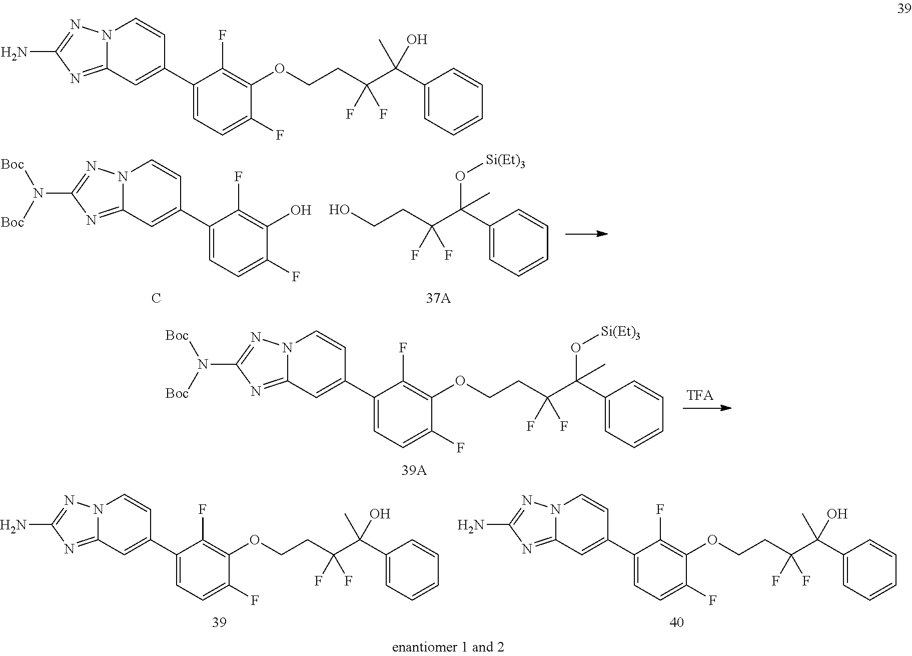

(The 2 unassigned enantiomers are:

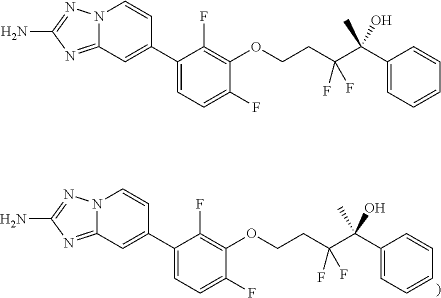 [SMILES: C[C@@](O)(c1ccccc1)C(F)(F)CCOc1c(F)ccc(-c2ccn3nc(N)nc3c2)c1F ; C[C@](O)(c1ccccc1)C(F)(F)CCOc1c(F)ccc(-c2ccn3nc(N)nc3c2)c1F]

39A: tert-butyl (tert-butoxycarbonyl)(7-(3-((3,3-difluoro-4-phenyl-4-((triethylsilyl)oxy)-pentyl)oxy)-2,4-difluorophenyl)-[1,2,4]triazolo[1,5-a]pyridin-2-yl)carbamate was prepared in a similar fashion to 8A using C and 37A as starting materials. 39 and 40: The two enantiomers of 5-(3-(2-amino-[1,2,4]triazolo[1,5-a]pyridin-7-yl)-2,6-difluorophenoxy)-3,3-difluoro-2-phenylpentan-2-ol were prepared in a similar fashion to 2 and 3

39 (74.7 mg, 40%, with TFA): ¹H NMR (499 MHz, METHANOL-d4) δ 8.49 (d, J=6.9 Hz, 1H), 7.59 (br d, J=7.5 Hz, 2H), 7.49 (s, 1H), 7.39-7.33 (m, 2H), 7.32-7.26 (m, 2H), 7.16-7.07 (m, 2H), 4.38-4.29 (m, 1H), 4.23 (td, J=9.3, 6.0 Hz, 1H), 2.57-2.39 (m, 1H), 2.17-1.99 (m, 1H), 1.67 (s, 3H).

¹⁹F NMR (470 MHz, methanol-d₄) δ −109.65 (br d, J=248.0 Hz, 1F), −113.95 (br d, J=248.0 Hz, 1F), −128.22 (br s, 1F), −132.69 (br d, J=8.5 Hz, 1F) (contains TFA).

MS ESI m/z 479.2 (M+H)⁺.

### Example 40: 5-(3-(2-amino-[1,2,4]triazolo[1,5-a]pyridin-7-yl)-2,6-difluorophenoxy)-3,3-difluoro-2-phenylpentan-2-ol (enantiomer 2)

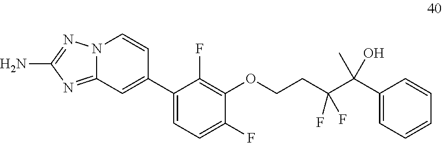 [SMILES: CC(O)(c1ccccc1)C(F)(F)CCOc1c(F)ccc(-c2ccn3nc(N)nc3c2)c1F]

40 (45.9 mg, 24%): ¹H NMR (499 MHz, methanol-d₄) δ 8.51-8.44 (m, 1H), 7.59 (br d, J=7.3 Hz, 2H), 7.48 (br s, 1H), 7.38-7.32 (m, 2H), 7.31-7.23 (m, 2H), 7.15-7.05 (m, 2H), 4.38-4.29 (m, 1H), 4.22 (td, J=9.1, 5.9 Hz, 1H), 2.56-2.39 (m, 1H), 2.16-2.00 (m, 1H), 1.67 (s, 3H);

¹⁹F NMR (470 MHz, methanol-d₄) δ −109.62 (br d, J=248.0 Hz, 1F), −112.35-−114.95 (m, 1F), −128.27 (br d, J=10.6 Hz, 1F), −132.71 (br d, J=10.6 Hz, 1F) (no TFA).

### Example 41: 5-(3-(2-amino-[1,2,4]triazolo[1,5-a]pyridin-7-yl)-6-chloro-2-fluorophenoxy)-3,3-difluoro-2-phenylpentan-2-ol (enantiomer 1)

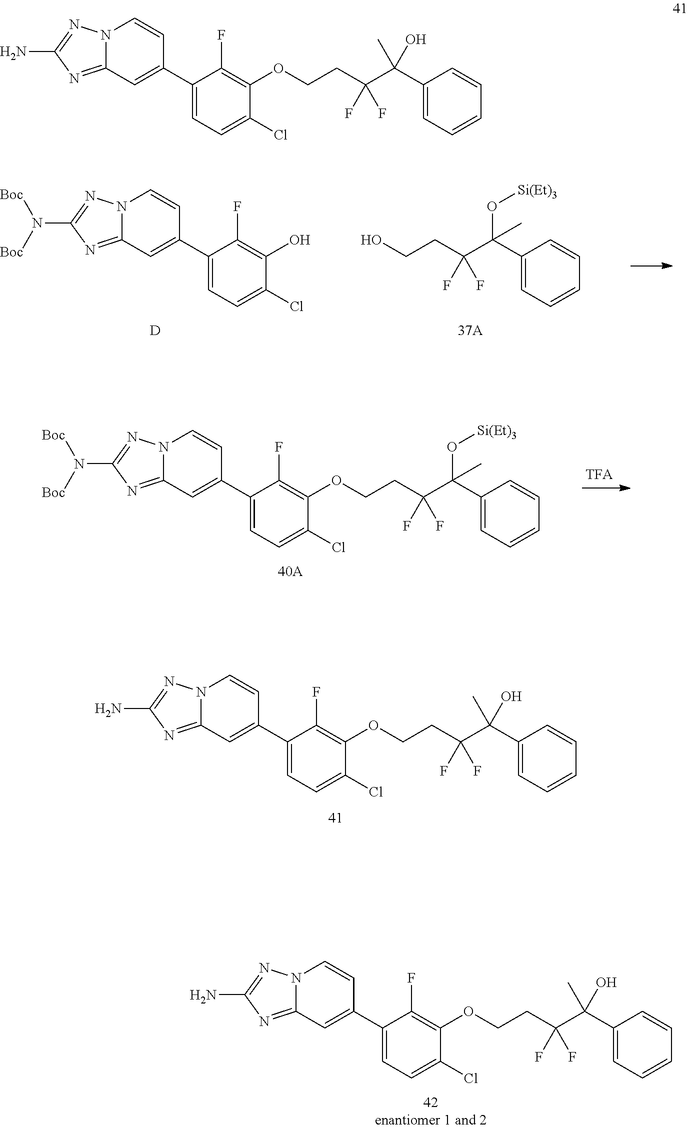

(The 2 unassigned enantiomers are:

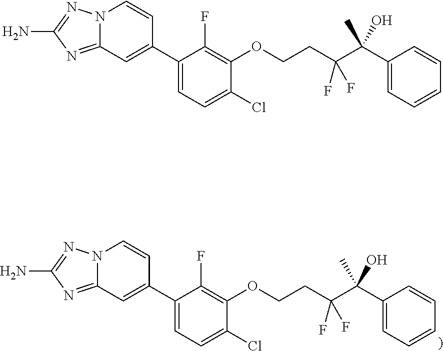 [SMILES: C[C@@](O)(c1ccccc1)C(F)(F)CCOc1c(Cl)ccc(-c2ccn3nc(N)nc3c2)c1F ; C[C@](O)(c1ccccc1)C(F)(F)CCOc1c(Cl)ccc(-c2ccn3nc(N)nc3c2)c1F]

41 and 42: The two enantiomers of 5-(3-(2-amino-[1,2,4]triazolo[1,5-a]pyridin-7-yl)-6-chloro-2-fluorophenoxy)-3,3-difluoro-2-phenylpentan-2-ol were prepared in a similar fashion to 39 and 40 using D and 37A as starting materials.

41 (16.3 mg, 17%): ¹H NMR (500 MHz, DMSO-d₆) δ 8.82 (d, J=6.7 Hz, 1H), 7.72 (s, 1H), 7.54 (br d, J=7.3 Hz, 2H), 7.49-7.40 (m, 2H), 7.40-7.34 (m, 3H), 7.33-7.26 (m, 1H), 4.30-4.23 (m, 1H), 4.22-4.15 (m, 1H), 2.49-2.40 (m, 1H), 2.16-2.06 (m, 1H), 1.61 (s, 3H);

¹⁹F NMR (471 MHz, DMSO-d₆) δ −106.91-−109.03 (m, 1F), −111.33 (br d, J=244.5 Hz, 1F), −130.73 (s, 1F).

MS ESI m/z 477.1 (M+H)⁺.

### Example 42: 5-(3-(2-amino-[1,2,4]triazolo[1,5-a]pyridin-7-yl)-6-chloro-2-fluorophenoxy)-3,3-difluoro-2-phenylpentan-2-ol (enantiomer 2)

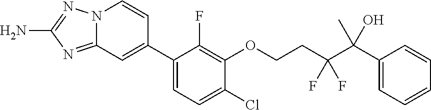 [SMILES: CC(O)(c1ccccc1)C(F)(F)CCOc1c(Cl)ccc(-c2ccn3nc(N)nc3c2)c1F]

42 (16.5 mg, 17%): ¹H NMR (500 MHz, DMSO-d₆) δ 8.81 (d, J=7.0 Hz, 1H), 7.70 (s, 1H), 7.55 (br d, J=7.6 Hz, 2H), 7.50-7.41 (m, 2H), 7.40-7.27 (m, 4H), 4.31-4.23 (m, 1H), 4.22-4.16 (m, 1H), 2.49-2.40 (m, 1H), 2.19-2.06 (m, 1H), 1.61 (s, 3H);

¹⁹F NMR (471 MHz, DMSO-d₆) δ −108.03 (br d, J=244.5 Hz, 1F), −111.33 (br d, J=242.8 Hz, 1F), −130.72 (s, 1F).

MS ESI m/z 477.1 (M+H)⁺.

### Example 43: 5-(3-(2-amino-[1,2,4]triazolo[1,5-a]pyridin-7-yl)-6-chloro-2-fluorophenoxy)-2-(4-chlorophenyl)-3,3-difluoropentan-2-ol (enantiomer 1)

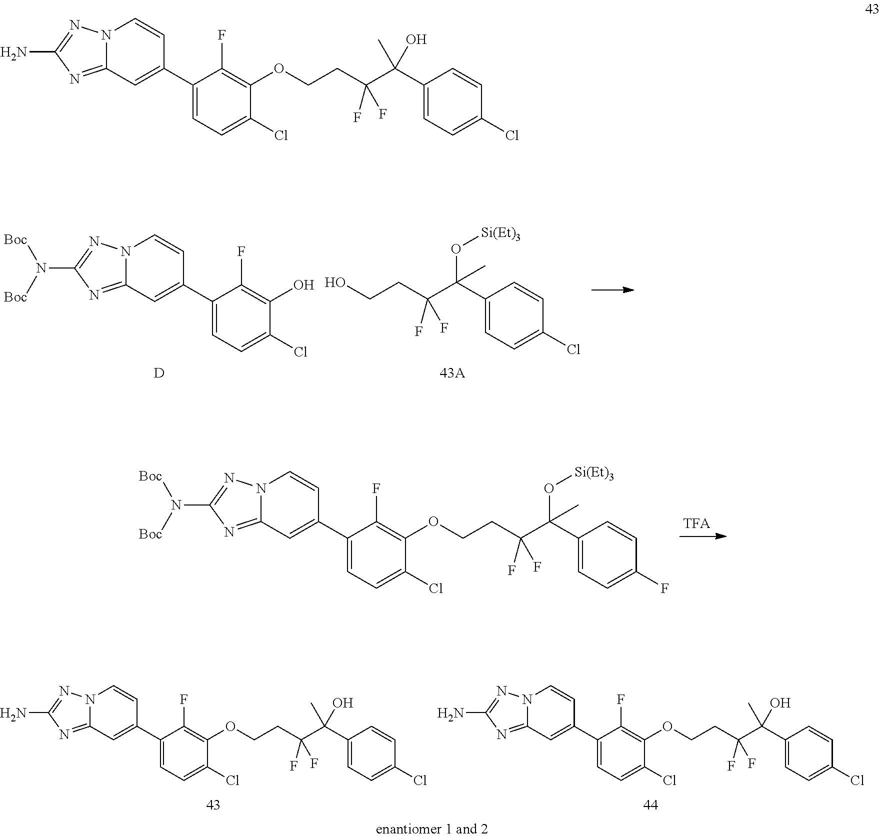

(The 2 unassigned enantiomers are:

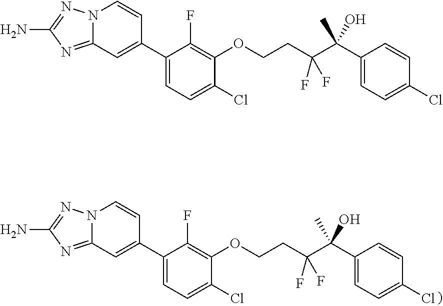 [SMILES: C[C@@](O)(c1ccc(Cl)cc1)C(F)(F)CCOc1c(Cl)ccc(-c2ccn3nc(N)nc3c2)c1F ; C[C@](O)(c1ccc(Cl)cc1)C(F)(F)CCOc1c(Cl)ccc(-c2ccn3nc(N)nc3c2)c1F]

43A: 4-(4-chlorophenyl)-3,3-difluoro-4-((triethylsilyl)oxy)pentan-1-ol was prepared in a similar fashion to F.

43 and 44: The two enantiomers of 5-(3-(2-amino-[1,2,4]triazolo[1,5-a]pyridin-7-yl)-6-chloro-2-fluorophenoxy)-2-(4-chlorophenyl)-3,3-difluoropentan-2-ol were prepared in a similar fashion to 39 and 40 using D and 43A as starting materials.

43 (8.4 mg, 27%): ¹H NMR (500 MHz, DMSO-d₆) δ 8.62 (d, J=7.0 Hz, 1H), 7.59-7.50 (m, 3H), 7.45-7.38 (m, 4H), 7.04 (br d, J=7.0 Hz, 1H), 6.25 (br s, 1H), 6.09 (s, 2H), 4.31-4.16 (m, 2H), 2.48-2.38 (m, 1H), 2.22-2.05 (m, 1H), 1.60 (s, 3H);

¹⁹F NMR (471 MHz, DMSO-d₆) δ −107.12-−109.03 (m, 1F), −111.12 (br d, J=246.2 Hz, 1F), −130.96 (s, 1F).

MS ESI m/z 511.1 (M+H)⁺.

### Example 44: 5-(3-(2-amino-[1,2,4]triazolo[1,5-a]pyridin-7-yl)-6-chloro-2-fluorophenoxy)-2-(4-chlorophenyl)-3,3-difluoropentan-2-ol (enantiomer 1)

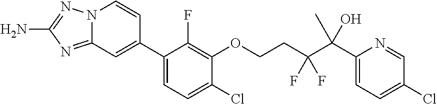 [SMILES: CC(O)(c1ccc(Cl)cn1)C(F)(F)CCOc1c(Cl)ccc(-c2ccn3nc(N)nc3c2)c1F]

44 (7.8 mg, 25%): ¹H NMR (500 MHz, DMSO-d₆) δ 8.62 (d, J=7.0 Hz, 1H), 7.60-7.50 (m, 3H), 7.47-7.37 (m, 4H), 7.04 (br d, J=6.4 Hz, 1H), 6.25 (br d, J=2.1 Hz, 1H), 6.09 (s, 2H), 4.30-4.16 (m, 2H), 2.49-2.39 (m, 1H), 2.20-2.07 (m, 1H), 1.60 (s, 3H);

¹⁹F NMR (471 MHz, DMSO-d₆) δ −107.12-−109.03 (m, 1F), −111.12 (br d, J=244.5 Hz, 1F), −130.96 (s, 1F).

MS ESI m/z 511.1 (M+H)⁺.

### Example 45: 5-(3-(2-amino-[1,2,4]triazolo[1,5-a]pyridin-7-yl)-2,6-difluorophenoxy)-2-(4-chlorophenyl)-3,3-difluoropentan-2-ol (enantiomer 1)

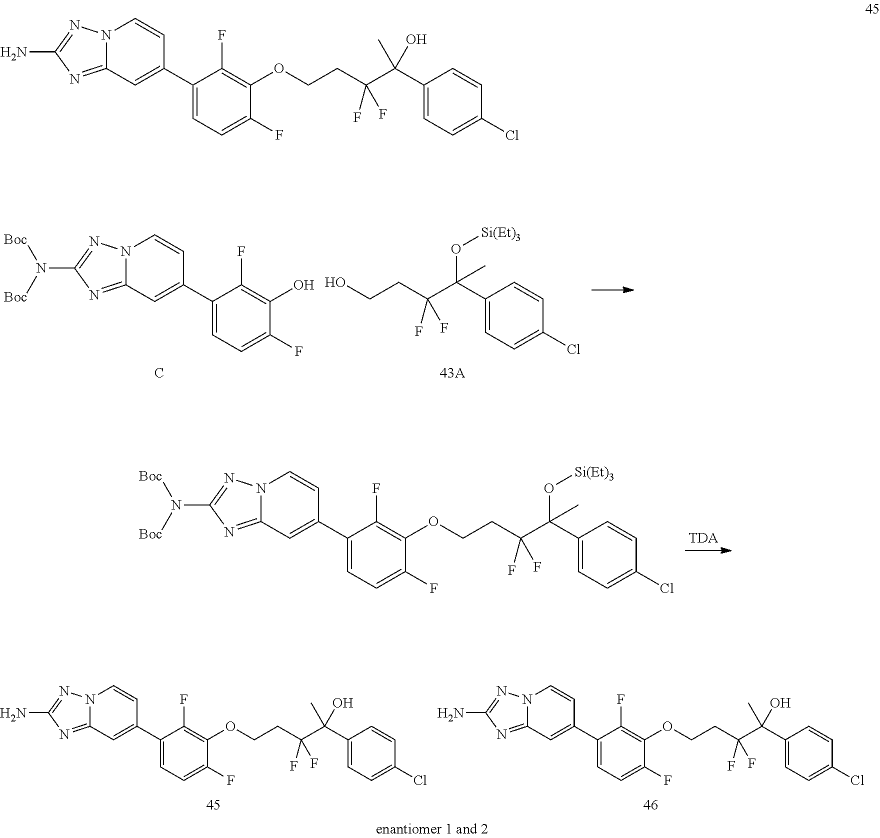

(The 2 unassigned enantiomers are:

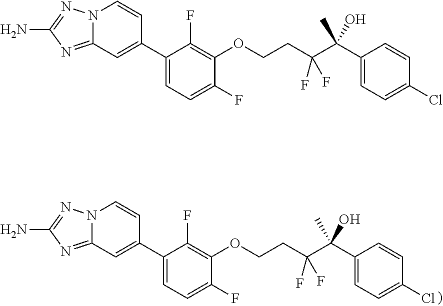 [SMILES: C[C@@](O)(c1ccc(Cl)cc1)C(F)(F)CCOc1c(F)ccc(-c2ccn3nc(N)nc3c2)c1F ; C[C@](O)(c1ccc(Cl)cc1)C(F)(F)CCOc1c(F)ccc(-c2ccn3nc(N)nc3c2)c1F]

45 and 46: The two enantiomers of 5-(3-(2-amino-[1,2,4]triazolo[1,5-a]pyridin-7-yl)-2,6-difluorophenoxy)-2-(4-chlorophenyl)-3,3-difluoropentan-2-ol were prepared in a similar fashion to 39 and 40 using C and 43A as starting materials.

45 (11.0 mg, 27%): ¹H NMR (500 MHz, DMSO-d₆) δ 8.60 (d, J=7.0 Hz, 1H), 7.54 (br d, J=8.5 Hz, 2H), 7.48 (s, 1H), 7.43-7.34 (m, 3H), 7.25 (br t, J=9.6 Hz, 1H), 7.02 (br d, J=6.7 Hz, 1H), 6.26 (s, 1H), 6.06 (s, 2H), 4.34-4.21 (m, 2H), 2.49-2.36 (m, 1H), 2.17-2.01 (m, 1H), 1.59 (s, 3H);

¹⁹F NMR (471 MHz, DMSO-d₆) δ −107.33-−109.24 (m, 1F), −111.21 (br d, J=246.2 Hz, 1F), −127.20 (br d, J=8.7 Hz, 1F), −131.22 (br d, J=8.7 Hz, 1F).

MS ESI m/z 495.1 (M+H)⁺.

### Example 46: 5-(3-(2-amino-[1,2,4]triazolo[1,5-a]pyridin-7-yl)-2,6-difluorophenoxy)-2-(4-chlorophenyl)-3,3-difluoropentan-2-ol (enantiomer 2)

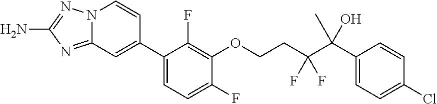 [SMILES: CC(O)(c1ccc(Cl)cc1)C(F)(F)CCOc1c(F)ccc(-c2ccn3nc(N)nc3c2)c1F]

46 (10.8 mg, 26%): ¹H NMR (500 MHz, DMSO-d₆) δ 8.61 (d, J=6.7 Hz, 1H), 7.55 (br d, J=7.9 Hz, 2H), 7.49 (s, 1H), 7.45-7.35 (m, 3H), 7.26 (br t, J=9.6 Hz, 1H), 7.02 (br d, J=6.7 Hz, 1H), 6.23 (br s, 1H), 6.08 (s, 2H), 4.36-4.22 (m, 2H), 2.49-2.37 (m, 1H), 2.17-2.02 (m, 1H), 1.60 (s, 3H);

¹⁹F NMR (471 MHz, DMSO-d₆) δ −107.12-−109.24 (m, 1F), −111.21 (br d, J=244.5 Hz, 1F), −127.28 (br d, J=8.7 Hz, 1F), −131.17 (br d, J=8.7 Hz, 1F).

MS ESI m/z 495.1 (M+H)⁺.

### Example 47: 5-(3-(2-amino-[1,2,4]triazolo[1,5-a]pyridin-7-yl)-2-fluorophenoxy)-2-(4-chlorophenyl)-3,3-difluoropentan-2-ol (enantiomer 1)

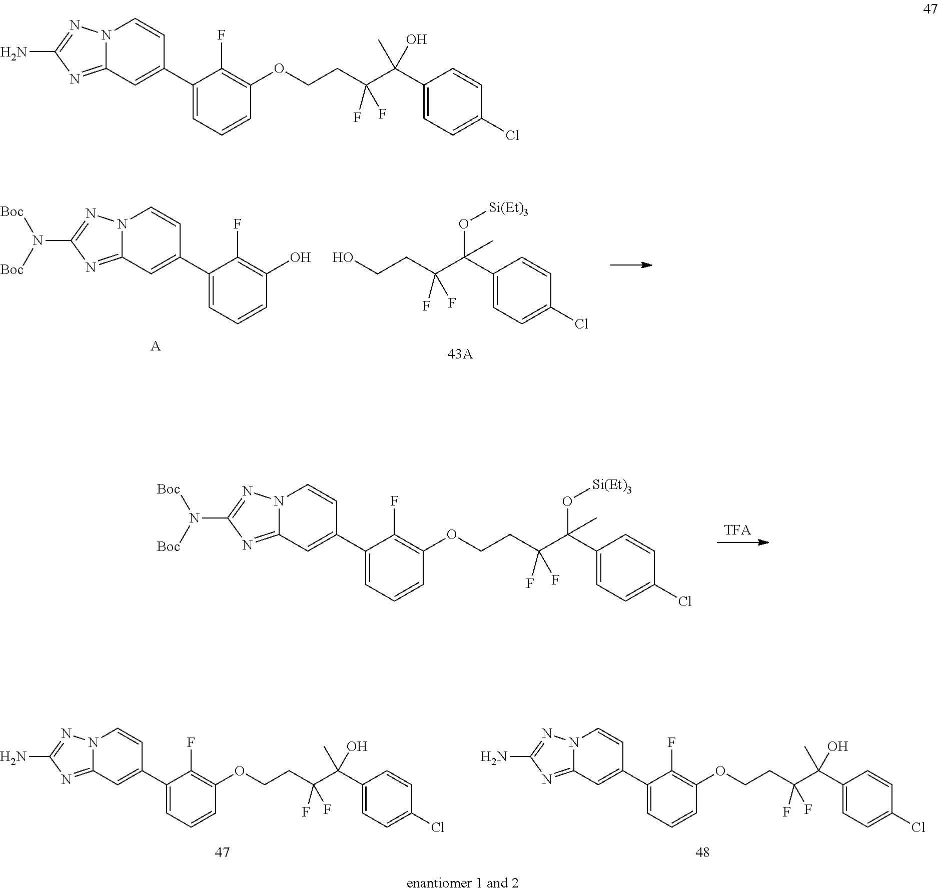

(The 2 unassigned enantiomers are:

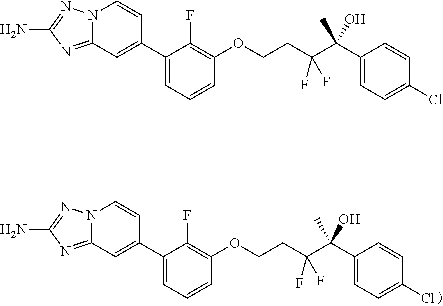 [SMILES: C[C@@](O)(c1ccc(Cl)cc1)C(F)(F)CCOc1cccc(-c2ccn3nc(N)nc3c2)c1F ; C[C@](O)(c1ccc(Cl)cc1)C(F)(F)CCOc1cccc(-c2ccn3nc(N)nc3c2)c1F]

47 and 48: The two enantiomers of 5-(3-(2-amino-[1,2,4]triazolo[1,5-a]pyridin-7-yl)-2-fluorophenoxy)-2-(4-chlorophenyl)-3,3-difluoropentan-2-ol were prepared in a similar fashion to 39 and 40 using A and 43A as starting materials.

47 (8.2 mg, 31%): ¹H NMR (500 MHz, DMSO-d₆) δ 8.59 (d, J=6.7 Hz, 1H), 7.57 (br d, J=8.2 Hz, 2H), 7.48 (s, 1H), 7.43 (d, J=8.5 Hz, 2H), 7.28-7.13 (m, 3H), 7.03 (br d, J=6.7 Hz, 1H), 6.27 (s, 1H), 6.18-5.95 (m, 2H), 4.31-4.18 (m, 2H), 2.50-2.39 (m, 1H), 2.18-2.04 (m, 1H), 1.61 (s, 3H;

¹⁹F NMR (471 MHz, DMSO-d₆) δ −107.75-−109.24 (m, 1F), −111.20 (br d, J=244.5 Hz, 1F), −139.64 (s, 1F).

MS ESI m/z 477.1 (M+H)⁺.

### Example 48: 5-(3-(2-amino-[1,2,4]triazolo[1,5-a]pyridin-7-yl)-2-fluorophenoxy)-2-(4-chlorophenyl)-3,3-difluoropentan-2-ol (enantiomer 2)

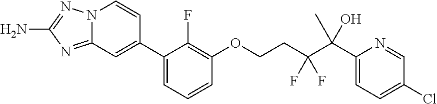 [SMILES: CC(O)(c1ccc(Cl)cn1)C(F)(F)CCOc1cccc(-c2ccn3nc(N)nc3c2)c1F]

48 (8.2 mg, 31%): ¹H NMR (500 MHz, DMSO-d₆) δ 8.58 (d, J=6.7 Hz, 1H), 7.57 (br d, J=8.2 Hz, 2H), 7.47 (s, 1H), 7.42 (d, J=8.5 Hz, 2H), 7.27-7.13 (m, 3H), 7.03 (br d, J=6.7 Hz, 1H), 6.27 (s, 1H), 6.05 (s, 2H), 4.29-4.18 (m, 2H), 2.49-2.39 (m, 1H), 2.17-2.02 (m, 1H), 1.60 (s, 3H);

¹⁹F NMR (471 MHz, DMSO-d₆) δ −107.33-−109.66 (m, 1F), −111.21 (br d, J=244.5 Hz, 1F), −139.65 (s, 1F).

MS ESI m/z 477.1 (M+H)⁺.

### Example 49: 5-(3-(2-amino-[1,2,4]triazolo[1,5-a]pyridin-7-yl)-2,6-difluorophenoxy)-3,3-difluoro-2-(4-fluorophenyl)pentan-2-ol (enantiomer 1)

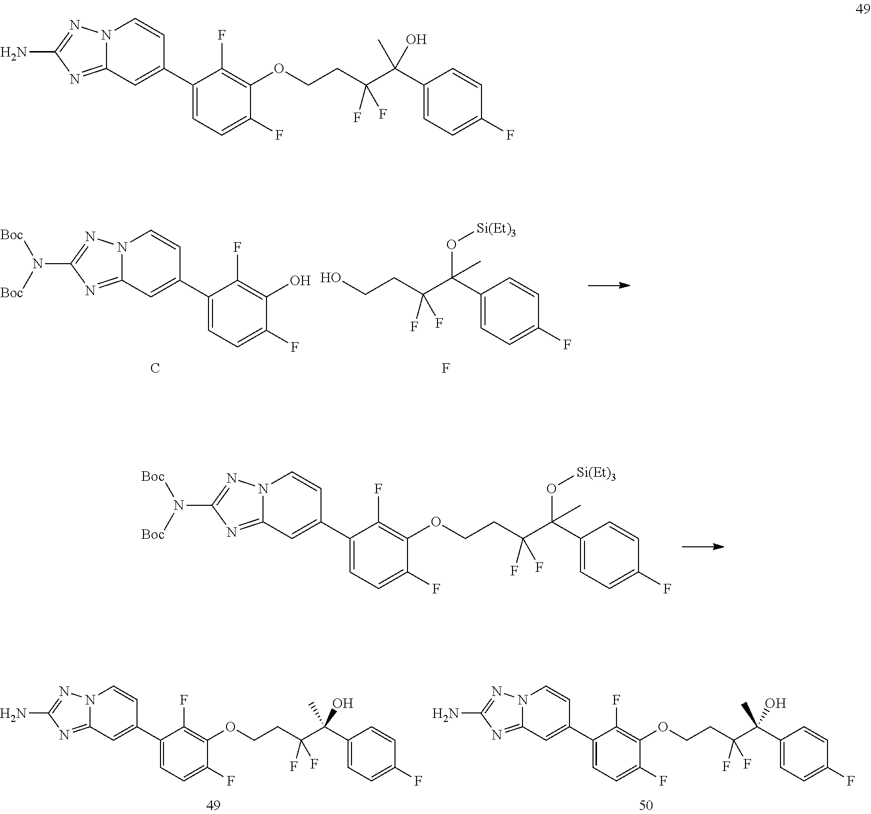

49 and 50: The two enantiomers of 5-(3-(2-amino-[1,2,4]triazolo[1,5-a]pyridin-7-yl)-2,6-difluorophenoxy)-3,3-difluoro-2-(4-fluorophenyl)pentan-2-ol were prepared in a similar fashion to 39 and 40 using C and F.

49 (4.4 mg, 3%): ¹H NMR (500 MHz, DMSO-d₆) δ 8.61 (d, J=6.7 Hz, 1H), 7.57 (br dd, J=7.8, 6.3 Hz, 2H), 7.49 (s, 1H), 7.43-7.35 (m, 1H), 7.30-7.23 (m, 1H), 7.18 (br t, J=8.9 Hz, 2H), 7.02 (br d, J=6.7 Hz, 1H), 6.08 (s, 2H), 4.27 (tdd, J=17.2, 10.1, 7.0 Hz, 2H), 2.49-2.37 (m, 1H), 2.17-2.02 (m, 1H), 1.60 (s, 3H);

¹⁹F NMR (471 MHz, DMSO-d₆) δ −107.33-−109.45 (m, 1F), −111.29 (br d, J=244.5 Hz, 1F), −115.68 (s, 1F), −127.26 (br s, 1F), −131.20 (br d, J=8.7 Hz, 1F).

MS ESI m/z 479.2 (M+H)⁺.

### Example 50: 5-(3-(2-amino-[1,2,4]triazolo[1,5-a]pyridin-7-yl)-2,6-difluorophenoxy)-3,3-difluoro-2-(4-fluorophenyl)pentan-2-ol (enantiomer 2)

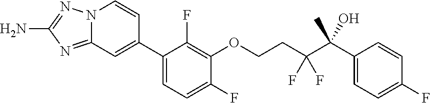 [SMILES: C[C@@](O)(c1ccc(F)cc1)C(F)(F)CCOc1c(F)ccc(-c2ccn3nc(N)nc3c2)c1F]

50 (4.3 mg, 3%): ¹H NMR (500 MHz, DMSO-d₆) δ 8.60 (d, J=7.0 Hz, 1H), 7.61-7.53 (m, 2H), 7.49 (s, 1H), 7.42-7.35 (m, 1H), 7.26 (br t, J=9.5 Hz, 1H), 7.18 (br t, J=8.9 Hz, 2H), 7.02 (br d, J=6.7 Hz, 1H), 6.07 (s, 2H), 4.35-4.20 (m, 2H), 2.50-2.37 (m, 1H), 2.16-2.01 (m, 1H), 1.60 (s, 3H);

¹⁹F NMR (471 MHz, DMSO-d₆) δ −107.12-−109.45 (m, 1F), −111.29 (br d, J=244.5 Hz, 1F), −115.66 (s, 1F), −127.21 (br d, J=8.7 Hz, 1F), −131.23 (br d, J=8.7 Hz, 1F).

MS ESI m/z 479.2 (M+H)⁺.

### Example 51: 5-(3-(2-amino-[1,2,4]triazolo[1,5-a]pyridin-7-yl)-6-chloro-2-fluorophenoxy)-3,3-difluoro-2-(4-fluorophenyl)pentan-2-ol (enantiomer 1)

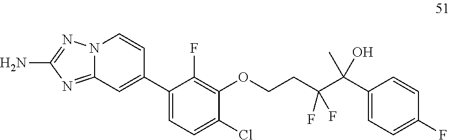 [SMILES: CC(O)(c1ccc(F)cc1)C(F)(F)CCOc1c(Cl)ccc(-c2ccn3nc(N)nc3c2)c1F]

(The 2 unassigned enantiomers are:

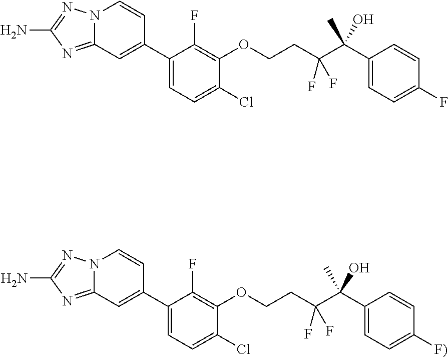

51 and 52: The two enantiomers of 5-(3-(2-amino-[1,2,4]triazolo[1,5-a]pyridin-7-yl)-6-chloro-2-fluorophenoxy)-3,3-difluoro-2-(4-fluorophenyl)pentan-2-ol were prepared in a similar fashion to 39 and 40 using D and F.

51, ¹H NMR (499 MHz, chloroform-d) δ 8.38 (d, J=7.0 Hz, 1H), 7.62-7.55 (m, 3H), 7.28 (dd, J=8.5, 1.7 Hz, 1H), 7.15 (dd, J=8.5, 7.5 Hz, 1H), 7.11 (dt, J=7.1, 1.7 Hz, 1H), 7.10-7.05 (m, 2H), 4.35-4.23 (m, 2H), 2.64-2.51 (m, 1H), 2.21-2.07 (m, 1H), 1.74 (s, 3H).

MS ESI m/z 495.1 (M+H)⁺.

### Example 52: 5-(3-(2-amino-[1,2,4]triazolo[1,5-a]pyridin-7-yl)-6-chloro-2-fluorophenoxy)-3,3-difluoro-2-(4-fluorophenyl)pentan-2-ol (enantiomer 2)

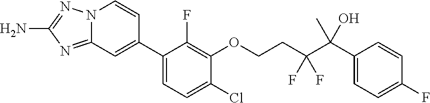 [SMILES: CC(O)(c1ccc(F)cc1)C(F)(F)CCOc1c(Cl)ccc(-c2ccn3nc(N)nc3c2)c1F]

52, ¹H NMR (499 MHz, chloroform-d) δ 8.39 (d, J=6.9 Hz, 1H), 7.64-7.55 (m, 3H), 7.28 (dd, J=8.5, 1.7 Hz, 1H), 7.18-7.11 (m, 2H), 7.10-7.05 (m, 2H), 4.36-4.23 (m, 2H), 2.65-2.51 (m, 1H), 2.21-2.07 (m, 1H), 1.75 (s, 3H).

MS ESI m/z 495.2 (M+H)⁺.

### Example 53: 4-(5-(2-amino-[1,2,4]triazolo[1,5-a]pyridin-7-yl)-2-fluorophenoxy)-2,2-difluoro-1-(4-fluorophenyl)butan-1-ol (enantiomer 1)

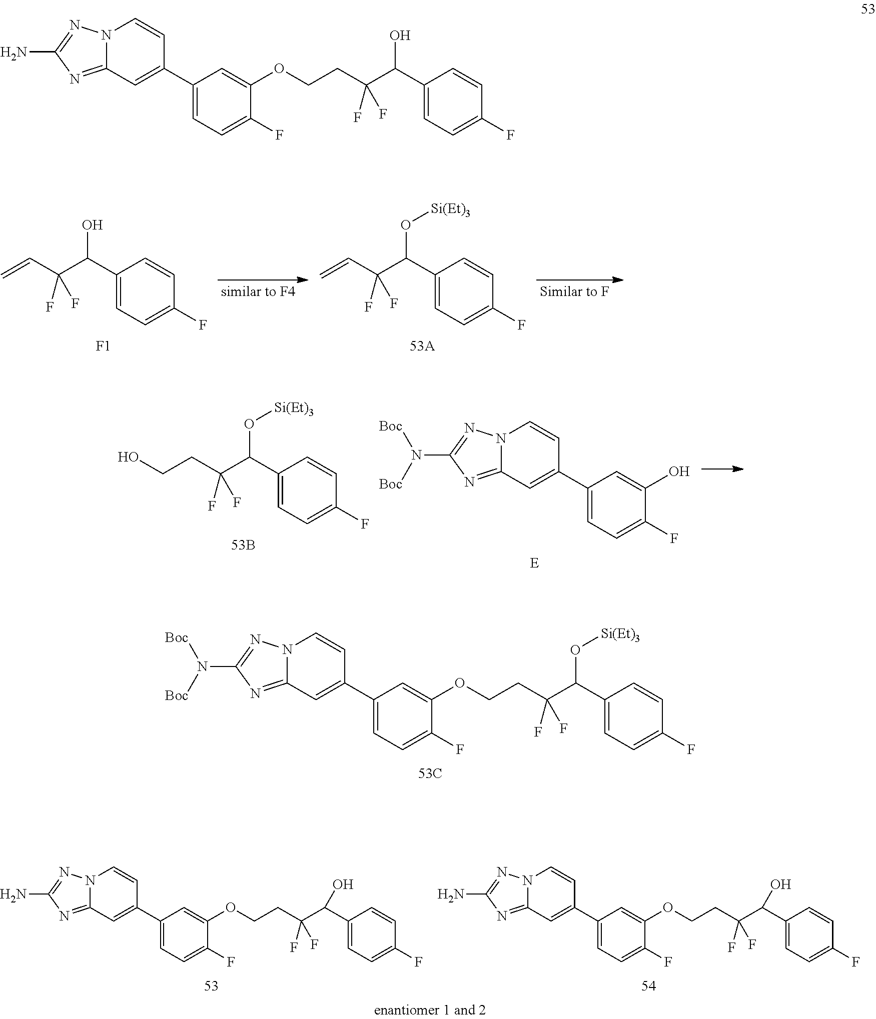

(The 2 unassigned enantiomers are:

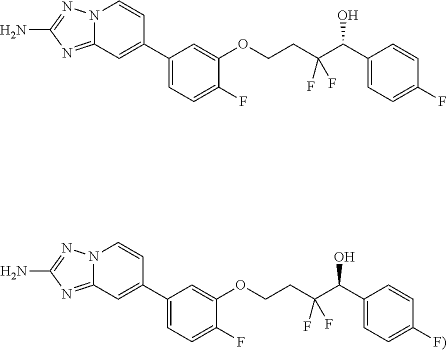

53A: ((2,2-difluoro-1-(4-fluorophenyl)but-3-en-1-yl)oxy)triethylsilane was prepared in a similar fashion to F4 starting from 2,2-difluoro-1-(4-fluorophenyl)but-3-en-1-ol.

53B: 3,3-difluoro-4-(4-fluorophenyl)-4-((triethylsilyl)oxy)butan-1-ol was prepared in a 15 similar fashion to F from 53A.

53C: tert-butyl (tert-butoxycarbonyl)(7-(3-(3,3-difluoro-4-(4-fluorophenyl)-4-((triethylsilyl)oxy)butoxy)-4-fluorophenyl)-[1,2,4]triazolo[1,5-a]pyridin-2-yl)carbamate was prepared in a similar fashion to 2A from 53B and E.

53 and 54: The two enantiomers of 4-(5-(2-amino-[1,2,4]triazolo[1,5-a]pyridin-7-yl)-2-fluorophenoxy)-2,2-difluoro-1-(4-fluorophenyl)butan-1-ol were prepared in a similar fashion to 39 and 40.

53 (5.5 mg, 22% yield): ¹H NMR (500 MHz, DMSO-d₆) δ 8.56 (d, J=7.0 Hz, 1H), 7.69 (s, 1H), 7.56 (br d, J=8.9 Hz, 1H), 7.52-7.46 (m, 2H), 7.41-7.30 (m, 2H), 7.26-7.16 (m, 3H), 6.01 (s, 2H), 4.98-4.88 (m, 1H), 4.40 (br t, J=6.6 Hz, 2H), 2.49-2.30 (m, 2H);

¹⁹F NMR (471 MHz, DMSO-d₆) δ −104.78-−106.69 (m, 1F), −109.19 (br d, J=246.2 Hz, 1F), −114.46 (s, 1F), −134.69 (s, 1F).

MS ESI m/z 447.1 (M+H)⁺.

### Example 54: 4-(5-(2-amino-[1,2,4]triazolo[1,5-a]pyridin-7-yl)-2-fluorophenoxy)-2,2-difluoro-1-(4-fluorophenyl)butan-1-ol (enantiomer 2)

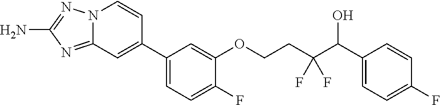 [SMILES: Nc1nc2cc(-c3ccc(F)c(OCCC(F)(F)C(O)c4ccc(F)cc4)c3)ccn2n1]

54 (5.7 mg, 23% yield): ¹H NMR (500 MHz, DMSO-d₆) δ 8.57 (d, J=7.3 Hz, 1H), 7.70 (s, 1H), 7.57 (br d, J=7.3 Hz, 1H), 7.50 (br dd, J=8.2, 5.8 Hz, 2H), 7.42-7.30 (m, 2H), 7.26-7.16 (m, 3H), 6.45 (d, J=5.2 Hz, 1H), 6.02 (s, 2H), 4.99-4.88 (m, 1H), 4.41 (br t, J=6.4 Hz, 2H), 2.48-2.28 (m, 2H);

¹⁹F NMR (471 MHz, DMSO-d₆) δ −105.64 (br d, J=248.0 Hz, 1F), −109.18 (br d, J=246.2 Hz, 1F), −114.47 (s, 1F), −134.71 (s, 1F).

MS ESI m/z 447.1 (M+H)⁺.

### Example 55: 4-(3-(2-amino-[1,2,4]triazolo[1,5-a]pyridin-7-yl)-2,6-difluorophenoxy)-2,2-difluoro-1-(4-fluorophenyl)butan-1-ol (enantiomer 1)

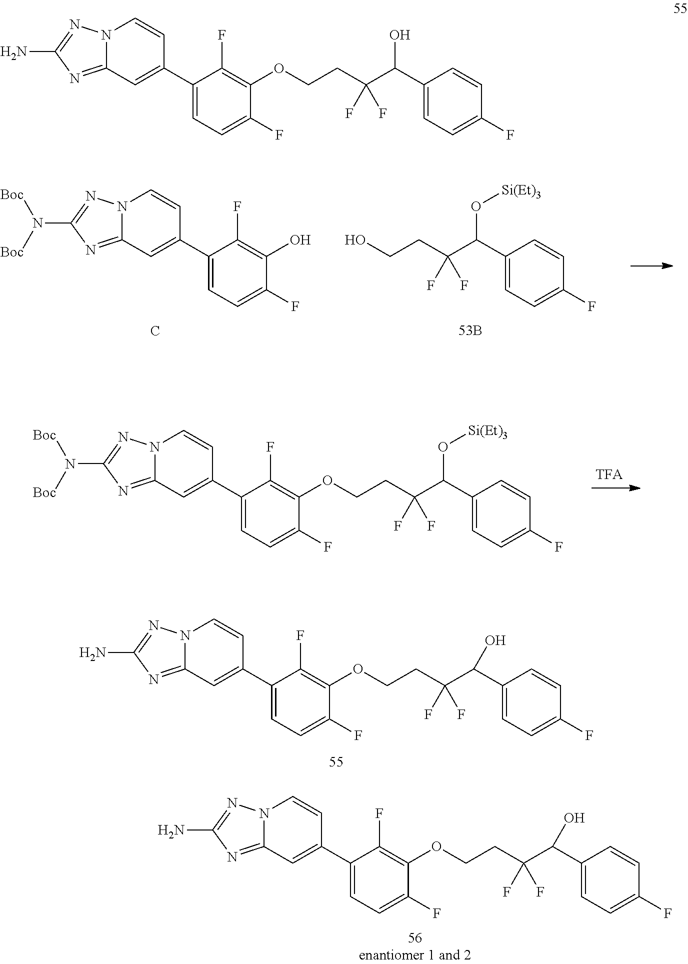

(The 2 unassigned enantiomers are:

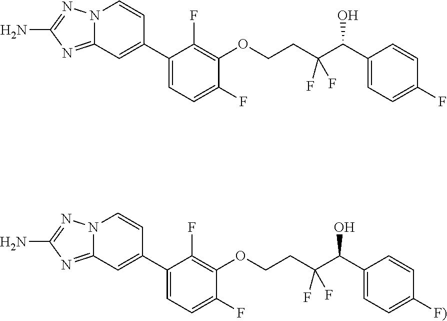

55 and 56: The two enantiomers of 4-(3-(2-amino-[1,2,4]triazolo[1,5-a]pyridin-7-yl)-2,6-difluorophenoxy)-2,2-difluoro-1-(4-fluorophenyl)butan-1-ol were prepared in a similar fashion to 39 and 40 using C and 53B as starting materials.

55 (4.5 mg, 19% yield): ¹H NMR (500 MHz, DMSO-d₆) δ 8.61 (d, J=7.0 Hz, 1H), 7.50 (s, 1H), 7.46 (br dd, J=7.5, 6.3 Hz, 2H), 7.43-7.37 (m, 1H), 7.28 (br t, J=9.0 Hz, 1H), 7.19 (br t, J=8.9 Hz, 2H), 7.04 (br d, J=7.3 Hz, 1H), 6.44 (d, J=5.5 Hz, 1H), 6.07 (s, 2H), 4.94-4.85 (m, 1H), 4.35 (br t, J=6.4 Hz, 2H), 2.49-2.28 (m, 2H);

¹⁹F NMR (471 MHz, DMSO-d₆) δ −105.39 (br d, J=246.2 Hz, 1F), −109.29 (br d, J=248.0 Hz, 1F), −114.48 (s, 1F), −127.17 (br d, J=8.7 Hz, 1F), −131.16 (br d, J=8.7 Hz, 1F).

MS ESI m/z 465.1 (M+H)⁺.

### Example 56: 4-(3-(2-amino-[1,2,4]triazolo[1,5-a]pyridin-7-yl)-2,6-difluorophenoxy)-2,2-difluoro-1-(4-fluorophenyl)butan-1-ol (enantiomer 2)

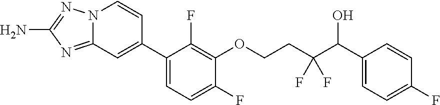 [SMILES: Nc1nc2cc(-c3ccc(F)c(OCCC(F)(F)C(O)c4ccc(F)cc4)c3F)ccn2n1]

56 (5.0 mg, 21% yield): ¹H NMR (500 MHz, DMSO-d₆) δ 8.62 (d, J=6.4 Hz, 1H), 7.51 (s, 1H), 7.47 (br dd, J=7.6, 5.8 Hz, 2H), 7.43-7.38 (m, 1H), 7.33-7.26 (m, 1H), 7.20 (br t, J=8.7 Hz, 2H), 7.08-7.02 (m, 1H), 6.08 (s, 2H), 4.96-4.86 (m, 1H), 4.36 (br t, J=6.0 Hz, 2H), 2.49-2.31 (m, 2H); ¹⁹F NMR (471 MHz, DMSO-d₆) δ −105.38 (br d, J=246.2 Hz, 1F), −109.28 (br d, J=248.0 Hz, 1F), −114.47 (s, 1F), −127.15 (br s, 1F), −131.16 (br d, J=8.7 Hz, 1F).

MS ESI m/z 465.1 (M+H)⁺.

### Example 57: 5-(5-(2-amino-[1,2,4]triazolo[1,5-a]pyridin-7-yl)-2-fluorophenoxy)-3,3-difluoro-2-(4-fluorophenyl)pentan-2-ol (enantiomer 1)

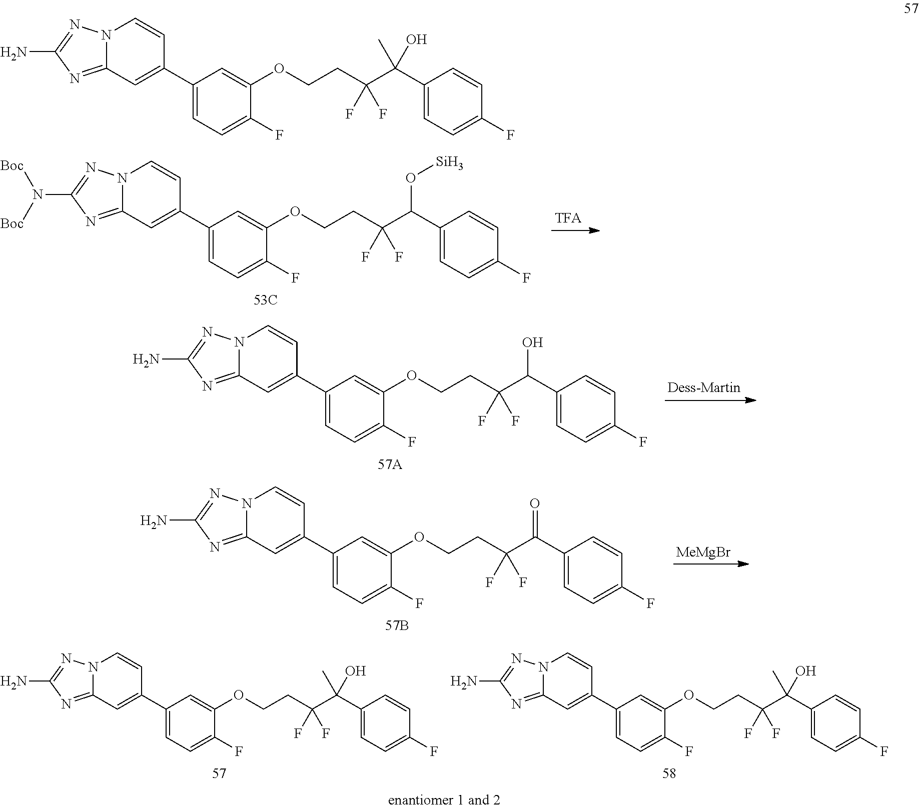

(The 2 unassigned enantiomers are:

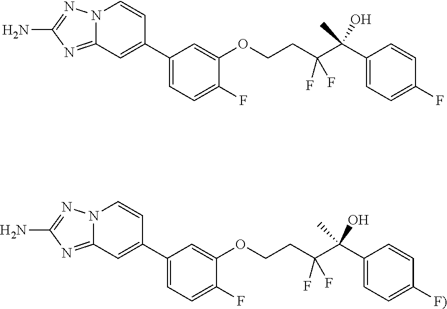

### 57A: 4-(5-(2-amino-[1,2,4]triazolo[1,5-a]pyridin-7-yl)-2-fluorophenoxy)-2,2-difluoro-1-(4-fluorophenyl)butan-1-ol

To a solution of 53C (352.6 mg, 0.463 mmol) in DCM (1.5 mL) was added TFA (1.5 mL, 19.47 mmol). The mixture was stirred at room temperature for 3 h. Volatiles were stripped off. The crude product was subject to next reaction.

MS ESI m/z 447.4 (M+H)⁺.

### 57B: 4-(5-(2-amino-[1,2,4]triazolo[1,5-a]pyridin-7-yl)-2-fluorophenoxy)-2,2-difluoro-1-(4-fluorophenyl)butan-1-one

A solution of 4-(5-(2-amino-[1,2,4]triazolo[1,5-a]pyridin-7-yl)-2-fluorophenoxy)-2,2-difluoro-1-(4-fluorophenyl)butan-1-ol (182 mg, 0.408 mmol) and dess-martinperiodinane (259 mg, 0.612 mmol) in a 50 mL flask in CH₂Cl₂ (3 mL) was stirred at rt for 3 h. More DMP (0.5 equiv) was added and the reaction continued for another 1 h. The reaction mixture was quenched with sodium thiofulfite and diluted with EtOAc. The layers were separated. The organic layer was washed with brine, dried with Na₂SO₄, and concentrated to the crude product (196 mg, 100% mass recovery) as an off white solid, which was used as was.

MS ESI m/z 445.4 (M+H)⁺.

### 57 and 58: 5-(5-(2-amino-[1,2,4]triazolo[1,5-a]pyridin-7-yl)-2-fluorophenoxy)-3,3-difluoro-2-(4-fluorophenyl)pentan-2-ol

To a solution of 4-(5-(2-amino-[1,2,4]triazolo[1,5-a]pyridin-7-yl)-2-fluorophenoxy)-2,2-difluoro-1-(4-fluorophenyl)butan-1-one (181 mg, 0.408 mmol) in THF (3 mL) in a 100 mL flask was added methylmagnesium bromide (0.544 mL, 1.632 mmol) dropwise. The mixture was stirred at rt for 2 h. The reaction was quenched with saturated NH₄C₁ solution and diluted with EtOAc. The layers were separated. The organic layer was washed with saturated NaHCO₃, brine, dried with Na₂SO₄, and concentrated. The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 28% B, 28-68% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The material was further purified using SFC-chiral chromatography. Instrument: Waters 100 Prep SFC; Column: Chiral AD, 30×250 mm. 5 micron; Mobile Phase: 60% CO2/40% MeOH-ACN 50-50; Flow Conditions: 100 mL/min; Detector Wavelength: 220 nm; Injection Details: 1000 μL 14.5 mg dissolved in 3 mL MeOH.

The first eluting isomer 57 (3.9 mg, 2%): ¹H NMR (500 MHz, DMSO-d₆) δ 8.57 (d, J=6.7 Hz, 1H), 7.68 (s, 2H), 7.62-7.56 (m, 2H), 7.50 (br d, J=7.0 Hz, 1H), 7.39-7.35 (m, 1H), 7.34-7.28 (m, 1H), 7.24-7.14 (m, 3H), 6.02 (s, 2H), 4.39-4.27 (m, 2H), 2.50-2.37 (m, 1H), 2.18-2.01 (m, 1H), 1.62 (s, 3H).

¹⁹F NMR (471 MHz, DMSO-d₆) δ −108.51 (br d, J=244.5 Hz, 1F), −111.37 (br d, J=244.5 Hz, 1F), −115.71 (s, 1F), −134.75 (s, 1F).

MS ESI m/z 461.1 (M+H)⁺.

### Example 58: 5-(5-(2-amino-[1,2,4]triazolo[1,5-a]pyridin-7-yl)-2-fluorophenoxy)-3,3-difluoro-2-(4-fluorophenyl)pentan-2-ol (enantiomer 2)

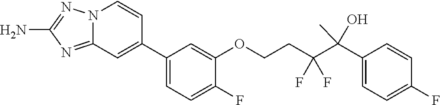 [SMILES: CC(O)(c1ccc(F)cc1)C(F)(F)CCOc1cc(-c2ccn3nc(N)nc3c2)ccc1F]

The second eluting isomer 58 (3.6 mg, 2%): ¹H NMR (500 MHz, DMSO-d₆) δ 8.58 (d, J=6.7 Hz, 1H), 7.69 (s, 1H), 7.63-7.57 (m, 2H), 7.51 (br d, J=6.7 Hz, 1H), 7.37 (br s, 1H), 7.34-7.27 (m, 1H), 7.25-7.14 (m, 3H), 6.03 (s, 2H), 4.40-4.28 (m, 2H), 2.50-2.41 (m, 1H), 2.19-2.02 (m, 1H), 1.63 (s, 3H).

¹⁹F NMR (471 MHz, DMSO-d₆) δ −107.54-−109.24 (m, 1F), −111.37 (br d, J=244.5 Hz, 1F), −115.73 (s, 1F), −134.79 (s, 1F).

MS ESI m/z 461.1 (M+H)⁺.

### Example 59: 4-(3-(2-amino-[1,2,4]triazolo[1,5-a]pyridin-7-yl)-6-chloro-2-fluorophenoxy)-2,2-difluoro-1-(4-fluorophenyl)butan-1-ol (enantiomer 1)

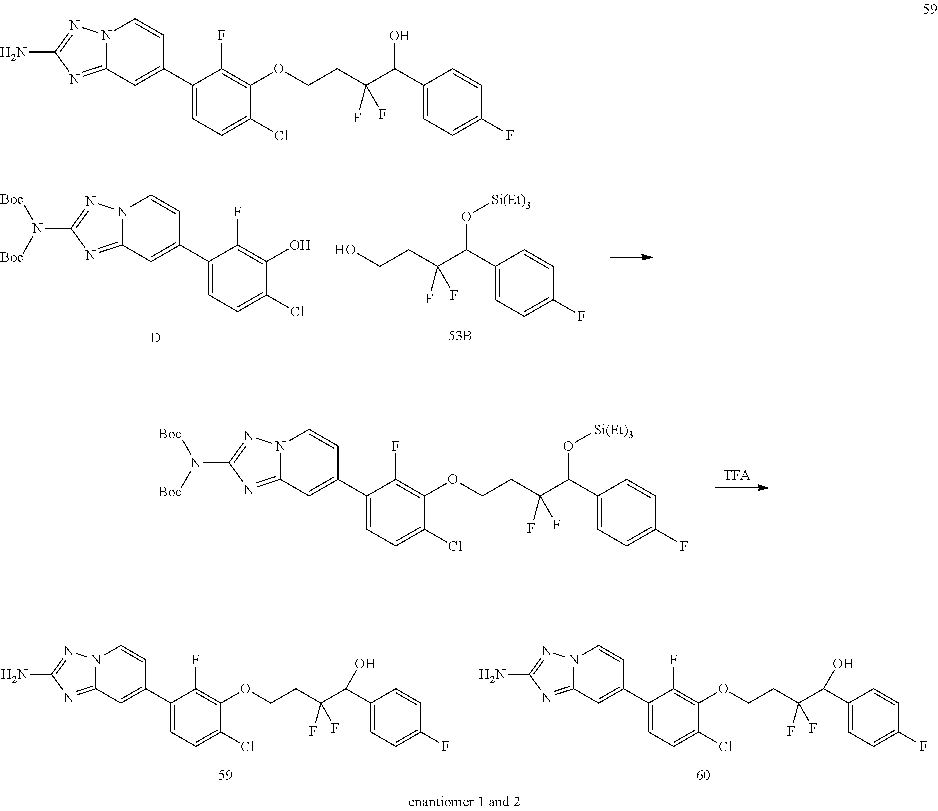

(The 2 unassigned enantiomers:

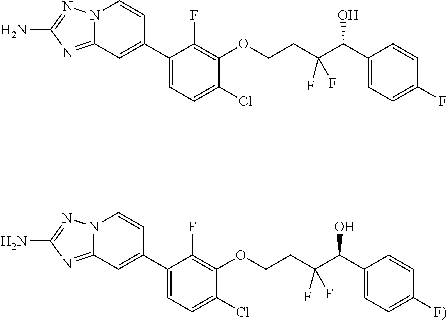

59 and 60: The two enantiomers of 4-(3-(2-amino-[1,2,4]triazolo[1,5-a]pyridin-7-yl)-6-chloro-2-fluorophenoxy)-2,2-difluoro-1-(4-fluorophenyl)butan-1-ol were prepared in a similar fashion to 39 and 40 using D and 53B as starting materials.

59 (2.8 mg, 21%): ¹H NMR (500 MHz, DMSO-d₆) δ 8.62 (d, J=7.0 Hz, 1H), 7.54 (s, 1H), 7.51-7.39 (m, 4H), 7.20 (t, J=8.9 Hz, 2H), 7.06 (br d, J=7.0 Hz, 1H), 6.09 (s, 2H), 4.97-4.87 (m, 1H), 4.38-4.26 (m, 2H), 2.50-2.31 (m, 2H).

¹⁹F NMR (471 MHz, DMSO-d₆) δ −105.30 (br d, J=248.0 Hz, 1F), −109.13 (br d, J=248.0 Hz, 1F), −114.48 (s, 1F), −130.90 (s, 1F).

MS ESI m/z 481.1 (M+H)⁺.

### Example 60: 4-(3-(2-amino-[1,2,4]triazolo[1,5-a]pyridin-7-yl)-6-chloro-2-fluorophenoxy)-2,2-difluoro-1-(4-fluorophenyl)butan-1-ol (enantiomer 2)

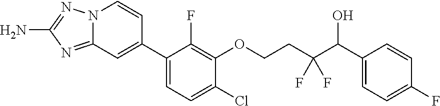 [SMILES: Nc1nc2cc(-c3ccc(Cl)c(OCCC(F)(F)C(O)c4ccc(F)cc4)c3F)ccn2n1]

60 (2.1 mg, 16%): ¹H NMR (500 MHz, DMSO-d₆) δ 8.63 (d, J=7.0 Hz, 1H), 7.55 (s, 1H), 7.51-7.39 (m, 4H), 7.20 (br t, J=8.9 Hz, 2H), 7.07 (br d, J=6.7 Hz, 1H), 6.10 (s, 2H), 4.97-4.88 (m, 1H), 4.37-4.27 (m, 2H), 2.50-2.32 (m, 2H).

¹⁹F NMR (471 MHz, DMSO-d₆) δ −105.29 (br d, J=248.0 Hz, 1F), −109.13 (br d, J=248.0 Hz, 1F), −114.48 (s, 1F), −130.88 (s, 1F).

MS ESI m/z 481.1 (M+H)⁺.

### Example 61: 7-(3-(4-amino-3,3-difluoro-4-(4-fluorophenyl)butoxy)-2-fluorophenyl)-[1,2,4]triazolo[1,5-a]pyridin-2-amine (enantiomer 1)

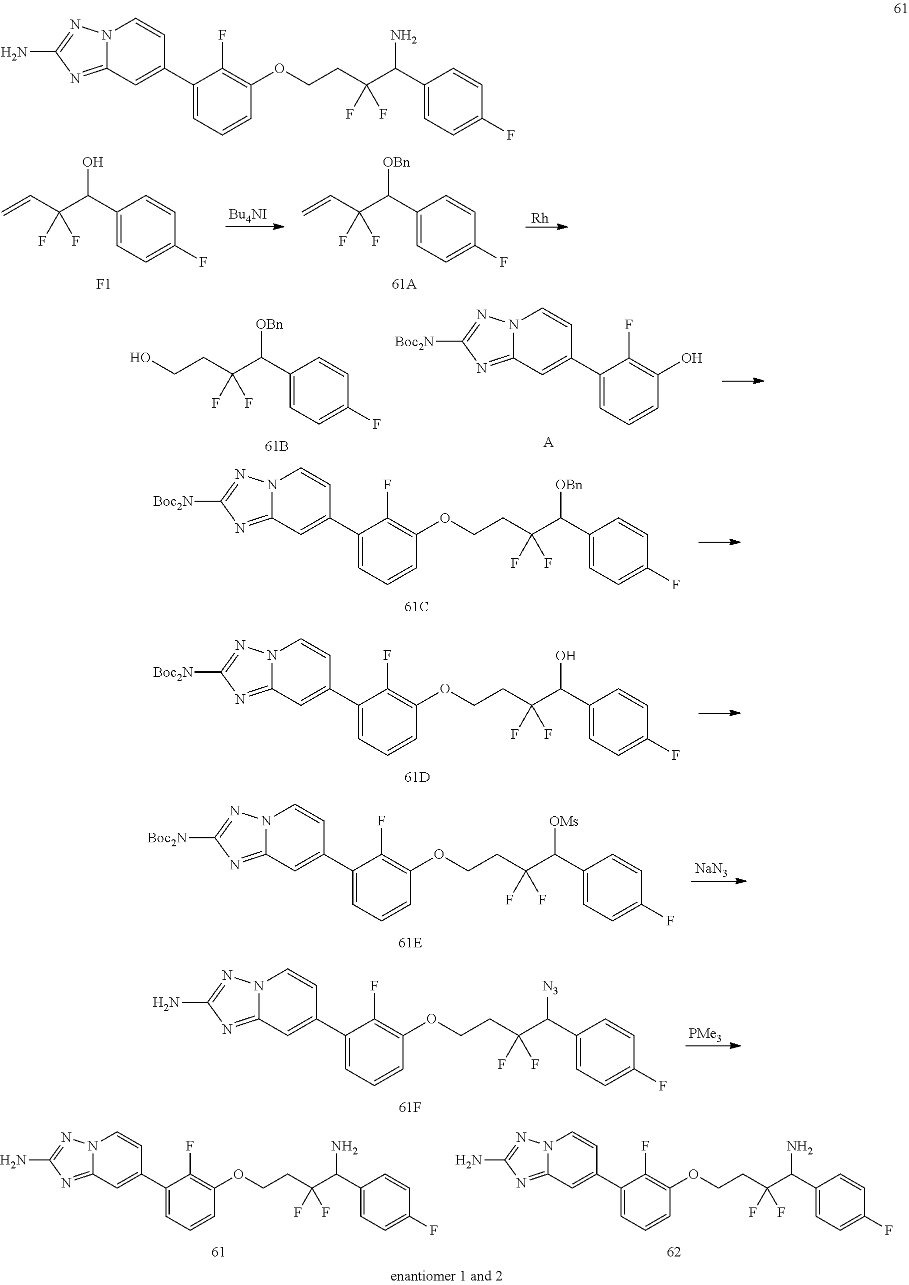

(The 2 unassigned enantiomers are:

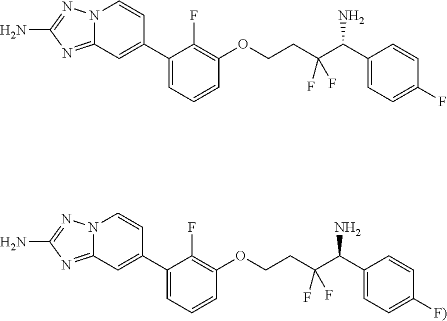

### 61A: 1-(1-(benzyloxy)-2,2-difluorobut-3-en-1-yl)-4-fluorobenzene

To a 250 ml flask under nitrogen charged with 2,2-difluoro-1-(4-fluorophenyl)but-3-en-1-ol (455 mg, 2.251 mmol) and tetrabutylammonium iodide (166 mg, 0.450 mmol) was added THF (7 mL). NaH (180 mg, 4.50 mmol) was added slowly. After stirring at rt for 10 min, benzyl bromide (0.321 mL, 2.70 mmol) was added. The mixture was stirred at rt for 18 h. TLC (4/1 Hexane/EtOAc) showed good conversion to a less polar spot. The reaction was carefully quenched with drops of water. The mixture was diluted with EtOAc and water. The layers were separated. The organic layer was washed with brine, dried with Na₂SO₄, and concentrated. The residue was purified by FCC (40 g silica gel column) up to 25% EtOAc/hexane to afford the desired product 1-(1-(benzyloxy)-2,2-difluorobut-3-en-1-yl)-4-fluorobenzene (561 mg, 85%) as a colorless oil.

¹H NMR (499 MHz, chloroform-d) δ 7.47-7.30 (m, 7H), 7.15-7.05 (m, 2H), 6.00 (ddt, J=17.4, 12.9, 11.0 Hz, 1H), 5.57 (dddd, J=17.4, 2.9, 2.2, 0.9 Hz, 1H), 5.52-5.46 (m, 1H), 4.65 (d, J=11.9 Hz, 1H), 4.62-4.53 (m, 1H), 4.44 (d, J=11.9 Hz, 1H).

¹⁹F NMR (470 MHz, chloroform-d) δ −103.97 (d, J=250.0 Hz), −109.71 (d, J=248.0 Hz), −113.17.

### 61B: 4-(benzyloxy)-3,3-difluoro-4-(4-fluorophenyl)butan-1-ol

To a one-dram vial under nitrogen charged with tris(triphenylphosphine)rhodium(I) chloride (12.66 mg, 0.014 mmol) and tris(triphenylphosphine)rhodium(I) chloride (105 mg, 0.821 mmol) was added THF (1 mL). After 5 min stirring, 1-(1-(benzyloxy)-2,2-difluorobut-3-en-1-yl)-4-fluorobenzene (200 mg, 0.684 mmol) was added. The mixture was stirred at rt for 20 h. TLC (9/1 hexane/EtOAc) showed a spot slightly more polar than the SM. More tris(triphenylphosphine)rhodium(I) chloride (12.66 mg, 0.014 mmol) and 4,4,5,5-tetramethyl-1,3,2-dioxaborolane (105 mg, 0.821 mmol) were added and the reaction continued for another 20 h. TLC showed better conversion. The boronate ester was then oxidized by adding 2 mL 1 N NaOH and hydrogen peroxide (0.6 mL, 5.87 mmol) dropwise. The mixture was diluted with EtOAc. TLC showed the desired product.

The layers were separated. The organic layer was washed with brine, dried with Na₂SO₄, and concentrated. The residue was purified by FCC (12 g silica) up to 50% EtOAc/hexane to afford the desired product (76.6 mg, 36%) as a colorless oil. 1H NMR (499 MHz, Chloroform-d) δ 7.52-7.30 (m, 7H), 7.19-7.03 (m, 2H), 4.64 (d, J=11.7 Hz, 1H), 4.58 (dd, J=13.2, 7.7 Hz, 1H), 4.42 (d, J=11.7 Hz, 1H), 3.87 (dh, J=11.9, 6.1 Hz, 2H), 2.40-2.24 (m, 1H), 2.23-2.07 (m, 1H), 1.91 (t, J=6.0 Hz, 1H).

¹⁹F NMR (470 MHz, Chloroform-d) δ −104.39 (d, J=247.6 Hz), −106.55 (d, J=256.1 Hz), −113.00.

### 61C: tert-butyl (7-(3-(4-(benzyloxy)-3,3-difluoro-4-(4-fluorophenyl)butoxy)-2-fluorophenyl)-[1,2,4]triazolo[1,5-a]pyridin-2-yl)(tert-butoxycarbonyl)carbamate

To a mixture of 61B (145 mg, 0.326 mmol), Ph₃P (117 mg, 0.444 mmol), and 4-(benzyloxy)-3,3-difluoro-4-(4-fluorophenyl)butan-1-ol (91.9 mg, 0.296 mmol) in THF (2 mL) was added DIAD (0.075 mL, 0.385 mmol). The mixture was stirred at room temperature for 18 h. LC-MS indicated the desired product (M+H=737.9). TLC (2/1 hexane/EtOAc) showed a blue spot slightly less polar than the phenol. Volatiles were stripped off. The residue was directly purified by FCC (24 g silica gel) up to 50% EtOAc/hexane to afford the desired product tert-butyl (7-(3-(4-(benzyloxy)-3,3-difluoro-4-(4-fluorophenyl)butoxy)-2-fluorophenyl)-[1,2,4]triazolo[1,5-a]pyridin-2-yl)(tert-butoxycarbonyl)carbamate as a crude colorless oil (265 mg, >100%).

¹H NMR (499 MHz, chloroform-d) δ 8.58 (dd, J=7.1, 0.9 Hz, 1H), 7.87 (dt, J=1.8, 0.8 Hz, 1H), 7.47 (dd, J=8.5, 5.4 Hz, 2H), 7.36-7.25 (m, 6H), 7.19 (td, J=8.0, 1.3 Hz, 1H), 7.11 (dddd, J=12.8, 7.9, 6.5, 1.8 Hz, 3H), 7.03 (td, J=8.0, 1.6 Hz, 1H), 4.67 (dd, J=13.9, 7.6 Hz, 1H), 4.62 (d, J=11.6 Hz, 1H), 4.44 (d, J=11.6 Hz, 1H), 4.35-4.25 (m, 2H), 2.67 (dtt, J=21.4, 14.4, 7.1 Hz, 1H), 2.54-2.39 (m, 1H), 1.50 (s, 18H).

¹⁹F NMR (470 MHz, chloroform-d) δ −105.45 (d, J=254.4 Hz), −107.96 (d, J=254.5 Hz), −112.94, −138.50.

MS ESI m/z 737.9 (M+H)⁺.

### 61D: tert-butyl (tert-butoxycarbonyl)(7-(3-(3,3-difluoro-4-(4-fluorophenyl)-4-hydroxybutoxy)-2-fluorophenyl)-[1,2,4]triazolo[1,5-a]pyridin-2-yl)carbamate

To a mixture of 61C (0.218 g, 0.296 mmol) in EtOAc (1 mL) and MeOH (1 mL) was added Pd—C (0.063 g, 0.059 mmol). The mixture was stirred at room temperature under a hydrogen balloon for 4 h. LC-MS indicated 2/3 conversion to the desired product (M+H=647.3). The reaction continued overnight for 18 h. LCMS showed complete conversion to the desired product. TLC (1/1 hexane/EtOAc) showed a blue spot (Rf~0.4). The reaction mixture was filtered. Volatiles were stripped off. The residue tert-butyl (tert-butoxycarbonyl)(7-(3-(3,3-difluoro-4-(4-fluorophenyl)-4-hydroxybutoxy)-2-fluorophenyl)-[1,2,4]triazolo[1,5-a]pyridin-2-yl)carbamate, a tan oil (220 mg, >100% mass recovery) was directly for the next step.

MS ESI m/z 647.6 (M+H)⁺.

### 61E: 4-(3-(2-(bis(tert-butoxycarbonyl)amino)-[1,2,4]triazolo[1,5-a]pyridin-7-yl)-2-fluorophenoxy)-2,2-difluoro-1-(4-fluorophenyl)butyl methanesulfonate

To a solution of 61D (83.3 mg, 0.107 mmol) and Et₃N (0.019 mL, 0.139 mmol) in DCM (0.5 mL) was added Ms-Cl (10.83 μl, 0.139 mmol). The mixture was stirred at room temperature for 4 h. Another drop of MsCl and Et₃N were added and the reaction continued for another hour. The reaction mixture was diluted with water and EtOAc. The layers were separated. The organic layer was washed with saturated NaHCO₃ solution, dried with Na₂SO₄, and concentrated to a colorless foam (100 mg, >100% mass recovery), which was used in the next step.

MS ESI m/z 725.2 (M+H)⁺.

### 61F: 7-(3-(4-azido-3,3-difluoro-4-(4-fluorophenyl)butoxy)-2-fluorophenyl)-[1,2,4]triazolo[1,5-a]pyridin-2-amine

To a solution of 61E (78 mg, 0.107 mmol) and tetrabutylammonium bromide (3.45 mg, 10.70 μmol) in DMF (0.5 mL) was added sodium azide (13.91 mg, 0.214 mmol). The mixture was stirred at 65° C. for 18 h. LC-MS indicated little conversion to the desired product (M+H=647.6). Another 2 equiv. of NaN₃ was added and the reaction continued for another day. LCMS showed a little better conversion, as well as some Boc-deprotected product (M-100). Another 5 equiv NaN₃ was added and the temperature was raised to 85° C. After 24 h, LCMS showed better conversion but with 6 peaks representing SM and product with mono-Boc deprotected (M-100) and bis-Boc deprotected (M-200). Another equiv of NaN₃ (35 mg) was added and the reaction continued for 3 more days. LCMS showed mainly the desired product. The reaction mixture was diluted with water and EtOAc. The layers were separated. The aqueous layer was extracted with EtOAc. The combined organic layers were washed with water, brine, dried with Na₂SO₄, and concentrated to the crude product 7-(3-(4-azido-3,3-difluoro-4-(4-fluorophenyl)butoxy)-2-fluorophenyl)-[1,2,4]triazolo[1,5-a]pyridin-2-amine (68 mg, >100% mass recovery) as a light tan oil, which was used in the next step.

MS ESI m/z 472.4 (M+H)⁺.

61 and 62: 7-(3-(4-amino-3,3-difluoro-4-(4-fluorophenyl)butoxy)-2-fluorophenyl)-[1,2,4]triazolo[1,5-a]pyridin-2-amine:

To a solution of 7-(3-(4-azido-3,3-difluoro-4-(4-fluorophenyl)butoxy)-2-fluorophenyl)-[1,2,4]triazolo[1,5-a]pyridin-2-amine (50.4 mg, 0.107 mmol) in THF (0.5 mL) was added trimethylphosphine solution, 1.0 M in THF (0.321 mL, 0.321 mmol). The mixture was stirred at rt for 4 h. H₂O (7.71 μl, 0.428 mmol) (one drop) was added and the mixture was stirred at rt for another 16 h. Volatiles were stripped off. The residue was dissolved in MeOH and submitted to chiral separation in a similar fashion to 16 and 17.

61 (3.4 mg, 7%): ¹H NMR (500 MHz, DMSO-d₆) δ 8.59 (d, J=7.0 Hz, 1H), 7.54-7.45 (m, 3H), 7.25 (br d, J=6.1 Hz, 2H), 7.21-7.13 (m, 3H), 7.04 (br d, J=7.0 Hz, 1H), 6.05 (s, 2H), 4.33-4.22 (m, 3H), 2.49-2.30 (m, 2H).

¹⁹F NMR (471 MHz, DMSO-d₆) δ −104.00 (br dd, J=246.2, 3.5 Hz, 1F), −106.27-−108.39 (m, 1F), −115.05 (br s, 1F), −139.75 (s, 1F).

MS ESI m/z 446.2 (M+H)⁺.

### Example 62: 7-(3-(4-amino-3,3-difluoro-4-(4-fluorophenyl)butoxy)-2-fluorophenyl)-[1,2,4]triazolo[1,5-a]pyridin-2-amine (enantiomer 2)

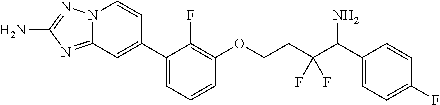 [SMILES: Nc1nc2cc(-c3cccc(OCCC(F)(F)C(N)c4ccc(F)cc4)c3F)ccn2n1]

62 (3.4 mg, 7%): ¹H NMR (500 MHz, DMSO-d₆) δ 8.60 (d, J=7.0 Hz, 1H), 7.53-7.47 (m, 3H), 7.31-7.22 (m, 2H), 7.21-7.14 (m, 3H), 7.04 (br d, J=7.0 Hz, 1H), 6.06 (s, 2H), 4.35-4.23 (m, 3H), 2.49-2.31 (m, 2H);

¹⁹F NMR (471 MHz, DMSO-d₆) δ −103.91 (br d, J=244.5 Hz, 1F), −107.32 (br d, J=244.5 Hz, 1F), −115.12 (s, 1F), −139.72 (s, 1F).

MS ESI m/z 446.2 (M+H)⁺.

### Example 63: 5-(3-(2-amino-[1,2,4]triazolo[1,5-a]pyridin-7-yl)-2-fluoro-6-methylphenoxy)-3,3-difluoro-2-(4-fluorophenyl)pentan-2-ol (enantiomer 1)

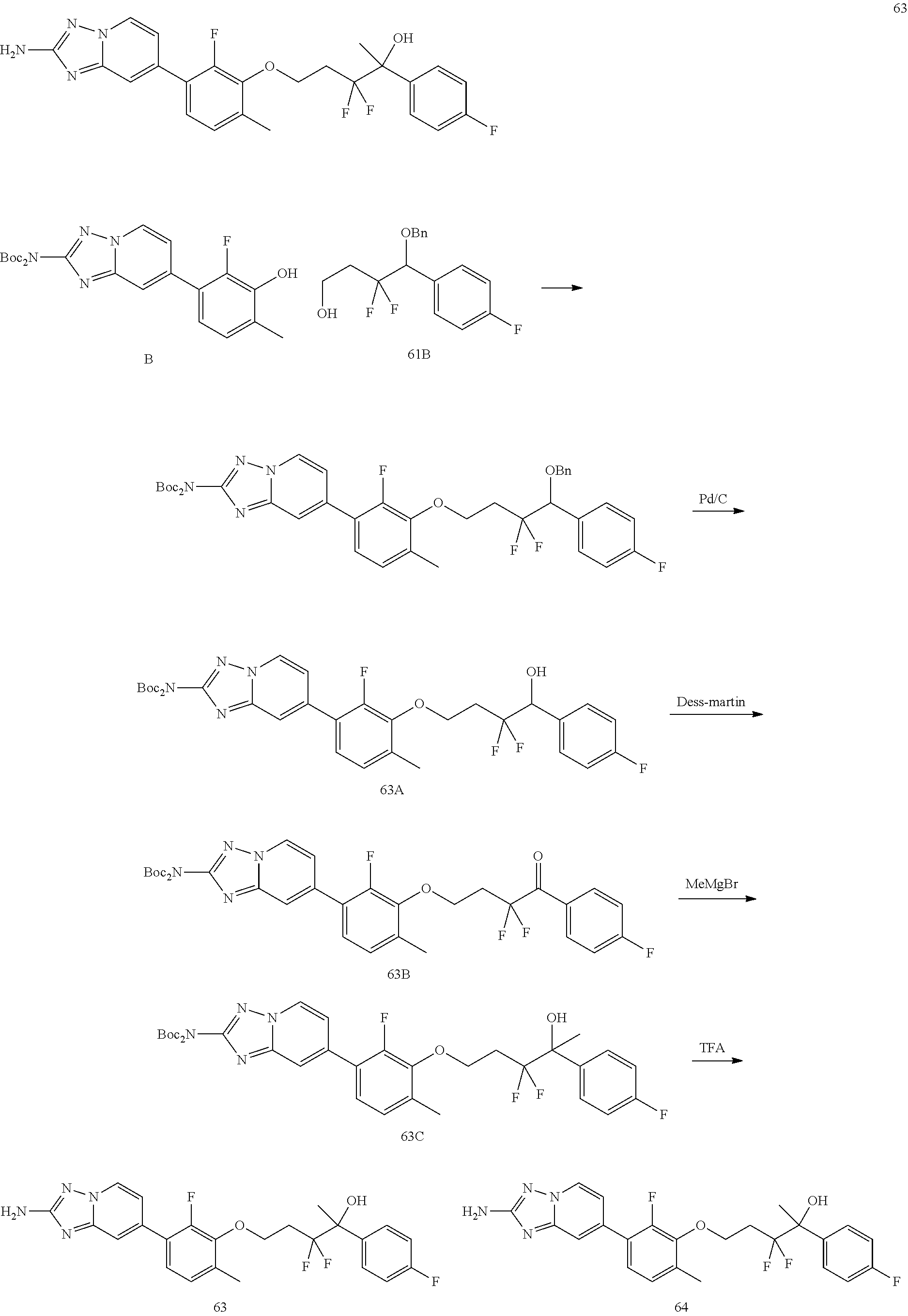

(The 2 unassigned enantiomers are:

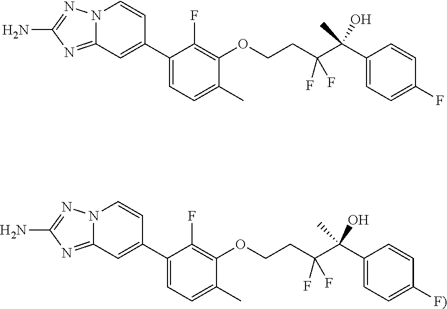

63A: tert-butyl (tert-butoxycarbonyl)(7-(3-(3,3-difluoro-4-(4-fluorophenyl)-4-hydroxybutoxy)-2-fluoro-4-methylphenyl)-[1,2,4]triazolo[1,5-a]pyridin-2-yl)carbamate was prepared in a similar fashion to 61D starting from B and 61B.

### 63B: tert-butyl (tert-butoxycarbonyl)(7-(3-(3,3-difluoro-4-(4-fluorophenyl)-4-oxobutoxy)-2-fluoro-4-methylphenyl)-[1,2,4]triazolo[1,5-a]pyridin-2-yl)carbamate

To a solution of 63A (365 mg, 0.552 mmol) in anhydrous DCM (4 mL) in a 50 mL flask was added dess-martin periodinane (351 mg, 0.829 mmol). The mixture was stirred at rt for 3 h. The reaction was quenched with aqueous sodium thiosulfite solution and diluted with EtOAc. The layers were separated. The organic layer was washed with saturated NaHCO₃ solution, water, brine, dried with Na₂SO₄, and concentrated to the crude product (394 mg, 100% assume 92% purity with 2-iodobenzoic acid as impurity) as a yellow solid. The material was used directly in the next step.

MS ESI m/z 659.6 (M+H)⁺.

### 63C: tert-butyl (7-(3-((3,3-difluoro-4-(4-fluorophenyl)-4-hydroxypentyl)oxy)-2-fluoro-4-methylphenyl)-[1,2,4]triazolo[1,5-a]pyridin-2-yl)carbamate

To a solution of 63B (227 mg, 0.345 mmol) in THF (3 mL) in a 50 mL flask was added methylmagnesium bromide (0.345 mL, 1.034 mmol). The mixture was stirred at rt for 2 h. LC-MS indicated good conversion to the desired mono-Boc product. The reaction was quenched with saturated NH₄C₁ solution and diluted with EtOAc. The layers were separated. The organic layer was washed with saturated NaHCO₃, brine, dried with Na₂SO₄, and concentrated to a tan oil (240 mg, >100% mass recovery). The residue was directly deprotected in next step.

MS ESI m/z 575.5 (M+H)⁺.

63 and 64: The two enantiomers of 5-(3-(2-amino-[1,2,4]triazolo[1,5-a]pyridin-7-yl)-2-fluoro-6-methylphenoxy)-3,3-difluoro-2-(4-fluorophenyl)pentan-2-ol were prepared in a similar fashion to 2 and 3 from 63C.

63: (24.0 mg, 24%): ¹H NMR (500 MHz, DMSO-d₆) δ 8.58 (d, J=6.9 Hz, 1H), 7.61-7.54 (m, 2H), 7.46 (s, 1H), 7.25 (br t, J=7.6 Hz, 1H), 7.18 (t, J=8.7 Hz, 2H), 7.12 (d, J=8.0 Hz, 1H), 7.01 (br d, J=6.6 Hz, 1H), 6.19-6.14 (m, 1H), 6.05 (s, 2H), 4.21-4.06 (m, 2H), 2.49-2.37 (m, 1H), 2.22 (s, 3H), 2.16-2.01 (m, 1H), 1.61 (s, 3H); ¹⁹F NMR (471 MHz, DMSO-d₆) δ −108.33 (br d, J=244.5 Hz, 1F), −111.27 (br d, J=242.8 Hz, 1F), −115.73 (br s, 1F), −135.64 (br s, 1F).

MS ESI m/z 475.2 (M+H)⁺.

### Example 64: 5-(3-(2-amino-[1,2,4]triazolo[1,5-a]pyridin-7-yl)-2-fluoro-6-methylphenoxy)-3,3-difluoro-2-(4-fluorophenyl)pentan-2-ol (enantiomer 2)

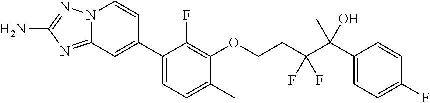 [SMILES: Cc1ccc(-c2ccn3nc(N)nc3c2)c(F)c1OCCC(F)(F)C(C)(O)c1ccc(F)cc1]

64: (23.0 mg, 23%): ¹H NMR (500 MHz, DMSO-d₆) δ 8.58 (d, J=7.0 Hz, 1H), 7.61-7.54 (m, 2H), 7.46 (s, 1H), 7.27-7.22 (m, 1H), 7.18 (t, J=8.9 Hz, 2H), 7.12 (d, J=7.9 Hz, 1H), 7.01 (br d, J=6.9 Hz, 1H), 6.16 (d, J=4.5 Hz, 1H), 6.05 (s, 2H), 4.20-4.06 (m, 2H), 2.49-2.35 (m, 1H), 2.22 (s, 3H), 2.17-2.00 (m, 1H), 1.61 (s, 3H); ¹⁹F NMR (471 MHz, DMSO-d₆) δ −108.32 (br d, J=242.8 Hz, 1F), −110.30-−112.42 (m, 1F), −115.73 (br s, 1F), −135.64 (br s, 1F).

MS ESI m/z 475.2 (M+H)⁺.

### Example 65: 7-(3-(3,3-difluoro-3-(4-fluorophenoxy)propoxy)-2-fluorophenyl)-[1,2,4]triazolo[1,5-a]pyridin-2-amine

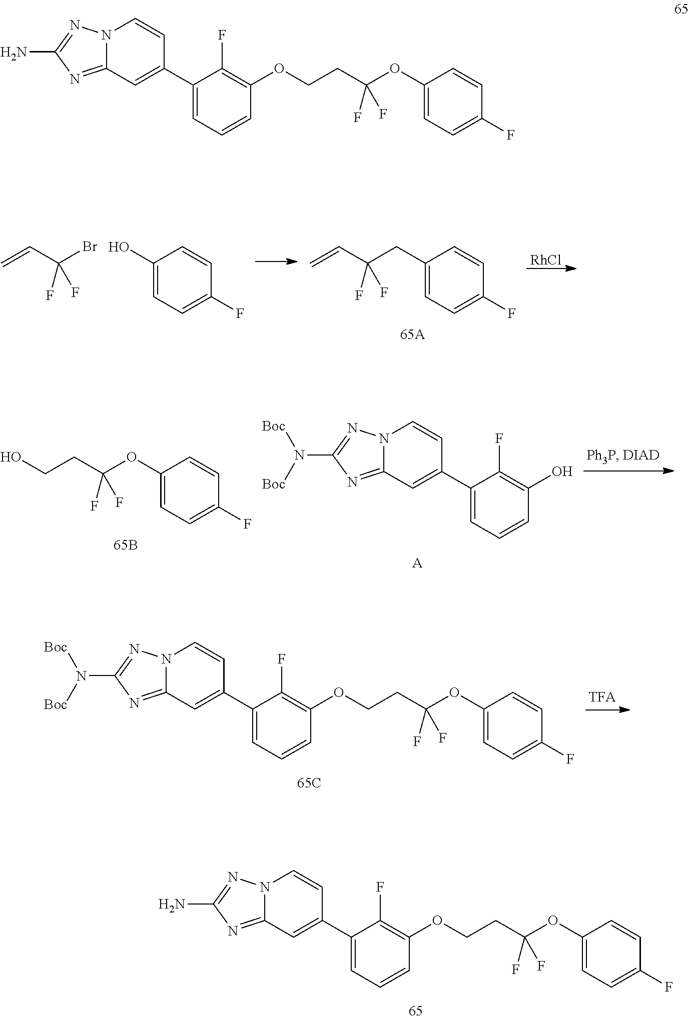

### 65A:1-((1,1-difluoroallyl)oxy)-4-fluorobenzene

To a solution of 4-fluorophenol (336 mg, 3 mmol) in anhydrous THF (6 mL) in a 50 mL pressure vial was slowly added NaH (126 mg, 3.15 mmol). PdOAc₂ (6.74 mg, 0.030 mmol) and Ph₃P (32.3 mg, 0.123 mmol) was added followed by addition of 3-bromo-3,3-difluoroprop-1-ene (471 mg, 3.00 mmol) in 2 mL THF. The mixture was stirred at 40° C. for 1 h. The reaction was quenched with phosphate buffer (pH 7) and diluted with EtOAc.

TLC (4/1 hexane/EtOAc) showed a less polar major spot (Rf~0.8). The layers were separated. The organic layer was washed with brine, dried with Na₂SO₄, and concentrated. The residue was purified by FCC up to 20% EtOAc/hexane to afford the desired product 1-((1,1-difluoroallyl)oxy)-4-fluorobenzene (452 mg, 80%) as a colorless oil.

¹H NMR (499 MHz, chloroform-d) δ 7.24-7.16 (m, 2H), 7.10-7.01 (m, 2H), 6.14-6.01 (m, 1H), 5.99-5.87 (m, 1H), 5.62 (d, J=10.7 Hz, 1H); ¹⁹F NMR (470 MHz, chloroform-d) 6-69.16 (s, 1F), −117.26 (s, 1F).

### 65B: 3,3-difluoro-3-(4-fluorophenoxy)propan-1-ol

To a 50 mL flask under nitrogen charged with 1-((1,1-difluoroallyl)oxy)-4-fluorobenzene (326 mg, 1.733 mmol) and 4,4,5,5-tetramethyl-1,3,2-dioxaborolane (443 mg, 3.47 mmol) was added THF (4 mL). After 5 min stirring, rhodium(I) tris(triphenylphosphine) chloride (64.1 mg, 0.069 mmol) was added. The mixture was stirred at rt for 18 h. TLC (9/1 hexane/EtOAc) showed a spot slightly more polar than the SM. The boronate ester was then oxidized by adding 2 mL 1N NaOH and hydrogen peroxide (2 mL, 19.58 mmol) dropwise. The mixture was stirred for 1 h and then diluted with EtOAc. TLC (2/1 hexane/EtOAc) showed the desired more polar product. The layers were separated. The organic layer was washed with brine, dried with Na₂SO₄, and concentrated. The residue was purified by FCC (24 g silica) up to 50% EtOAc/hexane to afford the desired product 3,3-difluoro-3-(4-fluorophenoxy)propan-1-ol (150 mg, 42%) as a colorless oil.

¹H NMR (499 MHz, chloroform-d) δ 7.23-7.11 (m, 2H), 7.10-7.00 (m, 2H), 4.00 (br t, J=5.8 Hz, 2H), 2.54-2.42 (m, 2H), 1.86 (br s, 1H); ¹⁹F NMR (470 MHz, CHLOROFORM-d) 6-69.00 (s, 1F), −116.99 (s, 1F).

65C: tert-butyl (tert-butoxycarbonyl)(7-(3-(3,3-difluoro-3-(4-fluorophenoxy)propoxy)-2-fluorophenyl)-[1,2,4]triazolo[1,5-a]pyridin-2-yl)carbamate prepared in a similar fashion to 2A from 65B and A.

MS ESI m/z 633.6 (M+H)⁺.

### 65: 7-(3-(3,3-difluoro-3-(4-fluorophenoxy)propoxy)-2-fluorophenyl)-[1,2,4]triazolo[1,5-a]pyridin-2-amine

A solution of 65C (63.3 mg, 0.1 mmol) in anhydrous DCM (0.5 mL) in a 50 mL flask was added TFA (500 μl, 6.49 mmol). The mixture was stirred at room temperature for 3 h. Volatiles were stripped off. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 26% B, 26-66% B over 20 minutes, then a 0-minute hold at 100% B; Flow Rate: 20 mL/min; Column Temperature: 25 C. Fraction collection was triggered by MS signals. Fractions containing the desired product were combined and dried via centrifugal evaporation. The yield of the product was 18.9 mg (44%).

¹H NMR (500 MHz, DMSO-d₆) δ 8.61 (d, J=7.0 Hz, 1H), 7.50 (s, 1H), 7.38-7.32 (m, 1H), 7.27 (br d, J=6.7 Hz, 5H), 7.24-7.19 (m, 1H), 7.05 (br d, J=6.7 Hz, 1H), 6.07 (s, 2H), 4.43 (br t, J=6.1 Hz, 2H), 2.80 (tt, J=11.2, 5.7 Hz, 2H); ¹⁹F NMR (471 MHz, DMSO-d₆) δ −67.54 (s, 1F), −116.79 (s, 1F), −139.55 (s, 1F).

MS ESI m/z 433.3 (M+H)⁺.

### Example 66: 4-(3-(2-amino-[1,2,4]triazolo[1,5-a]pyridin-7-yl)-2-fluoro-6-methylphenoxy)-2,2-difluoro-1-(4-fluorophenyl)butan-1-ol

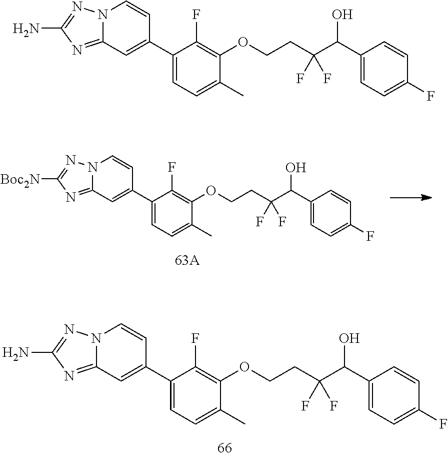

66: 4-(3-(2-amino-[1,2,4]triazolo[1,5-a]pyridin-7-yl)-2-fluoro-6-methylphenoxy)-2,2-difluoro-1-(4-fluorophenyl)butan-1-ol was prepared in a similar fashion to 65 from 14.8 mg of 63A yielding 66 (4.7 mg, 44%).

¹H NMR (500 MHz, DMSO-d₆) δ 8.60 (br d, J=5.5 Hz, 1H), 7.49 (br s, 3H), 7.32-7.24 (m, 1H), 7.23-7.11 (m, 3H), 7.04 (br d, J=5.8 Hz, 1H), 6.41 (br s, 1H), 6.06 (br d, J=0.6 Hz, 2H), 4.96-4.82 (m, 1H), 4.22 (br d, J=3.1 Hz, 2H), 2.49-2.31 (m, 2H), 2.28 (br s, 3H); ¹⁹F NMR (471 MHz, DMSO-d₆) δ −105.33 (br d, J=244.5 Hz, 1F), −109.15 (br d, J=248.0 Hz, 1F), −114.53 (br s, 1F), −135.59 (br s, 1F).

MS ESI m/z 461.4 (M+H)⁺.

### Example 67: 5-(3-(2-amino-[1,2,4]triazolo[1,5-a]pyridin-7-yl)-2-fluorophenoxy)-3,3-difluoro-2-phenylpentan-2-ol (enantiomer 1)

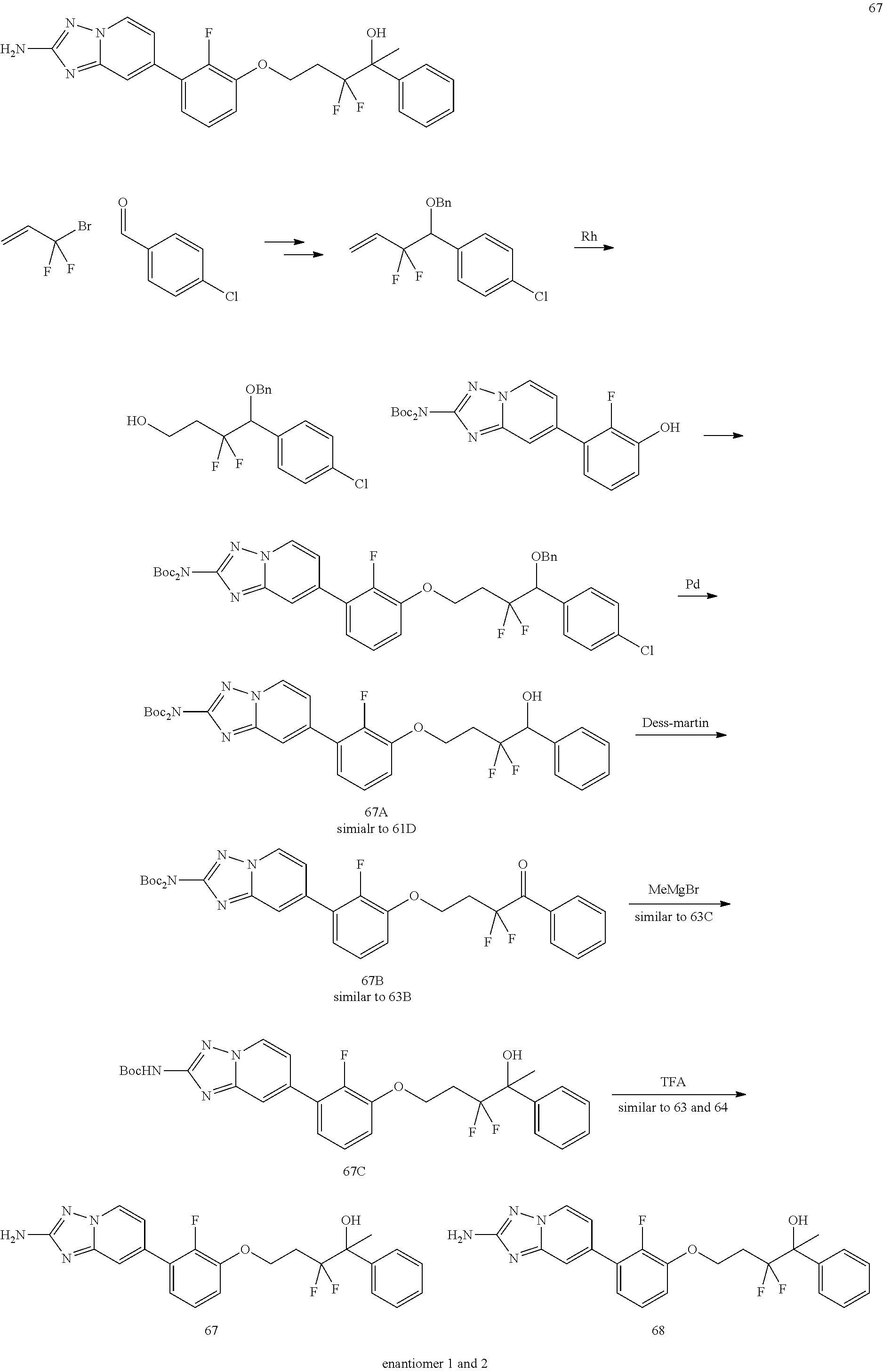

(The 2 unassigned enantiomers are:

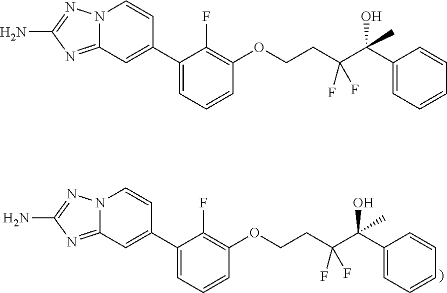 [SMILES: C[C@@](O)(c1ccccc1)C(F)(F)CCOc1cccc(-c2ccn3nc(N)nc3c2)c1F ; C[C@](O)(c1ccccc1)C(F)(F)CCOc1cccc(-c2ccn3nc(N)nc3c2)c1F]

67A: tert-butyl (tert-butoxycarbonyl)(7-(3-(3,3-difluoro-4-hydroxy-4-phenylbutoxy)-2-fluorophenyl)-[1,2,4]triazolo[1,5-a]pyridin-2-yl)carbamate was prepared in a similar fashion to 61D.

MS ESI m/z 629.6 (M+H)⁺.

67B. tert-butyl (tert-butoxycarbonyl)(7-(3-(3,3-difluoro-4-oxo-4-phenylbutoxy)-2-fluorophenyl)-[1,2,4]triazolo[1,5-a]pyridin-2-yl)carbamate was prepared in a similar fashion to 63B

67C: tert-butyl (7-(3-((3,3-difluoro-4-hydroxy-4-phenylpentyl)oxy)-2-fluorophenyl)-[1,2,4]triazolo[1,5-a]pyridin-2-yl)carbamate was prepared in a similar fashion to 63C MS ESI m/z 543.5 (M+H)⁺

67 and 68: The two enantiomers of tert-butyl (7-(3-((3,3-difluoro-4-hydroxy-4-phenylpentyl)oxy)-2-fluorophenyl)-[1,2,4]triazolo[1,5-a]pyridin-2-yl)carbamate were prepared in a similar fashion to 63 and 64.

67 (1.6 mg, 3%): ¹H NMR (500 MHz, DMSO-d₆) δ 8.62-8.51 (m, 1H), 7.56 (br d, J=6.4 Hz, 2H), 7.46 (br s, 1H), 7.37 (br t, J=6.7 Hz, 2H), 7.31 (br d, J=6.7 Hz, 1H), 7.25-7.12 (m, 3H), 7.03 (br d, J=6.1 Hz, 1H), 6.13 (br s, 1H), 6.05 (br s, 2H), 4.29-4.15 (m, 2H), 2.49-2.38 (m, 1H), 2.18-1.98 (m, 1H), 1.61 (br s, 3H); ¹⁹F NMR (471 MHz, DMSO-d₆) δ −108.25 (br d, J=244.5 Hz, 1F), −111.38 (br d, J=246.2 Hz, 1F), −139.67 (br s, 1F).

MS ESI m/z 443.2 (M+H)⁺.

### Example 68: 5-(3-(2-amino-[1,2,4]triazolo[1,5-a]pyridin-7-yl)-2-fluorophenoxy)-3,3-difluoro-2-phenylpentan-2-ol (enantiomer 2)

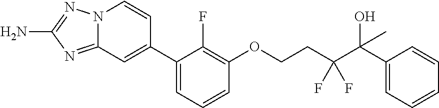 [SMILES: CC(O)(c1ccccc1)C(F)(F)CCOc1cccc(-c2ccn3nc(N)nc3c2)c1F]

68 (1.6 mg, 3%): ¹H NMR (500 MHz, DMSO-d₆) δ 8.59 (br d, J=6.4 Hz, 1H), 7.57 (br d, J=7.3 Hz, 2H), 7.47 (br s, 1H), 7.38 (br t, J=7.0 Hz, 2H), 7.33-7.28 (m, 1H), 7.25-7.11 (m, 3H), 7.02 (br d, J=7.0 Hz, 1H), 6.05 (br s, 2H), 4.32-4.12 (m, 2H), 2.49-2.37 (m, 1H), 2.17-1.97 (m, 1H), 1.62 (br s, 3H); ¹⁹F NMR (471 MHz, DMSO-d₆) δ −108.24 (br d, J=244.5 Hz, 1F), −111.37 (br d, J=242.8 Hz, 1F), −139.64 (br s, 1F).

MS ESI m/z 443.2 (M+H)⁺.

### Example 69: 4-(3-(2-amino-[1,2,4]triazolo[1,5-a]pyridin-7-yl)-2-fluorophenoxy)-2,2-difluoro-1-phenylbutan-1-ol

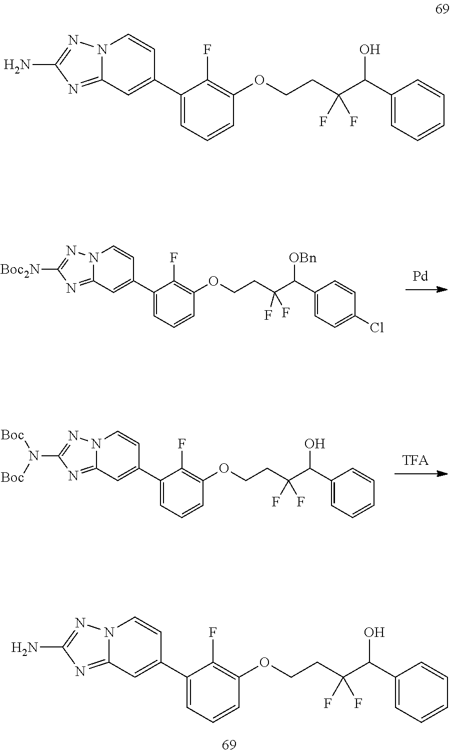

69: 4-(3-(2-amino-[1,2,4]triazolo[1,5-a]pyridin-7-yl)-2-fluorophenoxy)-2,2-difluoro-1-phenylbutan-1-ol was prepared in a similar fashion to 65 from 12 mg (0.019 mmol) of

### 67A Racemic 69 (1.6 mg, 19%) was obtained

¹H NMR (500 MHz, DMSO-d₆) δ 8.59 (d, J=7.0 Hz, 1H), 7.52-7.44 (m, 3H), 7.36 (dt, J=22.2, 7.2 Hz, 3H), 7.29-7.22 (m, 2H), 7.22-7.16 (m, 1H), 7.04 (d, J=7.1 Hz, 1H), 6.37 (d, J=5.4 Hz, 1H), 6.05 (s, 2H), 4.90 (dd, J=14.5, 7.9 Hz, 1H), 4.31 (t, J=6.7 Hz, 2H), 2.49-2.30 (m, 2H); ¹⁹F NMR (471 MHz, DMSO-d₆) δ −105.49 (d, J=246.1 Hz), −108.76 (d, J=246.3 Hz), −139.62.

MS ESI m/z 429.2 (M+H)⁺.

### Example 70: 4-(3-(2-amino-[1,2,4]triazolo[1,5-a]pyridin-7-yl)-2-fluorophenoxy)-1-(4-chlorophenyl)-2,2-difluorobutan-1-ol

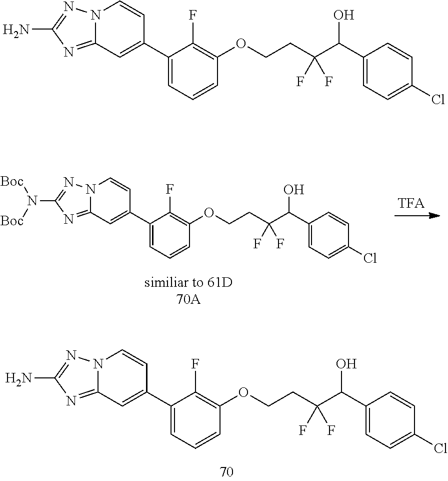 [SMILES: Nc1nc2cc(-c3cccc(OCCC(F)(F)C(O)c4ccc(Cl)cc4)c3F)ccn2n1 ; CC(C)(C)OC(=O)N(C(=O)OC(C)(C)C)c1nc2cc(-c3cccc(OCCC(F)(F)C(O)c4ccc(Cl)cc4)c3F)ccn2n1 ; Nc1nc2cc(-c3cccc(OCCC(F)(F)C(O)c4ccc(Cl)cc4)c3F)ccn2n1]

70A: tert-butyl (tert-butoxycarbonyl)(7-(3-(4-(4-chlorophenyl)-3,3-difluoro-4-hydroxybutoxy)-2-fluorophenyl)-[1,2,4]triazolo[1,5-a]pyridin-2-yl)carbamate was prepared in a similar fashion to 61D.

70: 4-(3-(2-amino-[1,2,4]triazolo[1,5-a]pyridin-7-yl)-2-fluorophenoxy)-1-(4-chlorophenyl)-2,2-difluorobutan-1-ol was prepared in a similar fashion to 65. Racemic 70 (2.9 mg, 20%) was obtained from 20 mg of crude 70A.

¹H NMR (500 MHz, DMSO-d₆) δ 8.62 (d, J=7.0 Hz, 1H), 7.51 (s, 1H), 7.46 (q, J=8.4 Hz, 4H), 7.31-7.23 (m, 2H), 7.19 (dd, J=9.9, 5.2 Hz, 1H), 7.07 (d, J=6.9 Hz, 1H), 4.93 (dd, J=15.5, 7.6 Hz, 1H), 4.32 (t, J=6.7 Hz, 2H), 2.48-2.31 (m, 2H); ¹⁹F NMR (471 MHz, DMSO-d₆) δ −105.42 (d, J=246.9 Hz), −109.20 (d, J=246.8 Hz), −139.56.

MS ESI m/z 463.1 (M+H)⁺.

### Example 71: 4-(3-(2-amino-[1,2,4]triazolo[1,5-a]pyridin-7-yl)-2-fluorophenoxy)-2,2-difluoro-1-(4-fluorophenyl)butan-1-ol (enantiomer 1)

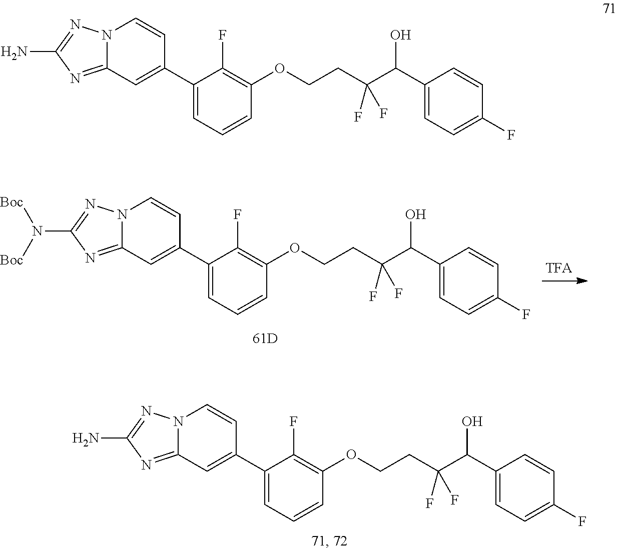 [SMILES: Nc1nc2cc(-c3cccc(OCCC(F)(F)C(O)c4ccc(F)cc4)c3F)ccn2n1 ; CC(C)(C)OC(=O)N(C(=O)OC(C)(C)C)c1nc2cc(-c3cccc(OCCC(F)(F)C(O)c4ccc(F)cc4)c3F)ccn2n1 ; Nc1nc2cc(-c3cccc(OCCC(F)(F)C(O)c4ccc(F)cc4)c3F)ccn2n1]

(The 2 unassigned enantiomers are:

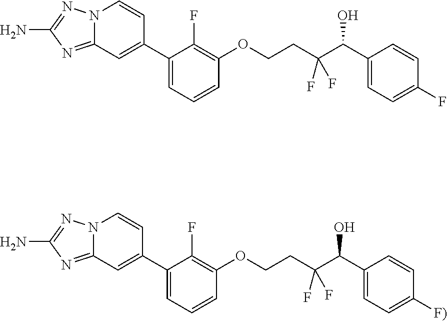

### 71 and 72: 4-(3-(2-amino-[1,2,4]triazolo[1,5-a]pyridin-7-yl)-2-fluorophenoxy)-2,2-difluoro-1-(4-fluorophenyl)butan-1-ol

To a solution of 61D (191 mg, 0.296 mmol) in DCM (2 mL) was added TFA (1.5 mL, 19.47 mmol). The mixture was stirred at rt for 2 h. LC-MS indicated complete conversion to the desired product (M+H=447.2). Volatiles were stripped off. The residue was dissolved in MeOH, filtered and purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 21% B, 21-61% B over 20 minutes, then a 0-minute hold at 100% B; Flow Rate: 20 mL/min; Column Temperature: 25 C. Fraction collection was triggered by MS and UV signals. Fractions containing the desired product were combined and dried via centrifugal evaporation to give the racemic material (63.6 mg).

The material was further separated using SFC-chiral chromatography with following conditions:

Instrument:Waters 100 Prep SFC; Column: Chiral IC, 21×250 mm. 5 micron; Mobile Phase: 70% C_{02/30}% MeOH w/0.1% DEA; Flow Conditions: 60 mL/min; Detector Wavelength: 220 nm; Injection Details: 600 L 61.6 mg dissolved in 3 mL MeOH.

Two enantiomers were separated:

71 (16.5 mg, 12% for three steps): 1H NMR (500 MHz, DMSO-d₆) δ 8.57 (d, J=7.0 Hz, 1H), 7.53-7.42 (m, 3H), 7.29-7.22 (m, 2H), 7.22-7.14 (m, 3H), 7.06 (d, J=7.1 Hz, 1H), 4.96-4.84 (m, 1H), 4.30 (t, J=6.5 Hz, 2H), 2.48-2.27 (m, 2H); ¹⁹F NMR (471 MHz, DMSO-d₆) δ −105.66 (d, J=246.7 Hz), −109.31 (d, J=247.2 Hz), −114.37, −139.73.

MS ESI m/z 447.2 (M+H)⁺.

### Example 72: 4-(3-(2-amino-[1,2,4]triazolo[1,5-a]pyridin-7-yl)-2-fluorophenoxy)-2,2-difluoro-1-(4-fluorophenyl)butan-1-ol (enantiomer 2)

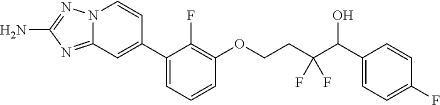 [SMILES: Nc1nc2cc(-c3cccc(OCCC(F)(F)C(O)c4ccc(F)cc4)c3F)ccn2n1]

72: (20.6 mg, 16% for three steps): 1H NMR (500 MHz, DMSO-d₆) δ 8.59 (d, J=7.0 Hz, 1H), 7.48 (d, J=7.7 Hz, 3H), 7.26 (dd, J=5.5, 3.5 Hz, 2H), 7.20 (h, J=7.5, 6.7 Hz, 3H), 7.05 (d, J=7.0 Hz, 1H), 6.45 (d, J=5.4 Hz, 1H), 6.05 (s, 1H), 4.92 (dt, J=14.7, 6.9 Hz, 1H), 4.31 (t, J=6.6 Hz, 2H), 2.49-2.29 (m, 2H); ¹⁹F NMR (471 MHz, DMSO-d₆) δ −105.60 (d, J=246.8 Hz), −109.28 (d, J=246.8 Hz), −114.46, −139.65.

MS ESI m/z 447.2 (M+H)⁺.

### Example 73: 5-(3-(2-amino-[1,2,4]triazolo[1,5-a]pyridin-7-yl)-2-fluorophenoxy)-3,3-difluoro-2-(4-fluorophenyl)pentan-2-ol (racemic)

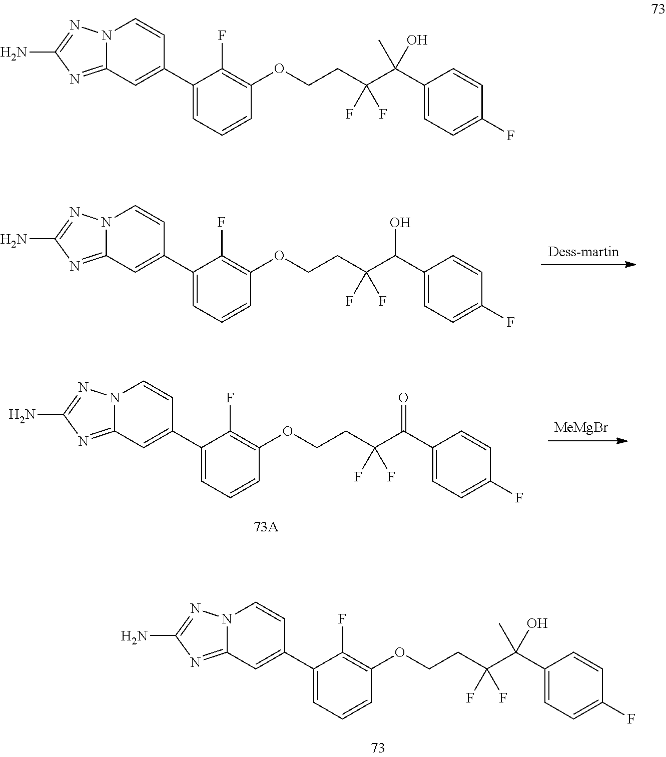

### 73A: 4-(3-(2-amino-[1,2,4]triazolo[1,5-a]pyridin-7-yl)-2-fluorophenoxy)-2,2-difluoro-1-(4-fluorophenyl)butan-1-one

A suspension of 4-(3-(2-amino-[1,2,4]triazolo[1,5-a]pyridin-7-yl)-2-fluorophenoxy)-2,2-difluoro-1-(4-fluorophenyl)butan-1-ol (30.5 mg, 0.068 mmol) and dess-martinperiodinane (43.5 mg, 0.102 mmol) in a 1-dram pressure vial in DCM (1 mL) was stirred at rt for 3 h.

LC-MS indicated complete conversion to the desired product (M+H=445.1). The reaction mixture was quenched with sodium thiosulfite and diluted with EtOAc. The layers were separated. The organic layer was washed with brine, dried with Na₂SO₄, and concentrated to the crude product (42.3 mg, 100% mass recovery) as a tan solid, which was directly used for the next step.

MS ESI m/z 445.1 (M+H)⁺

### 73: 5-(3-(2-amino-[1,2,4]triazolo[1,5-a]pyridin-7-yl)-2-fluorophenoxy)-3,3-difluoro-2-(4-fluorophenyl)pentan-2-ol

To a solution of 4-(3-(2-amino-[1,2,4]triazolo[1,5-a]pyridin-7-yl)-2-fluorophenoxy)-2,2-difluoro-1-(4-fluorophenyl)butan-1-one (39 mg, 0.088 mmol) in THF (1.0 mL) in a 50 mL flask was added methylmagnesium bromide (0.088 mL, 0.263 mmol). The mixture was stirred at rt for 2 h. LC-MS indicated good conversion to the desired product (M+H=461.1), but there was still SM. More reagent (3 equiv) was added and the reaction continued for another 3 h. The reaction was quenched with saturated NH₄C₁ solution and diluted with EtOAc. The layers were separated. The organic layer was washed with brine, dried with Na₂SO₄, and concentrated to a tan solid (33.3 mg). The residue was dissolved in DMF, filtered, and purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 23% B, 23-63% B over 20 minutes, then a 0-minute hold at 100% B; Flow Rate: 20 mL/min; Column; Temperature: 25 C. Fraction collection was triggered by MS signals. Fractions containing the desired product were combined and dried via centrifugal evaporation to give 73 (3.5 mg, 8% for two steps);

¹H NMR (500 MHz, DMSO-d₆) δ 8.60 (d, J=6.8 Hz, 1H), 7.59 (dd, J=8.5, 5.4 Hz, 2H), 7.47 (s, 1H), 7.28-7.13 (m, 5H), 7.02 (d, J=7.0 Hz, 1H), 6.06 (s, 2H), 4.25 (ddd, J=13.4, 10.1, 6.9 Hz, 2H), 2.46 (dd, J=15.4, 7.5 Hz, 1H), 2.10 (ddd, J=29.2, 14.9, 7.3 Hz, 1H), 1.62 (s, 3H);

¹⁹F NMR (471 MHz, DMSO-d₆) δ −108.42 (d, J=243.5 Hz), −111.30 (d, J=244.1 Hz), −115.74, −139.60.

MS ESI m/z 461.1 (M+H)⁺.

### Example 74: 7-(3-(3,3-difluoro-4-phenylbutoxy)-2-fluorophenyl)-[1,2,4]triazolo[1,5-a]pyridin-2-amine

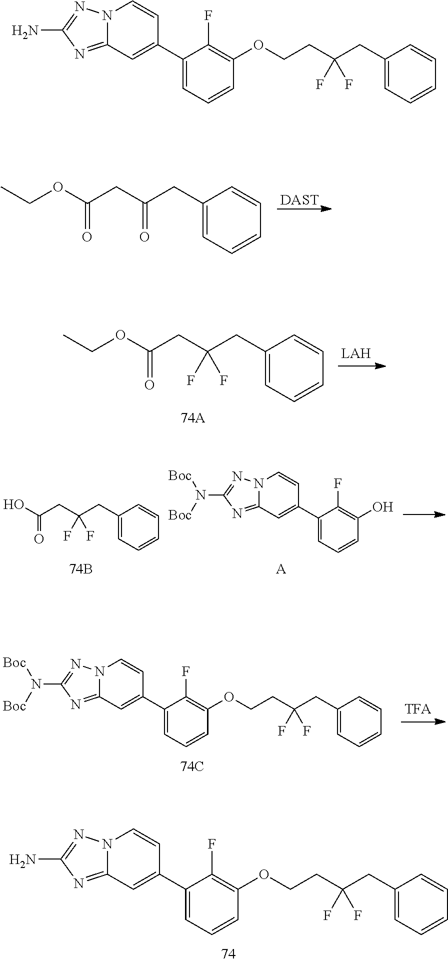

### 74A: ethyl 3,3-difluoro-4-phenylbutanoate

To a solution of ethyl 3-oxo-4-phenylbutanoate (1.52 g, 7.37 mmol) in a 50 mL flask in DCM (5 mL) was added DAST (0.974 mL, 7.37 mmol). The mixture was stirred at rt overnight for 18 h. TLC (4/1 hexane/EtOAc) showed two spots slightly above SM. Another 0.5 equiv. of DAST was added and the reaction continued for another day. TLC showed still some SM left. The reaction was left without stirring for a week. TLC showed no SM left. The reaction was carefully quenched with saturated NaHCO₃ solution and diluted with EtOAc. The layers were separated. The organic layer was washed with brine, dried with Na₂SO₄, and concentrated. The residue was purified by FCC (up to 40% EtOAc/hexane) to afford the desired product (375 mg, 22%) as an orange oil, which was used crude in the next step.

¹H NMR (499 MHz, Chloroform-d) δ 7.36-7.31 (m, 5H), 4.23 (q, J=7.2 Hz, 2H), 3.41 (t, J=16.4 Hz, 2H), 2.83 (t, J=14.5 Hz, 2H), 1.32 (t, J=7.1 Hz, 3H);

¹⁹F NMR (470 MHz, Chloroform-d) δ −92.14.

### 74B: 3,3-difluoro-4-phenylbutan-1-ol

To a solution of ethyl 3,3-difluoro-4-phenylbutanoate (375 mg, 1.643 mmol) in a 50 mL flask in THF (6 mL) was added LAH (0.986 mL, 1.972 mmol) dropwise. The mixture was stirred at rt for 2 h. TLC (4/1 hexane/EtOAc) showed complete consumption of SM.

The reaction was diluted with ether and carefully quenched with Na₂SO₄ and droplets of water. The mixture was filtered, washed with ether. The combined organic solution was concentrated to give the desired product (291.5 mg, 95%) as a tan oil, which was used crude in the next step.

¹H NMR (499 MHz, Chloroform-d) δ 7.36-7.29 (m, 5H), 3.89 (t, J=6.2 Hz, 2H), 3.22 (t, J=16.4 Hz, 2H), 2.10 (tt, J=17.1, 6.2 Hz, 2H), 1.70 (td, J=5.7, 2.7 Hz, 1H);

¹⁹F NMR (470 MHz, Chloroform-d) δ −95.67.

### 74C: tert-butyl (tert-butoxycarbonyl)(7-(3-(3,3-difluoro-4-phenylbutoxy)-2-fluorophenyl)-[1,2,4]triazolo[1,5-a]pyridin-2-yl)carbamate

To a mixture of 3,3-difluoro-4-phenylbutan-1-ol (0.028 g, 0.150 mmol), Ph₃P (0.059 g, 0.225 mmol), and A (0.067 g, 0.15 mmol) in THF (0.8 mL) was added DIAD (0.041 mL, 0.210 mmol). The mixture was stirred at room temperature for 18 h. LC-MS indicated good conversion to the desired product (M+H=613.3). Volatiles were stripped off. The residue was directly carried onto the next reaction.

MS ESI m/z 613.3 (M+H)⁺.

### 74: 7-(3-(3,3-difluoro-4-phenylbutoxy)-2-fluorophenyl)-[1,2,4]triazolo[1,5-a]pyridin-2-amine

To a solution of 10 (92 mg, 0.15 mmol) in DCM (0.8 mL) was added TFA (0.5 ml, 6.49 mmol). The mixture was stirred at room temperature for 1 h. LC-MS indicated good conversion to the desired product (M+H=413.1), along with some mono-Boc product (M+H=513.2). Volatiles were stripped off. The residue was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 33% B, 33-90% B over 20 minutes, then a 0-minute hold at 100% B; Flow Rate: 20 mL/min; Column Temperature: 25 C. Fraction collection was triggered by MS and UV signals.

Fractions containing the desired product were combined and dried via centrifugal evaporation to give 74 (20.4 mg, 32%):

¹H NMR (500 MHz, DMSO-d₆) δ 8.60 (d, J=7.0 Hz, 1H), 7.49 (s, 1H), 7.40-7.23 (m, 7H), 7.23-7.17 (m, 1H), 7.04 (d, J=6.9 Hz, 1H), 6.06 (s, 2H), 4.32 (t, J=6.4 Hz, 2H), 2.40 (td, J=16.1, 7.9 Hz, 2H) (missing CH₂).

¹⁹F NMR (471 MHz, DMSO-d₆) δ −94.53, −139.68.

MS ESI m/z 413.1 (M+H)⁺.

### Example 75: 5-(3-(2-amino-[1,2,4]triazolo[1,5-a]pyridin-6-yl)-2,6-difluorophenoxy)-3,3-difluoro-2-(4-fluorophenyl)pentan-2-ol (enantiomer 1)

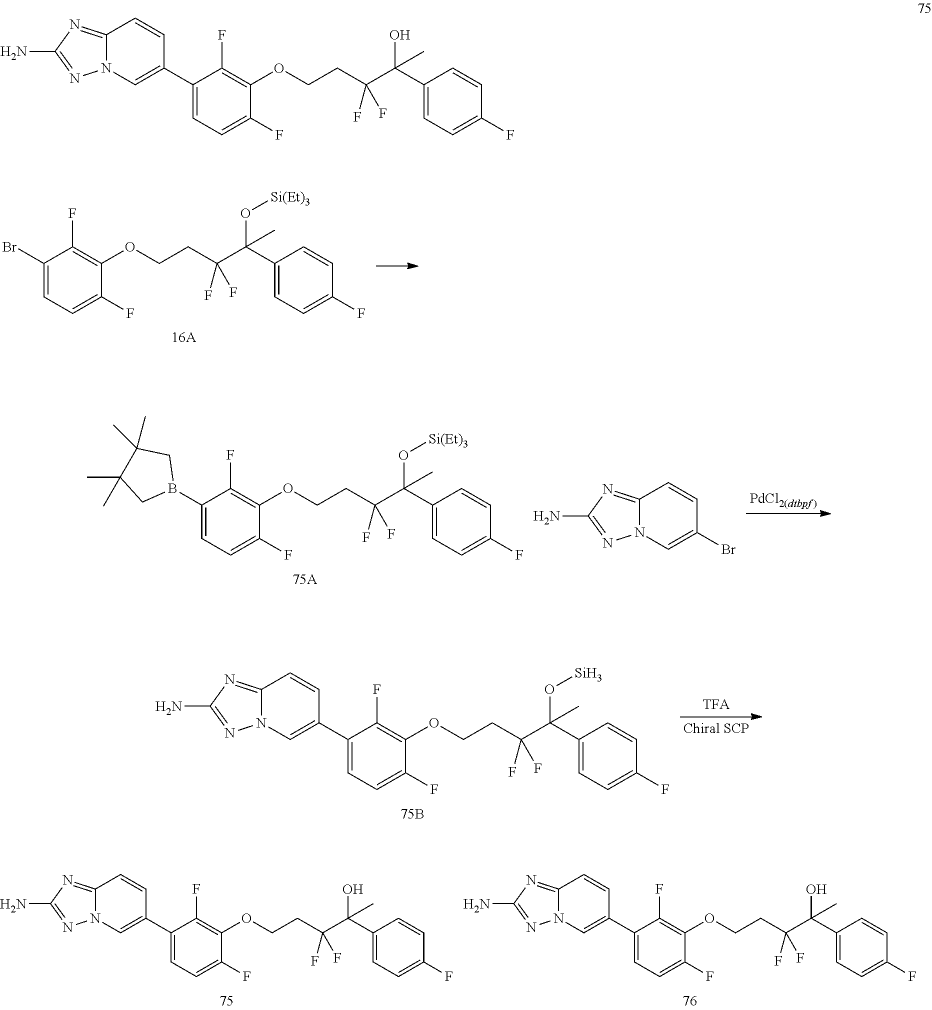

(The 2 unassigned enantiomers are:

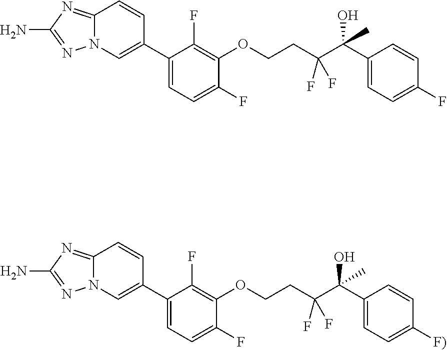

75A: ((5-(2,6-difluoro-3-(4,4,5,5-tetramethyl-1,3,2-dioxaborolan-2-yl)phenoxy)-3,3-difluoro-2-(4-fluorophenyl)pentan-2-yl)oxy)triethylsilane: A mixture of ((5-(3-bromo-2,6-difluorophenoxy)-3,3-difluoro-2-(4-fluorophenyl)pentan-2-yl)oxy)triethylsilane (360 mg, 0.667 mmol), bispin (186 mg, 0.734 mmol), potassium acetate (196 mg, 2.002 mmol) and PdCl₂(dppf)-CH₂Cl₂adduct (54.5 mg, 0.067 mmol) in dioxane (4 mL) was purged with nitrogen and stirred at 100° C. for 4 h. TLC (2% hexane/EtOAc) indicated a tailed spot, although it was comigrated with the SM. The mixture was directly used in the next step.

### 75B: 6-(3-((3,3-difluoro-4-(4-fluorophenyl)-4-((triethylsilyl)oxy)pentyl)oxy)-2,4-difluorophenyl)-[1,2,4]triazolo[1,5-a]pyridin-2-amine

To crude ((5-(2,6-difluoro-3-(4,4,5,5-tetramethyl-1,3,2-dioxaborolan-2-yl)phenoxy)-3,3-difluoro-2-(4-fluorophenyl)pentan-2-yl)oxy)triethylsilane (64.5 mg, 0.11 mmol) in dioxane (0.5 mL) was added bis(di-tert-butylphosphino)ferrocene]dichloropalladium(II) 98% (3.58 mg, 5.50 μmol), 6-bromo-[1,2,4]triazolo[1,5-a]pyridin-2-amine (23.43 mg, 0.110 mmol) and tripotassium phosphate (0.165 mL, 0.330 mmol). The mixture was purged with N₂, then stirred at 100° C. for about 30 min. LCMS indicated most of SM was consumed, and the desired product was formed. Water was added, and extracted with 2× EtOAc. The organic layer was concentrated. The residue was purified via silica gel chromatography (4 g, hexanes-100% EtOAc) to give 6-(3-((3,3-difluoro-4-(4-fluorophenyl)-4-((triethylsilyl)oxy)pentyl)oxy)-2,4-difluorophenyl)-[1,2,4]triazolo[1,5-a]pyridin-2-amine (40 mg, 0.067 mmol, 61.4% yield).

¹H NMR (499 MHz, CHLOROFORM-d) δ 8.47-8.44 (m, 1H), 7.54-7.45 (m, 4H), 7.10-6.97 (m, 4H), 4.59 (s, 2H), 4.35 (td, J=9.2, 6.0 Hz, 1H), 4.27 (td, J=9.3, 6.0 Hz, 1H), 2.62-2.47 (m, 1H), 2.12-1.98 (m, 1H), 1.77 (s, 3H), 0.96 (t, J=7.9 Hz, 9H), 0.72-0.61 (m, 6H).

MS ESI m/z 593.7 (M+H)⁺.

75 and 76: The two enantiomers of 5-(3-(2-amino-[1,2,4]triazolo[1,5-a]pyridin-6-yl)-2,6-difluorophenoxy)-3,3-difluoro-2-(4-fluorophenyl)pentan-2-ol were prepared in a similar fashion to 2 and 3.

The first eluting isomer 75, 5-(3-(2-amino-[1,2,4]triazolo[1,5-a]pyridin-6-yl)-2,6-difluorophenoxy)-3,3-difluoro-2-(4-fluorophenyl)pentan-2-ol (3.5 mg, 7.32 μmol, 10.84% yield) and the second eluting isomer 76, 5-(3-(2-amino-[1,2,4]triazolo[1,5-a]pyridin-6-yl)-2,6-difluorophenoxy)-3,3-difluoro-2-(4-fluorophenyl)pentan-2-ol (4 mg, 8.36 μmol, 12.39% yield) were obtained.

75, ¹H NMR (500 MHz, DMSO-d₆) δ 8.72 (s, 1H), 7.59-7.54 (m, 3H), 7.44 (d, J=9.1 Hz, 1H), 7.37-7.31 (m, 1H), 7.26-7.21 (m, 1H), 7.17 (t, J=8.9 Hz, 2H), 4.32-4.20 (m, 2H), 2.49-2.38 (m, 1H), 2.15-2.02 (m, 1H), 1.60 (s, 3H).

MS ESI m/z 479.2 (M+H)⁺.

### Example 76: 5-(3-(2-amino-[1,2,4]triazolo[1,5-a]pyridin-6-yl)-2,6-difluorophenoxy)-3,3-difluoro-2-(4-fluorophenyl)pentan-2-ol (enantiomer 2)

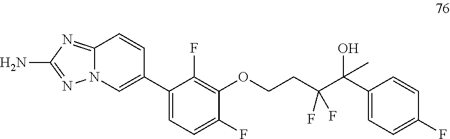 [SMILES: CC(O)(c1ccc(F)cc1)C(F)(F)CCOc1c(F)ccc(-c2ccc3nc(N)nn3c2)c1F]

76, ¹H NMR (500 MHz, DMSO-d₆) δ 8.72 (s, 1H), 7.59-7.54 (m, 3H), 7.44 (d, J=9.0 Hz, 1H), 7.36-7.31 (m, 1H), 7.26-7.21 (m, 1H), 7.17 (t, J=8.8 Hz, 2H), 6.19 (s, 1H), 6.09 (s, 2H), 4.26 (tdd, J=17.3, 10.2, 7.0 Hz, 2H), 2.49-2.36 (m, 1H), 2.15-2.02 (m, 1H), 1.59 (s, 3H).

MS ESI m/z 479.2 (M+H)⁺.

### Example 77: 5-((6-(2-amino-[1,2,4]triazolo[1,5-a]pyridin-7-yl)-5-fluoropyridin-2-yl)oxy)-3,3-difluoro-2-(4-fluorophenyl)pentan-2-ol (enantiomer 1)

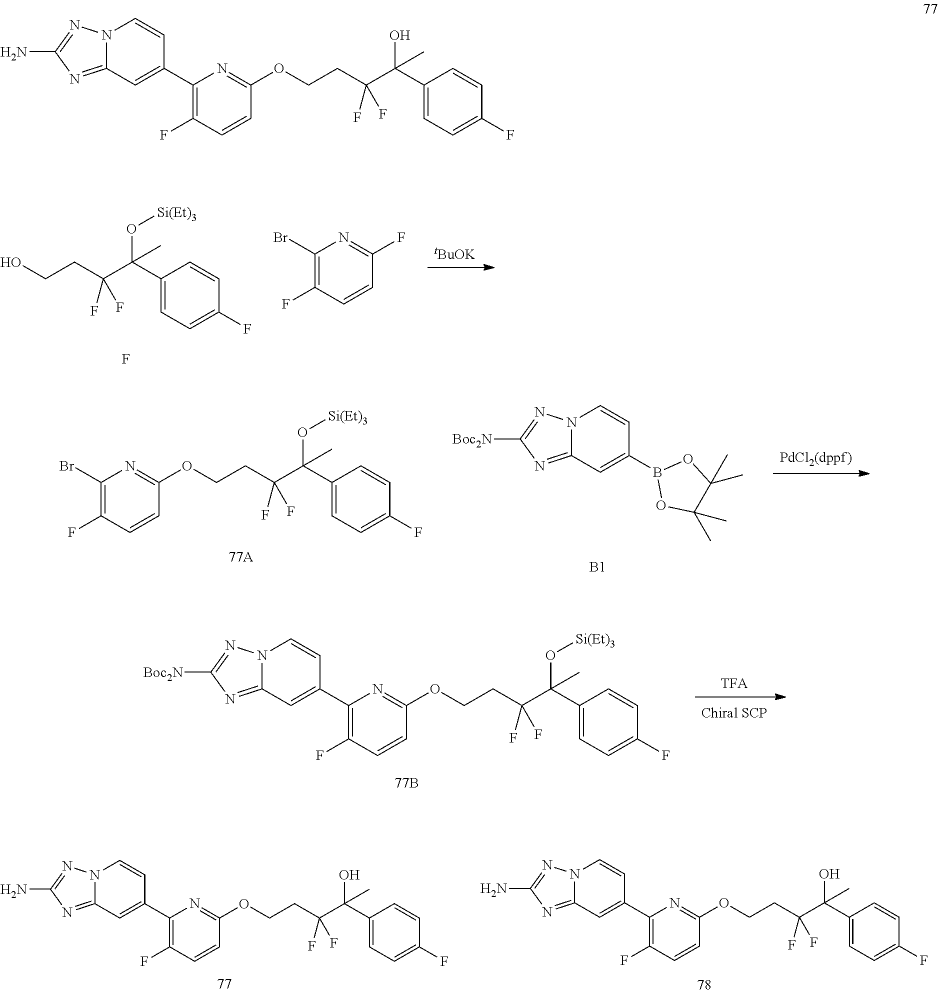

(The 2 unassigned enantiomers are:

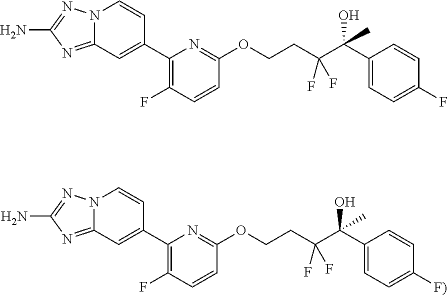

### 77A: 2-bromo-6-((3,3-difluoro-4-(4-fluorophenyl)-4-((triethylsilyl)oxy)pentyl)oxy)-3-fluoropyridine

To a solution of 2-bromo-3,6-difluoropyridine (83 mg, 0.430 mmol) and 3,3-difluoro-4-(4-fluorophenyl)-4-((triethylsilyl)oxy)pentan-1-ol F (125 mg, 0.359 mmol) in THF (2 mL) was added potassium tert-butoxide/THF (0.395 mL, 0.395 mmol) dropwise at rt. The mixture was stirred at rt for 2 days. LCMS indicated some of the product lost the TES group. Water was added, and extracted with 2× EtOAc. The organic layer was concentrated. The residue was purified via silica gel chromatography (4 g, hexanes-100% EtOAc) to give 2-bromo-6-((3,3-difluoro-4-(4-fluorophenyl)-4-((triethylsilyl)oxy)pentyl)oxy)-3-fluoropyridine (145 mg, 0.278 mmol, 77% yield).

MS ESI m/z 524.0 (M+H)⁺.

### 77B: tert-butyl (tert-butoxycarbonyl)(7-(6-((3,3-difluoro-4-(4-fluorophenyl)-4-((triethylsilyl)-oxy)pentyl)oxy)-3-fluoropyridin-2-yl)-[1,2,4]triazolo[1,5-a]pyridin-2-yl)carbamate

To a solution of 5-((6-bromo-5-fluoropyridin-2-yl)oxy)-3,3-difluoro-2-(4-fluorophenyl)pentan-2-ol (143 mg, 0.35 mmol), tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate B1 (209 mg, 0.455 mmol), and tripotassium phosphate (0.525 mL, 1.050 mmol) in dioxane (1.5 mL) was added PdCl₂(dppf)-CH₂Cl₂ adduct (14.29 mg, 0.018 mmol). The mixture was stirred at 100° C. for 1 h. LCMS indicated the SM was consumed. Water was added, and extracted with 2× EtOAc. The organic layer was concentrated to give a crude tert-butyl (tert-butoxycarbonyl)(7-(6-((3,3-difluoro-4-(4-fluorophenyl)-4-((triethylsilyl)oxy)pentyl)oxy)-3-fluoropyridin-2-yl)-[1,2,4]triazolo[1,5-a]pyridin-2-yl)carbamate 77B (185 mg, 0.238 mmol, 68.1% yield), which was used directly for the next step.

MS ESI m/z 776.8 (M+H)⁺.

77 and 78: The two enantiomers of 5-((6-(2-amino-[1,2,4]triazolo[1,5-a]pyridin-7-yl)-5-fluoropyridin-2-yl)oxy)-3,3-difluoro-2-(4-fluorophenyl)pentan-2-ol were prepared in a similar fashion to 2 and 3.

The first eluting isomer 5-((6-(2-amino-[1,2,4]triazolo[1,5-a]pyridin-7-yl)-5-fluoropyridin-2-yl)oxy)-3,3-difluoro-2-(4-fluorophenyl)pentan-2-ol (23.6 mg, 0.051 mmol, 21.22% yield) and the second eluting isomer 5-((6-(2-amino-[1,2,4]triazolo[1,5-a]pyridin-7-yl)-5-fluoropyridin-2-yl)oxy)-3,3-difluoro-2-(4-fluorophenyl)pentan-2-ol (23 mg, 0.049 mmol, 20.66% yield) were obtained.

77, ¹H NMR (500 MHz, DMSO-d₆) δ 8.58 (d, J=7.0 Hz, 1H), 7.84 (s, 1H), 7.73 (dd, J=10.4, 9.2 Hz, 1H), 7.57 (dd, J=8.3, 5.7 Hz, 2H), 7.40 (d, J=6.9 Hz, 1H), 7.11 (t, J=8.7 Hz, 2H), 6.83 (dd, J=8.8, 2.4 Hz, 1H), 6.15 (s, 1H), 4.51-4.41 (m, 2H), 2.49-2.39 (m, 1H), 2.17-2.02 (m, 1H), 1.60 (s, 3H).

MS ESI m/z 462.2 (M+H)⁺.

### Example 78: 5-((6-(2-amino-[1,2,4]triazolo[1,5-a]pyridin-7-yl)-5-fluoropyridin-2-yl)oxy)-3,3-difluoro-2-(4-fluorophenyl)pentan-2-ol (enantiomer 2)

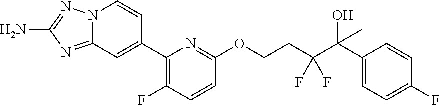 [SMILES: CC(O)(c1ccc(F)cc1)C(F)(F)CCOc1ccc(F)c(-c2ccn3nc(N)nc3c2)n1]

78, ¹H NMR (500 MHz, DMSO-d₆) δ 8.59 (d, J=7.1 Hz, 1H), 7.84 (s, 1H), 7.74 (dd, J=10.6, 9.1 Hz, 1H), 7.57 (br dd, J=8.2, 5.7 Hz, 2H), 7.40 (br d, J=6.6 Hz, 1H), 7.11 (t, J=8.9 Hz, 2H), 6.84 (dd, J=8.9, 2.4 Hz, 1H), 6.14 (s, 1H), 6.05 (s, 2H), 4.52-4.41 (m, 2H), 2.49-2.39 (m, 1H), 2.17-2.04 (m, 1H), 1.60 (s, 3H).

MS ESI m/z 462.2 (M+H)⁺.

### Example 79: 5-((6-(2-amino-[1,2,4]triazolo[1,5-a]pyridin-7-yl)-5-chloropyridin-2-yl)oxy)-3,3-difluoro-2-(4-fluorophenyl)pentan-2-ol (enantiomer 1)

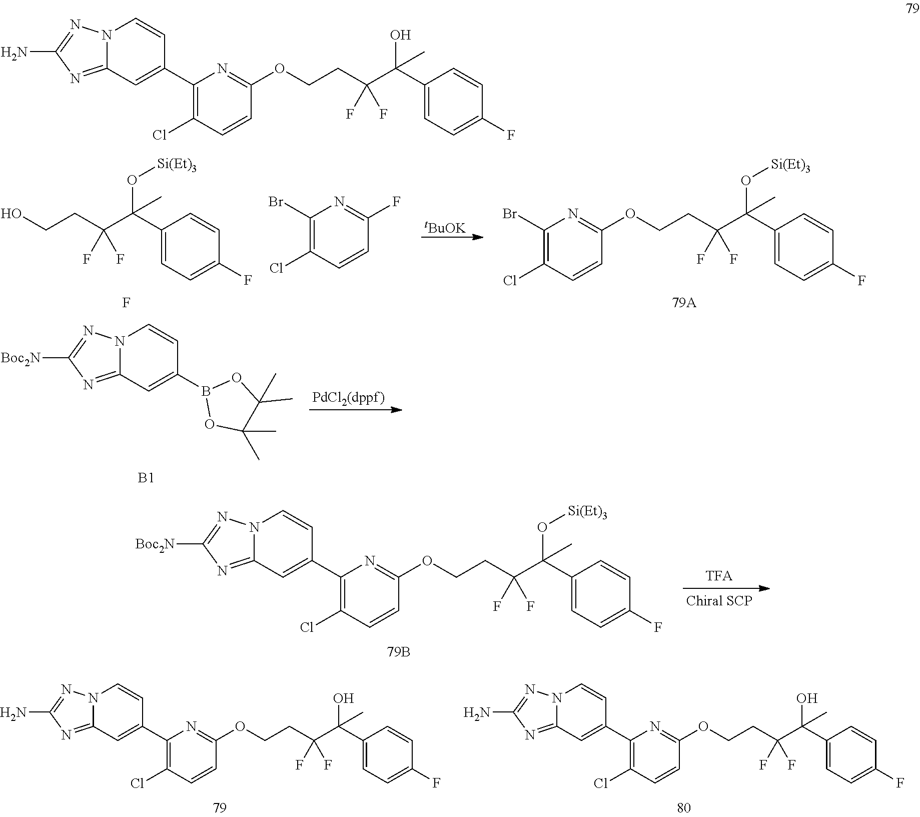

(The 2 unassigned enantiomers are:

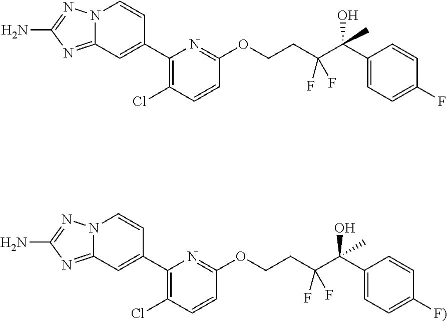

79 and 80: The two enantiomers of 5-((6-(2-amino-[1,2,4]triazolo[1,5-a]pyridin-7-yl)-5-chloropyridin-2-yl)oxy)-3,3-difluoro-2-(4-fluorophenyl)pentan-2-ol were prepared in a similar fashion to 77 and 78 starting from F and 2-bromo-3-chloro-6-fluoropyridine.

The first eluting isomer 79, 5-((6-(2-amino-[1,2,4]triazolo[1,5-a]pyridin-7-yl)-5-chloropyridin-2-yl)oxy)-3,3-difluoro-2-(4-fluorophenyl)pentan-2-ol (12.7 mg, 0.026 mmol, 11.02% yield) and the second eluting isomer 80, 5-((6-(2-amino-[1,2,4]triazolo[1,5-a]pyridin-7-yl)-5-chloropyridin-2-yl)oxy)-3,3-difluoro-2-(4-fluorophenyl)pentan-2-ol (12.9 mg, 0.027 mmol, 11.56% yield) were obtained.

79, ¹H NMR (500 MHz, DMSO-d₆) δ 8.60 (d, J=6.9 Hz, 1H), 7.88 (d, J=8.8 Hz, 1H), 7.67 (s, 1H), 7.57 (br dd, J=8.3, 5.9 Hz, 2H), 7.18-7.09 (m, 3H), 6.86 (d, J=8.8 Hz, 1H), 6.06 (s, 2H), 4.50-4.39 (m, 2H), 2.51-2.38 (m, 1H), 2.16-2.01 (m, 1H), 1.61 (s, 3H).

MS ESI m/z 478.1 (M+H)⁺.

### Example 80: 5-((6-(2-amino-[1,2,4]triazolo[1,5-a]pyridin-7-yl)-5-chloropyridin-2-yl)oxy)-3,3-difluoro-2-(4-fluorophenyl)pentan-2-ol (enantiomer 2)

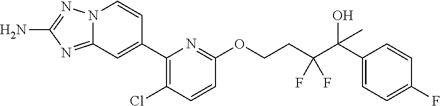 [SMILES: CC(O)(c1ccc(F)cc1)C(F)(F)CCOc1ccc(Cl)c(-c2ccn3nc(N)nc3c2)n1]

80, ¹H NMR (500 MHz, DMSO-d₆) δ 8.57 (d, J=6.8 Hz, 1H), 7.86 (d, J=8.7 Hz, 1H), 7.64 (s, 1H), 7.57-7.51 (m, 2H), 7.15-7.07 (m, 3H), 6.83 (d, J=8.7 Hz, 1H), 6.03 (s, 2H), 4.47-4.36 (m, 2H), 2.48-2.36 (m, 1H), 2.14-1.99 (m, 1H), 1.58 (s, 3H).

MS ESI m/z 478.1 (M+H)⁺.

### Example 81: 5-((6-(2-amino-[1,2,4]triazolo[1,5-a]pyridin-7-yl)-5-chloropyrazin-2-yl)oxy)-3,3-difluoro-2-(4-fluorophenyl)pentan-2-ol (racemic)

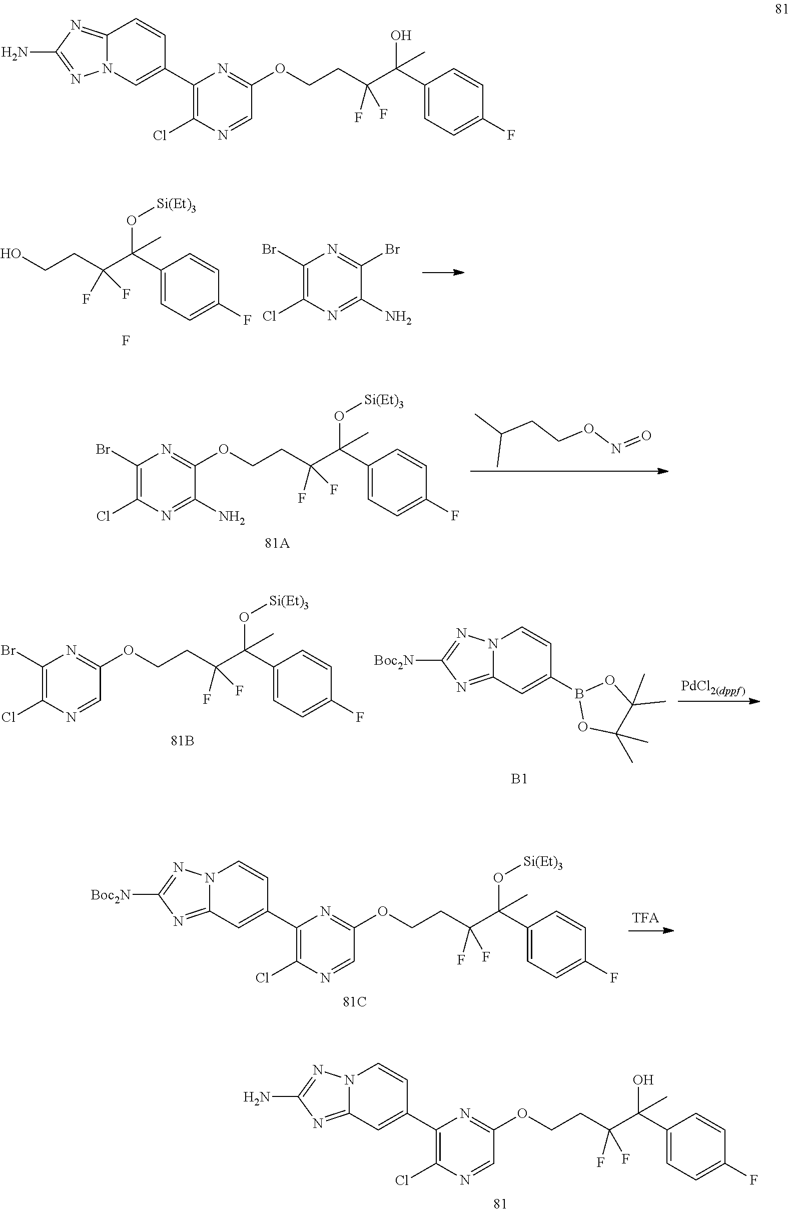

### 81A: 5-bromo-6-chloro-3-((3,3-difluoro-4-(4-fluorophenyl)-4-((triethylsilyl)oxy)pentyl)-oxy)pyrazin-2-amine

To a solution of 3,3-difluoro-4-(4-fluorophenyl)-4-((triethylsilyl)oxy)pentan-1-ol (105 mg, 0.3 mmol) and 3,5-dibromo-6-chloropyrazin-2-amine (103 mg, 0.360 mmol) in dioxane (2 mL) was added NaOH (36.0 mg, 0.900 mmol) at rt. The mixture was stirred at 75° C. for 18 h. Another 5 eq. of NaOH were added. The reaction was stirred at 105° C. for 1 h. TLC and LCMS indicated the SM was consumed. The mixture was concentrated. The residue was purified via silica gel chromatography (12 g, hexanes-30% EtOAc) to give desired 5-bromo-6-chloro-3-((3,3-difluoro-4-(4-fluorophenyl)-4-((triethylsilyl)oxy)pentyl)oxy)pyrazin-2-amine (82 mg, 0.148 mmol, 49.3% yield).

¹H NMR (499 MHz, chloroform-d) δ 7.51 (ddd, J=8.7, 5.3, 1.4 Hz, 2H), 7.08-7.03 (m, 2H), 4.89 (br s, 2H), 4.56-4.42 (m, 2H), 2.56-2.43 (m, 1H), 2.01-1.87 (m, 1H), 1.78 (s, 3H), 0.97 (t, J=7.9 Hz, 9H), 0.71-0.65 (m, 6H).

MS ESI m/z 556.2 (M+H)⁺.

### 81B: 3-bromo-2-chloro-5-((3,3-difluoro-4-(4-fluorophenyl)-4-((triethylsilyl)oxy)pentyl)oxy)-pyrazine

To a solution of 5-bromo-6-chloro-3-((3,3-difluoro-4-(4-fluorophenyl)-4-((triethylsilyl)oxy)-pentyl)oxy)pyrazin-2-amine (40 mg, 0.072 mmol) in THF (1 mL) was added isoamyl nitrite (0.029 mL, 0.216 mmol). The mixture was stirred at 45° C. for 3 h. LCMS indicated the SM was being consumed, and the product was formed. The reaction was stirred at rt for 20 h. LCMS indicated the SM was consumed. The mixture was concentrated. The residue was purified via silica gel chromatography (4 g, hexanes-10% EtOAc) to give crude 3-bromo-2-chloro-5-((3,3-difluoro-4-(4-fluorophenyl)-4-((triethylsilyl)oxy)pentyl)oxy)pyrazine (17 mg, 0.031 mmol, 43.7% yield).

MS ESI m/z 541.1 (M+H)⁺.

81C: tert-butyl (tert-butoxycarbonyl)(7-(3-chloro-6-((3,3-difluoro-4-(4-fluorophenyl)-4-((triethylsilyl)oxy)pentyl)oxy)pyrazin-2-yl)-[1,2,4]triazolo[1,5-a]pyridin-2-yl)carbamate A mixture of crude 3-bromo-2-chloro-5-((3,3-difluoro-4-(4-fluorophenyl)-4-((triethylsilyl)oxy)pentyl)oxy)pyrazine (17 mg, 0.031 mmol), B1 (14.49 mg, 0.031 mmol), PdCl₂(dppf)-CH₂Cl₂adduct (1.286 mg, 1.574 μmol) and tripotassium phosphate (0.047 mL, 0.094 mmol) in dioxane (0.2 mL) was stirred at 95° C. for 2 h. LCMS indicated the desired product was formed. Water was added and extracted with 2× EOAc.

The organic layer was concentrated to give crude product. The material was used for the next step.

MS ESI m/z 693.5 (M+H)⁺.

### 81: 5-((6-(2-amino-[1,2,4]triazolo[1,5-a]pyridin-7-yl)-5-chloropyrazin-2-yl)oxy)-3,3-difluoro-2-(4-fluorophenyl)pentan-2-ol

To a solution of a crude 81C (23 mg, 0.029 mmol) in DCM (0.3 mL) was added TFA (0.3 mL, 3.89 mmol). The mixture was stirred at 55° C. for 3 h. LCMS indicated the reaction was done. The mixture was concentrated. The crude material was purified via preparative Reverse Phase chromatography with the following conditions: Column: XBridge C18, 19 mm×200 mm, 5 m particles; Flow Rate: 20 mL/min; Column Temperature: 25° C.

Fraction collection was triggered by UV (220 nm) and MS (ESI+). Fractions containing the desired product were
combined and dried via centrifugal evaporation to give 5-((6-(2-amino-[1,2,4]triazolo[1,5-a]pyridin-7-yl)-5-chloropyrazin-2-yl)oxy)-3,3-difluoro-2-(4-fluorophenyl)pentan-2-ol (4.0 mg, 8.35 μmol, 28.8% yield).

¹H NMR (500 MHz, DMSO-d₆) δ 8.67 (d, J=6.9 Hz, 1H), 8.17 (s, 1H), 7.77 (s, 1H), 7.55 (br dd, J=7.3, 5.9 Hz, 2H), 7.26-7.23 (m, 1H), 7.17-7.12 (m, 2H), 4.53-4.45 (m, 2H), 2.50-2.40 (m, 1H), 2.19-2.05 (m, 1H), 1.59 (s, 3H).

MS ESI m/z 479.1 (M+H)⁺.

### Example 82: 4-(3-(2-amino-[1,2,4]triazolo[1,5-a]pyridin-7-yl)-6-chloro-2-fluorophenoxy)-2-fluoro-1-(4-fluorophenyl)butan-1-ol (isomer 1) Isomers 1 and 2 of 4-(3-(2-amino-[1,2,4]triazolo[1,5-a]pyridin-7-yl)-6-chloro-2-fluorophenoxy)-2-fluoro-1-(4-fluorophenyl)butan-1-ol

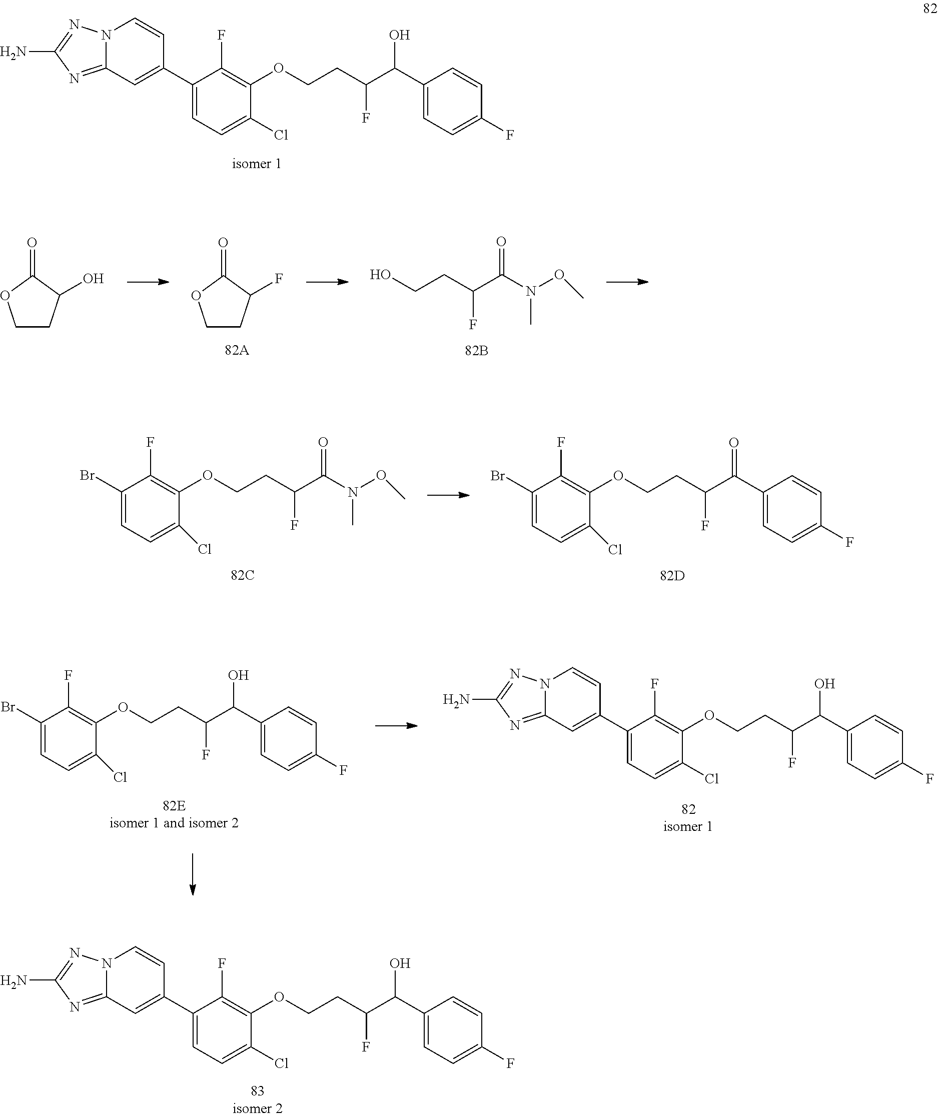

(The 4 unassigned diastereomers are:

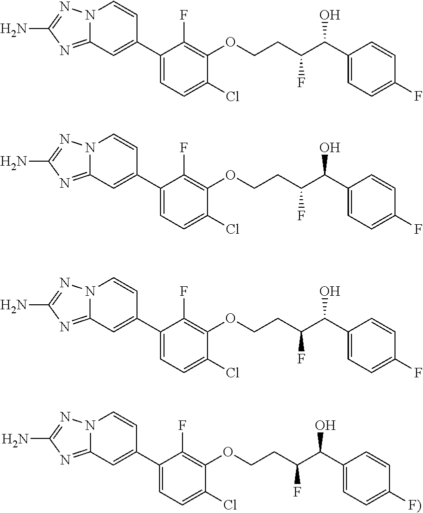

### 82A: (±)-3-fluorodihydrofuran-2(3H)-one

To a solution of DAST (16.69 ml, 126 mmol) in DCM (60 mL) at −78° C. behind a safety shield (face shield should be worn) was added a solution of 3-hydroxydihydrofuran-2(3H)-one (4.3 g, 42.1 mmol) in DCM (26 mL) dropwise over a period of about 15 min, and the reaction mixture was stirred at −78° C. for 1 h and then warmed up to rt and stirred at rt for 1 h. Reaction was monitored by TLC (40% EtOAc/Hex), KMnO₄ basic stain. No need for LC/MS. The reaction mixture was poured slowly to 200 mL water some ice in an ice bath with vigorous stirring (behind a safety shield and also wear face shield), and then 100 mL DCM was added. The aqueous layer was extracted with DCM (x3), and the combined organic layers were washed with brine. The solvent was removed in vacuo with bath temperature at 20° C. The crude product was purified by silica gel chromatography eluting with 0-40% ether/DCM to give the title compound (3.6 g, 93% purity, 7% DCM, 75% yield).

¹H NMR (400 MHz, chloroform-d) δ 5.30-5.09 (m, 1H), 4.52 (td, J=8.8, 4.1 Hz, 1H), 4.33 (ddd, J=9.3, 8.1, 6.9 Hz, 1H), 2.77-2.62 (m, 1H), 2.61-2.43 (m, 1H).

### 82B: (±)-2-fluoro-4-hydroxy-N-methoxy-N-methylbutanamide (A3E76-093)

To a suspension of N,O-dimethylhydroxylamine hydrochloride (2.68 g, 27.5 mmol) and (±)-3-fluorodihydrofuran-2(3H)-one (2.3 g, 18.34 mmol) in THF (70.5 ml) at −25° C. was added isopropylmagnesium chloride (27.5 ml, 55.0 mmol) dropwise over a period of 15 min. The reaction mixture was stirred at −25° C. for 30 min. Saturated NH₄C₁ was added, and the aqueous layer was extracted with EtOAc (x3). The combined organic layers were washed with brine, dried over anhydrous sodium sulfate, and filtered. The filtrate was evaporated in vacuo to give the crude product. The crude product was purified by silica gel chromatography eluting with 0-60% acetone/hexanes to give 2-fluoro-4-hydroxy-N-methoxy-N-methylbutanamide (807 mg, 27% yield) as a colorless oil.

¹H NMR (400 MHz, chloroform-d) δ 5.58-5.36 (m, 1H), 3.95-3.79 (m, 2H), 3.77 (s, 3H), 3.26 (s, 3H), 2.21-2.16 (m, 1H), 2.12 (q, J=5.9 Hz, 1H).

### 82C: (+)-4-(3-bromo-6-chloro-2-fluorophenoxy)-2-fluoro-N-methoxy-N-methylbutanamide

To a suspension of 2-fluoro-4-hydroxy-N-methoxy-N-methylbutanamide (29 mg, 0.176 mmol), triphenylphosphine (69.1 mg, 0.263 mmol), DIAD (51.2 μl, 0.263 mmol) in THF (702 μl) at rt was added DIAD (51.2 μl, 0.263 mmol), and the reaction mixture was stirred at rt for 12 h. The crude product was purified by preparative TLC on silica gel (0.50 mm thickness) eluting with 50% EtOAc/hexanes to give 4-(3-bromo-6-chloro-2-fluorophenoxy)-2-fluoro-N-methoxy-N-methylbutanamide (35 mg, 0.094 mmol, 53.5% yield).

¹H NMR (400 MHz, chloroform-d) δ 7.07-6.78 (m, 2H), 5.69-5.40 (m, 1H), 4.33-4.13 (m, 2H), 3.78 (s, 3H), 3.26 (s, 3H), 2.58-2.14 (m, 2H).

### 82D: (+)-4-(3-bromo-6-chloro-2-fluorophenoxy)-2-fluoro-1-(4-fluorophenyl)butan-1-one

To a solution of 4-(3-bromo-6-chloro-2-fluorophenoxy)-2-fluoro-N-methoxy-N-methylbutanamide (93 mg, 0.112 mmol) in THF (1.2 mL) at rt was added (4-fluorophenyl)-magnesium bromide (449 μl, 0.449 mmol) and the reaction mixture was stirred at rt for 1 h. Water was added and the aqueous layer was extracted with EtOAc (x3). The combined organic layers were washed with brine, dried over anhydrous sodium sulfate, and filtered, and the filtrate was evaporated in vacuo to give the crude product. The crude product was purified by preparative TLC on silica gel (0.50 mm thickness) eluting with 20% EtOAc/hexanes to give 4-(3-bromo-6-chloro-2-fluorophenoxy)-2-fluoro-1-(4-fluorophenyl)butan-1-one (33 mg, 0.081 mmol, 72.1% yield) as a colorless oil.

¹H NMR (500 MHz, CDCl₃) δ 8.17-8.03 (m, 2H), 7.29-7.24 (m, 1H), 7.24-7.16 (m, 2H), 7.11 (dd, J=8.8, 2.0 Hz, 1H), 6.19-6.01 (m, 1H), 4.55-4.20 (m, 2H), 2.72-2.48 (m, 1H), 2.43-2.21 (m, 1H).

### 82E: Isomers 1 and 2 of 4-(3-bromo-6-chloro-2-fluorophenoxy)-2-fluoro-1-(4-fluorophenyl)-butan-1-ol

To a solution of 4-(3-bromo-6-chloro-2-fluorophenoxy)-2-fluoro-1-(4-fluorophenyl)butan-1-one (33 mg, 0.081 mmol) in MeOH (810 μl) at rt was added sodium borohydride (4.59 mg, 0.121 mmol), and the reaction mixture was stirred at rt for 10 min. Water was added and the aqueous layer was extracted with EtOAc (x3). The combined organic layers were washed with brine, dried over anhydrous sodium sulfate, and filtered. The filtrate was evaporated in vacuo to give the crude product. The crude product was purified by preparative TLC on silica gel (0.50 mm thickness) eluting with 30% EtOAc/hexanes to give isomer 1(9 mg, 27% yield) and isomer 2 (7 mg, 21% yield).

Isomer 1 (first-eluting isomer): ¹H NMR (400 MHz, CDCl₃) δ 7.45-7.39 (m, 2H), 7.22 (dd, J=8.8, 6.7 Hz, 1H), 7.14-7.04 (m, 3H), 5.16-4.94 (m, 2H), 4.35-4.16 (m, 2H), 2.17-1.98 (m, 2H).

Isomer 2 (second-eluting isomer): ¹H NMR (500 MHz, CDCl₃) δ 7.45-7.38 (m, 2H), 7.22 (dd, J=8.8, 6.7 Hz, 1H), 7.13-7.04 (m, 3H), 5.10-4.90 (m, 1H), 4.82 (ddd, J=15.1, 6.2, 3.6 Hz, 1H), 4.24 (t, J=6.1 Hz, 2H), 2.10-1.93 (m, 2H).

### 82: Isomer 1 of 4-(3-(2-amino-[1,2,4]triazolo[1,5-a]pyridin-7-yl)-6-chloro-2-fluorophenoxy)-2-fluoro-1-(4-fluorophenyl)butan-1-ol

A mixture of isomer 1 of 4-(3-bromo-6-chloro-2-fluorophenoxy)-2-fluoro-1-(4-fluorophenyl)butan-1-ol (9 mg, 0.022 mmol), tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (10 mg, 0.022 mmol), PdCl₂(dtbpf) (1.4 mg, 2.197 μmol), and potassium phosphate (43.9 μl, 0.088 mmol) in dioxane (110 μL) was heated to 60° C. for 12 h under nitrogen. Brine and EtOAc (1 mL) were added and the aqueous layer was extracted twice with EtOAc. The organic phases were combined and concentrated to give a deep brown residue. The residue was dissolved in 50% TFA in DCM (0.5 mL) and stirred at rt for 12 h. The solvent was removed in vacuo, and the residue was purified via preparative LC/MS with the following conditions: Column: XBridge C18, 200 mm×19 mm, 5 m particles; Mobile Phase A: 5:95 acetonitrile: water with 10 mM ammonium acetate; Mobile Phase B: 95:5 acetonitrile: water with 10 mM ammonium acetate. Fractions containing the desired product were combined and dried to isomer 1 of the title compound (3.1 mg).

¹H NMR (500 MHz, DMSO-d₆) δ 8.62 (d, J=6.9 Hz, 1H), 7.52 (s, 1H), 7.48-7.36 (m, 4H), 7.17 (t, J=8.8 Hz, 2H), 7.03 (d, J=6.9 Hz, 1H), 4.97-4.74 (m, 2H), 4.31-4.15 (m, 2H), 2.25-1.94 (m, 2H).

MS ESI m/z 463.1 (M+H)⁺.

### Example 83: 4-(3-(2-amino-[1,2,4]triazolo[1,5-a]pyridin-7-yl)-6-chloro-2-fluorophenoxy)-2-fluoro-1-(4-fluorophenyl)butan-1-ol (isomer 2)

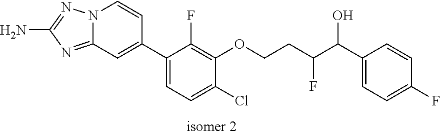 [SMILES: Nc1nc2cc(-c3ccc(Cl)c(OCCC(F)C(O)c4ccc(F)cc4)c3F)ccn2n1]

A mixture of isomer 2 of 4-(3-bromo-6-chloro-2-fluorophenoxy)-2-fluoro-1-(4-fluorophenyl)butan-1-ol (9 mg, 0.022 mmol), tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (10 mg, 0.022 mmol), PdCl₂(dtbpf) (1.4 mg, 2.197 μmol), and potassium phosphate (43.9 μL, 0.088 mmol) in dioxane (110 μL) was heated to 60° C. for 3 h under nitrogen. Brine and EtOAc (1 mL) were added and the aqueous layer was extracted twice with EtOAc. The organic phases were combined and concentrated to give a deep brown residue. The residue was dissolved in DCM (0.5 mL) and treated with 4 M HCl in dioxane at rt for 12 h. The solvent was removed in vacuo, and the residue was purified via preparative LC/MS with the following conditions: Column: XBridge C18, 200 mm×19 mm, 5 m particles; Mobile Phase A: 5:95 acetonitrile: water with 10 mM ammonium acetate; Mobile Phase B: 95:5 acetonitrile: water with 10 mM ammonium acetate. Fractions containing the desired product were combined and dried to give the title compound (1.5 mg).

¹H NMR (500 MHz, DMSO-d₆) δ 8.63 (d, J=7.0 Hz, 1H), 7.53 (s, 1H), 7.48-7.38 (m, 4H), 7.17 (t, J=8.9 Hz, 2H), 7.04 (br d, J=6.6 Hz, 1H), 5.78 (d, J=5.2 Hz, 1H), 4.98-4.68 (m, 2H), 4.31-4.13 (m, 2H), 2.10-1.93 (m, 2H).

MS ESI m/z 463.0 (M+H)⁺.

### Example 84: 4-(3-(2-amino-[1,2,4]triazolo[1,5-a]pyridin-7-yl)-2-fluorophenoxy)-2-fluoro-1-(4-fluorophenyl)butan-1-ol

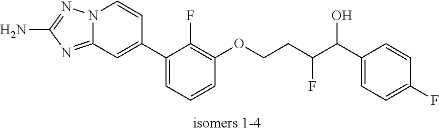 [SMILES: Nc1nc2cc(-c3cccc(OCCC(F)C(O)c4ccc(F)cc4)c3F)ccn2n1]

Isomers 1, 2, 3 and 4 of 4-(3-(2-amino-[1,2,4]triazolo[1,5-a]pyridin-7-yl)-2-fluorophenoxy)-2-fluoro-1-(4-fluorophenyl)butan-1-ol

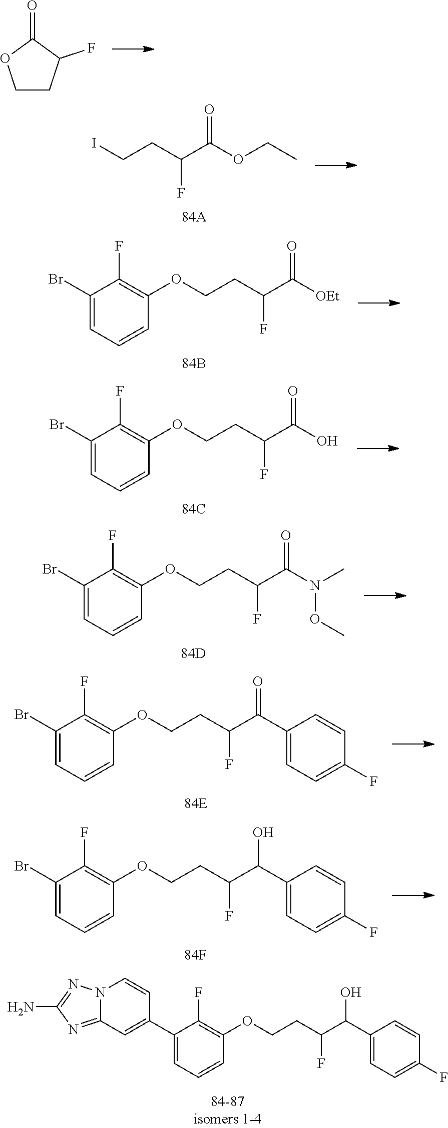

(The 4 unassigned diastereomers are:

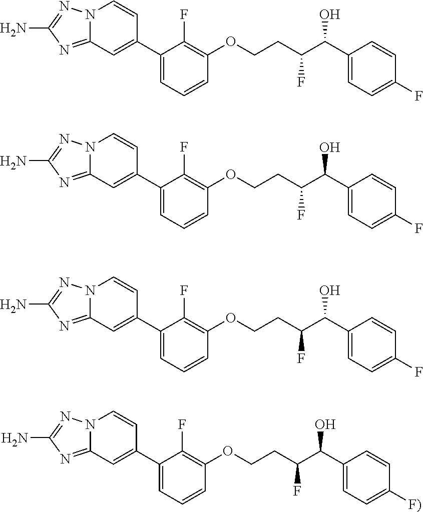

### 84A: (1)-ethyl 2-fluoro-4-iodobutanoate

To a solution of 3-fluorodihydrofuran-2(3H)-one (10.8 g, 97 mmol) in anhydrous DCM (322 ml) was added ethanol (16.90 ml, 290 mmol). The reaction was then treated with neat TMSI (20.60 ml, 145 mmol) over 2 min (via syringe). After 15 min the bath was removed and the reaction was allowed to stir and slowly warm up to rt. The reaction mixture was evaporated in vacuo, and the residue was dissolved in ether (425 mL) and washed with 5% sodium bisulfite (2×15 mL), and brine (1×5 mL). The crude product was purified by silica gel chromatography eluting with a linear gradient from 100% hexanes to 100% DCM to give the title compound as a pale orange liquid (9.3 g).

¹H NMR (500 MHz, CDCl₃) δ 5.09-4.93 (m, 1H), 4.30 (q, J=7.2 Hz, 2H), 3.40-3.22 (m, 2H), 2.52-2.31 (m, 2H), 1.38-1.30 (m, 3H).

### 84B: (1)-ethyl 4-(3-bromo-2-fluorophenoxy)-2-fluorobutanoate

A mixture of 3-bromo-2-fluorophenol (0.808 g, 4.23 mmol), ethyl 2-fluoro-4-iodobutanoate (1 g, 3.85 mmol), and potassium carbonate (0.797 g, 5.77 mmol) in acetone (19.23 ml) was heated at 60° C. for 12 h. The reaction mixture was filtered, and the solid was washed with acetone, and the filtrate was evaporated in vacuo. EtOAc was added, and this solution was washed with 1N NaOH 3 times. The organic layer was washed with brine, dried and then evaporated in vacuo to give ethyl 4-(3-bromo-2-fluorophenoxy)-2-fluorobutanoate (726 mg, 2.247 mmol, 58.4% yield) as a colorless oil.

¹H NMR (500 MHz, chloroform-d) δ 7.16 (td, J=6.6, 2.3 Hz, 1H), 7.02-6.86 (m, 2H), 5.32-5.12 (m, 1H), 4.34-4.28 (m, 2H), 4.26-4.17 (m, 2H), 2.58-2.33 (m, 2H), 1.36-1.31 (m, 3H)

### 83C: (±)-4-(3-bromo-2-fluorophenoxy)-2-fluorobutanoic acid

A mixture of ethyl 4-(3-bromo-2-fluorophenoxy)-2-fluorobutanoate (0.73 g, 2.259 mmol) and lithium hydroxide (0.108 g, 4.52 mmol) in THF/water (1:1) (11.30 ml) was stirred at rt for 2 h and LC/MS and TLC showed a complete reaction. HCl (5.65 ml, 5.65 mmol) was added, and white precipitate formed. The aqueous layer was extracted with EtOAc (x3). The combined organic layers were washed with brine, dried over anhydrous sodium sulfate, and filtered The filtrate was evaporated in vacuo to give 4-(3-bromo-2-fluorophenoxy)-2-fluorobutanoic acid (620 mg, 2.101 mmol, 93% yield) as a white solid.

¹H NMR (500 MHz, chloroform-d) δ 7.20-7.11 (m, 1H), 7.03-6.89 (m, 2H), 5.40-5.21 (m, 1H), 4.43-4.18 (m, 2H), 2.68-2.32 (m, 2H).

### 83D: (+)-4-(3-bromo-2-fluorophenoxy)-2-fluoro-N-methoxy-N-methylbutanamide

A mixture of 4-(3-bromo-2-fluorophenoxy)-2-fluorobutanoic acid (0.62 g, 2.101 mmol), N,O-dimethylhydroxylamine hydrochloride (0.246 g, 2.52 mmol), BOP (1.115 g, 2.52 mmol) and Hunig's base (1.284 ml, 7.35 mmol) in DCM (10.51 ml) was stirred at rt for 3 h. LC/MS showed a complete reaction. Water was added and the aqueous layer was extracted with EtOAc (x3). The combined organic layers were washed with brine, dried over anhydrous sodium sulfate, and filtered, and the filtrate was evaporated in vacuo to give the crude product. The crude product was purified by silica gel chromatography eluting with 0-60% EtOAc/hexanes to give the title compound (595 mg, 1.760 mmol, 84% yield) as a white solid.

¹H NMR (500 MHz, chloroform-d) δ 7.23-7.11 (m, 1H), 7.01-6.90 (m, 2H), 5.67-5.47 (m, 1H), 4.32-4.19 (m, 2H), 3.78 (s, 3H), 3.27 (s, 3H), 2.53-2.27 (m, 2H).

### 83E: (+)-4-(3-bromo-2-fluorophenoxy)-2-fluoro-1-(4-fluorophenyl)butan-1-one

To a solution of 4-(3-bromo-2-fluorophenoxy)-2-fluoro-N-methoxy-N-methylbutanamide (0.595 g, 1.760 mmol) at rt was added (4-fluorophenyl)magnesium bromide (2.64 ml, 2.64 mmol) dropwise over a period of 5 min, and the reaction mixture was stirred at rt for 30 min. LC/MS showed a complete reaction. Saturated NH₄C₁ was added and the aqueous layer was extracted with EtOAc (x3). The combined organic layers were washed with brine, dried over anhydrous sodium sulfate, and filtered. The filtrate was evaporated in vacuo to give the crude product. The crude product was purified by silica gel chromatography eluting with 0-40% EtOAc/hexanes to give the title compound (0.596 g, 1.597 mmol, 91% yield) as a colorless oil.

¹H NMR (500 MHz, CDCl₃) δ 8.16-8.04 (m, 2H), 7.25-7.14 (m, 3H), 7.01-6.92 (m, 2H), 6.15-5.78 (m, 1H), 4.39-4.19 (m, 2H), 2.69-2.49 (m, 1H), 2.46-2.33 (m, 1H).

### 84F: 4-(3-bromo-2-fluorophenoxy)-2-fluoro-1-(4-fluorophenyl)butan-1-ol

To a solution of 4-(3-bromo-2-fluorophenoxy)-2-fluoro-1-(4-fluorophenyl)butan-1-one (195 mg, 0.523 mmol) in MeOH (2613 μl) at rt was added NaBH₄ (29.7 mg, 0.784 mmol), and the reaction mixture was stirred at rt for 30 min. MeOH was removed in vacuo, water was added and the aqueous layer was extracted with EtOAc (x3). The combined organic layers were washed with brine, dried over anhydrous sodium sulfate, and filtered. The filtrate was evaporated in vacuo to give crude 4-(3-bromo-2-fluorophenoxy)-2-fluoro-1-(4-fluorophenyl)butan-1-ol (210 mg) as a white solid. This crude material as a mixture of diastereomers was used directly for the next step.

### 84: Isomers 1, 2, 3 and 4 of 4-(3-(2-amino-[1,2,4]triazolo[1,5-a]pyridin-7-yl)-2-fluorophenoxy)-2-fluoro-1-(4-fluorophenyl)butan-1-ol

A mixture of tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (282 mg, 0.613 mmol), 4-(3-bromo-2-fluorophenoxy)-2-fluoro-1-(4-fluorophenyl)butan-1-ol (209 mg, 0.557 mmol), and PdCl₂(dtbpf) (18.15 mg, 0.028 mmol), and potassium phosphate (1114 μl, 2.228 mmol) in dioxane (2785 μl) was heated under nitrogen at 60° C. for 1 h. Water was added and the aqueous layer was extracted with EtOAc (x3). The combined organic layers were washed with brine, dried over anhydrous sodium sulfate, and filtered The filtrate was evaporated in vacuo to give the crude product. The product was dissolved in MeOH (2.5 mL) and 4M HCl in dioxane (1393 μl, 5.57 mmol) was added. The reaction mixture was heated at 60° C. for 2.5 h. MeOH was removed in vacuo and the residue was purified via preparative LC/MS with the following conditions: Column: XBridge C18, 200 mm×19 mm, 5 m particles; mobile phase A: 5:95 acetonitrile: water with 10 mM ammonium acetate; mobile phase B: 95:5 acetonitrile: water with 10 mM ammonium acetate. Fractions containing the desired product were combined and dried to give the first-eluted isomer A (76 mg, 31% yield) and second-eluted isomer B (48 mg, 18% yield).

The first-eluted isomer A was purified via preparative SFC chromatography with the following conditions: Column: Chiralcel OJ-H, 30 mm×250 mm, 5 m particles; Flow Rate: 85 mL/min; Column Temperature: 45° C. Gradient: mobile phase A (CO2) and mobile phase B (MeOH with 0.1% DEA). SFC purification gave the first-eluted enantiomer as isomer 1 (21 mg) and the second-eluted enantiomer as isomer 2 of 4-(3-(2-amino-[1,2,4]triazolo[1,5-a]pyridin-7-yl)-2-fluorophenoxy)-2-fluoro-1-(4-fluorophenyl)butan-1-ol (21 mg).

### 84—Isomer 1

1H NMR (500 MHz, DMSO-d₆) δ 8.60 (d, J=6.8 Hz, 1H), 7.54-7.40 (m, 3H), 7.32-7.11 (m, 5H), 7.03 (br d, J=6.5 Hz, 1H), 6.05 (s, 2H), 5.78 (d, J=4.6 Hz, 1H), 4.95-4.69 (m, 2H), 4.32-4.07 (m, 2H).

MS ESI m/z 429.0 (M+H)⁺.

85—Isomer 2: the same MS and ¹H NMR data as isomer 1.

### Example 86-87

The second-eluted isomer B was purified via preparative SFC with the following conditions: Column: Chiralcel OJ-H, 30 mm×250 mm, 5 m particles; Flow Rate: 85 mL/min; Column Temperature: 45° C. Gradient: mobile phase A (CO2) and mobile phase B (MeOH with 0.1% DEA). SFC purification gave the first-eluted enantiomer as isomer 3 86 (11 mg) and the second-eluted enantiomer as isomer 4 of 4-(3-(2-amino-[1,2,4]triazolo[1,5-a]pyridin-7-yl)-2-fluorophenoxy)-2-fluoro-1-(4-fluorophenyl)butan-1-ol 87 (10 mg).

### 86—Isomer 3

¹H NMR (500 MHz, DMSO-d₆) δ 8.64-8.49 (m, 1H), 7.55-7.43 (m, 3H), 7.26-7.14 (m, 5H), 7.03 (br d, J=6.7 Hz, 1H), 6.05 (s, 2H), 5.84 (d, J=4.1 Hz, 1H), 4.90-4.70 (m, 2H), 4.32-4.12 (m, 2H).

MS ESI m/z 429.0 (M+H)⁺.

87—Isomer 4: the same MS and ¹H NMR data as isomer 3.

### Example 88: 5-(3-(2-amino-[1,2,4]triazolo[1,5-a]pyridin-7-yl)-2-fluorophenoxy)-3-fluoro-2-(4-fluorophenyl)pentan-2-ol

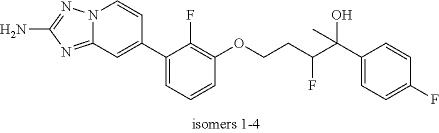 [SMILES: CC(O)(c1ccc(F)cc1)C(F)CCOc1cccc(-c2ccn3nc(N)nc3c2)c1F]

### Isomers 1, 2, 3 and 4 of 5-(3-(2-amino-[1,2,4]triazolo[1,5-a]pyridin-7-yl)-2-fluorophenoxy)-3-fluoro-2-(4-fluorophenyl)pentan-2-ol

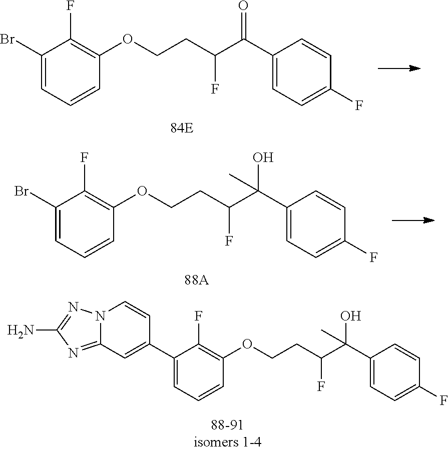 [SMILES: O=C(c1ccc(F)cc1)C(F)CCOc1cccc(Br)c1F ; CC(O)(c1ccc(F)cc1)C(F)CCOc1cccc(Br)c1F ; CC(O)(c1ccc(F)cc1)C(F)CCOc1cccc(-c2ccn3nc(N)nc3c2)c1F]

(The 4 unassigned diasteromers are:

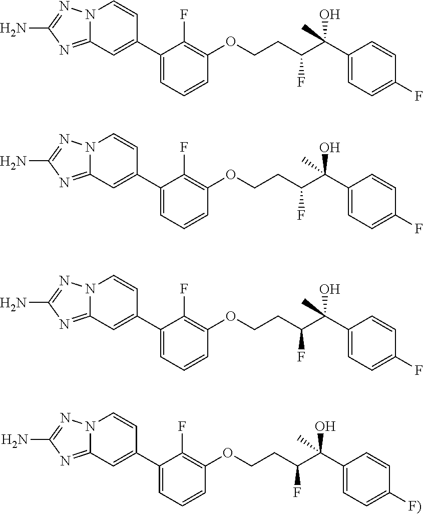

### 88A: 5-(3-bromo-2-fluorophenoxy)-3-fluoro-2-(4-fluorophenyl)pentan-2-ol

To a solution of 4-(3-bromo-2-fluorophenoxy)-2-fluoro-1-(4-fluorophenyl)butan-1-one 84E (195 mg, 0.523 mmol) in THF (2613 μl) at rt was added methylmagnesium bromide in ether (261 μl, 0.784 mmol), and the reaction mixture was stirred at rt for 2 h. Sat. NH₄C₁ was added and the aqueous layer was extracted with EtOAc (x3). The combined organic layers were washed with brine, dried over anhydrous sodium sulfate, and filtered The filtrate was evaporated in vacuo to give 5-(3-bromo-2-fluorophenoxy)-3-fluoro-2-(4-fluorophenyl)pentan-2-ol (205 mg). The crude product was used directly for the next step without further purification.

### 88: Isomers 1, 2, 3 and 4 of 5-(3-(2-amino-[1,2,4]triazolo[1,5-a]pyridin-7-yl)-2-fluorophenoxy)-3-fluoro-2-(4-fluorophenyl)pentan-2-ol

A mixture of 5-(3-bromo-2-fluorophenoxy)-3-fluoro-2-(4-fluorophenyl)pentan-2-ol (200 mg, 0.514 mmol), tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (260 mg, 0.565 mmol), PdCl₂(dtbpf) (16.75 mg, 0.026 mmol) and potassium phosphate (1.1 mL, 2.055 mmol) in dioxane (2.6 mL) was purged with N₂ for 3 min and then heated at 60° C. for 1 h. Water was added and the aqueous layer was extracted with EtOAc (x3). The combined organic layers were washed with brine, dried over anhydrous sodium sulfate, and filtered. The filtrate was evaporated in vacuo to give the crude product. The crude product was dissolved in MeOH (3 mL) and HCl, 4M in dioxane (1.3 mL, 5.14 mmol) was added. The reaction mixture was heated at 60° C. for 3 h. The crude material was purified via preparative LC/MS (Column: XBridge C18, 200 mm×19 mm, 5-μm particles; mobile phase A: 5:95 acetonitrile: water with 0.1% trifluoroacetic acid; mobile phase B: 95:5 acetonitrile: water with 0.1% trifluoroacetic acid) to give a mixture of two diastereomers (152 mg 58.0% yield). This mixture was further purified via preparative SFC chromatography (column: Chiralcel OJ-H, 30 mm×250 mm, 5 m particles; mobile phase A (C₀₂); mobile phase B (MeOH with 0.1% DEA)) to give the following four isomers of 5-(3-(2-amino-[1,2,4]triazolo[1,5-a]pyridin-7-yl)-2-fluorophenoxy)-3-fluoro-2-(4-fluorophenyl)pentan-2-ol:

88—Isomer 1 (4.5 mg): retention time=5.0 min

MS ESI m/z 443.1 (M+H)⁺

89—Isomer 2 (4.6 mg): retention time=5.4 min

MS ESI m/z 443.1 (M+H)⁺

90—Isomer 3 (51 mg): retention time=6.4 min

MS ESI m/z 443.1 (M+H)⁺

¹H NMR (500 MHz, DMSO-d₆) δ 8.66 (d, J=6.9 Hz, 1H), 7.60-7.47 (m, 3H), 7.28-7.07 (m, 6H), 4.91-4.66 (m, 1H), 2.09-1.95 (m, 1H), 1.88-1.67 (m, 1H), 1.54 (s, 3H).

91—Isomer 4 (54 mg): retention time=8.4 min

MS and ¹H MR data are the same as isomer 3.

Analytical SFC was used to determine the % ee and retention time. Conditions: Column: Chiralcel OJ-H, 4.6 mm×100 mm, 5 m particles; mobile phase A: CO2; mobile phase B: MeOH; temperature: 50° C.; Isocratic elution at 15% B over 12 min; Flow: 2 mL/min; Detection: UV (220 nm).

### Example 92: 5-(5-(2-amino-[1,2,4]triazolo[1,5-a]pyridin-7-yl)-2,3-difluorophenoxy)-3-fluoro-2-(4-fluorophenyl)pentan-2-ol

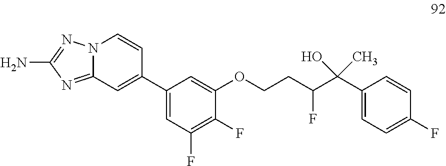 [SMILES: CC(O)(c1ccc(F)cc1)C(F)CCOc1cc(-c2ccn3nc(N)nc3c2)cc(F)c1F]

(The 4 unassigned diasteromers are:

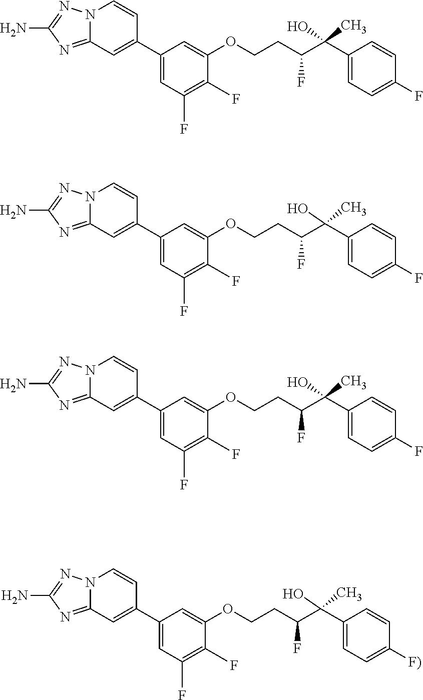

Preparation similar to example 84.

### 92A: ethyl 2-fluoro-4-iodobutanoate

To a solution of 3-fluorodihydrofuran-2(3H)-one (5.1 g, 49.0 mmol) in anhydrous DCM (100 mL) was added ethanol (8.6 mL, 147 mmol), followed by neat trimethylsilyl iodide (9.8 mL, 68.9 mmol) over 2 min. After the addition was complete, the reaction was placed into an ice/water bath for 15 min and then allowed to stir while slowly warming to room temp over 18 hours. The solvent was removed in vacuo and the residue was dissolved in EtOAc (425 mL) and the organic layer was washed with 5% sodium bisulfite in water (2×15 mL), brine (1×5 mL), dried over sodium sulfate, filtered and evaporated to dryness. The crude residue was purified by flash chromatography on silica gel, eluting with 0-100% gradient of dichloromethane in hexane to afford the title compound, ethyl 2-fluoro-4-iodobutanoate (9.28 g, 35.68 mmol, 72.8%).

¹H NMR (500 MHz, chloroform-d) δ 7.24 (dd, J=8.7, 6.7 Hz, 1H), 7.08 (dd, J=8.8, 1.9 Hz, 1H), 5.40-5.22 (m, 1H), 4.37-4.21 (m, 4H), 2.60-2.40 (m, 1H), 2.29 (ddt, J=17.6, 14.9, 4.6 Hz, 1H), 1.34 (t, J=7.2 Hz, 3H)

### 92B: (A3255-027): ethyl 4-(5-bromo-2,3-difluorophenoxy)-2-fluorobutanoate

To a 150 mL pressure bottle under N₂ was added ethyl 2-fluoro-4-iodobutanoate (1.0 g, 3.85 mmol), 5-bromo-2,3-difluorophenol (885 mg, 4.23 mmol), potassium carbonate (800 mg, 5.79 mmol) and acetone (30 mL). The reaction was securely capped and heated at 65° C. for 18 h. The reaction was filtered through a pad of celite. The pad was rinsed well with acetone and the solvent was removed in vacuo. The residue was dissolved in dichloromethane, filtered through a 45 millimicron frit and the solvent was removed in vacuo to give the title compound, ethyl 4-(5-bromo-2,3-difluorophenoxy)-2-fluorobutanoate (1.47 g, 4.31 mmol, quant) that was used without further purification.

¹H NMR (500 MHz, chloroform-d) δ 7.04-6.96 (m, 1H), 6.92 (dt, J=6.4, 2.1 Hz, 1H), 5.27-5.07 (m, 1H), 4.34-4.27 (m, 2H), 4.25-4.18 (m, 2H), 2.57-2.33 (m, 2H), 1.40-1.39 (m, 1H), 1.35 (t, J=7.2 Hz, 3H)

### 92C (A3255-028): 4-(5-bromo-2,3-difluorophenoxy)-2-fluorobutanoic acid

To a solution of ethyl 4-(5-bromo-2,3-difluorophenoxy)-2-fluorobutanoate (1.47 g, 4.31 mmol) in a mixture of THF (20 mL) and water (10 mL) was added lithium hydroxide (252.3 mg, 10.54 mmol). The reaction was sonicated briefly to break up the solid pieces of lithium hydroxide, then allowed to stir at rt for 70 min. Most of the solvent was removed in vacuo, and the residue was cooled in an ice/water bath and slowly treated with HCl, 1M in water (11 mL, 11.00 mmol) with rapid swirling. The reaction was diluted with EtOAc (150 mL), and the layers were separated. The water layer was back extracted with additional EtOAc (2×25 mL), the organic layers were combined, washed with brine (1×10 mL), dried over sodium sulfate, filtered and evaporated to dryness to give the title compound, 4-(5-bromo-2,3-difluorophenoxy)-2-fluorobutanoic acid (1.26 g, 4.02 mmol, 93%) that was used directly without further purification.

### 92D (A3255-029): 4-(5-bromo-2,3-difluorophenoxy)-2-fluoro-N-methoxy-N-methylbutanamide

To a solution of 4-(5-bromo-2,3-difluorophenoxy)-2-fluorobutanoic acid (1.26 g, 4.02 mmol) and N,O-dimethylhydroxylamine (475 mg, 4.87 mmol) in DCM was added N,N-diisopropyl-ethylamine (3.5 mL, 20.04 mmol). The reaction was flushed briefly with N₂, treated with benzotriazol-1-yloxytris(dimethylamino)phosphonium hexafluorophosphate (2.3 g, 5.20 mmol), capped and allowed to stir at rt for 18 h. The solvent was removed in vacuo and the crude residue was purified by flash chromatography on silica gel, eluting with 0-75% gradient of EtOAc in hexane to afford the title compound, 4-(5-bromo-2,3-difluorophenoxy)-2-fluoro-N-methoxy-N-methylbutanamide (1.01 g, 2.84 mmol, 70.5%).

¹H NMR (500 MHz, chloroform-d) δ 6.99 (ddd, J=9.2, 6.2, 2.3 Hz, 1H), 6.93 (dt, J=6.4, 2.2 Hz, 1H), 5.62-5.43 (m, 1H), 4.32-4.17 (m, 2H), 3.78 (s, 3H), 3.27 (s, 3H), 2.55-2.30 (m, 2H).

LCMS (ESI, m/z): 355.9, 357.9 [M+H]⁺

### 92E (A3255-032): 4-(5-bromo-2,3-difluorophenoxy)-2-fluoro-1-(4-fluorophenyl)butan-1-one

To an ice cold solution of 4-(5-bromo-2,3-difluorophenoxy)-2-fluoro-N-methoxy-N-methylbutanamide (1.01 g, 2.84 mmol) in anhydrous THF (20 mL) was slowly added 4-fluorophenylmagnesium bromide 1.0 M solution in THF (3.4 mL, 3.40 mmol). The reaction was stirred cold for 15 min, then the bath was removed and the reaction was allowed to stir at rt for 4 h 45 min. The reaction was quenched by the addition of aqueous saturated ammonium chloride (10 mL) and then diluted with EtOAc (40 mL). The aqueous layer was washed with EtOAc (3×40 mL), the organic layers were combined, washed with brine, dried over sodium sulfate, filtered and evaporated to dryness. The crude residue was purified by flash chromatography on silica gel, eluting with 0-65% gradient of DCM in hexane to afford the title compound, 4-(5-bromo-2,3-difluorophenoxy)-2-fluoro-1-(4-fluorophenyl)butan-1-one (931 mg, 2.38 mmol, 84%).

¹H NMR (500 MHz, chloroform-d) δ 8.16-8.04 (m, 2H), 7.26-7.15 (m, 2H), 7.02 (ddd, J=9.0, 6.4, 2.3 Hz, 1H), 6.94 (dt, J=6.4, 2.1 Hz, 1H), 6.03-5.81 (m, 1H), 4.40-4.20 (m, 2H), 2.70-2.50 (m, 1H), 2.50-2.32 (m, 1H).

### 92F (A3255-075): 5-(5-bromo-2,3-difluorophenoxy)-3-fluoro-2-(4-fluorophenyl)pentan-2-ol

To a solution of 4-(5-bromo-2,3-difluorophenoxy)-2-fluoro-1-(4-fluorophenyl)butan-1-one (150 mg, 0.383 mmol) in THF (3 mL) under N₂ was added methylmagnesium bromide 3.0 M solution in diethyl ether (385 μL, 1.155 mmol) with rapid swirling and the reaction was allowed to stand at room temp for 50 min. Additional methylmagnesium bromide 3.0 M solution in diethyl ether (385 μL, 1.155 mmol) was added and the reaction was allowed to stand at rt for 45 min. The reaction was quenched by the addition of aqueous saturated ammonium chloride (5 mL) and diluted with EtOAc (75 mL). The aqueous layer was extracted with EtOAc (15 mL). The organic layers were combined, washed with water (2×5 mL), brine (1×5 mL), dried over sodium sulfate, filtered and evaporated to dryness. The crude residue was purified by flash chromatography on silica gel, eluting with 0-100% gradient of DCM in hexane to afford the title compound, 5-(5-bromo-2,3-difluorophenoxy)-3-fluoro-2-(4-fluorophenyl)pentan-2-ol (112 mg, 0.275 mmol, 71.7%).

¹H NMR (500 MHz, chloroform-d) δ 7.52-7.38 (m, 2H), 7.14-7.02 (m, 2H), 6.96 (ddd, J=9.2, 6.2, 2.3 Hz, 1H), 6.82 (dt, J=6.4, 2.2 Hz, 1H), 4.99-4.76 (m, 1H), 4.21-3.95 (m, 2H), 2.13-2.08 (m, 1H), 1.92-1.74 (m, 1H), 1.70 (d, J=2.0 Hz, 3H).

### 92G (A3255-077): tert-butyl (tert-butoxycarbonyl)(7-(3,4-difluoro-5-((3-fluoro-4-(4-fluorophenyl)-4-hydroxypentyl)oxy)phenyl)-[1,2,4]triazolo[1,5-a]pyridin-2-yl)carbamate

To the flask containing 5-(5-bromo-2,3-difluorophenoxy)-3-fluoro-2-(4-fluorophenyl)pentan-2-ol (112 mg, 0.275 mmol) was added tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (140 mg, 0.304 mmol), dioxane (6 mL) and potassium phosphate tribasic, 2.0 M in water (550 μL, 1.100 mmol). The reaction was flushed with argon and stirred until all solids went into solution. The reaction was then treated with dichloro[1,1′-bis(di-t-butylphosphino)ferrocene]palladium(II), 99% (12 mg, 0.018 mmol), flushed well with argon again, equipped with a cold water condenser and heated at 115° C. for 2.5 h. The reaction was diluted with water (15 mL) and EtOAc (100 mL) and the layers were separated. The aqueous layer was back extracted with EtOAc (1×25 mL) and the organic layers were combined, washed with water (2×10 mL), brine (1×10 mL), dried over sodium sulfate, filtered and evaporated to dryness. The crude residue was purified by flash chromatography on silica gel, eluting with 0-100% gradient of EtOAc in hexane to afford the title compound, tert-butyl (tert-butoxycarbonyl)(7-(3,4-difluoro-5-((3-fluoro-4-(4-fluorophenyl)-4-hydroxypentyl)oxy)phenyl)-[1,2,4]triazolo[1,5-a]pyridin-2-yl)carbamate (144.3 mg, 0.218 mmol, 79%).

LCMS (ESI, m/z): 661.2 [M+H]+.

### 92: 5-(5-(2-amino-[1,2,4]triazolo[1,5-a]pyridin-7-yl)-2,3-difluorophenoxy)-3-fluoro-2-(4-fluorophenyl)pentan-2-ol enantiomer 1

To a solution of tert-butyl (tert-butoxycarbonyl)(7-(3,4-difluoro-5-((3-fluoro-4-(4-fluorophenyl)-4-hydroxypentyl)oxy)phenyl)-[1,2,4]triazolo[1,5-a]pyridin-2-yl)carbamate (144 mg, 0.218 mmol) in MeOH (2.0 mL) was added HCL 4.0 M solution in 1 4-dioxane (410 μL, 1.640 mmol). The reaction was capped and allowed to stand at room temp, for 35 min, then heated at 65° C. for 3 h 45 min. The reaction was then allowed to stand overnight at rt. The reaction was then heated to 60° C. for 3 h, or until judged to be complete by LC/MS. The volatiles were removed under a gentle stream of nitrogen and the crude material was purified via preparative LC/MS with the following conditions: Column: Waters XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 25-65% B over 20 minutes; Flow Rate: 20 mL/min; Column Temperature: 25 C. Fraction collection was triggered by MS and UV signals. Fractions containing the desired product were combined and dried via centrifugal evaporation to give racemic 5-(5-(2-amino-[1,2,4]triazolo[1,5-a]pyridin-7-yl)-2,3-difluorophenoxy)-3-fluoro-2-(4-fluorophenyl)pentan-2-ol. This racemic material was then further purified via preparative SFC with the following conditions: Column: Chiralpak IA, 30 mm×250 mm, 5 m particles; Mobile Phase A: supercritical CO2; Mobile Phase B: i-PrOH with 0.1% NH₄₀H; Elution gradient: isocratic 40% B over 25 minutes; Flow Rate: 85 mL/min; Column Temperature: 40° C. Fraction collection was triggered by UV (240 nm). Fractions containing the first isomer to elute from the preparative SFC column were combined and dried via centrifugal evaporation to provide 75-(5-(2-amino-[1,2,4]triazolo[1,5-a]pyridin-7-yl)-2,3-difluorophenoxy)-3-fluoro-2-(4-fluorophenyl)pentan-2-ol enantiomer 1 (27.3 mg, 0.059 mmol, 27.2%).

¹H NMR (500 MHz, DMSO-d₆) δ 8.59 (d, J=6.9 Hz, 1H), 7.73 (s, 1H), 7.56-7.44 (m, 3H), 7.35 (br d, J=6.3 Hz, 1H), 7.22 (dd, J=7.0, 1.7 Hz, 1H), 7.13 (t, J=8.8 Hz, 2H), 6.06 (s, 2H), 5.55 (s, 1H), 4.87-4.66 (m, 1H), 4.27 (br dd, J=5.7, 3.0 Hz, 2H), 2.13-1.92 (m, 1H), 1.91-1.68 (m, 1H), 1.54 (s, 3H).

LCMS (ESI, m/z): 461.1 [M+H]+.

### Example 93: 5-(5-(2-amino-[1,2,4]triazolo[1,5-a]pyridin-7-yl)-2,3-difluorophenoxy)-3-fluoro-2-(4-fluorophenyl)pentan-2-ol enantiomer 2

The title compound 5-(5-(2-amino-[1,2,4]triazolo[1,5-a]pyridin-7-yl)-2,3-difluorophenoxy)-3-fluoro-2-(4-fluorophenyl)pentan-2-ol enantiomer 2 (27.3 mg, 0.059 mmol, 27.2%)) was obtained as the second eluting isomer from the preparative SFC column in the preparation of Example 92.

¹H NMR (500 MHz, DMSO-d₆) δ 8.59 (d, J=6.9 Hz, 1H), 7.73 (s, 1H), 7.59-7.43 (m, 3H), 7.35 (br d, J=6.3 Hz, 1H), 7.22 (dd, J=7.0, 1.7 Hz, 1H), 7.13 (t, J=8.8 Hz, 2H), 6.06 (s, 2H), 5.55 (s, 1H), 4.84-4.69 (m, 1H), 4.37-4.20 (m, 2H), 2.10-1.95 (m, 1H), 1.91-1.70 (m, 1H), 1.54 (s, 3H).

MS ESI m/z 461.1 (M+H)⁺.

### Example 94: 5-(3-(2-amino-[1,2,4]triazolo-[1,5-a]pyridin-7-yl)-2,6-difluorophenoxy)-3-fluoro-2-(4-fluorophenyl)pentan-2-ol enantiomer 1

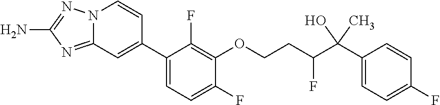 [SMILES: CC(O)(c1ccc(F)cc1)C(F)CCOc1c(F)ccc(-c2ccn3nc(N)nc3c2)c1F]

(The 4 unassigned diasteromers are:

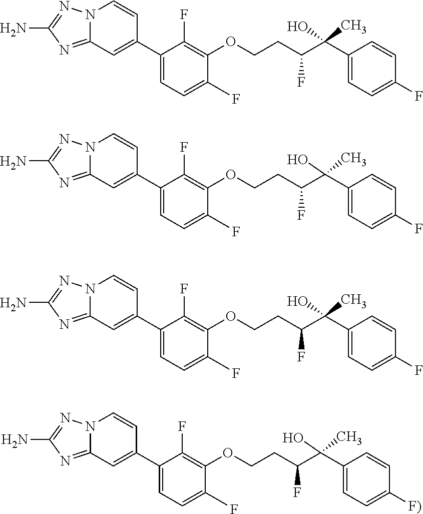

### 94A: ethyl 4-(3-bromo-2,6-difluorophenoxy)-2-fluorobutanoate

To a dry 100 mL flask under N₂ was added 3-bromo-2,6-difluorophenol (845 mg, 4.04 mmol), acetone (35 mL) and ethyl 2-fluoro-4-iodobutanoate (1.0 g, 3.85 mmol). The reaction was flushed briefly with N₂, treated with potassium carbonate (800 mg, 5.79 mmol), equipped with a cold water condenser and heated at 60° C. for 18 h. The reaction was filtered through a small pad of celite, the pad rinsed well with acetone and the solvent removed in vacuo. The residue was redissolved in EtOAc (200 mL) and the organic layer was washed with aqueous 1.0 M sodium hydroxide (3×10 mL), water (1×10 mL), brine (1×10 mL), dried over sodium sulfate, filtered and evaporated to dryness to give the title compound, ethyl 4-(3-bromo-2,6-difluorophenoxy)-2-fluorobutanoate (1.04 g, 3.05 mmol, 79%) that was used without further purification.

¹H NMR (500 MHz, chloroform-d) δ 7.25-7.17 (m, 1H), 6.93-6.77 (m, 1H), 5.38-5.14 (m, 1H), 4.44-4.24 (m, 4H), 2.58-2.22 (m, 2H), 1.40-1.28 (m, 3H).

### 94B: 4-(3-bromo-2,6-difluorophenoxy)-2-fluorobutanoic acid

To a solution of ethyl 4-(3-bromo-2,6-difluorophenoxy)-2-fluorobutanoate (1.04 g, 3.05 mmol) in a mixture of THF (15 mL) and water (7.5 mL) was added lithium hydroxide (175 mg, 7.31 mmol). The reaction was briefly flushed with N₂, capped and allowed to stir at rt for 18 h. Some of the solvent was removed in vacuo. The reaction was then cooled in an ice/water bath slowly treated with HCl, 1 M in water (7.5 mL, 7.50 mmol). The reaction was diluted with EtOAc (200 mL), and the organic layer was washed with with brine (1×10 mL), dried over sodium sulfate filtered and evaporated to dryness to give the title compound, 4-(3-bromo-2,6-difluorophenoxy)-2-fluorobutanoic acid (977 mg, 3.12 mmol, quant) that was used without further purification.

¹H NMR (500 MHz, chloroform-d) δ 7.24 (ddd, J=9.1, 7.2, 5.3 Hz, 1H), 6.87 (ddd, J=10.0, 9.1, 2.0 Hz, 1H), 5.47-5.25 (m, 1H), 4.47-4.28 (m, 2H), 2.62-2.25 (m, 2H)

### 94C: 4-(3-bromo-2,6-difluorophenoxy)-2-fluoro-N-methoxy-N-methylbutanamide

To a solution of 4-(3-bromo-2,6-difluorophenoxy)-2-fluorobutanoic acid (977 mg, 3.12 mmol) in DCM (25 mL) was added N,O-dimethylhydroxylamine (365 mg, 3.74 mmol) and N,N-diisopropylethylamine (2.75 mL, 15.75 mmol). The reaction was flushed briefly with N₂, then treated with benzotriazol-1-yloxytris(dimethylamino)phosphonium hexafluorophosphate (1.8 g, 4.07 mmol), capped and allowed to stir at room temp for 18 h. The reaction was treated with additional N,O-dimethylhydroxylamine (365 mg, 3.74 mmol), N,N-diisopropylethylamine (2.75 mL, 15.75 mmol) and benzotriazol-1-yloxytris(dimethylamino)phosphonium hexafluoro-phosphate (1.8 g, 4.07 mmol) and allowed to stir at rt for several hours, at which time the solvent was removed under a gentle stream of nitrogen. The crude residue was purified by flash chromatography on silica gel, eluting with 0-100% gradient of EtOAc in hexane to afford the title compound, 4-(3-bromo-2,6-difluorophenoxy)-2-fluoro-N-methoxy-N-methylbutanamide (800 mg, 2.25 mmol, 72%).

¹H NMR (500 MHz, chloroform-d) δ 7.23 (ddd, J=9.1, 7.2, 5.4 Hz, 1H), 6.91-6.81 (m, 1H), 5.71-5.52 (m, 1H), 4.47-4.30 (m, 2H), 3.79 (s, 3H), 3.27 (s, 3H), 2.47-2.19 (m, 2H)

LCMS (ESI, m/z): 355.8, 357.8 [M+H]⁺.

### 94D: 4-(3-bromo-2,6-difluorophenoxy)-2-fluoro-1-(4-fluorophenyl)butan-1-one

To a solution of 4-(3-bromo-2,6-difluorophenoxy)-2-fluoro-N-methoxy-N-methylbutanamide (800 mg, 2.246 mmol) in anhydrous THF (15 mL) at 0° C. was slowly added (over 3-4 min) 4-fluorophenylmagnesium bromide 1.0 M solution in THF (2.7 mL, 2.70 mmol). After the addition was complete, the reaction was stirred cold for 10 min, then the bath was removed and the reaction allowed to warm to rt over 3.5 h. The reaction was quenched by the addition of aqueous saturated NH₄C₁ (15 mL) and diluted with EtOAc (40 mL). The layers were separated and the water layer was back extracted with EtOAc (3×30 mL). The organic layers were combined, washed with brine (1×10 mL), dried over sodium sulfate, filtered and evaporated to dryness. The crude residue was purified by flash chromatography on silica gel, eluting with 0-100% gradient of DCM in hexane to afford the title compound, 4-(3-bromo-2,6-difluorophenoxy)-2-fluoro-1-(4-fluorophenyl)butan-1-one (815 mg, 2.08 mmol, 93%).

¹H NMR (600 MHz, chloroform-d) δ 8.16-8.04 (m, 2H), 7.28-7.18 (m, 3H), 6.89 (td, J=9.6, 2.1 Hz, 1H), 6.12-5.92 (m, 1H), 4.48-4.37 (m, 2H), 2.65-2.44 (m, 1H), 2.40-2.21 (m, 1H.)

### 94E: 5-(3-bromo-2,6-difluorophenoxy)-3-fluoro-2-(4-fluorophenyl)pentan-2-ol

To a solution of 4-(3-bromo-2,6-difluorophenoxy)-2-fluoro-1-(4-fluorophenyl)butan-1-one (173 mg, 0.442 mmol) in THF (3 mL) under N₂ was added methylmagnesium bromide 3.0 M solution in diethyl ether (450 μL, 1.350 mmol) with rapid swirling. The reaction was allowed to stand at rt for 90 min. The reaction was quenched with aqueous saturated NH₄C₁ (5 mL) and diluted with EtOAc (75 mL). The layers were separated and the water layer was back extracted with EtOAc (1×25 mL). The organic layers were combined, washed with water (1×5 mL), brine (1×5 mL), dried over sodium sulfate, filtered and evaporated to dryness to give the title compound, 5-(3-bromo-2,6-difluorophenoxy)-3-fluoro-2-(4-fluorophenyl)pentan-2-ol (205 mg, 0.503 mmol, quant) that was used without further purification.

¹H NMR (500 MHz, chloroform-d) δ 7.49-7.39 (m, 2H), 7.19 (ddd, J=9.1, 7.2, 5.3 Hz, 1H), 7.11-7.02 (m, 2H), 6.81 (ddd, J=10.1, 9.1, 2.1 Hz, 1H), 5.06-4.85 (m, 1H), 4.26-4.17 (m, 2H), 2.07-1.93 (m, 1H), 1.88-1.63 (m, 4H).

### 94F (A3255-076)

To a flask containing 5-(3-bromo-2,6-difluorophenoxy)-3-fluoro-2-(4-fluorophenyl)pentan-2-ol (205 mg, 0.503 mmol) was added tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate) (255 mg, 0.554 mmol), dioxane (7 mL) and potassium phosphate tribasic, 2.0 M in water (1.00 mL, 2.000 mmol). The reaction was flushed with argon and stirred until all solids went into solution. The reaction was then treated with dichloro[1,1′-bis(di-t-butylphosphino)ferrocene]palladium(II), (16 mg, 0.025 mmol), flushed well with argon, equipped with a cold water condenser and heated at 115° C. for 2.5 h. The reaction was diluted with water (15 mL) and EtOAc (100 mL) and the layers were separated. The water layer was back extracted with EtOAc (3×25 mL) and the organic layers were combines, washed with water (2×5 mL), brine (1×5 mL), dried over sodium sulfate, filtered and evaporated to dryness. The crude residue was purified by flash chromatography on silica gel, eluting with 0-100% gradient of EtOAc in hexane to afford the title compound, tert-butyl (tert-butoxycarbonyl)(7-(2,4-difluoro-3-((3-fluoro-4-(4-fluorophenyl)-4-hydroxypentyl)oxy)phenyl)-[1,2,4]triazolo[1,5-a]pyridin-2-yl)carbamate (333 mg, 0.504 mmol, quant).

LCMS (ESI, m/z): 661.20 [M+H]+.

### Example 94: 5-(3-(2-amino-[1,2,4]triazolo[1,5-a]pyridin-7-yl)-2,6-difluorophenoxy)-3-fluoro-2-(4-fluorophenyl)pentan-2-ol enantiomer 1

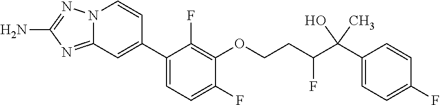 [SMILES: CC(O)(c1ccc(F)cc1)C(F)CCOc1c(F)ccc(-c2ccn3nc(N)nc3c2)c1F]

To a solution of, tert-butyl (tert-butoxycarbonyl)(7-(2,4-difluoro-3-((3-fluoro-4-(4-fluorophenyl)-4-hydroxypentyl)oxy)phenyl)-[1,2,4]triazolo[1,5-a]pyridin-2-yl)carbamate (333 mg, 0.504 mmol) in DCM (2 mL) was added TFA (4 mL, 51.9 mmol) and the resulting solution was allowed to stand at rt, for 1 h. The volatiles were removed under a gentle stream of nitrogen and the crude material was purified via preparative LC/MS with the following conditions: Column: Waters XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 29-69% B over 20 minutes; Flow Rate: 20 mL/min; Column Temperature: 25° C. Fraction collection was triggered by MS and UV signals. Fractions containing the desired product were combined and dried via centrifugal evaporation to give racemic 5-(5-(2-amino-[1,2,4]triazolo[1,5-a]pyridin-7-yl)-2,3-difluorophenoxy)-3-fluoro-2-(4-fluorophenyl)pentan-2-ol. This racemic material was then further purified via preparative SFC with the following conditions: Column: Chiralpak IC, 30 mm×250 mm, 5 m particles; Mobile Phase A: supercritical CO2; Mobile Phase B: i-PrOH with 0.1% NH4OH; Elution gradient: isocratic 25% B over 28 minutes; Flow Rate: 85 mL/min; Column Temperature: 40° C. Fraction collection was triggered by UV (240 nm). Fractions containing the first enantiomer to elute from the preparative SFC column were combined and dried via centrifugal evaporation to provide 5-(3-(2-amino-[1,2,4]triazolo[1,5-a]pyridin-7-yl)-2,6-difluorophenoxy)-3-fluoro-2-(4-fluorophenyl)pentan-2-ol enantiomer 1 (13.9 mg, 0.030 mmol, 28.4%).

¹H NMR (500 MHz, DMSO-d₆) δ 8.61 (d, J=7.0 Hz, 1H), 7.55-7.43 (m, 3H), 7.42-7.32 (m, 1H), 7.29-7.21 (m, 1H), 7.13 (t, J=8.9 Hz, 2H), 7.00 (br d, J=7.4 Hz, 1H), 6.08 (s, 2H), 5.53 (s, 1H), 4.87-4.68 (m, 1H), 4.28-4.13 (m, 2H), 2.02-1.83 (m, 1H), 1.81-1.62 (m, 1H), 1.54 (d, J=1.3 Hz, 3H).

LCMS (ESI, m/z): 461.2 [M+H]+.

### Example 95: 5-(5-(2-amino-[1,2,4]triazolo[1,5-a]pyridin-7-yl)-2,3-difluorophenoxy)-3-fluoro-2-(4-fluorophenyl)pentan-2-ol enantiomer 2

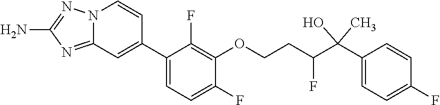 [SMILES: CC(O)(c1ccc(F)cc1)C(F)CCOc1c(F)ccc(-c2ccn3nc(N)nc3c2)c1F]

The title compound 5-(5-(2-amino-[1,2,4]triazolo[1,5-a]pyridin-7-yl)-2,3-difluorophenoxy)-3-fluoro-2-(4-fluorophenyl)pentan-2-ol enantiomer 2 (14.9 mg, 0.032 mmol, 30.4%)) was obtained as the second eluting enantiomer from the preparative SFC column in the preparation of Example 94.

1H NMR (500 MHz, DMSO-d₆) δ 8.61 (dd, J=7.0, 3.2 Hz, 1H), 7.49 (br d, J=2.1 Hz, 3H), 7.43-7.35 (m, 1H), 7.31-7.23 (m, 1H), 7.15 (td, J=8.6, 3.7 Hz, 2H), 7.01 (br d, J=5.6 Hz, 1H), 6.07 (br s, 2H), 5.59 (s, 1H), 4.87-4.65 (m, 1H), 4.28-4.14 (m, 2H), 2.17-1.95 (m, 2H), 1.67-1.47 (m, 4H).

MS ESI m/z 461.2 (M+H)⁺.

### Example 96: 5-(5-(2-amino-[1,2,4]triazolo[1,5-a]pyridin-7-yl)-2,3-difluorophenoxy)-3-fluoro-2-(4-fluorophenyl)pentan-2-ol enantiomer 3

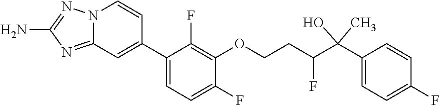 [SMILES: CC(O)(c1ccc(F)cc1)C(F)CCOc1c(F)ccc(-c2ccn3nc(N)nc3c2)c1F]

The title compound 5-(5-(2-amino-[1,2,4]triazolo[1,5-a]pyridin-7-yl)-2,3-difluorophenoxy)-3-fluoro-2-(4-fluorophenyl)pentan-2-ol enantiomer 2 (14.9 mg, 0.032 mmol, 30.4%)) was obtained as the third eluting enantiomer from the preparative SFC column in the preparation of Example 94.

1H NMR (500 MHz, DMSO-d₆) δ 8.61 (d, J=7.0 Hz, 1H), 7.57-7.44 (m, 3H), 7.41-7.31 (m, 1H), 7.28-7.19 (m, 1H), 7.13 (t, J=8.9 Hz, 2H), 7.00 (br d, J=7.4 Hz, 1H), 6.08 (s, 2H), 5.53 (s, 1H), 4.91-4.65 (m, 1H), 4.31-4.13 (m, 2H), 2.03-1.84 (m, 1H), 1.84-1.62 (m, 1H), 1.54 (d, J=1.3 Hz, 3H).

MS ESI m/z 461.2 (M+H)⁺.

### Example 97: 5-(5-(2-amino-[1,2,4]triazolo[1,5-a]pyridin-7-yl)-2,3-difluorophenoxy)-3-fluoro-2-(4-fluorophenyl)pentan-2-ol enantiomer 4

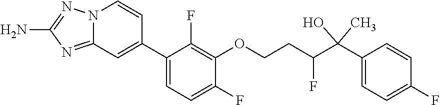 [SMILES: CC(O)(c1ccc(F)cc1)C(F)CCOc1c(F)ccc(-c2ccn3nc(N)nc3c2)c1F]

The title compound 5-(5-(2-amino-[1,2,4]triazolo[1,5-a]pyridin-7-yl)-2,3-difluorophenoxy)-3-fluoro-2-(4-fluorophenyl)pentan-2-ol enantiomer 2 (14.9 mg, 0.032 mmol, 30.4%)) was obtained as the fourth eluting enantiomer from the preparative SFC column in the preparation of Example 94.

¹H NMR (500 MHz, DMSO-d₆) δ 8.61 (br d, J=6.6 Hz, 1H), 7.56-7.44 (m, 3H), 7.37 (dt, J=8.3, 6.3 Hz, 1H), 7.30-7.20 (m, 1H), 7.18-7.09 (m, 2H), 7.00 (br d, J=5.3 Hz, 1H), 6.07 (br s, 2H), 5.53 (s, 1H), 4.88-4.68 (m, 1H), 4.17 (br d, J=3.9 Hz, 2H), 3.17 (d, J=5.2 Hz, 1H), 2.08 (d, J=2.4 Hz, 2H), 2.03-1.65 (m, 2H), 1.54 (br s, 3H) MS ESI m/z 461.2 (M+H)⁺.

### Example 98: 4-(3-(2-amino-[1,2,4]triazolo[1,5-a]pyridin-7-yl)-6-chloro-2-fluorophenoxy)-2-fluoro-1-(4-fluorophenyl)butan-1-ol enantiomer 1

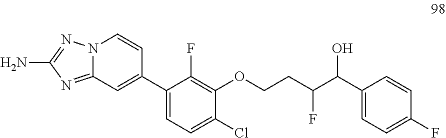 [SMILES: Nc1nc2cc(-c3ccc(Cl)c(OCCC(F)C(O)c4ccc(F)cc4)c3F)ccn2n1]

(The 4 unassigned diastereomers are:

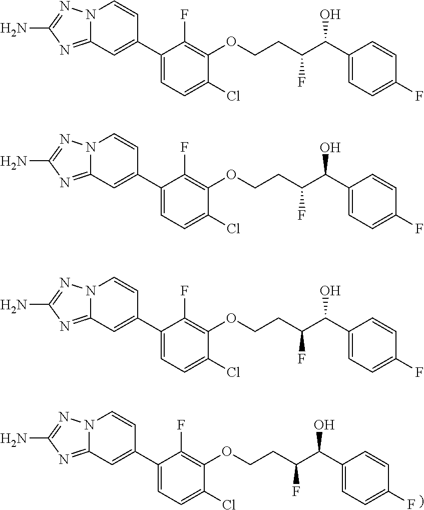 [SMILES: Nc1nc2cc(-c3ccc(Cl)c(OCC[C@@H](F)[C@H](O)c4ccc(F)cc4)c3F)ccn2n1 ; Nc1nc2cc(-c3ccc(Cl)c(OCC[C@@H](F)[C@@H](O)c4ccc(F)cc4)c3F)ccn2n1 ; Nc1nc2cc(-c3ccc(Cl)c(OCC[C@H](F)[C@H](O)c4ccc(F)cc4)c3F)ccn2n1 ; Nc1nc2cc(-c3ccc(Cl)c(OCC[C@H](F)[C@@H](O)c4ccc(F)cc4)c3F)ccn2n1]

### 98A: ethyl 4-(3-bromo-6-chloro-2-fluorophenoxy)-2-fluorobutanoate

To a dry 100 mL round bottom flask under N₂ was added ethyl 2-fluoro-4-iodobutanoate (1.0 g, 3.85 mmol), 3-bromo-6-chloro-2-fluorophenol (910 mg, 4.04 mmol), acetone (20 mL) and potassium carbonate (800 mg, 5.79 mmol). The reaction was flushed briefly with N₂, equipped with a cold water condenser and heated at 60° C. for 18 h. The reaction was filtered through a small pad of celite, rinsed well with acetone and the filtrate was evaporated to dryness. The residue was dissolved in EtOAc (225 mL) and the organic layer was washed with aqueous 1.0 M sodium hydroxide (3×10 mL), brine (1×10 mL), dried over sodium sulfate, filtered and evaporated to dryness to give the title compound, ethyl 4-(3-bromo-6-chloro-2-fluorophenoxy)-2-fluorobutanoate (1.36 g, 3.80 mmol, 99%) that was used without further purification.

¹H NMR (500 MHz, chloroform-d) δ 7.25 (dd, J=8.8, 6.7 Hz, 1H), 7.09 (dd, J=8.8, 2.0 Hz, 1H), 5.41-5.20 (m, 1H), 4.41-4.19 (m, 4H), 2.60-2.39 (m, 1H), 2.36-2.20 (m, 1H), 1.35 (t, J=7.1 Hz, 3H).

### 98B: 4-(3-bromo-6-chloro-2-fluorophenoxy)-2-fluorobutanoic acid

To a flask containing ethyl 4-(3-bromo-6-chloro-2-fluorophenoxy)-2-fluorobutanoate (1.05 g, 2.94 mmol) was added THF (15 mL) and water (7.5 mL). The reaction was flushed briefly with N₂, then treated with lithium hydroxide (170 mg, 7.10 mmol). The reaction was capped and allowed to stir at rt for 18 h. Some of the solvent was removed in vacuo and the reaction was cooled in an ice/water bath and slowly treated with HCl (1.0M in water, 7.5 mL, 7.5 mmol) until a pH of 2 was achieved. The reaction was diluted with EtOAc (200 mL) and the organic layer was washed with brine (1×10 mL), dried over sodium sulfate, filtered and evaporated to dryness to give the title compound, 4-(3-bromo-6-chloro-2-fluorophenoxy)-2-fluorobutanoic acid (931 mg, 2.83 mmol, 96%) that was used without further purification.

### 98C: 4-(3-bromo-6-chloro-2-fluorophenoxy)-2-fluoro-N-methoxy-N-methylbutanamide

To a flask containing 4-(3-bromo-6-chloro-2-fluorophenoxy)-2-fluorobutanoic acid (931 mg, 2.83 mmol) was added N,O-dimethylhydroxylamine (331 mg, 3.39 mmol), DCM (25 mL) and N,N-diisopropylethylamine (2.5 mL, 14.31 mmol). The reaction was flushed briefly with N₂, treated with BOP (1.63 g, 3.69 mmol), capped and allowed to stir at rt for 18 h. The crude reaction was purified by flash chromatography on silica gel, eluting with 0-100% gradient of EtOAc in hexane to afford the title compound 4-(3-bromo-6-chloro-2-fluorophenoxy)-2-fluoro-N-methoxy-N-methylbutanamide (904 mg, 2.43 mmol, 86%).

¹H NMR (400 MHz, chloroform-d) δ 7.24 (dd, J=8.8, 6.7 Hz, 1H), 7.09 (dd, J=8.8, 2.0 Hz, 1H), 5.83-5.56 (m, 1H), 4.43-4.26 (m, 2H), 3.79 (s, 3H), 3.28 (s, 3H), 2.57-2.21 (m, 2H)

MS ESI m/z 371.8, 373.8 (M+H)⁺.

### 98D: 4-(3-bromo-6-chloro-2-fluorophenoxy)-2-fluoro-1-(4-fluorophenyl)butan-1-one

To a solution of 4-(3-bromo-6-chloro-2-fluorophenoxy)-2-fluoro-N-methoxy-N-methylbutanamide (600 mg, 1.610 mmol) in anhydrous THF (8 mL) at 0° C. was added slowly (over 2-3 min) 4-fluorophenylmagnesium bromide 1.0 M solution in THF (1.9 mL, 1.900 mmol). The reaction was stirred cold for 10 min, then the bath was removed and the reaction was allowed to slowly warm to rt over 2 h. The reaction was quenched with aqueous saturated NH₄C₁ (10 mL) and diluted with EtOAc (35 mL). The aqueous layer was washed with EtOAc (3×30 mL) and the organic layers were combined. The organic layer was washed with brine (1×10 mL), dried over sodium sulfate, filtered and evaporated to dryness. The crude reaction was purified by flash chromatography on silica gel, eluting with 0-100% gradient of DCM in hexane to afford the title compound, 4-(3-bromo-6-chloro-2-fluorophenoxy)-2-fluoro-1-(4-fluorophenyl)butan-1-one (596.3 mg, 1.46 mmol, 91%).

¹H NMR (500 MHz, chloroform-d) δ 8.11 (dd, J=8.8, 5.5 Hz, 2H), 7.30-7.24 (m, 1H, and CDCl₃), 7.24-7.17 (m, 2H), 7.15-7.08 (m, 1H), 6.21-5.97 (m, 1H), 4.47-4.35 (m, 2H), 2.70-2.50 (m, 1H), 2.39-2.24 (m, 1H).

### 98E: 4-(3-bromo-6-chloro-2-fluorophenoxy)-2-fluoro-1-(4-fluorophenyl)butan-1-ol, Peak

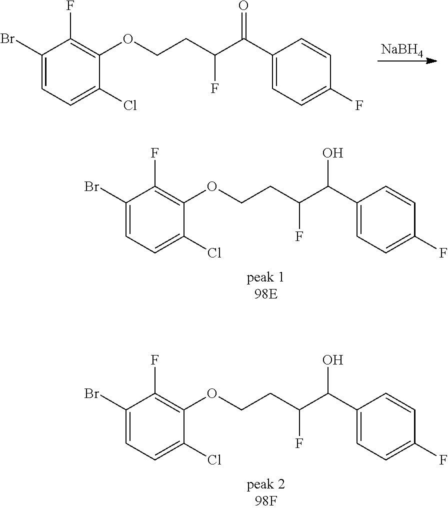 [SMILES: O=C(c1ccc(F)cc1)C(F)CCOc1c(Cl)ccc(Br)c1F ; "NaBH4" ; OC(c1ccc(F)cc1)C(F)CCOc1c(Cl)ccc(Br)c1F ; OC(c1ccc(F)cc1)C(F)CCOc1c(Cl)ccc(Br)c1F]

To a solution of 4-(3-bromo-6-chloro-2-fluorophenoxy)-2-fluoro-1-(4-fluorophenyl)butan-1-one (400 mg, 0.981 mmol) in MeOH (10 mL) was added sodium borohydride (56 mg, 1.480 mmol) over 1 min and the reaction was allowed to stir at rt for 10 min. The reaction was quenched with water (20 mL) and diluted with EtOAc (125 mL). The water layer was back extracted with EtOAc (3×25 mL) and the organic layers were combined, washed with brine (1×10 mL), dried over sodium sulfate, filtered and evaporated to dryness. The crude residue was purified via reverse phase chromatography using a Sunfire C18 Prep OBD 10 micron, 50×250 mm column, with a gradient (ACN/Water/0.05% TFA) from 40% B to 70% B over 40 min, (Solvent pair: Solvent A: 5% acetonitrile/95% water/0.05% TFA; Solvent B: 95% acetonitrile/5% water/0/05% TFA, UV detection=220 nm) to give the title compound, 4-(3-bromo-6-chloro-2-fluorophenoxy)-2-fluoro-1-(4-fluorophenyl)butan-1-ol, Peak 1 (144.8 mg, 0.353 mmol, 36%) as the first eluting material.

¹H NMR (500 MHz, CHLOROFORM-d) δ 7.48-7.35 (m, 2H), 7.27-7.17 (m, 1H), 7.15-7.01 (m, 3H), 5.10-4.88 (m, 1H), 4.87-4.74 (m, 1H), 4.24 (br t, J=5.4 Hz, 2H), 2.57 (br s, 1H), 2.13-1.91 (m, 2H)

### 98F: 4-(3-bromo-6-chloro-2-fluorophenoxy)-2-fluoro-1-(4-fluorophenyl)butan-1-ol, Peak 2

The title compound 4-(3-bromo-6-chloro-2-fluorophenoxy)-2-fluoro-1-(4-fluorophenyl)butan-1-ol, Peak 2 (127.3 mg, 0.311 mmol, 31.7%) was obtained as the second eluting material from the C₁₈ purification in the preparation of Example 98E.

¹H NMR (500 MHz, chloroform-d) δ 7.41 (ddd, J=8.5, 5.4, 2.7 Hz, 2H), 7.25-7.17 (m, 1H), 7.14-7.01 (m, 3H), 5.17-4.93 (m, 2H), 4.34-4.17 (m, 2H), 2.35 (br s, 1H), 2.19-1.97 (m, 2H)

### 98G: tert-butyl (tert-butoxycarbonyl)(7-(4-chloro-2-fluoro-3-(3-fluoro-4-(4-fluorophenyl)-4-hydroxybutoxy)phenyl)-[1,2,4]triazolo[1,5-a]pyridin-2-yl)carbamate

To a solution of 4-(3-bromo-6-chloro-2-fluorophenoxy)-2-fluoro-1-(4-fluorophenyl)butan-1-ol, Peak 1 98E (145 mg, 0.354 mmol), and tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (180 mg, 0.391 mmol) in dioxane (4 mL) was added potassium phosphate tribasic, 2M in water (710 μL, 1.420 mmol). The reaction was flushed very well with argon for 3-4 min, then treated with dichloro[1,1′-bis(di-t-butylphosphino)ferrocene]palladium(II), 99% (12 mg, 0.018 mmol). The reaction was again flushed very well with argon, securely capped and heated at 60° C. for 3 h. The reaction was diluted with water (10 mL) and the aqueous layer was washed with EtOAc (3×15 mL). The organic layers are combines, washed with brine (1×4 mL), dried over sodium sulfate, filtered and evaporated to dryness to give the title compound, tert-butyl (tert-butoxycarbonyl)(7-(4-chloro-2-fluoro-3-(3-fluoro-4-(4-fluorophenyl)-4-hydroxybutoxy)phenyl)-[1,2,4]triazolo[1,5-a]pyridin-2-yl)carbamate that was used without further purification.

MS ESI m/z 663.0, 665.0 (M+H)⁺.

### Example 98: 4-(3-(2-amino-[1,2,4]triazolo[1,5-a]pyridin-7-yl)-6-chloro-2-fluorophenoxy)-2-fluoro-1-(4-fluorophenyl)butan-1-ol enantiomer 1

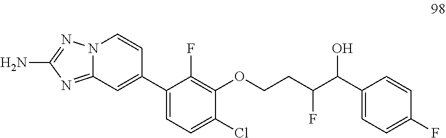 [SMILES: Nc1nc2cc(-c3ccc(Cl)c(OCCC(F)C(O)c4ccc(F)cc4)c3F)ccn2n1]

To a solution of tert-butyl (tert-butoxycarbonyl)(7-(4-chloro-2-fluoro-3-(3-fluoro-4-(4-fluorophenyl)-4-hydroxybutoxy)phenyl)-[1,2,4]triazolo[1,5-a]pyridin-2-yl)carbamate (235 mg, 0.354 mmol) in MeOH (1.5 mL) was added HCl, 4 M in dioxane (900 μL, 3.60 mmol) and the reaction was heated at 60° C. sand bath for 1 h. The reaction was treated with additional HCl, 4 M in dioxane (1.8 mL, 1.80 mmol) and heated at 60° C. for 1.5 hours. The volatiles were removed under a gentle stream of nitrogen and the crude material was purified via preparative SFC with the following conditions: Column: Chiralpak IC, 30 mm×250 mm, 5 m particles; Mobile Phase A: supercritical CO2; Mobile Phase B: MeOH with 0.1% DEA; Elution gradient: isocratic 40% B over 30 minutes; Flow Rate: 90 mL/min; Column Temperature: 40° C. Fraction collection was triggered by UV (240 nm). Fractions containing the first enantiomer to elute from the preparative SFC column were combined and dried via centrifugal evaporation. This material was then further purified via preparative SFC with the following conditions: Column: Whelk-01 (R,R), 30 mm×250 mm, 5 m particles; Mobile Phase A: supercritical CO2; Mobile Phase B: MeOH with 0.1% DEA; Elution gradient: isocratic 20% B over 30 minutes; Flow Rate: 90 mL/min; Column Temperature: 40° C. Fraction collection was triggered by UV (240 nm). Fractions containing the desired product were combined and dried via centrifugal evaporation. This material was then further purified via preparative LC/MS with the following conditions: Column: Waters BEH C18, 19 mm×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-59% B over 20 minutes; Flow Rate: 20 mL/min; Column Temperature: 25 C. Fraction collection was triggered by MS and UV signals. Fractions containing the desired product were combined and dried via centrifugal evaporation to give the title compound, 4-(3-(2-amino-[1,2,4]triazolo[1,5-a]pyridin-7-yl)-6-chloro-2-fluorophenoxy)-2-fluoro-1-(4-fluorophenyl)butan-1-ol enantiomer 1 (24.8 mg, 0.054 mmol, 15%).

MS ESI m/z 463.00, 464.95 (M+H)⁺.

### Example 99: 4-(3-(2-amino-[1,2,4]triazolo[1,5-a]pyridin-7-yl)-6-chloro-2-fluorophenoxy)-2-fluoro-1-(4-fluorophenyl)butan-1-ol enantiomer 2

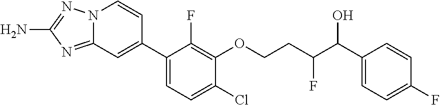 [SMILES: Nc1nc2cc(-c3ccc(Cl)c(OCCC(F)C(O)c4ccc(F)cc4)c3F)ccn2n1]

The title compound 4-(3-(2-amino-[1,2,4]triazolo[1,5-a]pyridin-7-yl)-6-chloro-2-fluorophenoxy)-2-fluoro-1-(4-fluorophenyl)butan-1-ol enantiomer 2 (31 mg, 0.067 mmol, 18%)) was obtained as the second eluting enantiomer from the preparative SFC column in the preparation of Example 98.

MS ESI m/z 463.00, 465.00 (M+H)⁺.

### Example 100: 4-(3-(2-amino-[1,2,4]triazolo[1,5-a]pyridin-7-yl)-6-chloro-2-fluorophenoxy)-2-fluoro-1-(4-fluorophenyl)butan-1-ol enantiomer 1

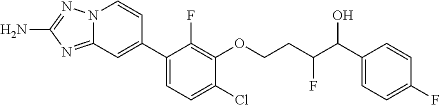 [SMILES: Nc1nc2cc(-c3ccc(Cl)c(OCCC(F)C(O)c4ccc(F)cc4)c3F)ccn2n1]

### 100A: tert-butyl (tert-butoxycarbonyl)(7-(4-chloro-2-fluoro-3-(3-fluoro-4-(4-fluorophenyl)-4-hydroxybutoxy)phenyl)-[1,2,4]triazolo[1,5-a]pyridin-2-yl)carbamate

To a solution of 4-(3-bromo-6-chloro-2-fluorophenoxy)-2-fluoro-1-(4-fluorophenyl)butan-1-ol (127.3 mg, 0.311 mmol), Peak 2 98F and tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (160 mg, 0.348 mmol) in dioxane (4 mL) was added potassium phosphate tribasic, 2M in water (620 μL, 1.240 mmol). The reaction was flushed very well with argon then treated with dichloro[1,1′-bis(di-t-butylphosphino)ferrocene]palladium(II), 99% (12 mg, 0.018 mmol). The reaction was again flushed very well with argon, securely capped and heated at 60° C. oil bath for 2.5 h. The reaction was diluted with water and the aqueous layer was washed with EtOAc (3×15 mL). The organic layers were combined, washed with brine, dried over sodium sulfate, filtered and evaporated to dryness to give the title compound, tert-butyl (tert-butoxycarbonyl)(7-(4-chloro-2-fluoro-3-(3-fluoro-4-(4-fluorophenyl)-4-hydroxybutoxy)phenyl)-[1,2,4]triazolo[1,5-a]pyridin-2-yl)carbamate, that was used without further purification MS ESI m/z 663.05 (M+H)⁺.

### Example 100: 4-(3-(2-amino-[1,2,4]triazolo[1,5-a]pyridin-7-yl)-6-chloro-2-fluorophenoxy)-2-fluoro-1-(4-fluorophenyl)butan-1-ol enantiomer 1

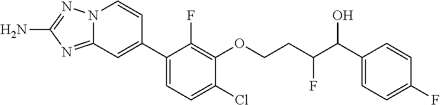 [SMILES: Nc1nc2cc(-c3ccc(Cl)c(OCCC(F)C(O)c4ccc(F)cc4)c3F)ccn2n1]

To a solution of tert-butyl (tert-butoxycarbonyl)(7-(4-chloro-2-fluoro-3-(3-fluoro-4-(4-fluorophenyl)-4-hydroxybutoxy)phenyl)-[1,2,4]triazolo[1,5-a]pyridin-2-yl)carbamate (206 mg, 0.311 mmol) in MeOH (1.5 mL) was added HCl, 4M in dioxane (800 μL, 3.20 mmol) and the reaction was heated at 60° C. for 1.5 h. The reaction was treated with additional HCl, 4M in dioxane (1.0 mL, 1.0 mmol) and heated at 60° C. until the reaction was determined to be complete by LC/MS analysis. The volatiles were removed under a gentle stream of nitrogen and the crude material was purified via preparative SFC with the following conditions: Column: Lux Cellulose-2, 30 mm×250 mm, 5 m particles; Mobile Phase A: supercritical CO2; Mobile Phase B: MeOH with 0.1% DEA; Elution gradient: isocratic 30% B over 50 minutes; Flow Rate: 85 mL/min; Column Temperature: 40° C. Fraction collection was triggered by UV (240 nm). Fractions containing the first enantiomer to elute from the preparative SFC column were combined and dried via centrifugal evaporation to provide 4-(3-(2-amino-[1,2,4]triazolo[1,5-a]pyridin-7-yl)-6-chloro-2-fluorophenoxy)-2-fluoro-1-(4-fluorophenyl)butan-1-ol enantiomer 1 (38.7 mg, 0.054 mmol, 15%).

MS ESI m/z 663.05 (M+H)⁺.

### Example 101: 4-(3-(2-amino-[1,2,4]triazolo[1,5-a]pyridin-7-yl)-6-chloro-2-fluorophenoxy)-2-fluoro-1-(4-fluorophenyl)butan-1-ol enantiomer 2

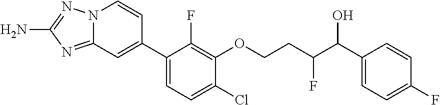 [SMILES: Nc1nc2cc(-c3ccc(Cl)c(OCCC(F)C(O)c4ccc(F)cc4)c3F)ccn2n1]

The title compound 4-(3-(2-amino-[1,2,4]triazolo[1,5-a]pyridin-7-yl)-6-chloro-2-fluorophenoxy)-2-fluoro-1-(4-fluorophenyl)butan-1-ol enantiomer 2 (49.4 mg, 0.107 mmol, 27.5%)) was obtained as the second eluting enantiomer from the preparative SFC column in the preparation of Example 100.

### Example 102: 4-(3-(2-amino-[1,2,4]triazolo[1,5-a]pyridin-7-yl)-2,6-difluorophenoxy)-2-fluoro-1-(4-fluorophenyl)butan-1-ol enantiomer 1

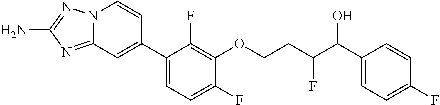 [SMILES: Nc1nc2cc(-c3ccc(F)c(OCCC(F)C(O)c4ccc(F)cc4)c3F)ccn2n1]

(The 4 unassigned enantiomers are:

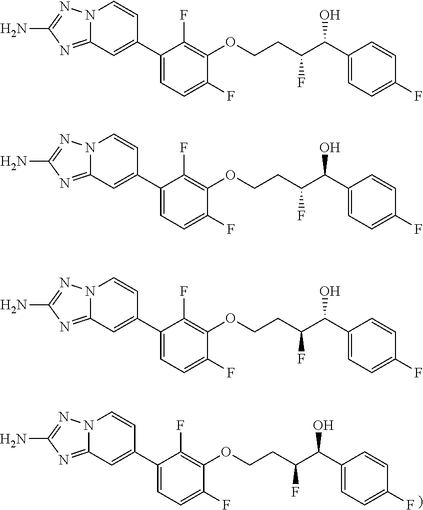 [SMILES: Nc1nc2cc(-c3ccc(F)c(OCC[C@@H](F)[C@H](O)c4ccc(F)cc4)c3F)ccn2n1 ; Nc1nc2cc(-c3ccc(F)c(OCC[C@@H](F)[C@@H](O)c4ccc(F)cc4)c3F)ccn2n1 ; Nc1nc2cc(-c3ccc(F)c(OCC[C@H](F)[C@H](O)c4ccc(F)cc4)c3F)ccn2n1 ; Nc1nc2cc(-c3ccc(F)c(OCC[C@H](F)[C@@H](O)c4ccc(F)cc4)c3F)ccn2n1]

### 102A: 4-(3-bromo-2,6-difluorophenoxy)-2-fluoro-1-(4-fluorophenyl)butan-1-ol

To a solution of 4-(3-bromo-2,6-difluorophenoxy)-2-fluoro-1-(4-fluorophenyl)butan-1-one (291 mg, 0.744 mmol) in MeOH (8 mL) was slowly added sodium borohydride (43 mg, 1.137 mmol) with rapid swirling and the reaction was allowed to stir at rt for 20 min. The reaction was carefully quenched with water (10 mL) and diluted with EtOAc (150 mL). The water layer was washed with EtOAc (3×20 mL), the organic layers were combined, washed with brine, dried over sodium sulfate, filtered and evaporated to dryness to give the title compound, 4-(3-bromo-2,6-difluorophenoxy)-2-fluoro-1-(4-fluorophenyl)butan-1-ol (296 mg, 0.753 mmol, quant) that was used without further purification.

### 102B: tert-butyl (tert-butoxycarbonyl)(7-(2,4-difluoro-3-(3-fluoro-4-(4-fluorophenyl)-4-hydroxybutoxy)phenyl)-[1,2,4]triazolo[1,5-a]pyridin-2-yl)carbamate

To a solution of 4-(3-bromo-2,6-difluorophenoxy)-2-fluoro-1-(4-fluorophenyl)butan-1-ol (200 mg, 0.509 mmol) and tert-butyl (tert-butoxycarbonyl)(7-(4,4,5,5-tetramethyl-1,3,2-dioxaborolan-2-yl)-[1,2,4]triazolo[1,5-a]pyridin-2-yl)carbam (258 mg, 0.560 mmol) in dioxane (6 mL) was added potassium phosphate tribasic, 2.0M in water (1.0 mL, 2.000 mmol). The reaction was flushed with argon, treated with dichloro[1,1′-bis(di-t-butylphosphino)ferrocene]palladium(II) (20 mg, 0.031 mmol), flushed with argon again, securely capped and heated at 60° C. for 100 min. The reaction was diluted with water (20 mL) and the aqueous layer was washed with EtOAc (4×50 mL). The organic layers were combined, washed with brine (1×10 mL), dried over sodium sulfate, filtered and evaporated to dryness. The crude residue was purified by flash chromatography on silica gel, eluting with 0-75% gradient of EtOAc in hexane to afford the title compound, tert-butyl (tert-butoxycarbonyl)(7-(2,4-difluoro-3-(3-fluoro-4-(4-fluorophenyl)-4-hydroxybutoxy)phenyl)-[1,2,4]triazolo[1,5-a]pyridin-2-yl)carbamate (326 mg, 0.504 mmol, 99%).

¹H NMR (500 MHz, chloroform-d) δ 8.11 (dd, J=8.8, 5.5 Hz, 2H), 7.30-7.24 (m, 1H, and CDCl₃), 7.24-7.17 (m, 2H), 7.15-7.08 (m, 1H), 6.21-5.97 (m, 1H), 4.47-4.35 (m, 2H), 2.70-2.50 (m, 1H), 2.39-2.24 (m, 1H) MS ESI m/z 647.15 (M+H)⁺.

### Example 102: 4-(3-(2-amino-[1,2,4]triazolo-[1,5-a]pyridin-7-yl)-2,6-difluorophenoxy)-2-fluoro-1-(4-fluorophenyl)butan-1-ol enantiomer 1

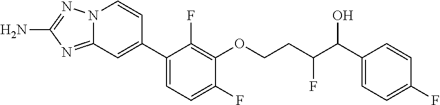 [SMILES: Nc1nc2cc(-c3ccc(F)c(OCCC(F)C(O)c4ccc(F)cc4)c3F)ccn2n1]

To a solution of tert-butyl (tert-butoxycarbonyl)(7-(2,4-difluoro-3-(3-fluoro-4-(4-fluorophenyl)-4-hydroxybutoxy)phenyl)-[1,2,4]triazolo[1,5-a]pyridin-2-yl)carbamate (326 mg, 0.504 mmol) in MeOH (2 mL) was added HCl, 4.0M in dioxane (3.0 mL, 12.00 mmol) and the reaction was heated at a 60° C. for 90 min. The volatiles were removed under a gentle stream of N₂ and the crude residue was purified via preparative SFC chromatography with the following conditions: Column: Chiralpak IC, 30 mm×250 mm, 5 μm particles; Mobile Phase A: supercritical CO2; Mobile Phase B: i-PrOH with 0.1% NH₄₀H; Elution gradient: isocratic 30% B over 31.5 minutes Flow Rate: 90 mL/min; Column Temperature: 40° C. Fraction collection was triggered by UV (220 nm).

Fractions containing the first enantiomer to elute from the preparative SFC column were combined and dried via centrifugal evaporation. The material was further purified via preparative SFC with the following conditions: Column: Chiralpak IA, 30 mm×250 mm, 5 m particles; Mobile Phase A: supercritical CO2; Mobile Phase B: i-PrOH with 0.1% NH₄₀H; Elution gradient: isocratic 35% B over 40 minutes Flow Rate: 90 mL/min; Column Temperature: 40° C. Fraction collection was triggered by UV (220 nm). Fractions containing the desired product were combined and dried via centrifugal evaporation to provide the title compound, 4-(3-(2-amino-[1,2,4]triazolo[1,5-a]pyridin-7-yl)-2,6-difluorophenoxy)-2-fluoro-1-(4-fluorophenyl)butan-1-ol enantiomer 1 (29.8 mg, 0.067 mmol, 13.1%).

MS ESI m/z 447.05 (M+H)⁺.

### Example 103: 4-(3-(2-amino-[1,2,4]triazolo-[1,5-a]pyridin-7-yl)-2,6-difluorophenoxy)-2-fluoro-1-(4-fluorophenyl)butan-1-ol enantiomer 2

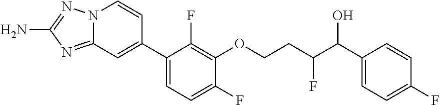 [SMILES: Nc1nc2cc(-c3ccc(F)c(OCCC(F)C(O)c4ccc(F)cc4)c3F)ccn2n1]

The title compound, 4-(3-(2-amino-[1,2,4]triazolo[1,5-a]pyridin-7-yl)-2,6-difluorophenoxy)-2-fluoro-1-(4-fluorophenyl)butan-1-ol enantiomer 2 (34.1 mg, 0.076 mmol, 15%) was obtained as the second eluting enantiomer from the preparative SFC column in the preparation of Example 102.

MS ESI m/z 447.05 (M+H)⁺.

### Example 104: 4-(3-(2-amino-[1,2,4]triazolo-[1,5-a]pyridin-7-yl)-2,6-difluorophenoxy)-2-fluoro-1-(4-fluorophenyl)butan-1-ol enantiomer 3

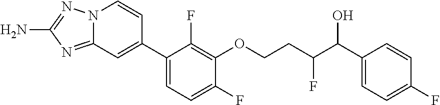 [SMILES: Nc1nc2cc(-c3ccc(F)c(OCCC(F)C(O)c4ccc(F)cc4)c3F)ccn2n1]

The title compound 4-(3-(2-amino-[1,2,4]triazolo[1,5-a]pyridin-7-yl)-2,6-difluorophenoxy)-2-fluoro-1-(4-fluorophenyl)butan-1-ol enantiomer 3 (20.9 mg, 0.047 mmol, 9.2%) was obtained as the third eluting enantiomer from the preparative SFC column in the preparation of Example 102.

MS ESI m/z 447.05 (M+H)⁺.

### Example 105: 4-(3-(2-amino-[1,2,4]triazolo-[1,5-a]pyridin-7-yl)-2,6-difluorophenoxy)-2-fluoro-1-(4-fluorophenyl)butan-1-ol enantiomer 4

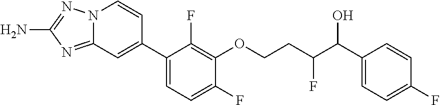 [SMILES: Nc1nc2cc(-c3ccc(F)c(OCCC(F)C(O)c4ccc(F)cc4)c3F)ccn2n1]

The title compound 4-(3-(2-amino-[1,2,4]triazolo[1,5-a]pyridin-7-yl)-2,6-difluorophenoxy)-2-fluoro-1-(4-fluorophenyl)butan-1-ol enantiomer 4 (49.2 mg, 0.110 mmol, 21.6%) was obtained as the fourth eluting enantiomer from the preparative SFC column in the preparation of Example 102.

MS ESI m/z 447.05 (M+H)⁺.

### Example 106: 4-(3-(2-amino-[1,2,4]triazolo[1,5-a]pyridin-7-yl)-2-fluoro-6-methylphenoxy)-2-fluoro-1-(4-fluorophenyl)butan-1-ol enantiomer 1

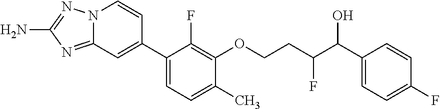 [SMILES: Cc1ccc(-c2ccn3nc(N)nc3c2)c(F)c1OCCC(F)C(O)c1ccc(F)cc1]

(The 4 unassigned enantiomers are:

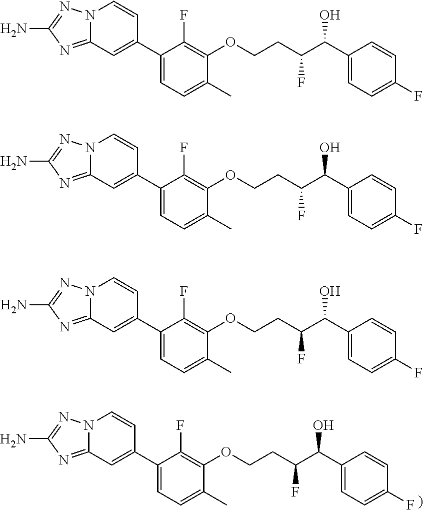 [SMILES: Cc1ccc(-c2ccn3nc(N)nc3c2)c(F)c1OCC[C@@H](F)[C@H](O)c1ccc(F)cc1 ; Cc1ccc(-c2ccn3nc(N)nc3c2)c(F)c1OCC[C@@H](F)[C@@H](O)c1ccc(F)cc1 ; Cc1ccc(-c2ccn3nc(N)nc3c2)c(F)c1OCC[C@H](F)[C@H](O)c1ccc(F)cc1 ; Cc1ccc(-c2ccn3nc(N)nc3c2)c(F)c1OCC[C@H](F)[C@@H](O)c1ccc(F)cc1]

### 106A: ethyl 4-(3-bromo-2-fluoro-6-methylphenoxy)-2-fluorobutanoate

To a dry 100 mL flask under N₂ was added 3-bromo-2-fluoro-6-methylphenol (828 mg, 4.04 mmol), acetone (35 mL) and ethyl 2-fluoro-4-iodobutanoate (1.0 g, 3.85 mmol). The reaction was flushed briefly with N₂, treated with potassium carbonate (800 mg, 5.79 mmol), equipped with a cold water condenser and heated at 60° C. for 18 h. The reaction was filtered through a small pad of celite and the filtrate was evaporated to dryness. The residue was dissolved in EtOAc (200 mL) and the organic layer was washed with 1.0M sodium hydroxide (3×10 mL), water (1×10 mL), brine (1×10 mL), dried over sodium sulfate, filtered and evaporated to dryness. ¹HNMR analysis indicated that the reaction had not gone to completion. The residue was redissolved in acetone (25 mL) and treated with additional 3-bromo-2-fluoro-6-mehtylphenol (41 mg, 0.2 mmol) and additional K₂CO₃ (800 mg, 5.79 mmol). The flask equipped with a cold water condenser was heated at 60° C. for 18 h. The reaction was filtered through a pad of celite and the filtrate was evaporated to dryness. The residue was dissolved in EtOAc (200 mL), the organic layer was washed with 1.0M sodium hydroxide (4×15 mL), brine (1×10 mL), dried over sodium sulfate, filtered and evaporated to dryness to give the title compound, ethyl 4-(3-bromo-2-fluoro-6-methylphenoxy)-2-fluorobutanoate (751 mg, 2.23 mmol, 57.9%) that was used without further purification.

¹H NMR (500 MHz, chloroform-d) δ 7.17 (dd, J=8.2, 6.4 Hz, 1H), 6.85 (dd, J=8.3, 0.9 Hz, 1H), 5.36-5.17 (m, 1H), 4.31 (q, J=7.2 Hz, 2H), 4.26-4.14 (m, 2H), 2.57-2.40 (m, 1H), 2.38-2.23 (m, 4H), 1.35 (t, J=7.2 Hz, 3H).

### 106B: 4-(3-bromo-2-fluoro-6-methylphenoxy)-2-fluorobutanoic acid

To a solution of ethyl 4-(3-bromo-2-fluoro-6-methylphenoxy)-2-fluorobutanoate (751 mg, 2.227 mmol) in a mixture of THF (15 mL) and water (7.5 mL) was added lithium hydroxide (128 mg, 5.35 mmol). The reaction was capped and allowed to stir at rt for 18 h. Some of the solvent was removed in vacuo and the reaction was then cooled in an ice/water bath and slowly treated with HCl, 1.0 M in water (5.4 mL, 5.40 mmol). The reaction was diluted with EtOAc (200 mL) and the organic layer was washed with brine, dried over sodium sulfate, filtered and evaporated to dryness to give the title compound, 4-(3-bromo-2-fluoro-6-methylphenoxy)-2-fluorobutanoic acid (705.5 mg, 2.28 mmol, quant) that was used without further purification.

¹H NMR (600 MHz, chloroform-d) δ 7.22-7.13 (m, 1H), 6.85 (d, J=8.2 Hz, 1H), 5.48-5.27 (m, 1H), 4.34-4.11 (m, 2H), 2.63-2.45 (m, 1H), 2.44-2.30 (m, 1H), 2.27 (s, 3H).

### 106C: 4-(3-bromo-2-fluoro-6-methylphenoxy)-2-fluoro-N-methoxy-N-methylbutanamide

To a solution of 4-(3-bromo-2-fluoro-6-methylphenoxy)-2-fluorobutanoic acid (705.5 mg, 2.282 mmol), N,O-dimethylhydroxylamine (275 mg, 2.82 mmol), and N,N-diisopropylethylamine (2.0 mL, 11.45 mmol) in DCM (20 mL) was added BOP (1.4 g, 3.17 mmol). The reaction was capped and allowed to stir at rt for 18 h. The solvent was removed in vacuo and the crude residue was purified by flash chromatography on silica gel, eluting with 0-100% gradient of EtOAc in hexane to afford the title compound, 4-(3-bromo-2-fluoro-6-methylphenoxy)-2-fluoro-N-methoxy-N-methylbutanamide (731 mg, 2.08 mmol, 91%).

¹H NMR (600 MHz, chloroform-d) δ 7.16 (dd, J=8.2, 6.4 Hz, 1H), 6.85 (d, J=8.2 Hz, 1H), 5.72-5.50 (m, 1H), 4.32-4.18 (m, 2H), 3.78 (s, 3H), 3.28 (s, 3H), 2.49-2.22 (m, 5H)

### 106D: 4-(3-bromo-2-fluoro-6-methylphenoxy)-2-fluoro-1-(4-fluorophenyl)-butan-1-one

To an ice cold solution of 4-(3-bromo-2-fluoro-6-methylphenoxy)-2-fluoro-N-methoxy-N-methylbutanamide (730 mg, 2.073 mmol) in anhydrous THF (10 mL) was slowly added (over 3-4 min) 4-fluorophenylmagnesium bromide 1.0M solution in THF (2.5 mL, 2.500 mmol). The reaction was stirred at 0° C. for 20 min, then the bath was removed and the reaction allowed to warm to rt and stirred at rt for 18 h. The reaction was quenched with aqueous saturated NH₄Cl (10 mL) and diluted with EtOAc (35 mL) and the aqueous layer was washed with EtOAc (3×50 mL). The organic layers were combined, washed with brine (1×10 mL) dried over sodium sulfate, filtered and evaporated to dryness. The crude residue was purified by flash chromatography on silica gel, eluting with 0-100% gradient of DCM in hexane to afford the title compound, 4-(3-bromo-2-fluoro-6-methylphenoxy)-2-fluoro-1-(4-fluorophenyl)butan-1-one (566 mg, 1.46 mmol, 70.3%).

¹H NMR (500 MHz, chloroform-d) δ 8.16-8.03 (m, 2H), 7.26-7.13 (m, 3H), 6.93-6.80 (m, 1H), 6.09-5.88 (m, 1H), 4.38-4.20 (m, 2H), 2.67-2.45 (m, 1H), 2.40-2.24 (m, 4H).

### 106E: 4-(3-bromo-2-fluoro-6-methylphenoxy)-2-fluoro-1-(4-fluorophenyl)-butan-1-ol

To a solution of 4-(3-bromo-2-fluoro-6-methylphenoxy)-2-fluoro-1-(4-fluorophenyl)butan-1-one (358.5 mg, 0.926 mmol) in methanol (9 mL) was slowly added sodium borohydride (53 mg, 1.401 mmol) with rapid stirring. After the addition was complete, the reaction was stirred at rt for 15 min. The reaction was quenched with water (15 mL) and diluted with EtOAc (50 mL). The aqueous layer was washed with EtOAc (3×50 mL) and the organic layers were combined, washed with brine (1×5 mL), dried over sodium sulfate, filtered and evaporated to dryness to give the title compound, 4-(3-bromo-2-fluoro-6-methylphenoxy)-2-fluoro-1-(4-fluorophenyl)butan-1-ol (355 mg, 0.912 mmol, 99%) that was used without further purification.

### 106F: tert-butyl (tert-butoxycarbonyl)(7-(2-fluoro-3-(3-fluoro-4-(4-fluorophenyl)-4-hydroxybutoxy)-4-methylphenyl)-[1,2,4]triazolo[1,5-a]pyridin-2-yl)carbamate

To the flask containing 4-(3-bromo-2-fluoro-6-methylphenoxy)-2-fluoro-1-(4-fluorophenyl)-butan-1-ol (360 mg, 0.925 mmol) was added tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (468 mg, 1.017 mmol), dioxane (9 mL) and potassium phosphate tribasic (1.85 mL, 3.70 mmol). The reaction was flushed very well with argon, then treated with dichloro[1,1′-bis(di-t-butylphosphino)ferrocene]palladium(II), 99% (33 mg, 0.051 mmol). The reaction was again flushed very well with argon, equipped with a condenser (flushed with argon) and heated at 115° C. for 2.5 h. The reaction was diluted with water (20 mL) and the aqueous layer was washed with EtOAc (4×50 mL). The organic layers were combined, washed with brine (1×10 mL), dried over sodium sulfate, filtered and evaporated to dryness. The crude residue was purified by flash chromatography on silica gel, eluting with 0-65% gradient of EtOAc in DCM to afford the title compound, tert-butyl (tert-butoxycarbonyl)(7-(2-fluoro-3-(3-fluoro-4-(4-fluorophenyl)-4-hydroxybutoxy)-4-methylphenyl)-[1,2,4]triazolo[1,5-a]pyridin-2-yl)carbamate (585 mg, 0.910 mmol, 98%).

MS ESI m/z 643.25 (M+H)⁺.

### Example 106: 4-(3-(2-amino-[1,2,4]triazolo[1,5-a]pyridin-7-yl)-2-fluoro-6-methylphenoxy)-2-fluoro-1-(4-fluorophenyl)butan-1-ol enantiomer 1

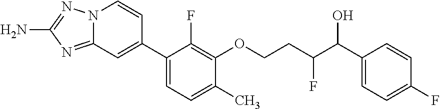 [SMILES: Cc1ccc(-c2ccn3nc(N)nc3c2)c(F)c1OCCC(F)C(O)c1ccc(F)cc1]

To a solution of tert-butyl (tert-butoxycarbonyl)(7-(2-fluoro-3-(3-fluoro-4-(4-fluorophenyl)-4-hydroxybutoxy)-4-methylphenyl)-[1,2,4]triazolo[1,5-a]pyridin-2-yl)carbamate (585 mg, 0.910 mmol) in MeOH (5 mL) was added HCl 4.0M solution in 1 4-dioxane (3 mL, 12.00 mmol) and the reaction was heated at 65° C. for 2.5 h. The reaction was treated with additional HCl/dioxane (3 mL, 12 mmol) and heated at 60° C. for 2.5 h. The solvent was removed under a gentle stream of nitrogen and the crude residue was purified via preparative SFC chromatography with the following conditions: Column: Chiralpak IC, 30 mm×250 mm, 5 m particles; Mobile Phase A: supercritical CO2; Mobile Phase B: i-PrOH with 0.1% NH₄₀H; Elution gradient: isocratic 30% B over 39 minutes Flow Rate: 90 mL/min; Column Temperature: 40° C. Fraction collection was triggered by UV (220 nm). Fractions containing the first enantiomer to elute from the preparative SFC column were combined and dried via centrifugal evaporation provide the title compound, 4-(3-(2-amino-[1,2,4]triazolo[1,5-a]pyridin-7-yl)-2-fluoro-6-methylphenoxy)-2-fluoro-1-(4-fluorophenyl)butan-1-ol enantiomer 1 (46.0 mg, 0.104 mmol, 11.3%)

MS ESI m/z 443.10 (M+H)⁺

### Example 107: 4-(3-(2-amino-[1,2,4]triazolo[1,5-a]pyridin-7-yl)-2-fluoro-6-methylphenoxy)-2-fluoro-1-(4-fluorophenyl)butan-1-ol enantiomer 2

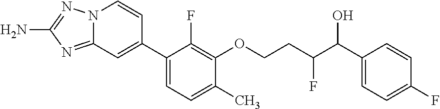 [SMILES: Cc1ccc(-c2ccn3nc(N)nc3c2)c(F)c1OCCC(F)C(O)c1ccc(F)cc1]

The title compound 4-(3-(2-amino-[1,2,4]triazolo[1,5-a]pyridin-7-yl)-2-fluoro-6-methylphenoxy)-2-fluoro-1-(4-fluorophenyl)butan-1-ol enantiomer 2 (49.2 mg, 0.104 mmol, 11.3%) was obtained as the second eluting enantiomer from the preparative SFC column in the preparation of Example 106.

MS ESI m/z 443.10 (M+H)⁺.

### Example 108: 4-(3-(2-amino-[1,2,4]triazolo[1,5-a]pyridin-7-yl)-2-fluoro-6-methylphenoxy)-2-fluoro-1-(4-fluorophenyl)butan-1-ol enantiomer 3

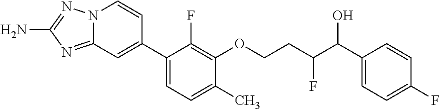 [SMILES: Cc1ccc(-c2ccn3nc(N)nc3c2)c(F)c1OCCC(F)C(O)c1ccc(F)cc1]

The title compound 4-(3-(2-amino-[1,2,4]triazolo[1,5-a]pyridin-7-yl)-2-fluoro-6-methylphenoxy)-2-fluoro-1-(4-fluorophenyl)butan-1-ol enantiomer 3 (49.2 mg, 0.104 mmol, 11.3%) was obtained as the third eluting enantiomer from the preparative SFC column in the preparation of Example 106.

MS ESI m/z 443.10 (M+H)⁺.

### Example 109: 4-(3-(2-amino-[1,2,4]triazolo[1,5-a]pyridin-7-yl)-2-fluoro-6-methylphenoxy)-2-fluoro-1-(4-fluorophenyl)butan-1-ol enantiomer 4

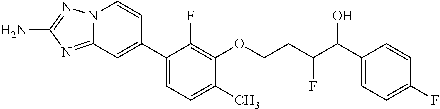 [SMILES: Cc1ccc(-c2ccn3nc(N)nc3c2)c(F)c1OCCC(F)C(O)c1ccc(F)cc1]

The title compound 4-(3-(2-amino-[1,2,4]triazolo[1,5-a]pyridin-7-yl)-2-fluoro-6-methylphenoxy)-2-fluoro-1-(4-fluorophenyl)butan-1-ol enantiomer 4 (49.2 mg, 0.104 mmol, 11.3%) was obtained as the fourth eluting enantiomer from the preparative SFC column in the preparation of Example 106.

MS ESI m/z 443.10 (M+H)⁺.

### Example 110: 4-(3-(2-amino-[1,2,4]triazolo[1,5-a]pyridin-7-yl)-2-fluoro-6-(trifluoromethyl)phenoxy)-2-fluoro-1-(4-fluorophenyl)butan-1-ol enantiomer 1

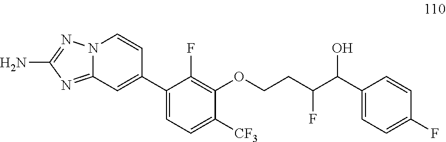 [SMILES: Nc1nc2cc(-c3ccc(C(F)(F)F)c(OCCC(F)C(O)c4ccc(F)cc4)c3F)ccn2n1]

(The unassigned 4 diasteromers are:

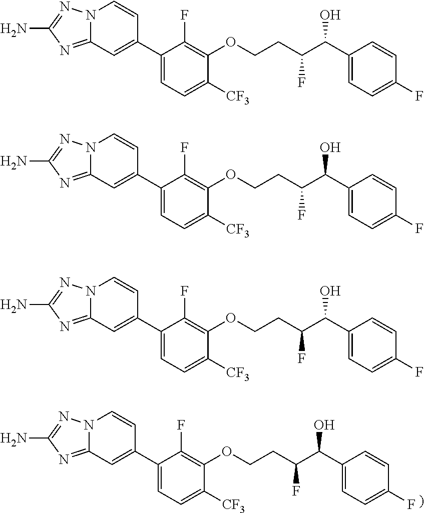 [SMILES: Nc1nc2cc(-c3ccc(C(F)(F)F)c(OCC[C@@H](F)[C@H](O)c4ccc(F)cc4)c3F)ccn2n1 ; Nc1nc2cc(-c3ccc(C(F)(F)F)c(OCC[C@@H](F)[C@@H](O)c4ccc(F)cc4)c3F)ccn2n1 ; Nc1nc2cc(-c3ccc(C(F)(F)F)c(OCC[C@H](F)[C@H](O)c4ccc(F)cc4)c3F)ccn2n1 ; Nc1nc2cc(-c3ccc(C(F)(F)F)c(OCC[C@H](F)[C@@H](O)c4ccc(F)cc4)c3F)ccn2n1]

### 110A: ethyl 4-(3-bromo-2-fluoro-6-(trifluoromethyl)phenoxy)-2-fluorobutanoate

To a dry 100 mL round bottom flask under N₂ was added ethyl 2-fluoro-4-iodobutanoate (930 mg, 3.58 mmol), 3-bromo-2-fluoro-6-(trifluoromethyl)phenol (1.0 g, 3.86 mmol) and acetone (20 mL). The reaction was flushed briefly with N₂, treated with potassium carbonate (740 mg, 5.35 mmol), equipped with a cold water condenser and heated at 60° C. for 18 h. The reaction was filtered through a pad of celite and the filtrate was evaporated to dryness. The residue was dissolved in EtOAc (225 mL) and the organic layer was washed with 1.0 M sodium hydroxide (2×10 mL), water (1×10 mL), brine (1×10 mL), dried over sodium sulfate, filtered and evaporated to dryness to give the title compound, ethyl 4-(3-bromo-2-fluoro-6-(trifluoromethyl)phenoxy)-2-fluorobutanoate (1.44 g, 3.68 mmol, quant.) that was used without further purification.

¹H NMR (600 MHz, chloroform-d) δ 7.39 (dd, J=8.4, 6.1 Hz, 1H), 7.30-7.26 (m, 1H), 5.29-5.13 (m, 1H), 4.41-4.25 (m, 4H), 2.60-2.41 (m, 1H), 2.38-2.21 (m, 1H), 1.35 (t, J=7.2 Hz, 3H)

### 110B: 4-(3-bromo-2-fluoro-6-(trifluoromethyl)phenoxy)-2-fluorobutanoic acid

To a solution of ethyl 4-(3-bromo-2-fluoro-6-(trifluoromethyl)phenoxy)-2-fluorobutanoate (1.44 g, 3.68 mmol) in a mixture of THF (20 mL) and water (10 mL) was added lithium hydroxide (211 mg, 8.81 mmol). The reaction was flushed briefly with N₂, capped and allowed to stir at room temp for 18 h. Some of the solvent was removed in vacuo and the reaction was cooled in an ice/water bath and carefully treated with HCl, 1.0M in water (9.2 mL, 9.20 mmol). The reaction was diluted with EtOAc (225 mL) and the organic layer was washed with brine (1×10 mL), dried over sodium sulfate, filtered and evaporated to dryness to give the title compound, -(3-bromo-2-fluoro-6-(trifluoromethyl)phenoxy)-2-fluorobutanoic acid (1.38 g, 3.85 mmol, quant).

¹H NMR (600 MHz, chloroform-d) δ 7.40 (dd, J=8.4, 6.1 Hz, 1H), 7.32-7.27 (m, 1H), 5.41-5.24 (m, 1H), 4.49-4.32 (m, 2H), 2.67-2.49 (m, 1H), 2.37 (dddt, J=18.8, 14.1, 9.4, 4.7 Hz, 1H).

### 110C: 4-(3-bromo-2-fluoro-6-(trifluoromethyl)phenoxy)-2-fluoro-N-methoxy-N-methylbutanamide

To a solution of 4-(3-bromo-2-fluoro-6-(trifluoromethyl)phenoxy)-2-fluorobutanoic acid (1.38 g, 3.80 mmol), N,O-dimethylhydroxylamine (445 mg, 4.56 mmol), and N,N-diisopropyl-ethylamine (3.3 mL, 18.89 mmol) in anhydrous DCM (25 mL) was added BOP (2.2 g, 4.97 mmol). The reaction was flushed briefly with N₂, capped and allowed to stir at rt for 18 h. The solvent was removed in vacuo and the crude residue purified by flash chromatography on silica gel, eluting with 0-65% gradient of EtOAc in hexane to afford the title compound, 4-(3-bromo-2-fluoro-6-(trifluoromethyl)phenoxy)-2-fluoro-N-methoxy-N-methylbutanamide (1.21 g, 2.99 mmol, 78%).

¹H NMR (500 MHz, chloroform-d) δ 7.44-7.32 (m, 1H), 7.30-7.25 (m, 1H), 5.76-5.48 (m, 1H), 4.53-4.33 (m, 2H), 3.78 (s, 3H), 3.28 (s, 3H), 2.54-2.21 (m, 2H).

### 110D: 4-(3-bromo-2-fluoro-6-(trifluoromethyl)phenoxy)-2-fluoro-1-(4-fluorophenyl)butan-1-one

To an ice cold solution of 4-(3-bromo-2-fluoro-6-(trifluoromethyl)phenoxy)-2-fluoro-N-methoxy-N-methylbutanamide (1.21 g, 2.98 mmol) in anhydrous THF (15 mL) was slowly added 4-fluorophenylmagnesium bromide 1.0M solution in THF (3.6 mL, 3.60 mmol). The reaction was stirred at 0° C. for 20 min, then the bath was removed and the reaction was allowed to stir at rt for 4.5 h. The reaction was quenched with aqueous saturated ammonium chloride (8 mL) and diluted with EtOAc (40 mL). The aqueous layer was washed with EtOAc (4×40 mL), the organic layers were combined, washed with brine (1×5 mL), dried over sodium sulfate, filtered and evaporated to dryness. The crude residue was purified by flash chromatography on silica gel, eluting with 0-65% gradient of DCM in hexane to afford the title compound, 4-(3-bromo-2-fluoro-6-(trifluoromethyl)phenoxy)-2-fluoro-1-(4-fluorophenyl)butan-1-one (1.18 g, 2.67 mmol, 90%).

¹H NMR (500 MHz, chloroform-d) δ 8.14-8.03 (m, 2H), 7.46-7.36 (m, 1H), 7.33-7.28 (m, 1H), 7.25-7.15 (m, 2H), 6.12-5.90 (m, 1H), 4.59-4.36 (m, 2H), 2.71-2.46 (m, 1H), 2.41-2.23 (m, 1H).

### 110E: 4-(3-bromo-2-fluoro-6-(trifluoromethyl)phenoxy)-2-fluoro-1-(4-fluorophenyl)butan-1-ol

To a solution of 4-(3-bromo-2-fluoro-6-(trifluoromethyl)phenoxy)-2-fluoro-1-(4-fluorophenyl)-butan-1-one (616 mg, 1.396 mmol) in MeOH (15 mL) was slowly added sodium borohydride (80 mg, 2.115 mmol) with rapid stirring. After the addition was complete, the reaction was stirred at rt for 15 min. The reaction was quenched with water (15 mL) and diluted with EtOAc (100 mL). The aqueous layer was extracted with EtOAc (4×25 mL), the organic layers were combined, washed with brine (1×10 mL), dried over sodium sulfate, filtered and evaporated to dryness to give the title compound, 4-(3-bromo-2-fluoro-6-(trifluoromethyl)phenoxy)-2-fluoro-1-(4-fluorophenyl)butan-1-ol (653 mg, 1.47 mmol, quant) that was used without further purification.

### 110F: tert-butyl (tert-butoxycarbonyl)(7-(2-fluoro-3-(3-fluoro-4-(4-fluorophenyl)-4-hydroxybutoxy)-4-(trifluoromethyl)phenyl)-[1,2,4]triazolo[1,5-a]pyridin-2-yl)carbamate

To a flask containing 4-(3-bromo-2-fluoro-6-(trifluoromethyl)phenoxy)-2-fluoro-1-(4-fluorophenyl)butan-1-ol (453 mg, 1.022 mmol) was added tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (518 mg, 1.125 mmol), dioxane (10 mL) and potassium phosphate tribasic (2.1 mL, 4.20 mmol). The reaction was purged very well with argon, then treated with dichloro[1,1′-bis(di-t-butyl-phosphino)ferrocene]palladium(II), 99% (35 mg, 0.054 mmol). The reaction was again flushed very well with argon, equipped with a cold water condenser and heated at 115° C. for 2.5 h. The reaction was diluted with water and the aqueous layer was washed with EtOAc (4×50 mL). The organic layers were combined, washed with brine (1×10 mL), dried over sodium sulfate, filtered and evaporated to dryness. The crude residue was purified by flash chromatography on silica gel, eluting with 0-65% gradient of EtOAc in DCM to afford the title compound, tert-butyl (tert-butoxycarbonyl)(7-(2-fluoro-3-(3-fluoro-4-(4-fluorophenyl)-4-hydroxybutoxy)-4-(trifluoromethyl)phenyl)-[1,2,4]triazolo[1,5-a]pyridin-2-yl)carbamate (640 mg, 0.9196 mmol, 90%).

MS ESI m/z 697.15 (M+H)⁺.

### Example 110: 4-(3-(2-amino-[1,2,4]triazolo[1,5-a]pyridin-7-yl)-2-fluoro-6-(trifluoromethyl)phenoxy)-2-fluoro-1-(4-fluorophenyl)butan-1-ol enantiomer 1

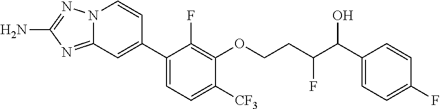 [SMILES: Nc1nc2cc(-c3ccc(C(F)(F)F)c(OCCC(F)C(O)c4ccc(F)cc4)c3F)ccn2n1]

To a solution of tert-butyl (tert-butoxycarbonyl)(7-(2-fluoro-3-(3-fluoro-4-(4-fluorophenyl)-4-hydroxybutoxy)-4-(trifluoromethyl)phenyl)-[1,2,4]triazolo[1,5-a]pyridin-2-yl)carbamate (640 mg, 0.919 mmol) in MeOH (4 mL) was added HCl 4.0M solution in dioxane (8 mL, 32.0 mmol) and the reaction was heated at 60° C. oil bath for 90 min. The volatiles were removed under a gentle stream of nitrogen and the crude residue was purified via preparative SFC with the following conditions: Column: Chiralpak IC, 30 mm×250 mm, 5 m particles; Mobile Phase A: supercritical C₀₂; Mobile Phase B: MeOH with 0.1% NH₄₀H; Elution gradient: isocratic 18% B over 29 minutes Flow Rate: 90 mL/min; Column Temperature: 40° C. Fraction collection was triggered by UV (220 nm). Fractions containing the first enantiomer to elute from the preparative SFC column were combined and dried via centrifugal evaporation. The material was further purified via preparative SFC with the following conditions: Column: Lux Cellulose-2, 30 mm×250 mm, 5 m particles; Mobile Phase A: supercritical C₀₂; Mobile Phase B: MeOH with 0.1% NH₄₀H; Flow Rate: 90 mL/min; Column Temperature: 45° C. Fraction collection was triggered by UV (220 nm). Fractions containing the desired product were combined and dried via centrifugal evaporation. provide the title compound, 4-(3-(2-amino-[1,2,4]triazolo[1,5-a]pyridin-7-yl)-2-fluoro-6-(trifluoromethyl)phenoxy)-2-fluoro-1-(4-fluorophenyl)butan-1-ol enantiomer 1 (58.8 mg, 0.118 mmol, 12.8%).

MS ESI m/z 497.10 (M+H)⁺.

### Example 111: 4-(3-(2-amino-[1,2,4]triazolo[1,5-a]pyridin-7-yl)-2-fluoro-6-(trifluoromethyl)phenoxy)-2-fluoro-1-(4-fluorophenyl)butan-1-ol enantiomer 2

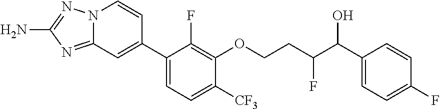 [SMILES: Nc1nc2cc(-c3ccc(C(F)(F)F)c(OCCC(F)C(O)c4ccc(F)cc4)c3F)ccn2n1]

The title compound 4-(3-(2-amino-[1,2,4]triazolo[1,5-a]pyridin-7-yl)-2-fluoro-6-(trifluoromethyl)phenoxy)-2-fluoro-1-(4-fluorophenyl)butan-1-ol enantiomer 2 (83.2 mg, 0.168 mmol, 17.7%) was obtained as the second eluting enantiomer from the preparative SFC column in the preparation of Example 110.

MS ESI m/z 497.10 (M+H)⁺.

### Example 112: 4-(3-(2-amino-[1,2,4]triazolo[1,5-a]pyridin-7-yl)-2-fluoro-6-(trifluoromethyl)phenoxy)-2-fluoro-1-(4-fluorophenyl)butan-1-ol enantiomer 3

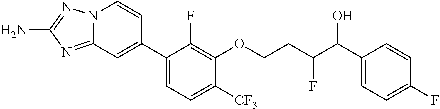 [SMILES: Nc1nc2cc(-c3ccc(C(F)(F)F)c(OCCC(F)C(O)c4ccc(F)cc4)c3F)ccn2n1]

The title compound 4-(3-(2-amino-[1,2,4]triazolo[1,5-a]pyridin-7-yl)-2-fluoro-6-(trifluoromethyl)phenoxy)-2-fluoro-1-(4-fluorophenyl)butan-1-ol enantiomer 3 (81.5 mg, 0.164 mmol, 17.5%) was obtained as the third eluting enantiomer from the preparative SFC column in the preparation of Example 110.

MS ESI m/z 497.10 (M+H)⁺.

### Example 113: 4-(3-(2-amino-[1,2,4]triazolo[1,5-a]pyridin-7-yl)-2-fluoro-6-(trifluoromethyl)phenoxy)-2-fluoro-1-(4-fluorophenyl)butan-1-ol enantiomer 4

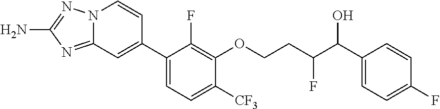 [SMILES: Nc1nc2cc(-c3ccc(C(F)(F)F)c(OCCC(F)C(O)c4ccc(F)cc4)c3F)ccn2n1]

The title compound 4-(3-(2-amino-[1,2,4]triazolo[1,5-a]pyridin-7-yl)-2-fluoro-6-(trifluoromethyl)phenoxy)-2-fluoro-1-(4-fluorophenyl)butan-1-ol enantiomer 4 (62.4 mg, 0.126 mmol, 13.6%) was obtained as the forth eluting enantiomer from the preparative SFC column in the preparation of Example 110.

MS ESI m/z 497.10 (M+H)⁺.

### Example 114: 5-(3-(2-amino-[1,2,4]triazolo[1,5-a]pyridin-7-yl)-2-fluoro-6-methylphenoxy)-3-fluoro-2-(4-fluorophenyl)pentan-2-ol enantiomer 1

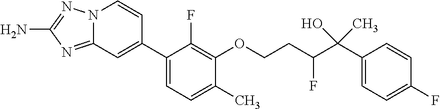 [SMILES: Cc1ccc(-c2ccn3nc(N)nc3c2)c(F)c1OCCC(F)C(C)(O)c1ccc(F)cc1]

(The 4 unassigned diasteromers are:

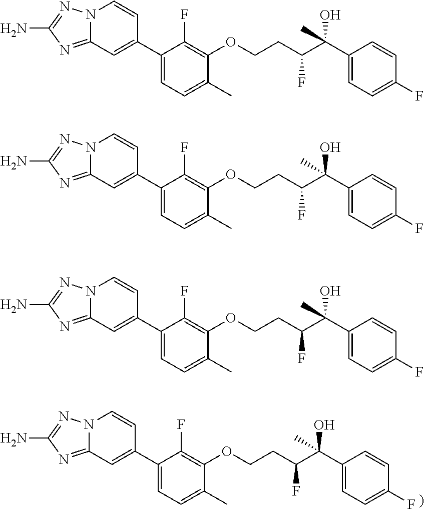 [SMILES: Cc1ccc(-c2ccn3nc(N)nc3c2)c(F)c1OCC[C@@H](F)[C@](C)(O)c1ccc(F)cc1 ; Cc1ccc(-c2ccn3nc(N)nc3c2)c(F)c1OCC[C@@H](F)[C@@](C)(O)c1ccc(F)cc1 ; Cc1ccc(-c2ccn3nc(N)nc3c2)c(F)c1OCC[C@H](F)[C@](C)(O)c1ccc(F)cc1 ; Cc1ccc(-c2ccn3nc(N)nc3c2)c(F)c1OCC[C@H](F)[C@@](C)(O)c1ccc(F)cc1]

### 114A: 5-(3-bromo-2-fluoro-6-methylphenoxy)-3-fluoro-2-(4-fluorophenyl)-pentan-2-ol

To a solution of 4-(3-bromo-2-fluoro-6-methylphenoxy)-2-fluoro-1-(4-fluorophenyl)butan-1-one (207.5 mg, 0.536 mmol) in THF (3 mL) under N₂ was added methylmagnesium bromide 3.0 M solution in diethyl ether (0.54 mL, 1.620 mmol). The reaction was stirred at rt for 20 min, then quenched with aqueous saturated ammonium chloride (5 mL) and diluted with EtOAc (50 mL). The aqueous layer was washed with EtOAc (2×25 mL), the organic layers were combined, washed with brine (1×10 mL), dried over sodium sulfate, filtered and evaporated to dryness to give the title compound, 5-(3-bromo-2-fluoro-6-methylphenoxy)-3-fluoro-2-(4-fluorophenyl)-pentan-2-ol (228 mg, 0.565 mmol, quant) that was used without further purification.

¹H NMR (600 MHz, chloroform-d) δ 7.51-7.40 (m, 2H), 7.18-7.03 (m, 3H), 6.81 (d, J=8.4 Hz, 1H), 5.02-4.82 (m, 1H), 4.17-4.08 (m, 1H), 4.04 (dt, J=9.4, 4.9 Hz, 1H), 2.22-2.14 (m, 4H), 2.06-1.97 (m, 1H), 1.74-1.74 (m, 1H), 1.87-1.74 (m, 1H), 1.71 (d, J=2.0 Hz, 3H).

### 114B: tert-butyl (tert-butoxycarbonyl)(7-(2-fluoro-3-((3-fluoro-4-(4-fluorophenyl)-4-hydroxypentyl)oxy)-4-methylphenyl)-[1,2,4]triazolo[1,5-a]pyridin-2-yl)carbamate

To a solution of 5-(3-bromo-2-fluoro-6-methylphenoxy)-3-fluoro-2-(4-fluorophenyl)pentan-2-ol (228 mg, 0.565 mmol), tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (286 mg, 0.621 mmol) in dioxane (8 mL) was added potassium phosphate tribasic, 2.0 M in water (1.15 mL, 2.300 mmol). The reaction was purged well with argon, then treated with dichloro[1,1′-bis(di-t-butyl-phosphino)ferrocene]palladium(II), 99% (20 mg, 0.031 mmol). The reaction was again purged very well with argon, equipped with a cold water condenser and heated at 115° C. for 4 h. The reaction was diluted with water (20 mL) and the aqueous layer was washed with EtOAc (4×50 mL). The organic layers were combined, washed with brine (1×15 mL), dried over sodium sulfate, filtered and evaporated to dryness. The crude residue was purified by flash chromatography on silica gel, eluting with 0-50% gradient of EtOAc in DCM to afford the title compound, tert-butyl (tert-butoxycarbonyl)(7-(2-fluoro-3-((3-fluoro-4-(4-fluorophenyl)-4-hydroxypentyl)oxy)-4-methylphenyl)-[1,2,4]triazolo[1,5-a]pyridin-2-yl)carbamate (259 mg, 0.394 mmol, 69.8%).

MS ESI m/z 657.20 (M+H)⁺.

### Example 114: 5-(3-(2-amino-[1,2,4]triazolo[1,5-a]pyridin-7-yl)-2-fluoro-6-methylphenoxy)-3-fluoro-2-(4-fluorophenyl)pentan-2-ol enantiomer 1

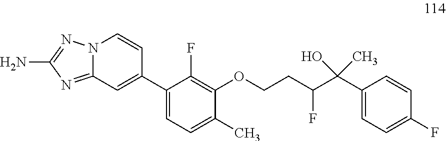 [SMILES: Cc1ccc(-c2ccn3nc(N)nc3c2)c(F)c1OCCC(F)C(C)(O)c1ccc(F)cc1]

To a solution of tert-butyl (tert-butoxycarbonyl)(7-(2-fluoro-3-((3-fluoro-4-(4-fluorophenyl)-4-hydroxypentyl)oxy)-4-methylphenyl)-[1,2,4]triazolo[1,5-a]pyridin-2-yl)carbamate (259 mg, 0.394 mmol) in MeOH (5 mL) was added HCl 4.0M solution in dioxane (3 mL, 12.00 mmol). The reaction was securely capped and heated at 60° C. for 45 min. The reaction was treated with additional HCl 4.0M solution in dioxane (3 mL, 12.00 mmol) and heated at 60° C. for 30 min. The volatiles were removed under a gentle stream of nitrogen and the crude residue was purified via preparative SFC with the following conditions: Column: Chiralpak IC, 30 mm×250 mm, 5 m particles; Mobile Phase A: supercritical CO2; Mobile Phase B: i-PrOH with 0.1% NH₄₀H; Elution gradient: isocratic 30% B over 40 minutes Flow Rate: 90 mL/min; Column Temperature: 40° C. Fraction collection was triggered by UV (220 nm). Fractions containing the first enantiomer to elute from the preparative SFC column were combined and dried via centrifugal evaporation to give the title compound, 5-(3-(2-amino-[1,2,4]triazolo[1,5-a]pyridin-7-yl)-2-fluoro-6-(trifluoromethyl)phenoxy)-3-fluoro-2-(4-fluorophenyl)pentan-2-ol enantiomer 1 (61.8 mg, 0.135 mmol, 32.6%) MS ESI m/z 457.15 (M+H)⁺.

### Example 115: 5-(3-(2-amino-[1,2,4]triazolo[1,5-a]pyridin-7-yl)-2-fluoro-6-methylphenoxy)-3-fluoro-2-(4-fluorophenyl)pentan-2-ol enantiomer 2

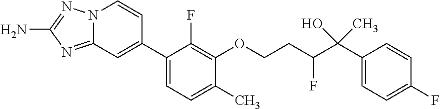 [SMILES: Cc1ccc(-c2ccn3nc(N)nc3c2)c(F)c1OCCC(F)C(C)(O)c1ccc(F)cc1]

The title compound, 5-(3-(2-amino-[1,2,4]triazolo[1,5-a]pyridin-7-yl)-2-fluoro-6-methylphenoxy)-3-fluoro-2-(4-fluorophenyl)pentan-2-ol enantiomer 2 (81.1 mg, 0.178 mmol, 44.6%) was obtained as the second eluting enantiomer from the preparative SFC column in the preparation of Example 114 MS ESI m/z 457.15 (M+H)⁺.

### Example 116: 4-(5-(2-amino-[1,2,4]triazolo[1,5-a]pyridin-7-yl)-2,3-difluorophenoxy)-2-fluoro-1-(4-fluorophenyl)butan-1-ol enantiomer 1

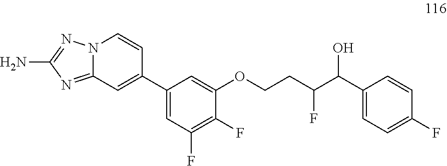 [SMILES: Nc1nc2cc(-c3cc(F)c(F)c(OCCC(F)C(O)c4ccc(F)cc4)c3)ccn2n1]

(The 4 unassigned diasteromers are:

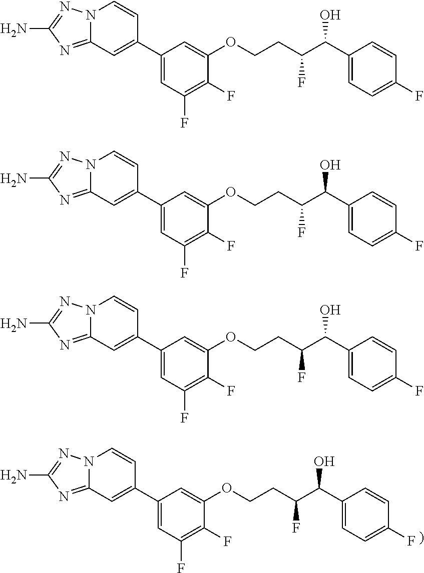 [SMILES: Nc1nc2cc(-c3cc(F)c(F)c(OCC[C@@H](F)[C@H](O)c4ccc(F)cc4)c3)ccn2n1 ; Nc1nc2cc(-c3cc(F)c(F)c(OCC[C@@H](F)[C@@H](O)c4ccc(F)cc4)c3)ccn2n1 ; Nc1nc2cc(-c3cc(F)c(F)c(OCC[C@H](F)[C@H](O)c4ccc(F)cc4)c3)ccn2n1 ; Nc1nc2cc(-c3cc(F)c(F)c(OCC[C@H](F)[C@@H](O)c4ccc(F)cc4)c3)ccn2n1]

### 116A: ethyl 4-(5-bromo-2,3-difluorophenoxy)-2-fluorobutanoate

To a 150 mL pressure bottle under N₂ was added ethyl 2-fluoro-4-iodobutanoate (1.0 g, 3.85 mmol), 5-bromo-2,3-difluorophenol (885 mg, 4.23 mmol), potassium carbonate (800 mg, 5.79 mmol) and acetone (30 mL). The reaction was securely capped and heated at 65° C. for 18 h. The reaction was filtered through a small pad of celite and the filtrate was evaporated to dryness to give the title compound, ethyl 4-(5-bromo-2,3-difluorophenoxy)-2-fluorobutanoate (1.47 g, 4.31 mmol, quant) that was used without further purification.

¹H NMR (500 MHz, chloroform-d) δ 7.00 (ddd, J=9.2, 6.2, 2.3 Hz, 1H), 6.92 (dt, J=6.4, 2.1 Hz, 1H), 5.26-5.10 (m, 1H), 4.36-4.27 (m, 2H), 4.26-4.14 (m, 2H), 2.60-2.31 (m, 2H), 1.35 (t, J=7.2 Hz, 3H).

### 116B: 4-(5-bromo-2,3-difluorophenoxy)-2-fluorobutanoic acid

To a solution of ethyl 4-(5-bromo-2,3-difluorophenoxy)-2-fluorobutanoate (1.47 g, 4.31 mmol) in a mixture of THF (20 mL) and water (10 mL) was added lithium hydroxide (252.3 mg, 10.54 mmol). The reaction was sonicated briefly and allowed to stir at rt for 70 min., then most of the solvent was removed in vacuo. The reaction was cooled in an ice/water bath and treated with HCl, 1M in water (11 mL, 11.00 mmol) and then diluted with EtOAc (150 ml). The aqueous layer was washed with EtOAc (2×25 mL), the organic layers were combined, washed with brine (1×10 mL), dried over sodium sulfate, filtered and evaporated to dryness to give the title compound, 4-(5-bromo-2,3-difluorophenoxy)-2-fluorobutanoic acid (1.26 g, 4.02 mmol, 99%) that was used without further purification.

### 116C: 4-(5-bromo-2,3-difluorophenoxy)-2-fluoro-N-methoxy-N-methylbutanamide

To a solution of 4-(5-bromo-2,3-difluorophenoxy)-2-fluorobutanoic acid (1.26 g, 4.02 mmol) and N,O-dimethylhydroxylamine (475 mg, 4.87 mmol) in DCM was added N,N-diisopropyl-ethylamine (3.5 mL, 20.04 mmol). The reaction was flushed briefly with N₂, treated with BOP (2.3 g, 5.20 mmol), capped and allowed to sit at rt for 18 h. The solvent was removed in vacuo and the crude residue was purified by flash chromatography on silica gel, eluting with 0-75% gradient of EtOAc in hexanes to afford the title compound, 4-(5-bromo-2,3-difluorophenoxy)-2-fluoro-N-methoxy-N-methylbutanamide (1.01 g, 2.84 mmol, 70.5%)

¹H NMR (500 MHz, chloroform-d) δ 6.99 (ddd, J=9.2, 6.2, 2.3 Hz, 1H), 6.93 (dt, J=6.4, 2.2 Hz, 1H), 5.62-5.43 (m, 1H), 4.32-4.17 (m, 2H), 3.78 (s, 3H), 3.27 (s, 3H), 2.55-2.30 (m, 2H)

MS ESI m/z 355.85 (M+H)⁺.

### 116D: 4-(5-bromo-2,3-difluorophenoxy)-2-fluoro-1-(4-fluorophenyl)butan-1-one

To an ice cold solution of 4-(5-bromo-2,3-difluorophenoxy)-2-fluoro-N-methoxy-N-methyl-butanamide (1.01 g, 2.84 mmol) in anhydrous THF (20 mL) was slowly added 4-fluorophenyl-magnesium bromide 1.0 M solution in THF (3.4 mL, 3.40 mmol). The reaction was stirred cold for 15 min, then the bath was removed and the reaction was allowed to stir at rt for 4 h 45 min. The reaction was quenched with aqueous saturated NH₄C₁ (10 mL) and diluted with EtOAc (40 mL). The aqueous layer was washed with EtOAc (3×40 mL) and the organic layers were combined. The organic layer was washed with brine, dried over sodium sulfate, filtered and evaporated to dryness. The crude residue was purified by flash chromatography on silica gel, eluting with 0-65% gradient of dichloromethane in hexanes to afford the title compound, 4-(5-bromo-2,3-difluorophenoxy)-2-fluoro-1-(4-fluorophenyl)butan-1-one (931 mg, 2.38 mmol, 84%)

¹H NMR (500 MHz, chloroform-d) δ 8.16-8.04 (m, 2H), 7.26-7.15 (m, 2H), 7.02 (ddd, J=9.0, 6.4, 2.3 Hz, 1H), 6.94 (dt, J=6.4, 2.1 Hz, 1H), 6.03-5.81 (m, 1H), 4.40-4.20 (m, 2H), 2.70-2.50 (m, 1H), 2.50-2.32 (m, 1H).

### 116E: 4-(5-bromo-2,3-difluorophenoxy)-2-fluoro-1-(4-fluorophenyl)butan-1-ol

To a solution of 4-(5-bromo-2,3-difluorophenoxy)-2-fluoro-1-(4-fluorophenyl)butan-1-one (300 mg, 0.767 mmol) in MeOH (8 mL) was added sodium borohydride (44 mg, 1.163 mmol) in small portions over 15 min. After the addition was complete, the reaction was stirred at rt for 15 min. Then some of the solvent was removed under a gentle stream of nitrogen. The reaction was quenched with water (10 mL) and diluted with EtOAc (100 mL). The water layer was washed with EtOAc (4×25 mL) and the organic layers were combined, washed with brine, dried over sodium sulfate, filtered and evaporated to dryness to give the title compound, 4-(5-bromo-2,3-difluorophenoxy)-2-fluoro-1-(4-fluorophenyl)butan-1-ol (308.3 mg, 0.784 mmol, quant) that was used without further purification.

### 116F: tert-butyl (tert-butoxycarbonyl)(7-(3,4-difluoro-5-(3-fluoro-4-(4-fluorophenyl)-4-hydroxybutoxy)phenyl)-[1,2,4]triazolo[1,5-a]pyridin-2-yl)carbamate

To a solution of 4-(5-bromo-2,3-difluorophenoxy)-2-fluoro-1-(4-fluorophenyl)butan-1-ol (200 mg, 0.509 mmol) and tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate in dioxane was added potassium phosphate tribasic, 2.0 M in water (1.02 mL, 2.040 mmol). The reaction was purged with argon, then treated with dichloro[1,1′-bis(di-t-butylphosphino)ferrocene]palladium(II), 99% (20 mg, 0.031 mmol). The reaction was again purged well with argon, equipped with a cold water condenser and heated at 65° C. for 2.5 h. The reaction was quenched with water (20 mL) and diluted with EtOAc (100 mL). The aqueous layer was washed with EtOAc (4×25 mL) and the organic layers were combined, washed with brine, dried over sodium sulfate, filtered and evaporated to dryness. The crude residue was purified by flash chromatography on silica gel, eluting with 0-100% gradient of EtOAc in dichloromethane to afford the title compound, tert-butyl (tert-butoxycarbonyl)(7-(3,4-difluoro-5-(3-fluoro-4-(4-fluorophenyl)-4-hydroxybutoxy)phenyl)-[1,2,4]triazolo[1,5-a]pyridin-2-yl)carbamate (230 mg, 0.356 mmol, 69.9%).

MS ESI m/z 647.20 (M+H)⁺.

### Example 116: 4-(5-(2-amino-[1,2,4]triazolo[1,5-a]pyridin-7-yl)-2,3-difluorophenoxy)-2-fluoro-1-(4-fluorophenyl)butan-1-ol enantiomer 1

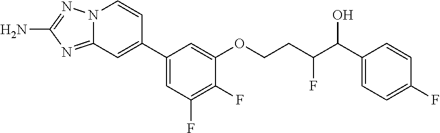 [SMILES: Nc1nc2cc(-c3cc(F)c(F)c(OCCC(F)C(O)c4ccc(F)cc4)c3)ccn2n1]

To a solution of tert-butyl (tert-butoxycarbonyl)(7-(3,4-difluoro-5-(3-fluoro-4-(4-fluorophenyl)-4-hydroxybutoxy)phenyl)-[1,2,4]triazolo[1,5-a]pyridin-2-yl)carbamate (230 mg, 0.356 mmol) in MeOH (4 mL) was added HCl 4.0M solution in dioxane (4 mL, 16.00 mmol) and the reaction was heated at 60° C. for 2.5 hours. The volatiles were removed under a gentle stream of nitrogen and the crude residue was purified via preparative SFC chromatography with the following conditions: Column: Chiralpak IC, 30 mm×250 mm, 5 m particles; Mobile Phase A: supercritical CO2; Mobile Phase B: MeOH with 0.1% NH₄₀H; Elution gradient: isocratic 30% B over 30 minutes Flow Rate: 90 mL/min; Column Temperature: 40° C. Fraction collection was triggered by UV (220 nm). Fractions containing the first enantiomer to elute from the preparative SFC column were combined and dried via centrifugal evaporation. The material was further purified via preparative SFC with the following conditions: Column: Chiralpak IG, 30 mm×250 mm, 5 m particles; Mobile Phase A: supercritical CO2; Mobile Phase B: MeOH with 0.1% NH4OH; Flow Rate: 90 mL/min; Column Temperature: 45° C. Fraction collection was triggered by UV (220 nm). Fractions containing the desired product were combined and dried via centrifugal evaporation to give the title compound, 4-(5-(2-amino-[1,2,4]triazolo[1,5-a]pyridin-7-yl)-2,3-difluorophenoxy)-2-fluoro-1-(4-fluorophenyl)butan-1-ol enantiomer 1 (15.9 mg, 0.036 mmol, 10.0%).

MS ESI m/z 447.05 (M+H)⁺.

### Example 117: 4-(5-(2-amino-[1,2,4]triazolo[1,5-a]pyridin-7-yl)-2,3-difluorophenoxy)-2-fluoro-1-(4-fluorophenyl)butan-1-ol enantiomer 2

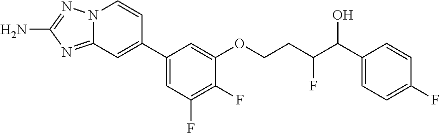 [SMILES: Nc1nc2cc(-c3cc(F)c(F)c(OCCC(F)C(O)c4ccc(F)cc4)c3)ccn2n1]

The title compound, 54-(5-(2-amino-[1,2,4]triazolo[1,5-a]pyridin-7-yl)-2,3-difluorophenoxy)-2-fluoro-1-(4-fluorophenyl)butan-1-ol enantiomer 2 (15.6 mg, 0.035 mmol, 9.8%) was obtained as the second eluting enantiomer from the preparative SFC column in the preparation of Example 116.

MS ESI m/z 457.15 (M+H)⁺.

### Example 118: 4-(5-(2-amino-[1,2,4]triazolo[1,5-a]pyridin-7-yl)-2,3-difluorophenoxy)-2-fluoro-1-(4-fluorophenyl)butan-1-ol enantiomer 3

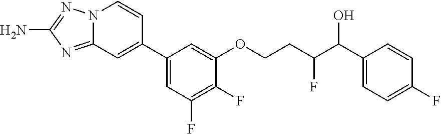 [SMILES: Nc1nc2cc(-c3cc(F)c(F)c(OCCC(F)C(O)c4ccc(F)cc4)c3)ccn2n1]

The title compound, 54-(5-(2-amino-[1,2,4]triazolo[1,5-a]pyridin-7-yl)-2,3-difluorophenoxy)-2-fluoro-1-(4-fluorophenyl)butan-1-ol enantiomer 3 (25.5 mg, 0.057 mmol, 16.1%) was obtained as the third eluting enantiomer from the preparative SFC column in the preparation of Example 116.

MS ESI m/z 457.15 (M+H)⁺.

### Example 119: 4-(5-(2-amino-[1,2,4]triazolo[1,5-a]pyridin-7-yl)-2,3-difluorophenoxy)-2-fluoro-1-(4-fluorophenyl)butan-1-ol enantiomer 4

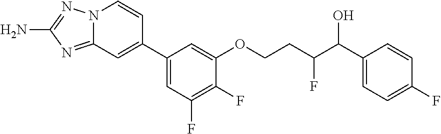 [SMILES: Nc1nc2cc(-c3cc(F)c(F)c(OCCC(F)C(O)c4ccc(F)cc4)c3)ccn2n1]

The title compound, 54-(5-(2-amino-[1,2,4]triazolo[1,5-a]pyridin-7-yl)-2,3-difluorophenoxy)-2-fluoro-1-(4-fluorophenyl)butan-1-ol enantiomer 4 (25.4 mg, 0.057 mmol, 16.0%) was obtained as the fourth eluting enantiomer from the preparative SFC column in the preparation of Example 116.

MS ESI m/z 457.15 (M+H)⁺.

### Example 120: 7-(2-fluoro-4-methyl-3-(3,4,4-trifluoro-4-(4-fluorophenyl)butoxy)phenyl)-[1,2,4]triazolo[1,5-a]pyridin-2-amine enantiomer 1

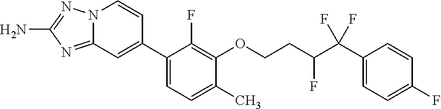 [SMILES: Cc1ccc(-c2ccn3nc(N)nc3c2)c(F)c1OCCC(F)C(F)(F)c1ccc(F)cc1]

(The 2 unassigned enantiomers are:

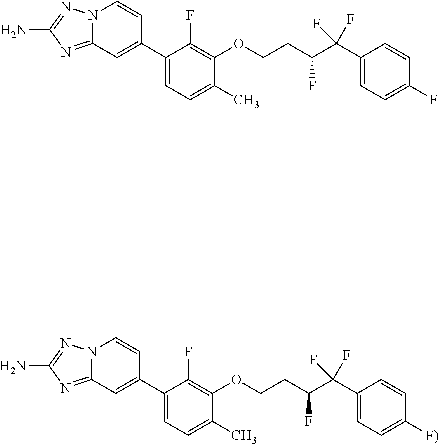

### 120A: 1-bromo-2-fluoro-4-methyl-3-(3,4,4-trifluoro-4-(4-fluorophenyl)-butoxy)benzene

To a solution of 4-(3-bromo-2-fluoro-6-methylphenoxy)-2-fluoro-1-(4-fluorophenyl)butan-1-one (200 mg, 0.517 mmol) in anhydrous DCM (5 mL) at rt was added DAST (545 μL, 4.13 mmol). The reaction was stirred at rt for 15 min, then heated at 65° C. for 8 h. The reaction was treated with additional DAST (1.0 mL, 7.57 mmol) and heated at 65° C. for 9 h. The reaction was cooled in an ice/water bath, quenched with aqueous saturated sodium bicarbonate (15 mL) and diluted with DCM (100 mL). The aqueous layer was washed with DCM (4×20 mL) and the organic layers were combined, washed with brine, dried over sodium sulfate, filtered and evaporated to dryness. The crude residue was purified by flash chromatography on silica gel, eluting with 0-100% gradient of DCM in hexane to afford the title compound, 1-bromo-2-fluoro-4-methyl-3-(3,4,4-trifluoro-4-(4-fluorophenyl)butoxy)benzene (142.1 mg, 0.347 mmol, 67.2%).

¹H NMR (500 MHz, chloroform-d) δ 7.56 (dd, J=8.7, 5.2 Hz, 2H), 7.23-7.12 (m, 3H), 6.84 (dt, J=8.3, 0.8 Hz, 1H), 5.22-5.01 (m, 1H), 4.19 (dd, J=7.9, 3.9 Hz, 2H), 2.24 (s, 4H), 2.16-1.97 (m, 1H).

### 120B: tert-butyl (tert-butoxycarbonyl)(7-(2-fluoro-4-methyl-3-(3,4,4-trifluoro-4-(4-fluorophenyl)butoxy)phenyl)-[1,2,4]triazolo[1,5-a]pyridin-2-yl)carbamate

To a suspension of 1-bromo-2-fluoro-4-methyl-3-(3,4,4-trifluoro-4-(4-fluorophenyl)butoxy)-benzene (65.6 mg, 0.160 mmol), tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (80 mg, 0.174 mmol) in dioxane (1.6 mL) was added potassium phosphate tribasic, 2.0M in water (0.320 mL, 0.641 mmol). The reaction was flushed very well with argon, then treated with dichloro[1,1′-bis(di-t-butyl-phosphino)ferrocene]palladium(II), 99% (7 mg, 10.74 μmol). The reaction was again flushed well with argon, equipped with a cold water condenser and heated at 65° C. for 2 h 15 min. The reaction was treated with additional catalyst (7 mg, 10.7 micromol), flushed with argon and heated at 65° C. for 5.5 h. The reaction was quenched with water (10 mL) and diluted with EtOAc (35 mL). The aqueous layer was washed with EtOAc (4×35 mL) and the organic layers were combined, washed with brine (1×10 mL), dried over sodium sulfate, filtered and evaporated to dryness. The crude residue was purified by flash chromatography on silica gel, eluting with 0-100% gradient of EtOAc in hexane to afford the title compound, tert-butyl (tert-butoxycarbonyl)(7-(2-fluoro-4-methyl-3-(3,4,4-trifluoro-4-(4-fluorophenyl)butoxy)phenyl)-[1,2,4]triazolo[1,5-a]pyridin-2-yl)carbamate (59.9 mg, 0.090 mmol, 56.4%).

MS ESI m/z 663.25 (M+H)⁺.

### Example 120: 7-(2-fluoro-4-methyl-3-(3,4,4-trifluoro-4-(4-fluorophenyl)butoxy)phenyl)-[1,2,4]triazolo[1,5-a]pyridin-2-amine enantiomer 1

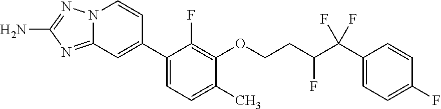 [SMILES: Cc1ccc(-c2ccn3nc(N)nc3c2)c(F)c1OCCC(F)C(F)(F)c1ccc(F)cc1]

To a solution of tert-butyl (tert-butoxycarbonyl)(7-(2-fluoro-4-methyl-3-(3,4,4-trifluoro-4-(4-fluorophenyl)butoxy)phenyl)-[1,2,4]triazolo[1,5-a]pyridin-2-yl)carbamate (59.9 mg, 0.090 mmol) in MeOH (3 mL) as added HCl 4.0M solution in dioxane (4.5 mL, 18.00 mmol) and the reaction was heated at 65° C. for 90 min. The volatiles were removed under a gentle stream of nitrogen and the crude residue was purified via preparative SFC with the following conditions: Column: Chiralpak IA, 30 mm×250 mm, 5 m particles; Mobile Phase A: supercritical CO₂; Mobile Phase B: MeOH with 0.1% NH₄OH; Elution gradient: isocratic 30% B over 15 minutes Flow Rate: 100 mL/min; Column Temperature: 40° C. Fraction collection was triggered by UV (220 nm). Fractions containing the first enantiomer to elute from the preparative SFC column were combined and dried via centrifugal evaporation to give the title compound, 7-(2-fluoro-4-methyl-3-(3,4,4-trifluoro-4-(4-fluorophenyl)butoxy)phenyl)-[1,2,4]triazolo[1,5-a]pyridin-2-amine enantiomer 1 (14.0 mg, 0.030 mmol, 33.1%).

MS ESI m/z 463.2 (M+H)⁺.

### Example 121: 7-(2-fluoro-4-methyl-3-(3,4,4-trifluoro-4-(4-fluorophenyl)butoxy)phenyl)-[1,2,4]triazolo[1,5-a]pyridin-2-amine enantiomer 2

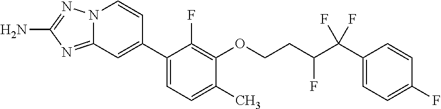 [SMILES: Cc1ccc(-c2ccn3nc(N)nc3c2)c(F)c1OCCC(F)C(F)(F)c1ccc(F)cc1]

The title compound, 7-(2-fluoro-4-methyl-3-(3,4,4-trifluoro-4-(4-fluorophenyl)butoxy)phenyl)-[1,2,4]triazolo[1,5-a]pyridin-2-amine enantiomer 2 (27.5 mg, 0.059 mmol, 65.3%) was obtained as the second eluting enantiomer from the preparative SFC column in the preparation of Example 120.

MS ESI m/z 463.1 (M+H)⁺.

### Example 122: 5-(3-(2-amino-[1,2,4]triazolo[1,5-a]pyridin-7-yl)-2,6-difluorophenoxy)-3,3-difluoro-2-(3-fluorophenyl)pentan-2-ol, enantiomer 1

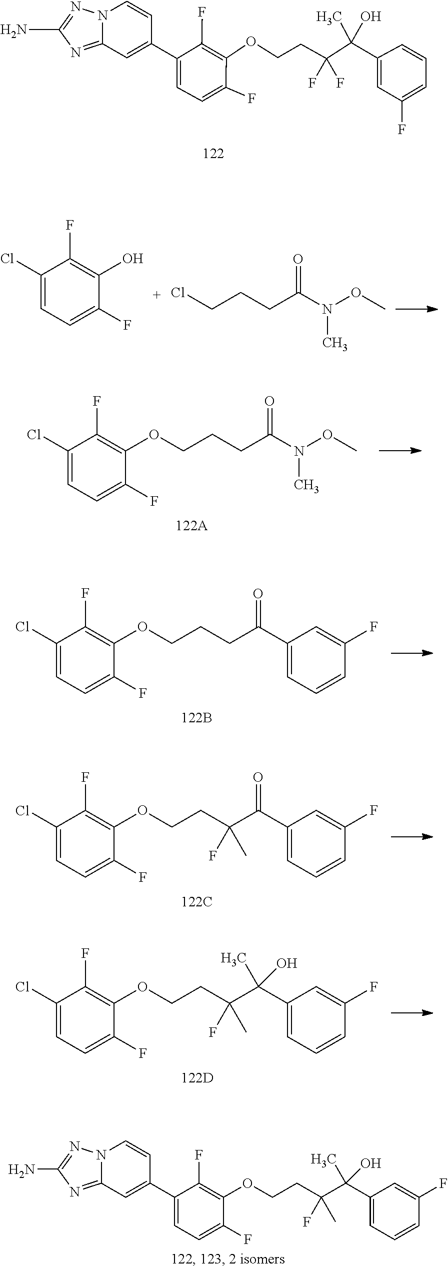

122A: To a solution of 4-chloro-N-methoxy-N-methylbutanamide (1.933 g, 11.67 mmol) in DMF (15 mL) was added 3-chloro-2,6-difluorophenol (1.6 g, 9.72 mmol), K₂CO₃ (1.882 g, 13.61 mmol) and KI (0.807 g, 4.86 mmol). The mixture was stirred at 60° C. for 18 h. Another 0.3 eq. of 4-chloro-N-methoxy-N-methylbutanamide was added. The mixture was stirred at 95° C. for 4 h. Et₂O was added. The mixture was washed 3× with water, then concentrated. The residue was purified via silica gel chromatography (80 g, hexanes-80% EtOAc) to give 4-(3-chloro-2,6-difluorophenoxy)-N-methoxy-N-methylbutanamide (2.3 g, 7.83 mmol, 81% yield).

¹H NMR (499 MHz, chloroform-d) δ 7.05 (ddd, J=9.1, 7.7, 5.3 Hz, 1H), 6.87 (ddd, J=10.1, 9.2, 2.2 Hz, 1H), 4.25 (t, J=6.0 Hz, 2H), 3.73 (s, 3H), 3.22 (s, 3H), 2.72 (br t, J=7.1 Hz, 2H), 2.15-2.09 (m, 2H).

MS ESI m/z 293.9 (M+H)⁺.

122B: To a solution of 4-(3-chloro-2,6-difluorophenoxy)-N-methoxy-N-methylbutanamide (294 mg, 1 mmol) in THF (3 mL) was added (3-fluorophenyl)magnesium bromide (4.00 mL, 2.0 mmol) at −10° C. under N₂. The mixture was stirred at 0° C. for 1 h. Another portion of (3-fluorophenyl)-magnesium bromide (4.00 mL, 2.0 mmol) was added at −10° C. After stirring at 0° C. for 1 h, Et₂O was added. The mixture was washed with NH₄Cl/water and water, dried over Na₂SO₄, then concentrated.

The residue was purified via silica gel chromatography (12 g, hexanes-15% EtOAc) to give 4-(3-chloro-2,6-difluorophenoxy)-1-(3-fluorophenyl)butan-1-one (290 mg, 0.882 mmol, 88% yield).

¹H NMR (499 MHz, chloroform-d) δ 7.81 (dt, J=7.8, 1.2 Hz, 1H), 7.72-7.68 (m, 1H), 7.48 (td, J=8.0, 5.5 Hz, 1H), 7.32-7.28 (m, 2H), 7.06 (ddd, J=9.2, 7.7, 5.2 Hz, 1H), 6.87 (ddd, J=10.0, 9.2, 2.2 Hz, 1H), 4.28 (t, J=5.9 Hz, 2H), 3.27 (t, J=7.1 Hz, 2H), 2.28-2.20 (m, 2H).

MS ESI m/z 328.9 (M+H)⁺.

122C: To a solution of 4-(3-chloro-2,6-difluorophenoxy)-1-(3-fluorophenyl)butan-1-one (280 mg, 0.852 mmol) in THF (3.0 mL) was added LHMDS (3.07 mL, 3.07 mmol) at −10° C. under N₂. The mixture was stirred at −10° C. to 0° C. for 20 min, then N-fluorobenzenesulfonimide (967 mg, 3.07 mmol) was added at −10° C. The mixture was stirred from −10° C. to 0° C. over 30 min. The reaction was quenched with 0.2N NaOH. Et₂O was added. The mixture was washed with 0.2 N NaOH and 2× water, then concentrated. The residue was purified via silica gel chromatography (12 g, hexanes-10% EtOAc) to give desired 4-(3-chloro-2,6-difluorophenoxy)-2,2-difluoro-1-(3-fluoro-phenyl)butan-1-one (220 mg, 0.603 mmol, 70.8% yield).

¹H NMR (499 MHz, chloroform-d) δ 7.95 (dt, J=7.8, 1.1 Hz, 1H), 7.82 (dd, J=9.4, 0.8 Hz, 1H), 7.52 (td, J=8.1, 5.5 Hz, 1H), 7.38 (tdd, J=8.2, 2.6, 0.9 Hz, 1H), 7.08 (ddd, J=9.2, 7.6, 5.3 Hz, 1H), 6.88 (ddd, J=10.0, 9.2, 2.2 Hz, 1H), 4.47 (t, J=6.7 Hz, 2H), 2.79 (tt, J=17.1, 6.7 Hz, 2H).

122D: To a solution of 4-(3-chloro-2,6-difluorophenoxy)-2,2-difluoro-1-(3-fluorophenyl)butan-1-one (220 mg, 0.603 mmol) in THF (3.0 mL) was added 3.0 M methylmagnesium bromide/Et₂O (0.402 mL, 1.206 mmol) at −10° C. under N₂. The mixture was stirred from −10° C. to rt for 30 min. The reaction was quenched with a couple of drops of water. Et₂O was added, dried over Na₂SO₄, then concentrated. The residue was purified via silica gel chromatography (12 g, hexanes-15% EtOAc) to give 5-(3-chloro-2,6-difluorophenoxy)-3,3-difluoro-2-(3-fluorophenyl)pentan-2-ol (210 mg, 0.552 mmol, 91% yield).

¹H NMR (499 MHz, chloroform-d) δ 7.39-7.30 (m, 3H), 7.09-7.01 (m, 2H), 6.85 (ddd, J=10.0, 9.2, 2.2 Hz, 1H), 4.37-4.25 (m, 2H), 2.59-2.48 (m, 1H), 2.14-2.01 (m, 1H), 1.73 (s, 3H).

122 and 123: A mixture of 7-bromo-[1,2,4]triazolo[1,5-a]pyridin-2-amine (10 g, 46.9 mmol), bis(pinacolato)diboron (14.30 g, 56.3 mmol), potassium acetate (13.82 g, 141 mmol), and [1,1′-bis(diphenylphosphino)ferrocene]dichloropalladium(II) (1.717 g, 2.347 mmol) in 1,4-dioxane (100 mL) was purged with N₂ and stirred at 100° C. for 2 h. LCMS showed that the reaction was complete. The reaction mixture was used as crude (2-amino-[1,2,4]triazolo[1,5-a]pyridin-7-yl)boronic acid.

To a solution of 5-(3-chloro-2,6-difluorophenoxy)-3,3-difluoro-2-(3-fluorophenyl)pentan-2-ol (120 mg, 0.315 mmol) in dioxane (1.5 mL) was added crude (2-amino-[1,2,4]triazolo[1,5-a]pyridin-7-yl)boronic acid (112 mg, 0.630 mmol), Xphos Pd G4 (27.2 mg, 0.032 mmol) and tripotassium phosphate (0.473 mL, 0.946 mmol). The mixture was stirred at 105° C. for 1 h. Water was added and extracted twice with EtOAc. The organic layer was concentrated. The residue was purified via preparative reverse phase chromatography with the following conditions: Column: XBridge C18, 19 mm×200 mm, 5 m particles; Flow Rate: 20 mL/min; Column Temperature: 25° C. Fraction collection was triggered by MS (ESI+). Fractions containing the desired product were combined and dried via centrifugal evaporation.

The material was further purified via preparative SFC with the following conditions: Column: Chiralpak AD, 30 mm×250 mm, 5 m particles; Flow Rate: 100.00 mL/min; Column Temperature: 50° C. Fraction collection was triggered by PDA (220 nm) and MS (ESI+). Fractions containing the desired product were combined and dried via centrifugal evaporation. Gradient: Time(min) Mobile Phase A (C₀₂) Mobile Phase B (MeOH/ACN (50:50)): 20.00 55% 45%. The first eluting isomer 122 5-(3-(2-amino-[1,2,4]triazolo[1,5-a]pyridin-7-yl)-2,6-difluorophenoxy)-3,3-difluoro-2-(3-fluorophenyl)pentan-2-ol (25.4 mg, 0.053 mmol, 16.66% yield), and the second eluting isomer 123 5-(3-(2-amino-[1,2,4]triazolo[1,5-a]pyridin-7-yl)-2,6-difluorophenoxy)-3,3-difluoro-2-(3-fluorophenyl)pentan-2-ol (25.6 mg, 0.054 mmol, 16.98% yield) were obtained.

### Example 122: 5-(3-(2-amino-[1,2,4]triazolo[1,5-a]pyridin-7-yl)-2,6-difluorophenoxy)-3,3-difluoro-2-(3-fluorophenyl)pentan-2-ol, enantiomer 1

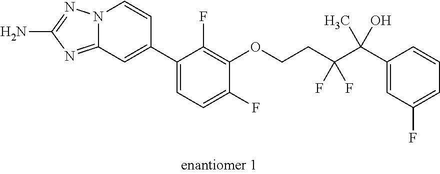 [SMILES: CC(O)(c1cccc(F)c1)C(F)(F)CCOc1c(F)ccc(-c2ccn3nc(N)nc3c2)c1F]

122, ¹H NMR (500 MHz, DMSO-d₆) δ 8.60 (d, J=6.9 Hz, 1H), 7.47 (s, 1H), 7.44-7.28 (m, 4H), 7.25 (br t, J=9.5 Hz, 1H), 7.16-7.11 (m, 1H), 7.01 (br d, J=6.9 Hz, 1H), 6.07 (s, 2H), 4.33-4.22 (m, 2H), 2.49-2.37 (m, 1H), 2.16-2.02 (m, 1H), 1.60 (s, 3H).

MS ESI m/z 478.9 (M+H)⁺.

### Example 123: 5-(3-(2-amino-[1,2,4]triazolo[1,5-a]pyridin-7-yl)-2,6-difluorophenoxy)-3,3-difluoro-2-(3-fluorophenyl)pentan-2-ol, enantiomer 2

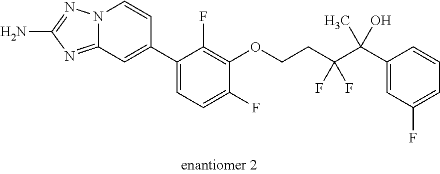 [SMILES: CC(O)(c1cccc(F)c1)C(F)(F)CCOc1c(F)ccc(-c2ccn3nc(N)nc3c2)c1F]

123, ¹H NMR (500 MHz, DMSO-d₆) δ 8.60 (br d, J=6.8 Hz, 1H), 7.48 (br s, 1H), 7.44-7.28 (m, 4H), 7.25 (br t, J=8.9 Hz, 1H), 7.17-7.11 (m, 1H), 7.01 (br d, J=6.9 Hz, 1H), 6.07 (br s, 2H), 4.33-4.21 (m, 2H), 2.48-2.37 (m, 1H), 2.17-2.01 (m, 1H), 1.60 (s, 3H).

MS ESI m/z 479.3 (M+H)⁺.

### Example 124: 5-(3-(2-amino-[1,2,4]triazolo[1,5-a]pyridin-7-yl)-2,6-difluorophenoxy)-3,3-difluoro-2-(2-fluorophenyl)pentan-2-ol, enantiomer 1

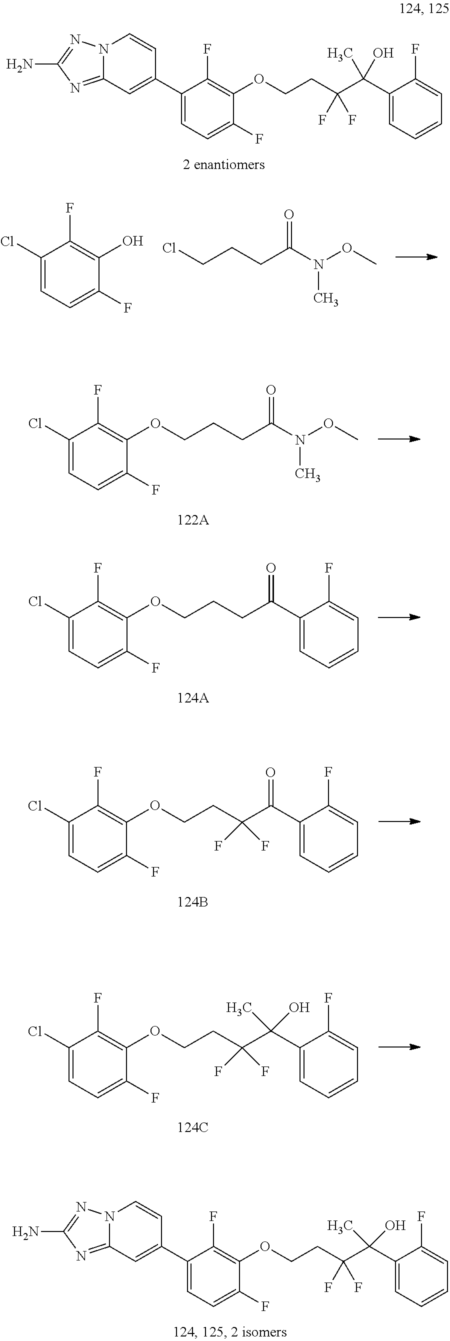

124 and 125 were prepared in a similar fashion to 122 and 123.

124A: ¹H NMR (499 MHz, chloroform-d) δ 7.89 (td, J=7.6, 1.8 Hz, 1H), 7.56-7.50 (m, 1H), 7.25 (td, J=7.6, 1.0 Hz, 1H), 7.16 (ddd, J=11.2, 8.3, 0.9 Hz, 1H), 7.04 (ddd, J=9.1, 7.7, 5.3 Hz, 1H), 6.85 (ddd, J=10.0, 9.2, 2.2 Hz, 1H), 4.27 (t, J=6.1 Hz, 2H), 3.27 (td, J=7.1, 3.1 Hz, 2H), 2.22 (quin, J=6.6 Hz, 2H).

MS ESI m/z 328.9 (M+H)⁺.

124B: ¹H NMR (499 MHz, chloroform-d) δ 7.87-7.82 (m, 1H), 7.64-7.58 (m, 1H), 7.31-7.26 (m, 1H), 7.21 (ddd, J=10.8, 8.4, 1.0 Hz, 1H), 7.08 (ddd, J=9.1, 7.6, 5.2 Hz, 1H), 6.88 (ddd, J=10.0, 9.2, 2.2 Hz, 1H), 4.45 (s, 2H), 2.80 (tt, J=16.7, 6.7 Hz, 2H). 124C: ¹H NMR (499 MHz, chloroform-d) δ 7.64 (td, J=8.0, 1.6 Hz, 1H), 7.38-7.33 (m, 1H), 7.22-7.18 (m, 1H), 7.12-7.03 (m, 2H), 6.86 (ddd, J=9.9, 9.2, 2.2 Hz, 1H), 4.42-4.32 (m, 2H), 2.92 (d, J=6.7 Hz, 1H), 2.61-2.47 (m, 1H), 2.44-2.29 (m, 1H), 1.85 (d, J=1.2 Hz, 3H).

### Example 124: 5-(3-(2-amino-[1,2,4]triazolo[1,5-a]pyridin-7-yl)-2,6-difluorophenoxy)-3,3-difluoro-2-(2-fluorophenyl)pentan-2-ol, enantiomer 1

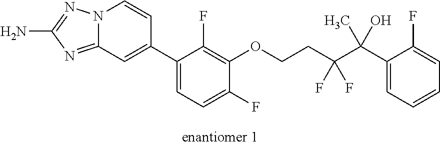 [SMILES: CC(O)(c1ccccc1F)C(F)(F)CCOc1c(F)ccc(-c2ccn3nc(N)nc3c2)c1F]

124: the first eluting isomer, ¹H NMR (500 MHz, DMSO-d₆) δ 8.61 (br d, J=6.9 Hz, 1H), 7.77-7.71 (m, 1H), 7.48 (s, 1H), 7.41-7.35 (m, 2H), 7.29-7.20 (m, 2H), 7.15 (br dd, J=11.7, 8.9 Hz, 1H), 7.02 (br d, J=6.6 Hz, 1H), 6.28 (s, 1H), 6.07 (s, 2H), 4.36-4.24 (m, 2H), 2.49-2.41 (m, 1H), 2.26-2.13 (m, 1H), 1.73 (br s, 3H).

MS ESI m/z 478.9 (M+H)⁺.

### Example 125: 5-(3-(2-amino-[1,2,4]triazolo[1,5-a]pyridin-7-yl)-2,6-difluorophenoxy)-3,3-difluoro-2-(2-fluorophenyl)pentan-2-ol, enantiomer 2

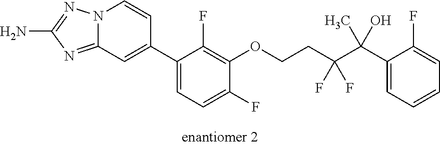 [SMILES: CC(O)(c1ccccc1F)C(F)(F)CCOc1c(F)ccc(-c2ccn3nc(N)nc3c2)c1F]

125: the second eluting isomer, ¹H NMR (500 MHz, DMSO-d₆) δ 8.61 (d, J=6.7 Hz, 1H), 7.74 (br t, J=7.7 Hz, 1H), 7.48 (s, 1H), 7.41-7.35 (m, 2H), 7.29-7.20 (m, 2H), 7.15 (dd, J=12.2, 8.6 Hz, 1H), 7.02 (br d, J=6.9 Hz, 1H), 6.07 (s, 2H), 4.35-4.24 (m, 2H), 2.49-2.43 (m, 1H), 2.25-2.12 (m, 1H), 1.73 (br s, 3H).

MS ESI m/z 479.1 (M+H)⁺.

### Example 126: 5-(3-(2-amino-[1,2,4]triazolo[1,5-a]pyridin-7-yl)-2,6-difluorophenoxy)-3,3-difluoro-2-(4-fluorophenyl)hexan-2-ol, isomer 1

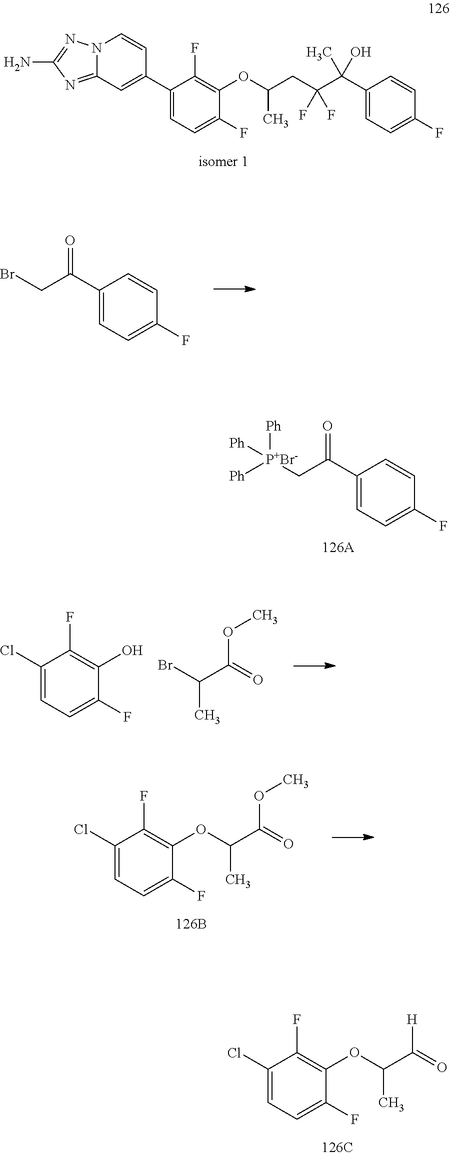

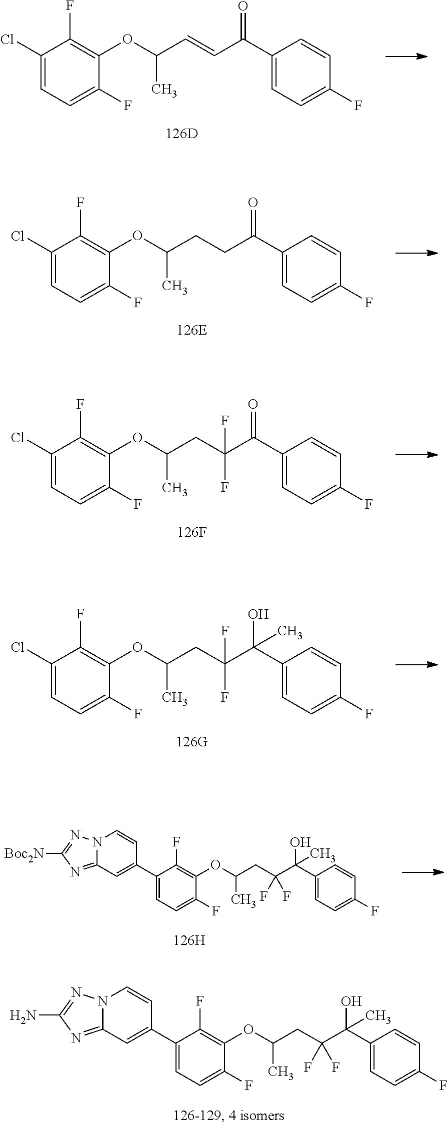

126A: To a solution of 2-bromo-1-(4-fluorophenyl)ethan-1-one (2.16 g, 9.95 mmol) in DCM (30 mL) was added Ph₃P (2.61 g, 9.95 mmol). The mixture was stirred at rt for 2 days. The solid was filtered out, washed with Et₂O and air dried to give (2-(4-fluorophenyl)-2-oxoethyl)-triphenylphosphonium bromide (4.7 g, 9.81 mmol, 99% yield).

126B: To a solution of 3-chloro-2,6-difluorophenol (500 mg, 3.04 mmol) in DMF (10 mL) was added K₂CO₃ (840 mg, 6.08 mmol) followed by methyl 2-bromopropanoate (1522 mg, 9.12 mmol). The mixture was stirred at rt for 2 h. EtOAc was added, washed with 3× water, then concentrated. The residue was purified via silica gel chromatography (24 g, hexanes-40% EtOAc) to give methyl 2-(3-chloro-2,6-difluorophenoxy)propanoate (740 mg, 2.95 mmol, 97% yield).

¹H NMR (499 MHz, chloroform-d) δ 7.09 (ddd, J=9.2, 7.6, 5.1 Hz, 1H), 6.88 (ddd, J=10.1, 9.2, 2.2 Hz, 1H), 4.86 (q, J=6.9 Hz, 1H), 3.80 (s, 3H), 1.66 (d, J=6.8 Hz, 3H).

MS ESI m/z 251.1 (M+H)⁺.

126C: To a solution of methyl 2-(3-chloro-2,6-difluorophenoxy)propanoate (740 mg, 2.95 mmol) in DCM (10 mL) was added 1.0 M DIBAL-H/THF (3.54 mL, 3.54 mmol) at −78° C. under N₂. The mixture was stirred at −78° C. for 30 min. The reaction was quenched with 0.5 mL water at −78° C. Et₂O was added. The mixture was stirred at rt for 1 h, then dried over Na₂SO₄. The solid was filtered out. The filtrate was concentrated to give crude 2-(3-chloro-2,6-difluorophenoxy)propanal (600 mg, 2.72 mmol, 92% yield). The material was used right away as was for the next step.

126D: To a solution of (2-(4-fluorophenyl)-2-oxoethyl)triphenylphosphonium bromide (400 mg, 0.834 mmol) in DCM (3 mL) was added NaOH (1.669 mL, 1.669 mmol). The mixture was stirred at rt for 15 min, then extracted with 3× DCM. The organic layer was dried over Na₂SO₄, then concentrated to about 3 mL. The crude 2-(3-chloro-2,6-difluorophenoxy)propanal (460 mg, 2.086 mmol) was added. The mixture was stirred at 38° C. for 18 h. After concentration, the residue was purified via silica gel chromatography (24 g, hexanes-40% EtOAc) to give (E)-4-(3-chloro-2,6-difluorophenoxy)-1-(4-fluorophenyl)pent-2-en-1-one (280 mg, 0.822 mmol, 98% yield).

MS ESI m/z 341.0 (M+H)⁺.

126E: To a solution of (E)-4-(3-chloro-2,6-difluorophenoxy)-1-(4-fluorophenyl)pent-2-en-1-one (280 mg, 0.822 mmol) in MeOH (3 mL) was added 10% wt Pd—C (45 mg, 0.042 mmol) followed by diphenylsulfane (1.531 mg, 8.22 μmol) under N₂. The mixture was stirred under a H₂ balloon at rt for 2.5 h. The solid was filtered out. The filtrate was concentrated. The residue was purified via silica gel chromatography (12 g, hexanes-40% EtOAc) to give clean 4-(3-chloro-2,6-difluoro-phenoxy)-1-(4-fluorophenyl)pentan-1-one (110 mg, 0.321 mmol, 39.1% yield).

¹H NMR (499 MHz, chloroform-d) δ 8.07-8.02 (m, 2H), 7.18-7.12 (m, 2H), 7.06 (ddd, J=9.1, 7.7, 5.3 Hz, 1H), 6.90-6.83 (m, 1H), 4.46-4.39 (m, 1H), 3.28 (t, J=7.3 Hz, 2H), 2.22-2.08 (m, 2H), 1.35 (d, J=6.2 Hz, 3H).

MS ESI m/z 343.0 (M+H)⁺.

126F: To a solution of 4-(3-chloro-2,6-difluorophenoxy)-1-(4-fluorophenyl)pentan-1-one (110 mg, 0.321 mmol) in THF (1.0 mL) was added LHMDS (1.284 mL, 1.284 mmol) at −10° C. under N₂. The mixture was stirred at −10° C. to 0° C. for 20 min, then N-fluorobenzenesulfonimide (405 mg, 1.284 mmol) was added at −10° C. The mixture was stirred from −10° C. to 0° C. over 30 min. The reaction was quenched with 0.2N NaOH (1.685 mL, 0.337 mmol). Et₂O was added. The mixture was washed with 0.2 N NaOH and 2× water, then concentrated. The residue was purified via silica gel chromatography (12 g, hexanes-10% EtOAc) to give desired 4-(3-chloro-2,6-difluoro-phenoxy)-2,2-difluoro-1-(4-fluorophenyl)pentan-1-one (73 mg, 0.193 mmol, 60.1% yield).

¹H NMR (499 MHz, chloroform-d) δ 8.16 (dd, J=8.9, 5.4 Hz, 2H), 7.21-7.16 (m, 2H), 7.07 (ddd, J=9.1, 7.6, 5.2 Hz, 1H), 6.86 (ddd, J=9.9, 9.2, 2.2 Hz, 1H), 4.76 (sxt, J=6.1 Hz, 1H), 2.92-2.79 (m, 1H), 2.61-2.50 (m, 1H), 1.44 (d, J=6.2 Hz, 3H).

126G: To a solution of 4-(3-chloro-2,6-difluorophenoxy)-2,2-difluoro-1-(4-fluorophenyl)pentan-1-one (73 mg, 0.193 mmol) in THF (1.5 mL) was added 3.0 M methylmagnesium bromide/Et₂O (0.129 mL, 0.386 mmol) at −10° C. under N₂. The mixture was stirred from −10° C. to rt for 30 min. The reaction was quenched with NH₄Cl/water.

Et₂O was added. The mixture was washed with 2× water, then concentrated. The residue was purified via silica gel chromatography (12 g, hexanes-40% EtOAc) to give 5-(3-chloro-2,6-difluorophenoxy)-3,3-difluoro-2-(4-fluorophenyl)hexan-2-ol (72 mg, 0.182 mmol, 95% yield).

MS ESI m/z 395.1 (M+H)⁺.

126H: To a solution of 5-(3-chloro-2,6-difluorophenoxy)-3,3-difluoro-2-(4-fluorophenyl)hexan-2-ol (70 mg, 0.177 mmol) in dioxane (1. mL) was added the intermediate tert-butyl (tert-butoxycarbonyl)(7-(4,4,5,5-tetramethyl-1,3,2-dioxaborolan-2-yl)-[1,2,4]triazolo[1,5-a]pyridin-2-yl)carbamate (90 mg, 0.195 mmol), XPhos Pd G2 (6.98 mg, 8.87 μmol) and tripotassium phosphate (0.266 mL, 0.532 mmol). The mixture was stirred at 75° C. for 40 min. EtOAc was added. The mixture was washed with 2× water, then concentrated to give the crude product (120 mg, 0.173 mmol, 98% yield).

MS ESI m/z 693.3 (M+H)⁺.

### 126-129

To a solution of 126H (120 mg, 0.173 mmol) in DCM (1.0 mL) was added TFA (0.3 mL, 3.89 mmol) at rt. The mixture was stirred at rt for 2 h. The mixture was concentrated. The residue was purified via preparative reverse phase chromatography with the following conditions: Column: XBridge C18, 19 mm×200 mm, 5 m particles; Flow Rate: 20 mL/min; Column Temperature: 25° C. Fraction collection was triggered by UV (220 nm) and MS (ESI+). Fractions containing the desired product were combined and dried via centrifugal evaporation.

The material was further purified via preparative SFC with the following conditions: Column: Chiralpak AD, 30 mm×250 mm, 5 m particles; Flow Rate:100.00 mL/min; Column Temperature: 50° C. Fraction collection was triggered by PDA (220 nm) and MS (ESI+). Fractions containing the desired product were combined and dried via centrifugal evaporation. Gradient: Time(min) Mobile Phase A (CO2) Mobile Phase B (MeOH/ACN (50:50)), 45.00 70% 30%. Four isomers were obtained.

### Example 126: 5-(3-(2-amino-[1,2,4]triazolo[1,5-a]pyridin-7-yl)-2,6-difluorophenoxy)-3,3-difluoro-2-(4-fluorophenyl)hexan-2-ol, isomer 1

The first eluting isomer 126: ¹H NMR (500 MHz, DMSO-d₆) δ 8.58 (d, J=7.1 Hz, 1H), 7.51 (br dd, J=7.7, 5.8 Hz, 2H), 7.45 (s, 1H), 7.38-7.33 (m, 1H), 7.22 (br t, J=9.3 Hz, 1H), 7.12 (br t, J=8.8 Hz, 2H), 6.99 (br d, J=6.9 Hz, 1H), 4.54-4.47 (m, 1H), 2.38-2.23 (m, 1H), 2.13-2.00 (m, 1H), 1.56 (s, 3H), 1.30 (br d, J=6.2 Hz, 3H).

MS ESI m/z 492.9 (M+H)⁺.

### Example 127: 5-(3-(2-amino-[1,2,4]triazolo[1,5-a]pyridin-7-yl)-2,6-difluorophenoxy)-3,3-difluoro-2-(4-fluorophenyl)hexan-2-ol, isomer 2

The second eluting isomer 127, ¹H NMR (500 MHz, DMSO-d₆) δ 8.59 (d, J=6.9 Hz, 1H), 7.53-7.45 (m, 3H), 7.42-7.35 (m, 1H), 7.25 (br t, J=9.4 Hz, 1H), 7.12 (t, J=8.9 Hz, 2H), 7.01 (br d, J=7.2 Hz, 1H), 6.21 (s, 1H), 6.06 (s, 2H), 4.60-4.53 (m, 1H), 2.49-2.37 (m, 1H), 1.94-1.81 (m, 1H), 1.56 (s, 3H), 1.28 (br d, J=6.0 Hz, 3H).

MS ESI m/z 492.9 (M+H)⁺.

### Example 128: 5-(3-(2-amino-[1,2,4]triazolo[1,5-a]pyridin-7-yl)-2,6-difluorophenoxy)-3,3-difluoro-2-(4-fluorophenyl)hexan-2-ol, isomer 3

The third eluting isomer 128, ¹H NMR (500 MHz, DMSO-d₆) δ 8.60 (d, J=6.9 Hz, 1H), 7.52-7.46 (m, 3H), 7.42-7.36 (m, 1H), 7.25 (t, J=9.5 Hz, 1H), 7.12 (t, J=8.9 Hz, 2H), 7.01 (br d, J=6.9 Hz, 1H), 6.06 (s, 2H), 4.60-4.53 (m, 1H), 2.49-2.38 (m, 1H), 1.94-1.82 (m, 1H), 1.56 (s, 3H), 1.28 (d, J=6.0 Hz, 3H).

MS ESI m/z 493.1 (M+H)⁺.

### Example 129: 5-(3-(2-amino-[1,2,4]triazolo[1,5-a]pyridin-7-yl)-2,6-difluorophenoxy)-3,3-difluoro-2-(4-fluorophenyl)hexan-2-ol, isomer 4

The fourth eluting isomer 129, ¹H NMR (500 MHz, DMSO-d₆) δ 8.58 (br d, J=6.9 Hz, 1H), 7.54-7.47 (m, 2H), 7.44 (s, 1H), 7.38-7.32 (m, 1H), 7.25-7.19 (m, 1H), 7.12 (br t, J=8.9 Hz, 2H), 6.99 (br d, J=6.8 Hz, 1H), 6.05 (s, 2H), 4.53-4.47 (m, 1H), 2.38-2.24 (m, 1H), 2.13-1.99 (m, 1H), 1.56 (s, 3H), 1.30 (br d, J=6.1 Hz, 3H).

MS ESI m/z 492.9 (M+H)⁺.

### Example 130: 5-(3-(2-amino-[1,2,4]triazolo[1,5-a]pyridin-7-yl)-2,6-difluorophenoxy)-3,3-difluoro-2-(4-fluorophenyl)-5-methylhexan-2-ol, enantiomer 1

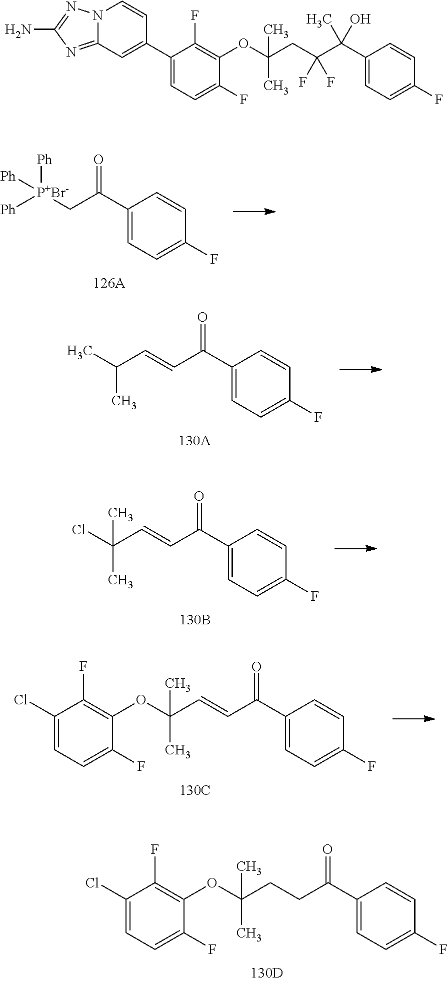

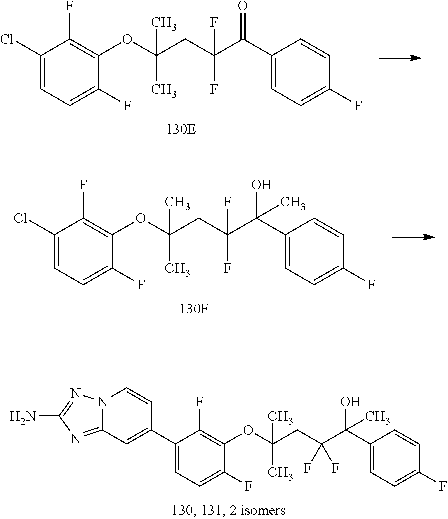 [SMILES: CC(C)(CC(F)(F)C(=O)c1ccc(F)cc1)Oc1c(F)ccc(Cl)c1F ; CC(C)(CC(F)(F)C(C)(O)c1ccc(F)cc1)Oc1c(F)ccc(Cl)c1F ; CC(C)(CC(F)(F)C(C)(O)c1ccc(F)cc1)Oc1c(F)ccc(-c2ccn3nc(N)nc3c2)c1F]

130A: To a solution of (2-(4-fluorophenyl)-2-oxoethyl)triphenylphosphonium bromide (10.55 g, 22 mmol) in DCM (50 mL) was added NaOH (44.0 mL, 44.0 mmol). The mixture was stirred at rt for 1 h, then extracted with 3× 50 mL of DCM. The organic layer was dried over Na₂SO₄, then concentrated to about 50 mL. Then isobutyraldehyde (7.93 g, 110 mmol) was added. The mixture was stirred at rt for 3 days. The mixture was concentrated. The residue was purified via silica gel chromatography (40 g, hexanes-40% EtOAc) to give (E)-1-(4-fluorophenyl)-4-methylpent-2-en-1-one (3.4 g, 17.69 mmol, 80% yield).

¹H NMR (499 MHz, chloroform-d) δ 8.01-7.96 (m, 2H), 7.19-7.14 (m, 2H), 7.06 (dd, J=15.4, 6.7 Hz, 1H), 6.82 (dd, J=15.4, 1.4 Hz, 1H), 2.60 (ddd, J=13.5, 6.7, 1.4 Hz, 1H), 1.16 (d, J=6.8 Hz, 6H).

MS ESI m/z 193.2 (M+H)⁺.

130B: To a solution of (E)-1-(4-fluorophenyl)-4-methylpent-2-en-1-one (3.4 g, 17.69 mmol) in MeCN (60 mL) was added copper(I) trifluoromethanesulfonate toluene complex (1.098 g, 2.122 mmol), N,N-dichloro-4-methylbenzenesulfonamide (5.10 g, 21.22 mmol), and sodium bicarbonate (2.229 g, 26.5 mmol). The mixture was stirred at rt for 18 h, then filtered. Water was added to the filtrate and extracted with 2× EtOAc. The organic layer was concentrated. The residue was purified via silica gel chromatography (40 g, hexanes-40% EtOAc) to give 2.4 g of crude (E)-4-chloro-1-(4-fluorophenyl)-4-methylpent-2-en-1-one.

130C: To a solution of the crude (E)-4-chloro-1-(4-fluorophenyl)-4-methylpent-2-en-1-one (200 mg, 0.882 mmol) and 3-chloro-2,6-difluorophenol (218 mg, 1.323 mmol) in DMSO (3 mL) was added K₂CO₃ (610 mg, 4.41 mmol) and potassium iodide (439 mg, 2.65 mmol). The mixture was stirred at 40° C. for 20 h and at 60° C. for 6 h. EtOAc was added. The mixture was washed with 3× water, then concentrated. The residue was purified via silica gel chromatography (24 g, hexanes-20% EtOAc) to give desired (E)-4-(3-chloro-2,6-difluorophenoxy)-1-(4-fluorophenyl)-4-methyl-pent-2-en-1-one (60 mg, 0.169 mmol, 19.17% yield).

¹H NMR (499 MHz, chloroform-d) δ 8.02-7.98 (m, 2H), 7.20-7.11 (m, 5H), 6.91 (td, J=9.3, 2.2 Hz, 1H), 1.57 (s, 6H).

MS ESI m/z 355.0 (M+H)⁺.

130D: prepared in a similar fashion to 126E.

¹H NMR (499 MHz, CHLOROFORM-d) δ 8.10-8.06 (m, 2H), 7.19-7.09 (m, 3H), 6.89 (td, J=9.4, 2.2 Hz, 1H), 3.35-3.30 (m, 2H), 2.24-2.19 (m, 2H), 1.38 (s, 6H).

130E: prepared in a similar fashion to 126F.

¹H NMR (499 MHz, CHLOROFORM-d) δ 8.17-8.11 (m, 2H), 7.18-7.08 (m, 3H), 6.85 (td, J=9.3, 2.2 Hz, 1H), 2.83 (t, J=18.4 Hz, 2H), 1.48 (s, 6H).

130F: prepared in a similar fashion to 126G.

¹H NMR (499 MHz, CHLOROFORM-d) δ 7.59-7.53 (m, 2H), 7.14-7.04 (m, 3H), 6.90-6.79 (m, 1H), 2.66-2.53 (m, 1H), 2.48 (s, 1H), 2.04-1.91 (m, 1H), 1.72 (s, 3H), 1.47-1.37 (m, 6H). 130 and 131 were prepared from 130F in a similar fashion to 122 and 123 from 122D.

### Example 130: 5-(3-(2-amino-[1,2,4]triazolo[1,5-a]pyridin-7-yl)-2,6-difluorophenoxy)-3,3-difluoro-2-(4-fluorophenyl)-5-methylhexan-2-ol, enantiomer 1

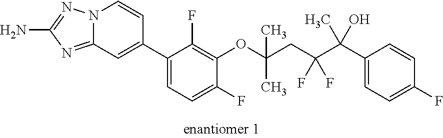 [SMILES: CC(C)(CC(F)(F)C(C)(O)c1ccc(F)cc1)Oc1c(F)ccc(-c2ccn3nc(N)nc3c2)c1F]

90: the first eluting isomer, ¹H NMR (500 MHz, DMSO-d₆) δ 8.59 (d, J=6.8 Hz, 1H), 7.57-7.51 (m, 2H), 7.46 (s, 1H), 7.44-7.38 (m, 1H), 7.24 (br t, J=9.0 Hz, 1H), 7.15 (br t, J=8.8 Hz, 2H), 6.99 (br d, J=6.5 Hz, 1H), 6.20 (s, 1H), 6.06 (s, 2H), 2.61-2.55 (m, 1H), 2.06-1.90 (m, 1H), 1.58 (s, 3H), 1.38 (s, 3H), 1.32 (s, 3H).

MS ESI m/z 507.2 (M+H)⁺.

### Example 131: 5-(3-(2-amino-[1,2,4]triazolo[1,5-a]pyridin-7-yl)-2,6-difluorophenoxy)-3,3-difluoro-2-(4-fluorophenyl)-5-methylhexan-2-ol, enantiomer 2

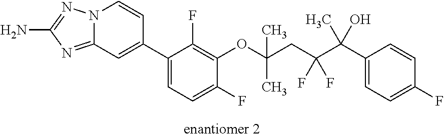 [SMILES: CC(C)(CC(F)(F)C(C)(O)c1ccc(F)cc1)Oc1c(F)ccc(-c2ccn3nc(N)nc3c2)c1F]

131: the second eluting isomer, ¹H NMR (500 MHz, DMSO-d₆) δ 8.59 (d, J=6.7 Hz, 1H), 7.57-7.51 (m, 2H), 7.46 (s, 1H), 7.44-7.38 (m, 1H), 7.24 (br t, J=9.5 Hz, 1H), 7.15 (br t, J=8.6 Hz, 2H), 6.99 (br d, J=6.6 Hz, 1H), 6.20 (s, 1H), 6.06 (s, 2H), 2.61-2.56 (m, 1H), 2.05-1.92 (m, 1H), 1.58 (s, 3H), 1.38 (s, 3H), 1.32 (br s, 3H).

MS ESI m/z 507.2 (M+H)⁺.

Numerous modifications and variations of the present invention are possible in light of the above teachings. It is therefore to be understood that within the scope of the appended claims, the invention may be practiced otherwise than as specifically described herein.

1-17. (canceled)

18. A compound having the structure 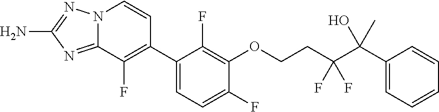 [SMILES: CC(O)(c1ccccc1)C(F)(F)CCOc1c(F)ccc(-c2ccn3nc(N)nc3c2F)c1F]
or stereoisomer or salt thereof.

19. A compound having the structure 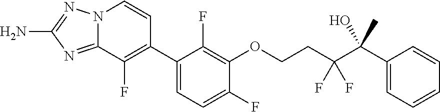 [SMILES: C[C@@](O)(c1ccccc1)C(F)(F)CCOc1c(F)ccc(-c2ccn3nc(N)nc3c2F)c1F]
or salt thereof.

20. A compound having the structure

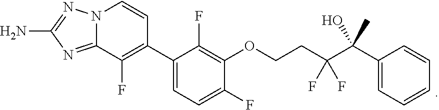 [SMILES: C[C@@](O)(c1ccccc1)C(F)(F)CCOc1c(F)ccc(-c2ccn3nc(N)nc3c2F)c1F]

21. A salt of a compound having the structure

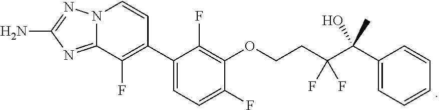 [SMILES: C[C@@](O)(c1ccccc1)C(F)(F)CCOc1c(F)ccc(-c2ccn3nc(N)nc3c2F)c1F]

22. A pharmaceutical composition comprising the compound of claim 19, or pharmaceutically acceptable salt thereof, and a pharmaceutically acceptable carrier.

23. A method of inhibiting casein kinase RIPK1 activity in a patient, comprising administering to the patient in need thereof, a therapeutically effective amount of the compound of claim 19.

24. A method for treating a disease comprising the administration to a subject in need thereof a therapeutically-effective amount of the compound of claim 19, or pharmaceutically acceptable salt thereof, wherein the disease is selected from inflammatory bowel disease, ulcerative colitis, Crohn's disease, psoriasis, rheumatoid arthritis (RA), NASH, and heart failure.

25. A method for treating a disease comprising the administration to a subject in need thereof a therapeutically-effective amount of the compound of claim 19, or pharmaceutically acceptable salt thereof, wherein the disease is selected from multiple sclerosis, amyotrophic lateral sclerosis, or Alzheimers.

26. A method for treating a disease comprising the administration to a subject in need thereof a therapeutically-effective amount of the compound of claim 19, or pharmaceutically acceptable salt thereof, wherein the disease is selected from multiple sclerosis.

27. A method for treating a disease comprising the administration to a subject in need thereof a therapeutically-effective amount of the compound of claim 19, or pharmaceutically acceptable salt thereof, wherein the disease is selected from amyotrophic lateral sclerosis.

28. A method for treating a disease comprising the administration to a subject in need thereof a therapeutically-effective amount of the compound of claim 19, or pharmaceutically acceptable salt thereof, wherein the disease is selected from Alzheimers.

29. A pharmaceutical composition comprising the compound of claim 20, and a pharmaceutically acceptable carrier.

30. A method of inhibiting casein kinase RIPK1 activity in a patient, comprising administering to the patient in need thereof, a therapeutically effective amount of the compounds according to claim 20.

31. A method for treating a disease comprising the administration to a subject in need thereof a therapeutically-effective amount of the compound of claim 20, wherein the disease is selected from inflammatory bowel disease, ulcerative colitis, Crohn's disease, psoriasis, rheumatoid arthritis (RA), NASH, and heart failure.

32. A method for treating a disease comprising the administration to a subject in need thereof a therapeutically-effective amount of the compound of claim 20, wherein the disease is selected from multiple sclerosis, amyotrophic lateral sclerosis, or Alzheimers.

33. A method for treating a disease comprising the administration to a subject in need thereof a therapeutically-effective amount of the compound of claim 20, wherein the disease is selected from multiple sclerosis.

34. A method for treating a disease comprising the administration to a subject in need thereof a therapeutically-effective amount of the compound of claim 20, wherein the disease is selected from amyotrophic lateral sclerosis.

35. A method for treating a disease comprising the administration to a subject in need thereof a therapeutically-effective amount of the compound of claim 20, wherein the disease is selected from Alzheimers.

36. A pharmaceutical composition comprising a pharmaceutically acceptable salt of the compound of claim 21, and a pharmaceutically acceptable carrier.

37. A method of inhibiting casein kinase RIPK1 activity in a patient, comprising administering to the patient in need thereof, a therapeutically effective amount of a pharmaceutically acceptable salt of the compound according to claim 21.

38. A method for treating a disease comprising the administration to a subject in need thereof a therapeutically-effective amount of a pharmaceutically acceptable salt of the compound of claim 21, wherein the disease is selected from inflammatory bowel disease, ulcerative colitis, Crohn's disease, psoriasis, rheumatoid arthritis (RA), NASH, and heart failure.

39. A method for treating a disease comprising the administration to a subject in need thereof a therapeutically-effective amount of a pharmaceutically acceptable salt of the compound of claim 21, wherein the disease is selected from multiple sclerosis, amyotrophic lateral sclerosis, or Alzheimers.

40. A method for treating a disease comprising the administration to a subject in need thereof a therapeutically-effective amount of a pharmaceutically acceptable salt of the compound of claim 21, wherein the disease is selected from multiple sclerosis.

41. A method for treating a disease comprising the administration to a subject in need thereof a therapeutically-effective amount of a pharmaceutically acceptable salt of the compound of claim 21, wherein the disease is selected from amyotrophic lateral sclerosis.

42. A method for treating a disease comprising the administration to a subject in need thereof a therapeutically-effective amount of a pharmaceutically acceptable salt of the compound of claim 21, wherein the disease is selected from Alzheimers.
